(19) United States
(12) Patent Application Publication
(10) Pub. No.: US 2012/0237987 A1
(43) Pub. Date: Sep. 20, 2012

(54) BACTERIUM FOR PRODUCTION OF FATTY ACIDS

(75) Inventors: Roy Curtiss, III, Paradise Valley, AZ (US); Xinyao Liu, Chandler, AZ (US)

(73) Assignee: The Arizona Board of Regents for and on behalf of Arizona State University, Tempe, AZ (US)

(21) Appl. No.: 13/459,774

(22) Filed: Apr. 30, 2012

Related U.S. Application Data
(63) Continuation-in-part of application No. PCT/US2010/054494, filed on Oct. 28, 2010.
(60) Provisional application No. 61/255,729, filed on Oct. 28, 2009.
(60) Provisional application No. 61/307,185, filed on Feb. 23, 2010.
(60) Provisional application No. 61/382,156, filed on Sep. 13, 2010.

(51) Int. Cl. C12N 1/13; C12P 7/64

(52) U.S. Cl. 435/134; 435/257.2

(57) ABSTRACT

The present invention encompasses a bacterium that produces fatty acids.

### CROSS REFERENCE TO RELATED APPLICATIONS

This application is a Continuation in Part of PCT application PCT/US2010/054494, filed Oct. 28, 2010, which claims the priority of U.S. provisional application No. 61/255,729, filed Oct. 28, 2009, U.S. provisional application No. 61/307,185, filed Feb. 23, 2010, and U.S. provisional application No. 61/382,156, filed Sep. 13, 2010, each of which is hereby incorporated by reference in its entirety.

### FIELD OF THE INVENTION

The invention encompasses a recombinant bacterium for the production of fatty acids. In particular, the invention encompasses a bacterium capable of both generating fatty acids and releasing the fatty acids into the culture medium.

### BACKGROUND OF THE INVENTION

Cheap renewable energy is a major goal of many countries and hardly needs a justification. Not only does renewable energy provide energy independence and security but can often provide carbon neutral or better clean energy. But not all forms of renewable energy are equal. Currently wind, solar, hydro and geothermal power are excellent supplements to the power grid, but cannot be efficiently converted into liquid biofuels needed for our cars, ships and planes. There will be a high demand for biodiesel and other liquid biofuels into the foreseeable future.

Liquid biofuels can be roughly classified by the source (feedstock) and production methods. First generation biofuels like ethanol from corn are now established industries, but the trend is to move away from using food crops as the primary feedstock for energy production. Second generation biofuels like cellulosic ethanol use biomass as the primary feedstock. The biomass is obtained from the “waste” left over after food crops have been processed, or from other energy crops like switchgrass, Miscanthus, and “energycane”. While eliminating the ethical dilemma of tapping into the world's food supply for fuel, cellulosic ethanol is still neither the most efficient way to produce biofuels nor a viable replacement for the majority of the nations fuel needs. Recycling biomass waste into fuel is a great idea, but growing energy crops just to harvest and digest them back into biofuel is not the most efficient use of energy, water, or arable land.

Third generation biofuels have typically been defined as fuel from photosynthetic organisms. The organisms are grown, harvested, and then the fuel precursors are extracted from the biomass. Photosynthetic microbes such as algae and cyanobacteria are the most efficient organisms for solar energy conversion, typically yielding lipids in the range of about 20-30% of dry weight. While photosynthetic microorganisms will theoretically out produce any plant based biofuel systems, costs associated with extraction continue to be a barrier to making these biofuels competitive with fossil fuels.

Hence, there is a need in the art for a microbe capable of producing fatty acids while decreasing processing and extraction costs.

### SUMMARY OF THE INVENTION

One aspect of the present invention encompasses a recombinant bacterium. The bacterium is capable of producing fatty acids and comprises at least one modified polar cell layer.

Another aspect of the present invention encompasses a recombinant bacterium. The bacterium is capable of producing fatty acids and is capable of the inducible release of fatty acids from a cellular membrane.

Another aspect of the present invention encompasses a recombinant bacterium. The bacterium is modified to encode multiple thioesterases that specify synthesis and secretion of saturated C10 to C14 chain fatty acids that are secreted more efficiently than saturated and unsaturated C16 and C18 chain fatty acids.

Another aspect of the present invention encompasses a recombinant bacterium. The bacterium is modified to encode multiple thioesterases that specify synthesis and secretion of saturated C10 to C14 chain fatty acids that are secreted more efficiently than saturated and unsaturated C16 and C18 chain fatty acids and comprises at least one modified polar cell layer.

Another aspect of the present invention encompasses a recombinant bacterium. The bacterium is modified to encode multiple thioesterases that specify synthesis and secretion of saturated C10 to C14 chain fatty acids that are secreted more efficiently than saturated and unsaturated C16 and C18 chain fatty acids and is capable of the inducible release of fatty acids from a cellular membrane.

Yet another aspect of the present invention encompasses a method of producing fatty acids. The method comprises culturing a bacterium capable of producing fatty acids and that comprises at least one modified polar cell layer.

Still another aspect of the present invention encompasses a method of producing fatty acids. The method comprises culturing a bacterium capable of producing fatty acids and that is capable of the inducible release of fatty acids from a cellular membrane.

Still another aspect of the present invention encompasses a method of producing fatty acids. The method comprises culturing a bacterium modified to encode multiple thioesterases that specify synthesis and secretion of saturated C10 to C14 chain fatty acids that are secreted more efficiently than saturated and unsaturated C16 and C18 chain fatty acids.

Still another aspect of the present invention encompasses a method of producing fatty acids. The method comprises culturing a bacterium modified to encode multiple thioesterases that specify synthesis and secretion of saturated C10 to C14 chain fatty acids that are secreted more efficiently than saturated and unsaturated C16 and C18 chain fatty acids and comprises at least one modified polar cell layer.

Still another aspect of the present invention encompasses a method of producing fatty acids. The method comprises culturing a bacterium modified to encode multiple thioesterases that specify synthesis and secretion of saturated C10 to C14 chain fatty acids that are secreted more efficiently than saturated and unsaturated C16 and C18 chain fatty acids and is capable of the inducible release of fatty acids from a cellular membrane.

Other aspects and iterations of the invention are described more thoroughly below.

### REFERENCE TO COLOR FIGURES

The application file contains at least one photograph executed in color. Copies of this patent application publication with color photographs will be provided by the Office upon request and payment of the necessary fee.

### BRIEF DESCRIPTION OF THE FIGURES

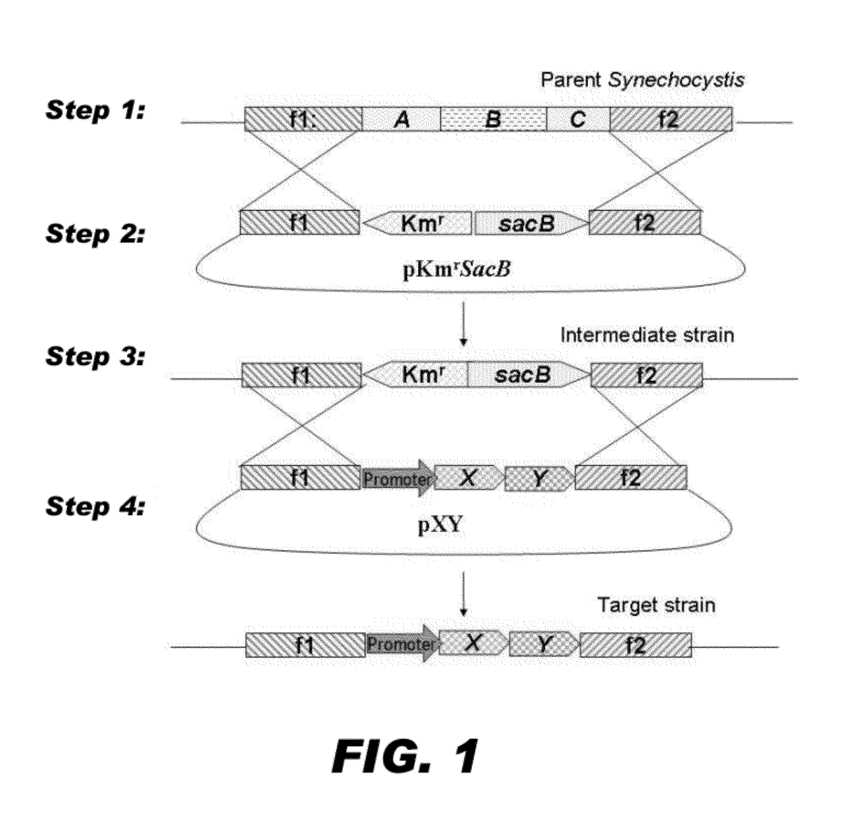

FIG. 1 depicts the recombinant strategy used in this project for genetic engineering of 6803. Step 1: Transform parent Synechocystis cells with a suicide vector containing Km^{r}-sacB. Step 2: Select for kanamycin resistance for the intermediate strain. Step 3: Transform the intermediate strain with a markerless suicide vector, pXY containing genes of interest. Step 4: Select the right recombinants on sucrose plates after segregation.

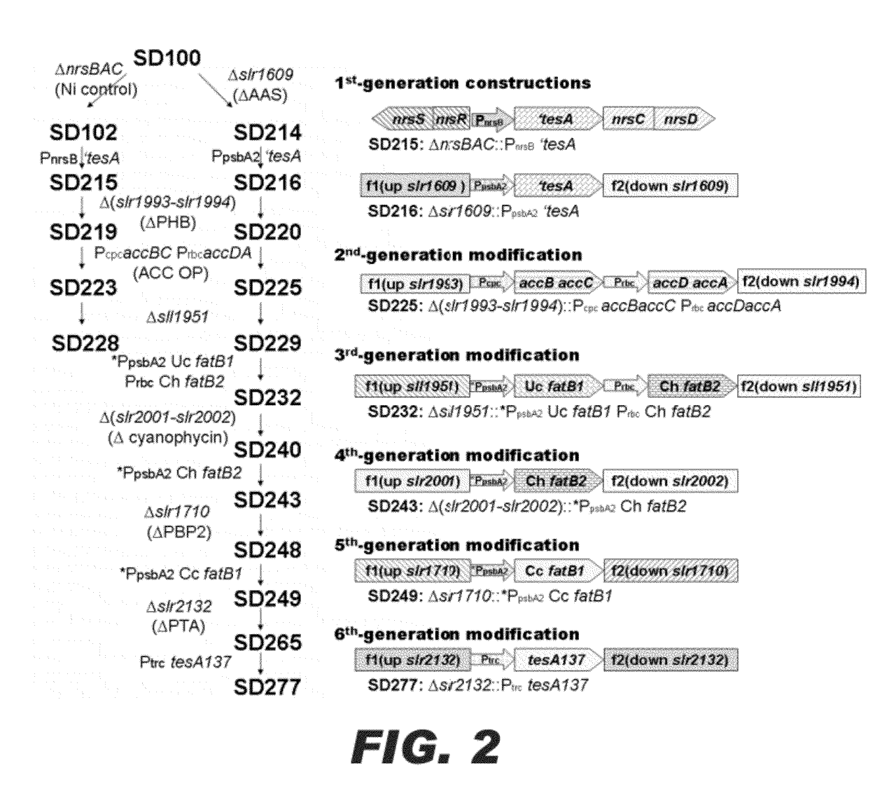

FIG. 2 depicts genetic modifications in the SD strains for FFA secretion. Six sequential genetic modifications were successively made to 6803 to increase FFA production and secretion. Their genealogy is shown in the left, and detailed modifications are shown in the right. As shown in SD215, P_{nrsB} is the nickel-inducible promoter and also serves as the upstream flanking region for 'tesA insertion, nrsCD is the downstream flanking region for the 'tesA insertion, nrsBAC are three deleted nickel resistance genes. In SD216, f1 and f2 are the upstream and downstream flanking regions (up slr1609 and down slr1609) for deletion of slr1609 (aas) and insertion of the P_{psbA2} 'tesA cassette, of which, f1 contains the residual promoter of slr1609 (Pₐₐₛ), and P_{psbA2} is the promoter of the 6803 psbA2 gene. In SD225, f1 and f2 are the flanking regions (up slr1993 and down slr1994) for deletion of slr1993 and slr1994; P_{cpc} is the promoter of the cpc operon; P_{rbc} is the promoter of the rbc operon; accB, accC, accD, and accA are the genes coding for ACC subunits. In SD232, f1 and f2 are the flanking regions (up sll1951 and down sll1951) for deletion of sll1951 encoding the surface-layer protein; *P_{psbA2} is an improved promoter from P_{psbA2}; and Uc fatB1 is a TE gene from Umbellularia californica. In SD 243, f1 and f2 are the flanking regions (up slr2001 and down slr2002) for deletion of slr2001 encoding cyanophycinase and slr2002 cyanophycin synthetase; Ch fatB2 is a TE gene from Cuphea hookeriana. In SD 249, f1 and f2 are the flanking regions (up slr1710 and down slr1710) for deletion of slr1710 encoding penicillin binding protein 2; Cc fatB1 is a TE gene from Cinnamomum camphorum. In SD277, f1 and f2 are the flanking regions (up slr2132 and down slr2132) for deletion of slr2132 encoding a phosphotransacetylase; tesA137 is a truncated 'tesA gene from E. coli with codon optimization for high-level expression in Synechocystis.

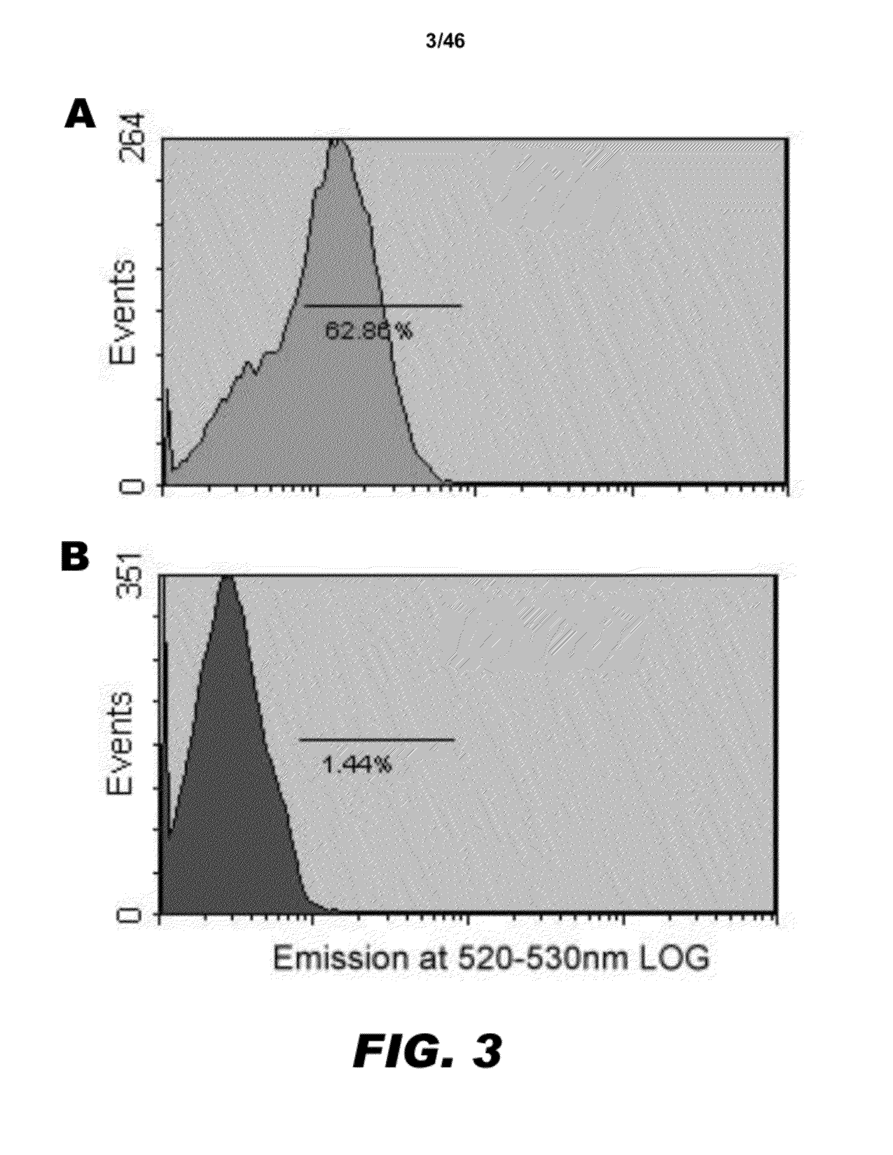

FIG. 3 depicts a comparison of the PHA accumulation in 6803 WT (A) and PHA synthesis deficient strain SD207 (B). Cells were stained by Nile Red and analyzed by flow cytometry, which shows that 62.86% of the WT cell contains high emission of PHA inclusions, while only 1.44% of the SD207 cells have high emission of PHA inclusions.

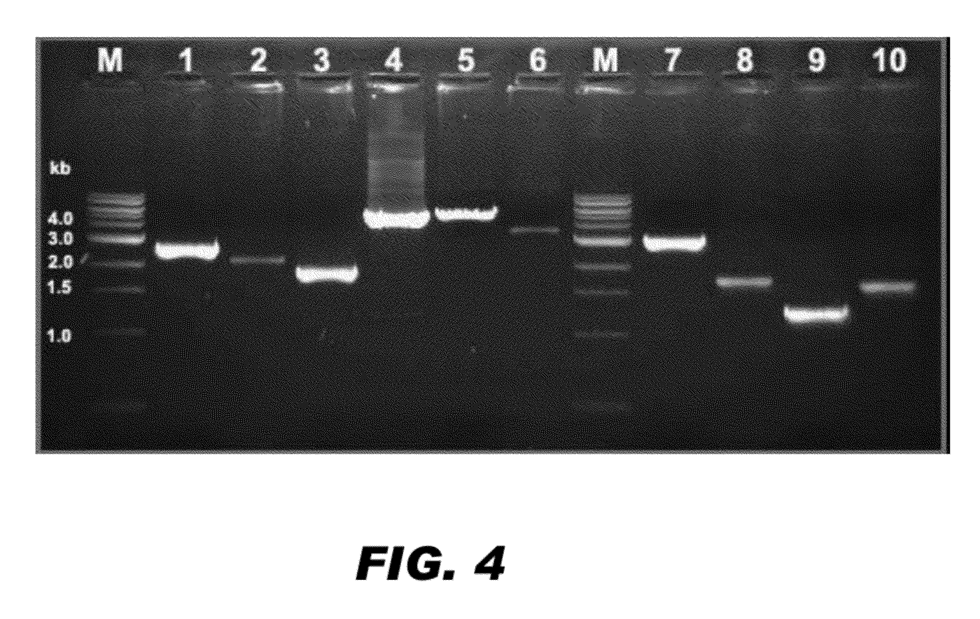

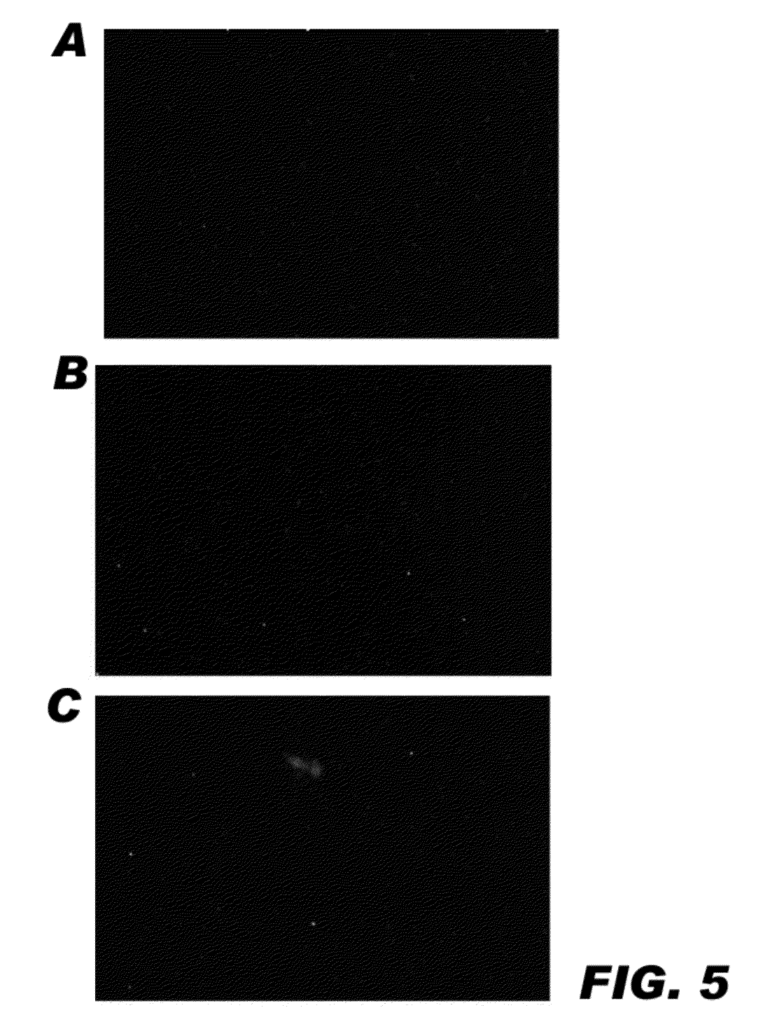

FIG. 4 depicts PCR identification of deletions and insertions in SD249. The segregation checking primers used in the PCR reactions are listed in Table 14. Wild-type DNA was used as the template for reactions loaded in the odd lanes. SD249 cell lysate prepared by freeze-thaw cycles was used as the template for reactions loaded in the even lanes. Lanes 1 and 2 used primers FadD-F1-Sequ and FadD-F2-A. Lane 1 indicated the wild-type slr1609 region to be deleted in SD249. Lane 2 indicated the Δslr1609::P_{psbA2} 'tesA cassette inserted in SD249. Lanes 3 and 4 used primers S4-seg100-S and S4-seg100-A. Lane 3 indicated the wild-type slr1993-slr1994 region to be deleted in SD249. Lane 4 indicated the Δ(slr1993-slr1994)::P_{cpc}accBC P_{rbc} accDA cassette inserted in SD249. Lanes 5 and 6 used primers S5100S and S5100A. Lane 5 indicated the wild-type sll1951 region to be deleted in SD249. Lane 6 indicated the Δsll1951::*P_{psbA2} Uc fatB1 cassette inserted into SD249. Lanes 7 and 8 used the primers S7 Seg 51S and S7 Seg 90A. Lane 7 indicated the wild-type slr2001-slr2002 region to be deleted in SD249. Lane 8 indicated the Δ(slr200′-slr2002)::*P_{psbA2} Ch fatB2 cassette inserted into SD249. Lanes 9 and 10 used the primers S9-S68 and S9-A71. Lane 9 indicated the wild-type slr1710 region to be deleted in SD249. Lane 10 indicated the Aslr1710::P_{psbA2}* Cc fatB1 cassette inserted into SD249. Sequencing analysis of these PCR products proved that all the inserted sequences were correct in SD249 as expected.

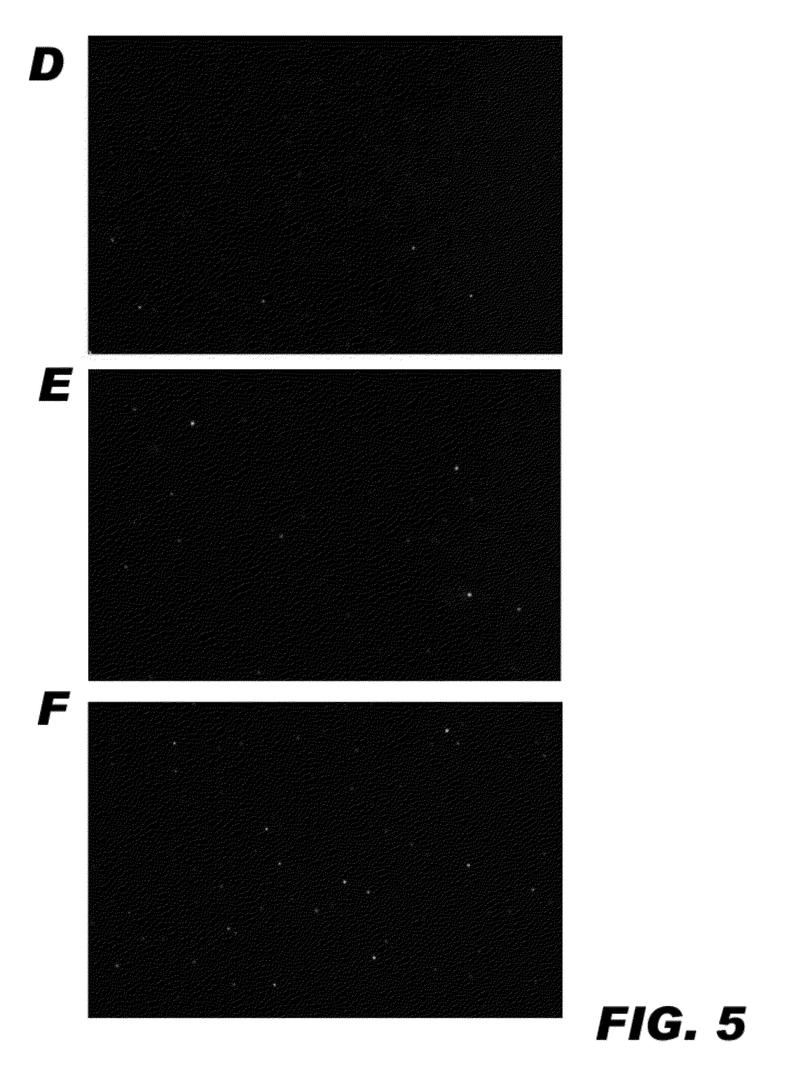

FIG. 5 depicts membrane damage of SD232 cells grown in different stages from a single cell. A and B, cells from a single cell derived colony had been growing on a BG-11 agar plate for 7 days (0.2% damage) (A) and 10 days (about 10⁶ cells/colony) (0.5% damage) (B); C, cells in a single cell derived colony were inoculated into 1 mL BG-11 medium in a glass tube and grown for 3 days with intermittent shaking (about 8×10⁶ cells/mL) (0.4% damage); D, the 1 mL SD232 culture was inoculated into 9 mL BG-11 medium in a flask and grown for 3 days with 60 rpm shaking (about 4×10⁷ cells/mL) (0.8% damage); E and F, the 10 mL SD232 was inoculated into 200 mL BG-11 medium and grown for 1 day and 2 days, respectively, with 100 mL/min aeration of 1% CO₂-enriched air. The high cell damage percentages in E (14.7% damage) and F (33.7% damage) indicated that the dilution into 200 mL for 4×10⁸ cells was too much and too dilute. In the fluorescence dark optic field, membrane damaged cells are permeable to SYTOX green and fluoresce green, while healthy cells fluoresce red. Blue, purple and yellow cells are all counted as damaged cells. The damaged cell percentages based on counting of at least 400 cells are indicated.

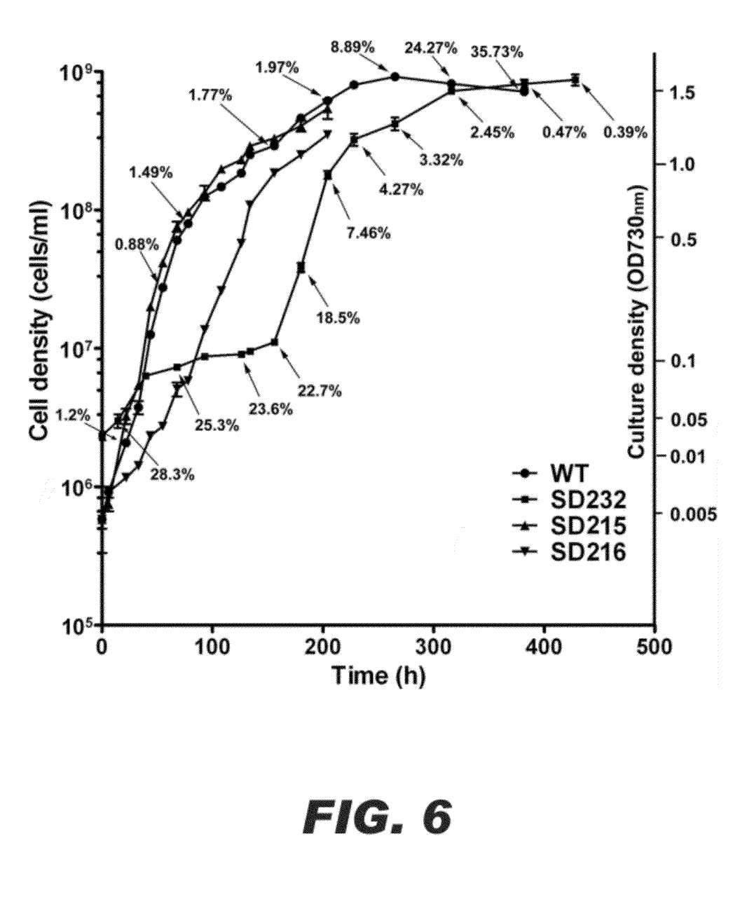

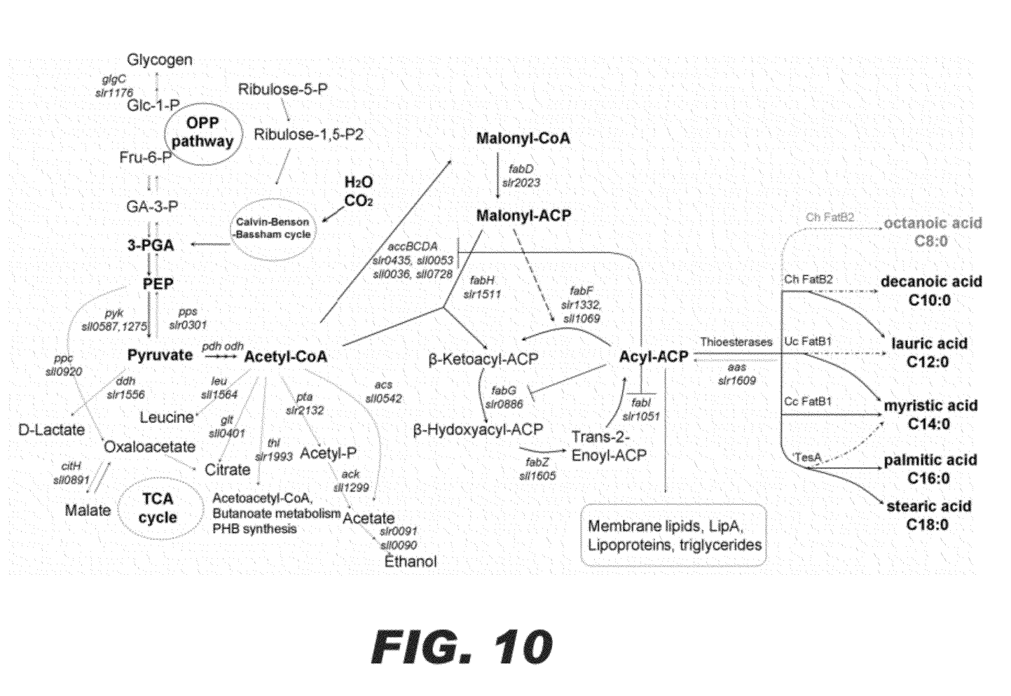

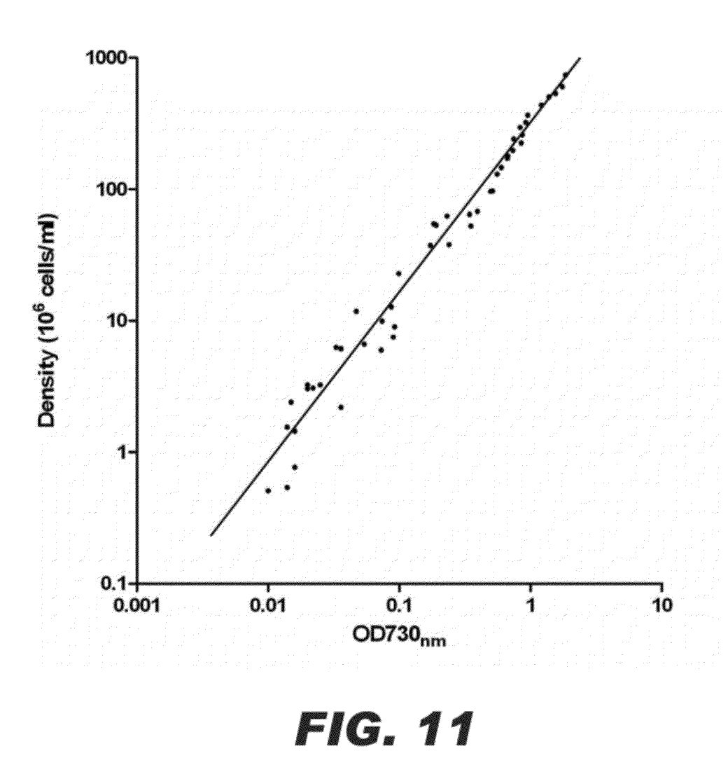

FIG. 6 depicts growth curves for SD strains. Cultures were grown at 30° C. in BG-11 medium and bubbled with 1% CO₂-enriched air. Cell density was transformed from culture optical density according to FIG. 11. The numbers pointed out by arrows are the damaged cell percentages in the SD232 and wild-type (WT) cultures at the specified times.

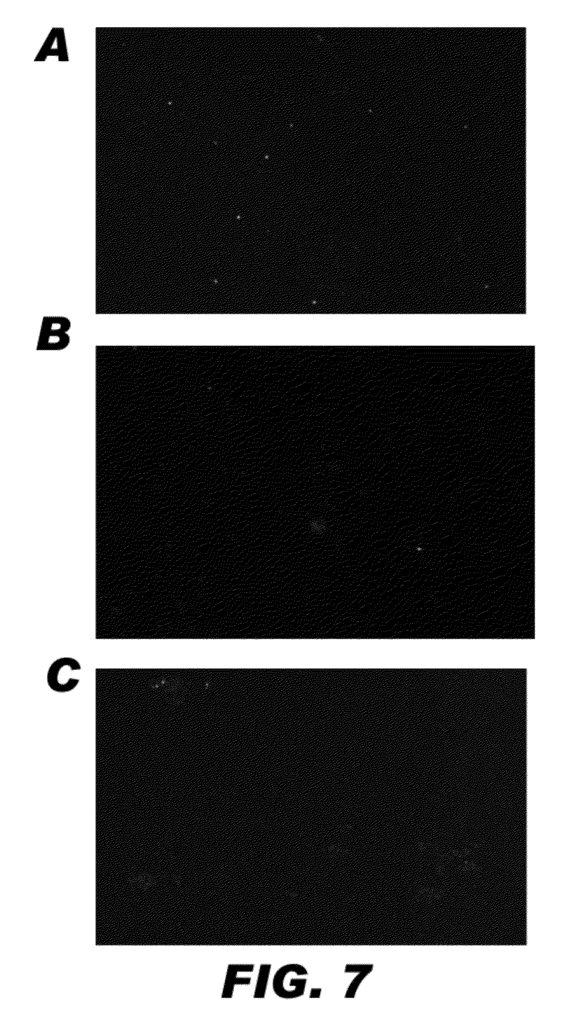

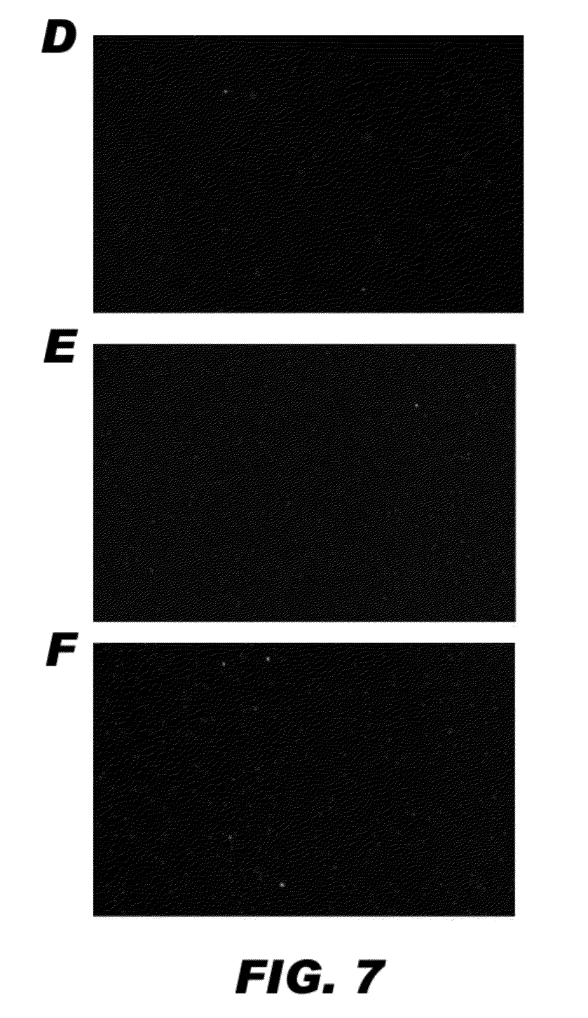

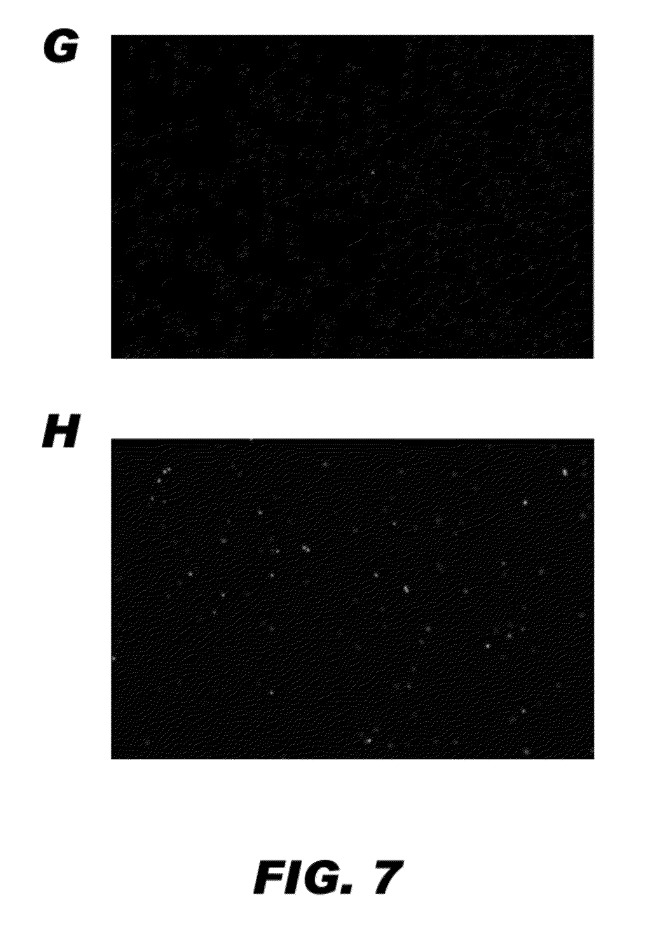

FIG. 7 depicts membrane damages during the growth of SD strains bubbled with 1% CO₂ enriched air. SD232 (A, C, E, and G) and WT (B, D, F, and H) cultures were started at 10⁶ cells/mL with 1% CO₂ aeration. Cell membrane damages were indicated by SYTOX green staining. The time for growing after inoculation and damaged cell percentages based on counting at least 400 cells are as follows. Lag phase: (A) 14 h, 25%, (B) 9 h, 1.2%. Early exponential phase: (C) 180 h, 22%, (D) 22 h, 0.9%. Exponential phase: (E) 224 h, 2.3%, (F) 78 h, 4.5%. Stationary phase: (G) 428 h, 0.4%, and (H) 382 h, 36%.

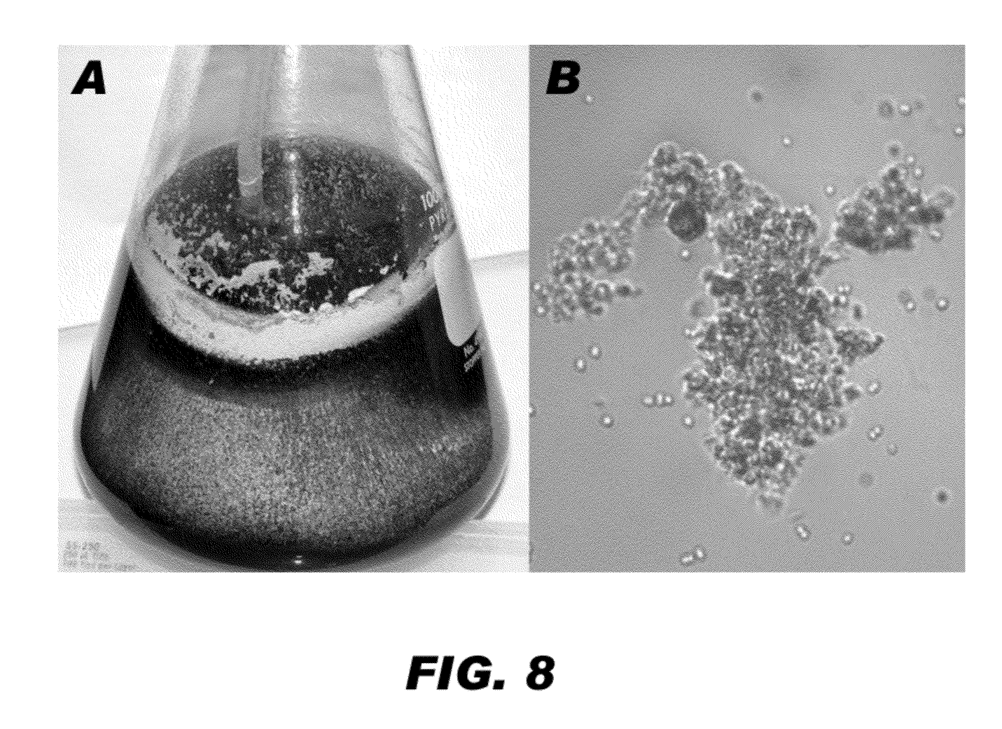

FIG. 8 depicts secreted FFAs (white deposit) from an SD232 culture. (A) shows an 800 mL culture of SD232 grown in an aeration flask for 4 days. Notice the secreted FFAs precipitated out of the culture medium and forming a granular ‘ring’ on the flask wall above the aqueous phase. (B) shows the microscopy of FFA secretion. Bar=10 μm.

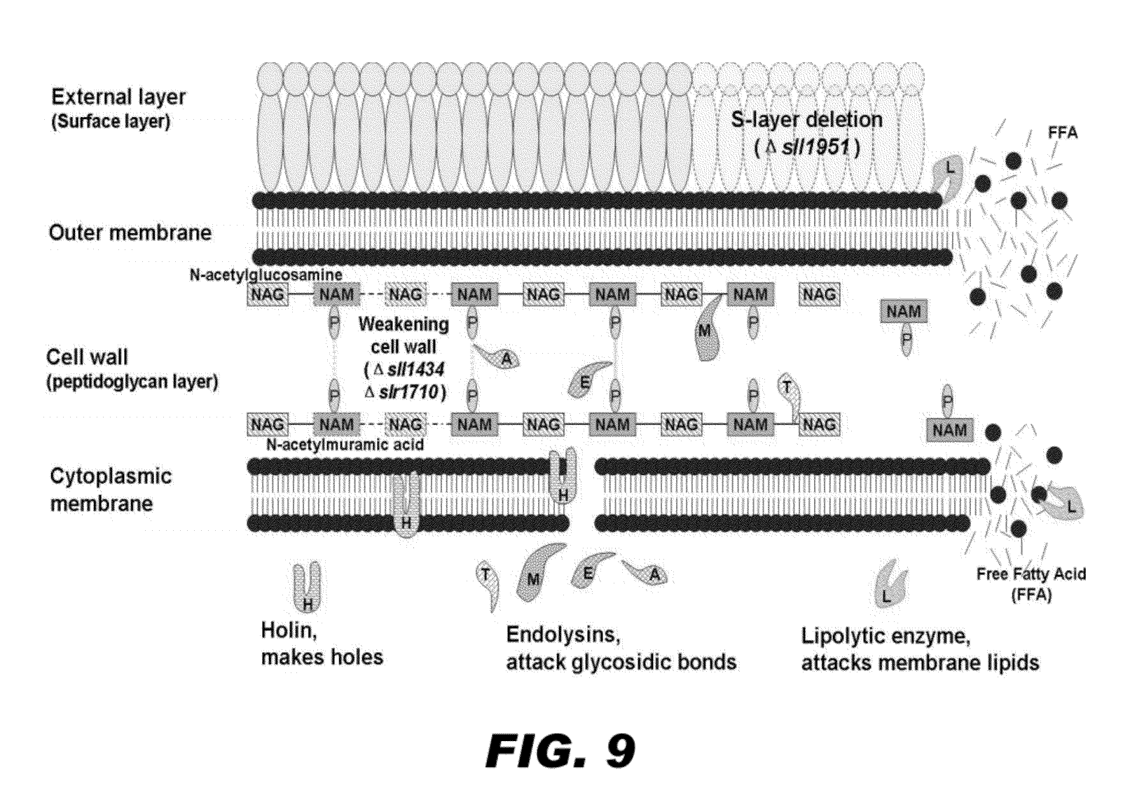

FIG. 9 depicts a diagram of the Synechocystis cell surface layers and the enzymes that can be used to introduce holes (H=holins) in the cytoplasmic membrane to enable escape of endolysins (A, amidase; M, muramidase; T, transglycosylase; and E, endopeptidase) to damage/degrade the peptidoglycan layer and cleave glycosidic bonds between N-acetylglucosamine (NAG) and N-acetylmuramic acid (NAM) (by M or T), and also break the peptide cross-bridges (P-P) that link the rows of sugars together (by A or E). Deletion of S-layer (Δsll1951) and/or compromising the peptidoglycan layer (by deleting a penicillin-binding protein gene, e.g., slr1710 and sll1434) facilitate FFA secretion and yields (by removing feedback inhibition). Inducible synthesis of lipolytic enzymes hydrolyze lipid membranes into FFA.

FIG. 10 depicts Synechocystis sp. PCC 6803 fatty acid synthesis pathways and modifications for FFA over production. The molecules and reactions in the primary pathways towards FFA overproduction are indicated as bold text, while those in the competing pathways which uncouple the carbon flux from FFA over production are indicated as regular unbolded text. Abbreviations: OPP, oxidative pentose phosphate; TCA, tricarboxylic acid; GA-3-P, Glyceraldehyde-3-Phosphate; 3-PGA, 3-phosphoglycerate; PEP, phosphoenolpyruvic acid; Ch FatB2, thioesterase from Cuphea hookeriana; Uc FatB2, thioesterase from Umbellularia californica; Cc FatB1, thioesterase from Cinnamomum camphorum. The bold lines with arrow heads point to the major products of these TEs in 6803, while the dashed lines with arrow heads point to the minor products in 6803.

FIG. 11 depicts the relationship between 6803 culture cell density and optical density. Forty-five samples from 6803 exponentially growing cultures were measured. Cell density was counted in a haemacytometer (Neubauer, 0.1 mm×0.0025 mm², China). OD_{730 nm} was measured in a spectrometer (Genesys 10 VIS, Thermo Spectronic, NY, USA).

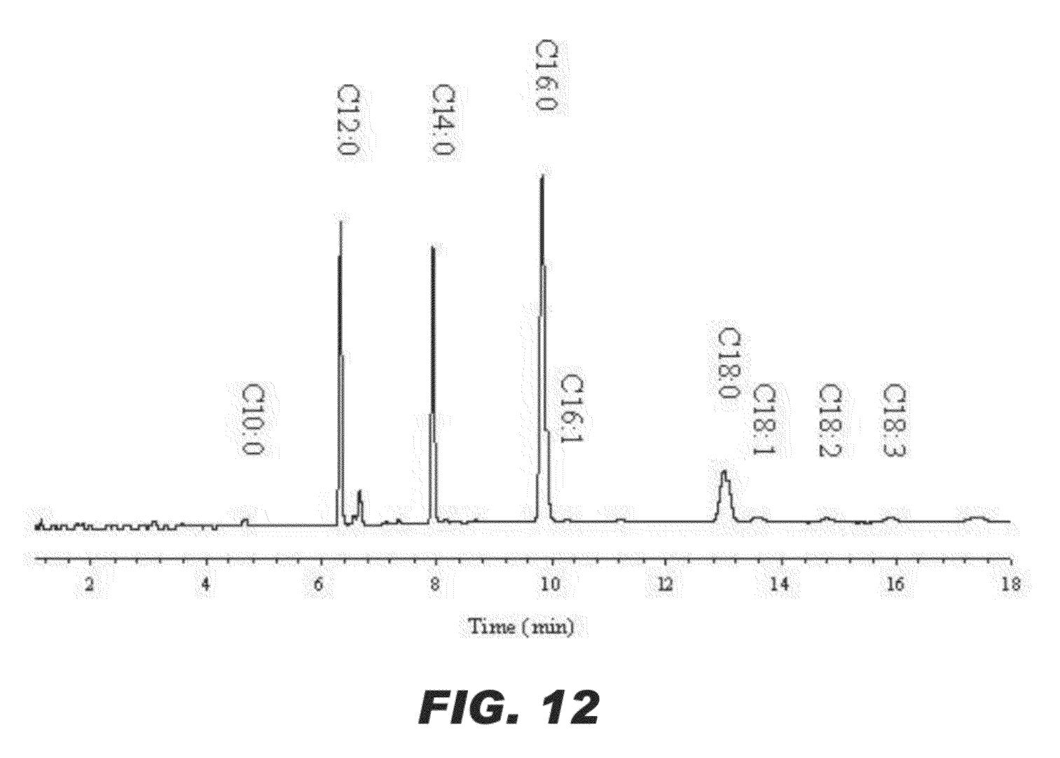

FIG. 12 depicts the GC analysis of one secreted FFA sample from SD249. The types of FFA are noted on their peaks.

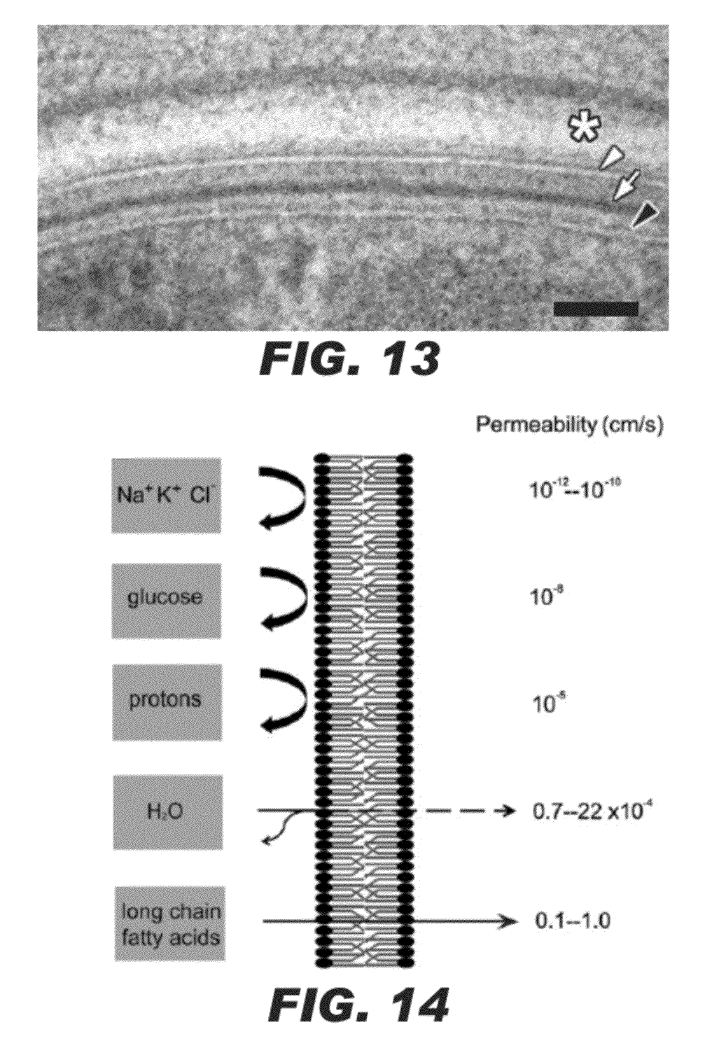

FIG. 13 depicts an electron microscope image of the envelope layers of a wild-type 6803 cell. The surface-layer proteins (asterisk), outer membrane (white arrowhead), peptidoglycan (cell wall) layer (arrow), and cytoplasmic membrane (black arrowhead) are pointed out. Bar=50 nm.

FIG. 14 depicts a diagram of a phospholipid bilayer and permeabilities of ions and molecules through the phospholipid bilayer.

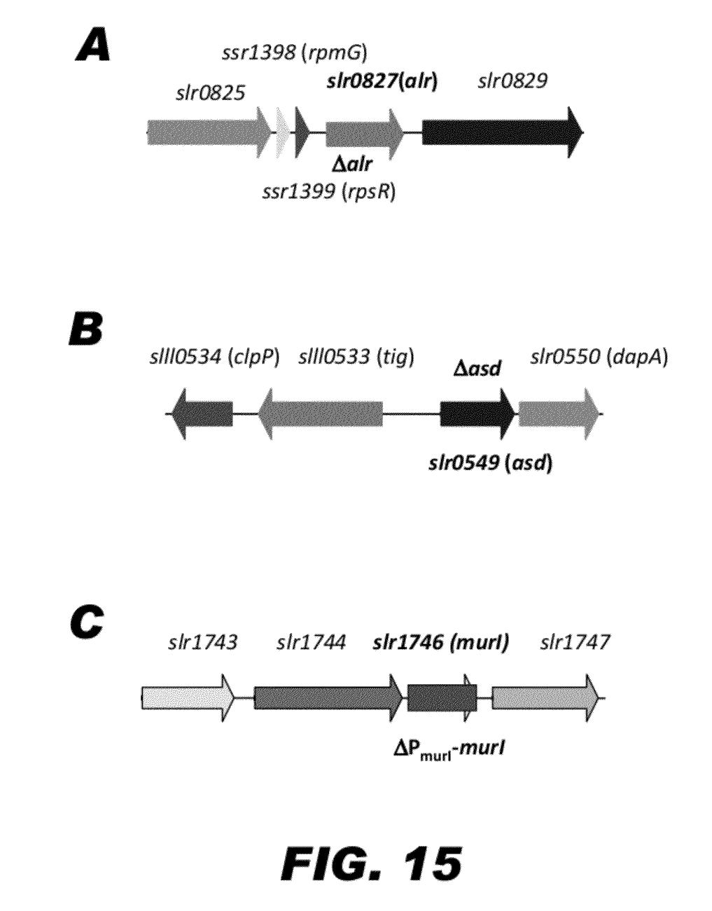

FIG. 15 depicts deletions of the (A) alr, (B) asdA and (C) murl genes. (A) shows the deletion of alr₁ to afr₁₂₀₀. (B) shows the deletion of asd₁ to asd₈₄₃. (C) shows the deletion of 983 bp including the promoter region of murl (86 bp) and the whole ORF of 897 bp murl.

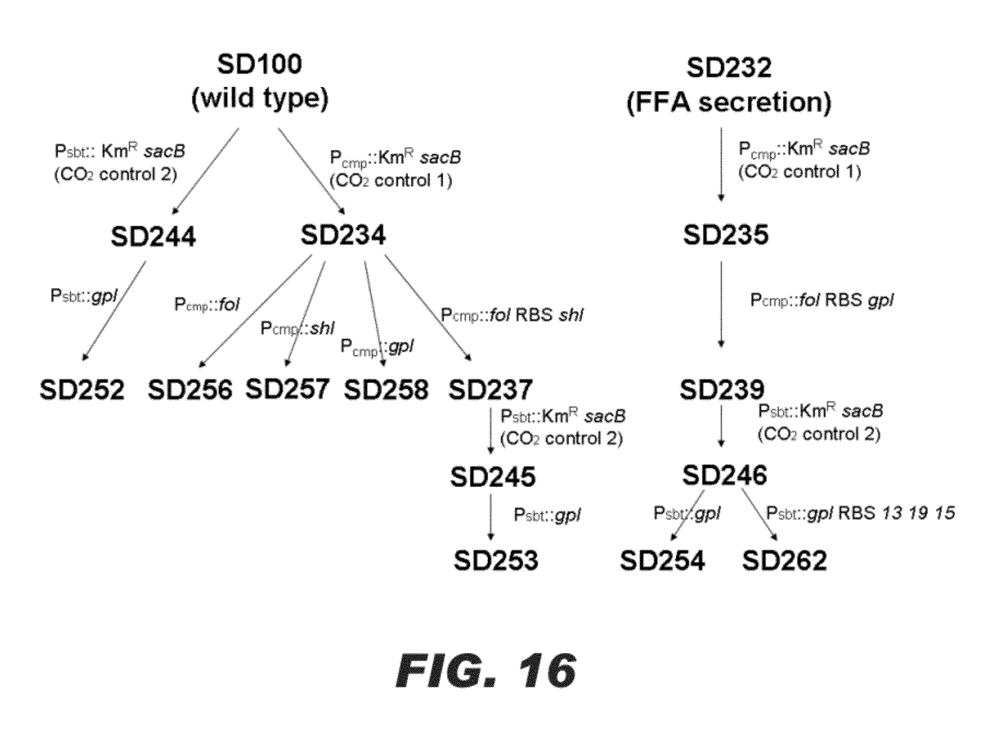

FIG. 16 depicts genealogies of the Green Recovery constructions. Three lipolytic genes (fol, shl, and gpl) were inserted into SD100 (6803 wild-type) and SD232 (an FFA secretion 6803 strain), and controlled by two CO₂ limitation inducible promoters (P_{cmp} and P_{sbt}). Detailed genetic information of the strains is described in Table 11.

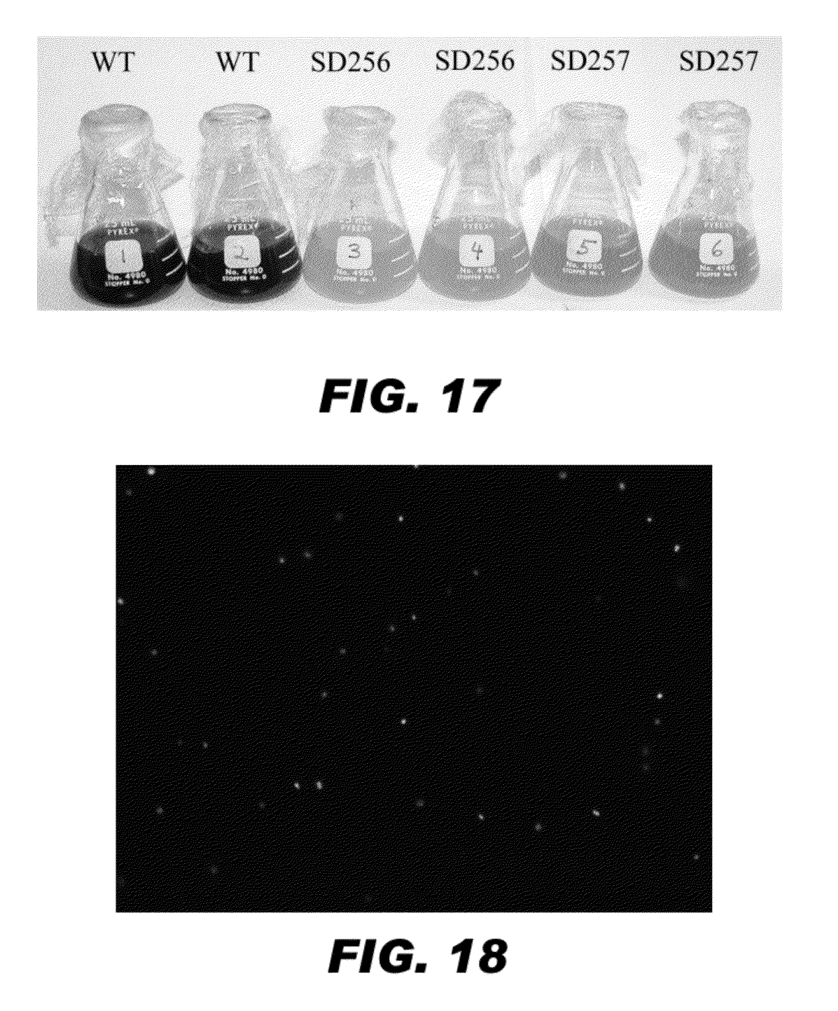

FIG. 17 depicts duplicate cultures of 6803 wild type, SD256 and SD257 in sealed flasks four days after CO₂ limitation.

FIG. 18 depicts a fluorescence microscopy picture of a Sytox Green stained SD256 culture two days after CO₂ limitation. Under a fluorescence microscope with 460 nm-490 nm excitation, cells in a 6803 culture usually have three colors. Red cells are counted as membrane intact cells, where red is the autofluorescence of cyanobacterial phycobilisomes. Green cells are counted as membrane damaged cells, where green is the fluorescence of Sytox Green penetrating inside the cells and binding with DNA. Blue cells are also counted as damaged cells, which could be ghost cells with DNA and pigments already leaked out. For each sample of 6803 and 6803-derived strains, at least 200 cells were counted and analyzed for membrane permeability.

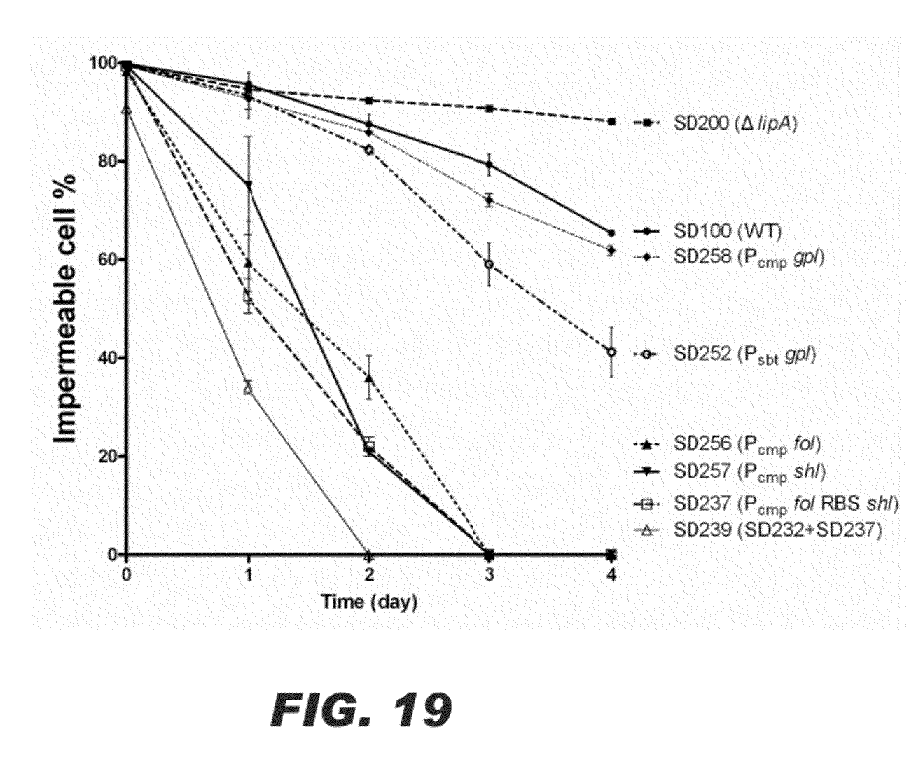

FIG. 19 depicts membrane damage after CO₂ limitation for different SD strains. The membrane damage was revealed by Sytox staining. The starting cell densities and the estimated damage rates are listed in Table 11.

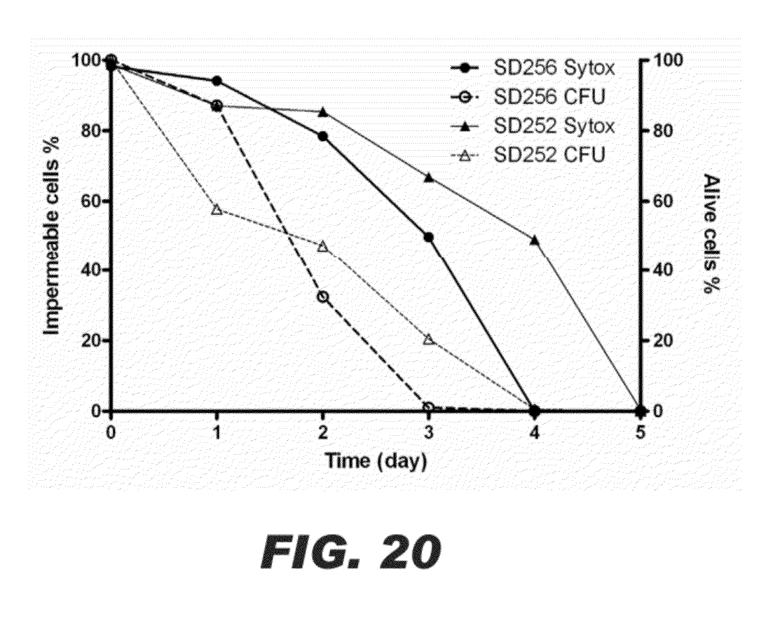

FIG. 20 depicts the relationship between membrane permeability (revealed by Sytox staining) and cell viability (revealed by CFU) during Green Recovery after CO₂ limitation.

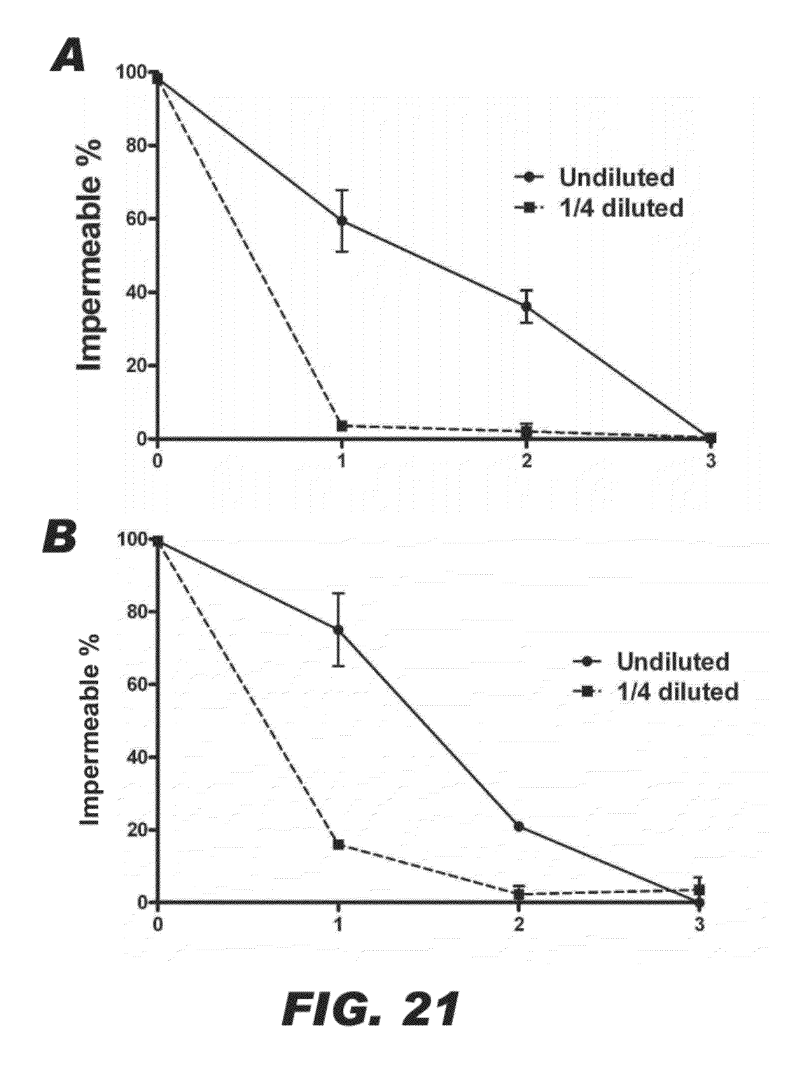

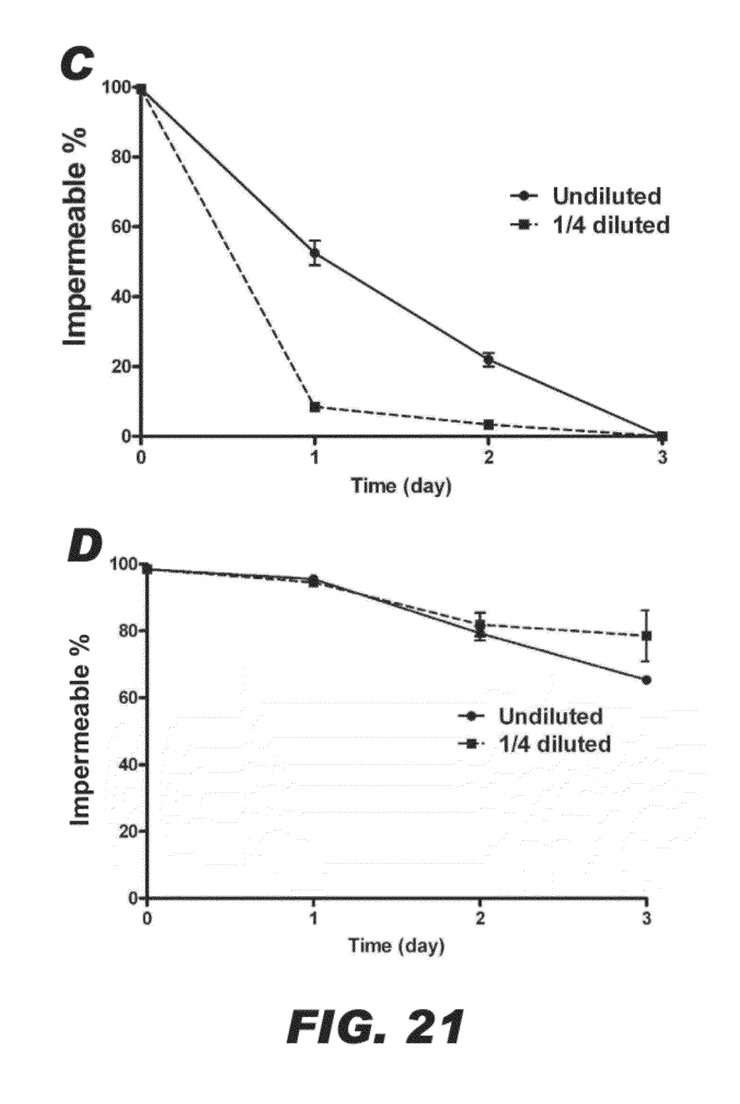

FIG. 21 depicts the membrane damage of different SD strains after CO₂ limitation with ¼ and 1/16 dilution of the original culture. (A) SD256, (B) SD257, (C) SD237, and (D) WT.

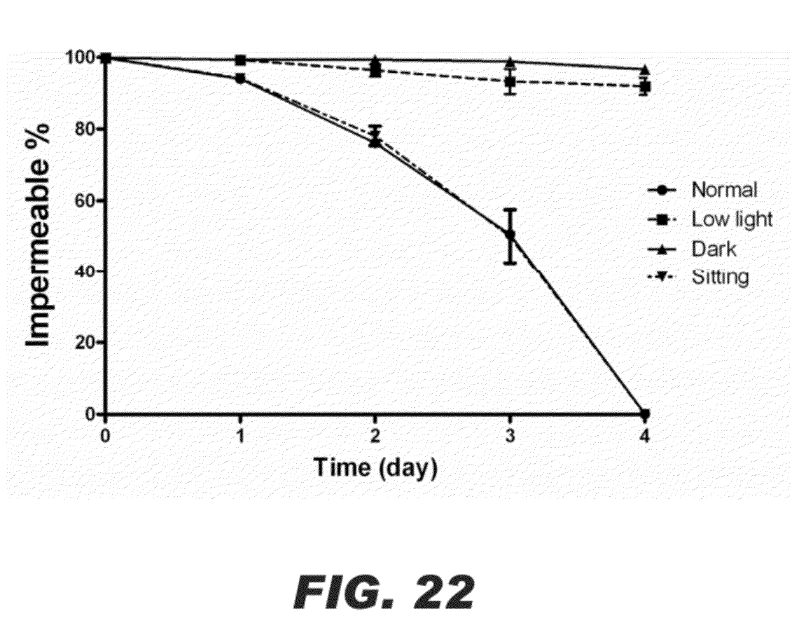

FIG. 22 depicts the membrane damage of SD256 after CO₂ limitation under different conditions. Normal means the CO₂ limited cultures were rotated at 100 rpm under continuous illumination (140 μmol photons m⁻² s⁻¹); low light means the CO₂ limited cultures were rotated at 100 rpm under illumination (20 μmol photons m⁻² s⁻¹); dark means the CO₂ limited cultures were rotated at 100 rpm under illumination (2 μmol photons m⁻² s⁻¹); sitting means the CO₂ limited cultures were shaken only once per day before sampling and under illumination (140 μmol photons m⁻² s⁻¹).

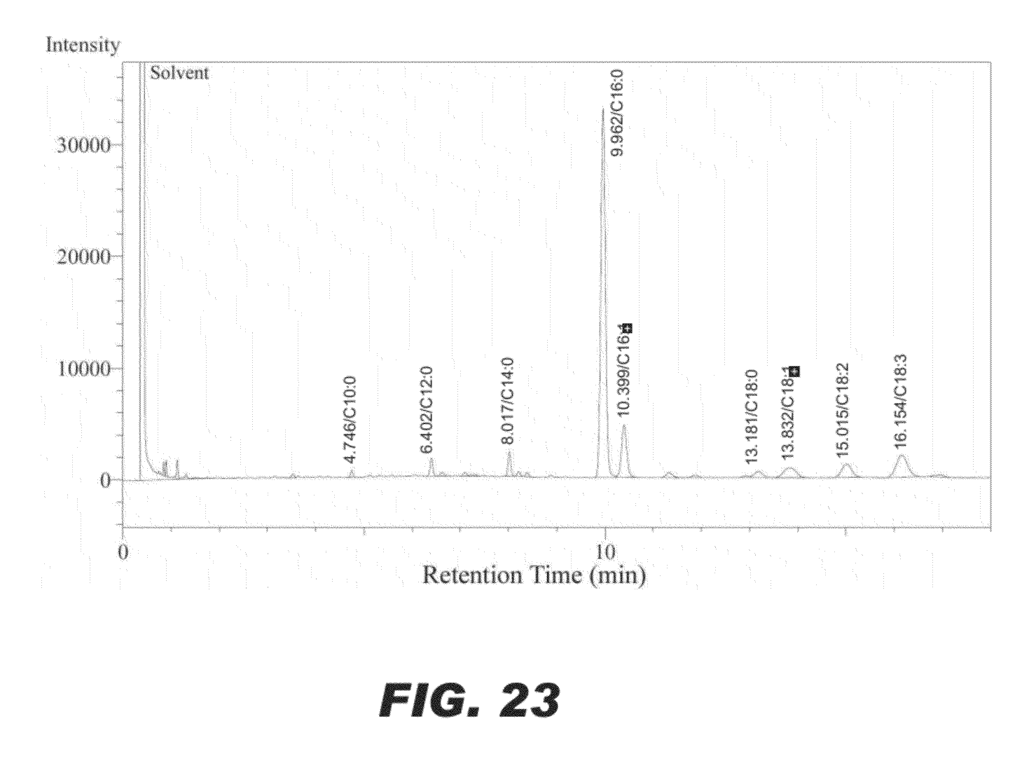

FIG. 23 depicts the GC analysis of the FFA samples extracted by hexane from the SD237 culture 3 days after CO₂ limitation. The retention time and the types of released FFAs are marked on the peaks.

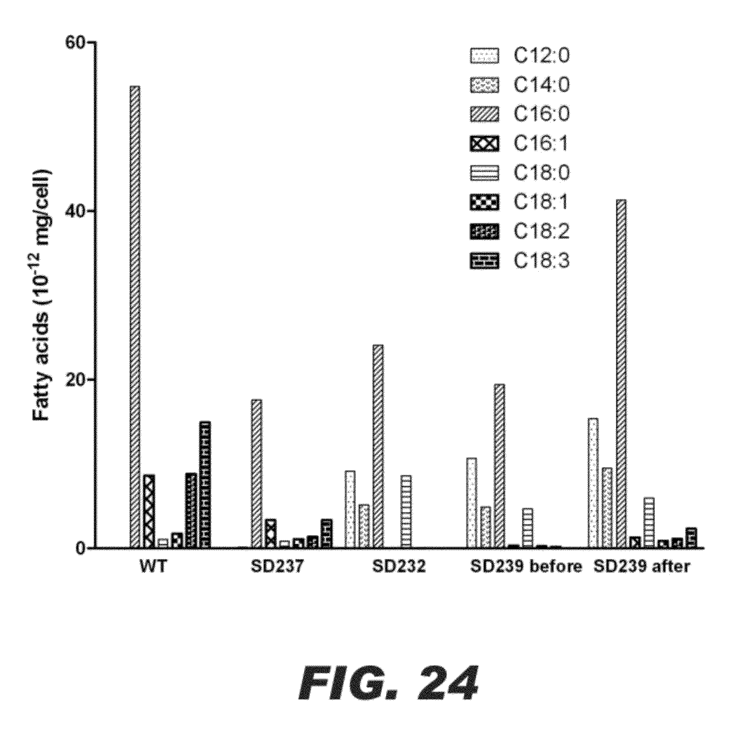

FIG. 24 depicts the fatty acid profiles of SD strains. All the cultures were grown to about 4×10⁸ cells/ml at 30° C. For wild-type, the columns show the fatty acid profile of total membrane lipids. For SD237, the columns show the released FFA profile by Green Recovery, which is similar to that of wild-type with abundant unsaturated fatty acids. For SD232, the columns show the profile of secreted FFAs, which are highly saturated with significant amounts of C12:0 and C14:0. For SD239 before (Green Recovery), the columns show the profile of secreted FFAs before CO₂ limitation, which is similar to that of the FFA secretion strain SD232. For SD239 after (Green Recovery), the columns show the profile of all the FFAs contributed by SD239 after CO₂ limitation, which is a mixture of secreted FFAs (e.g., C12:0 and C14:0) and released FFAs (e.g., C18:2 and C18:3).

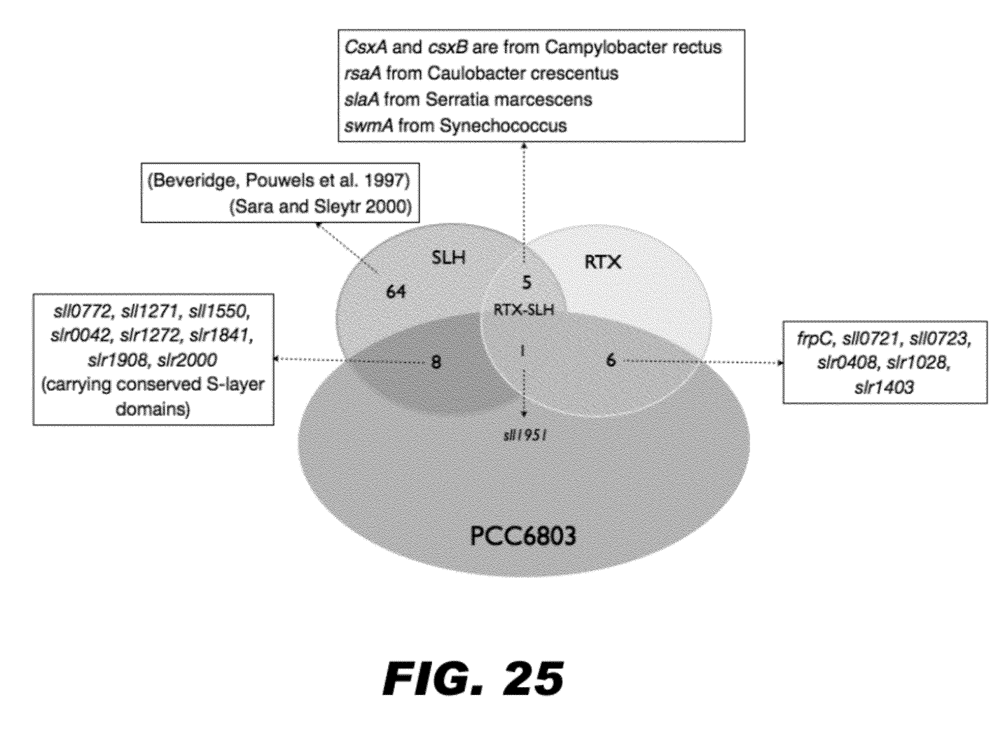

FIG. 25 depicts an overview of surface layer protein candidates determined to be present in 6803.

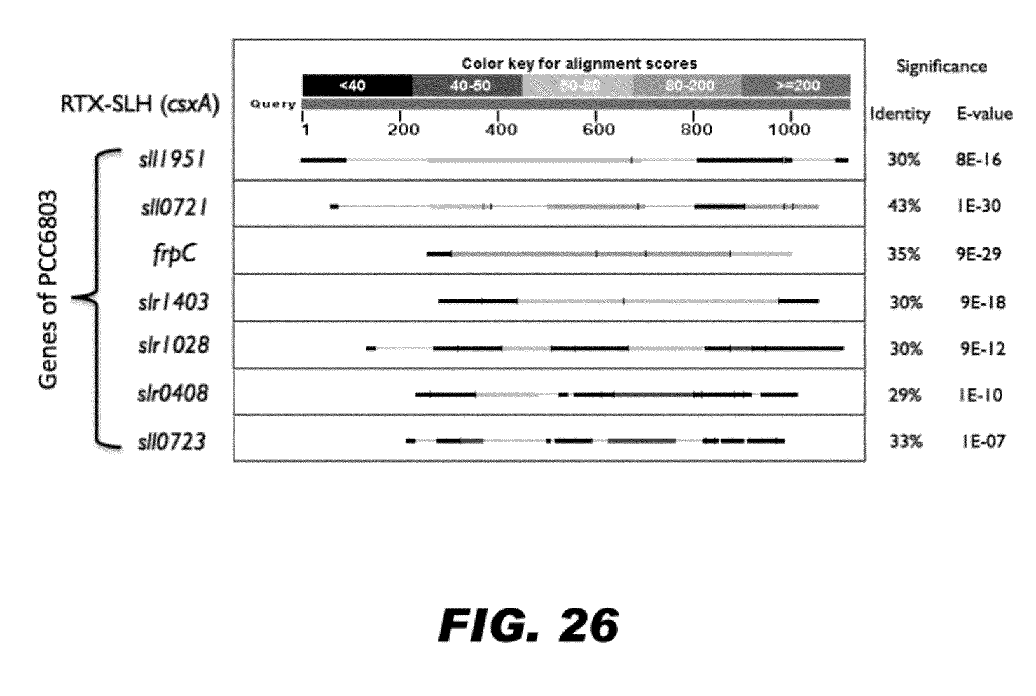

FIG. 26 depicts the alignment of the RTX surface layer gene csxA from Campylobacter rectus with its homologous genes found in 6803.

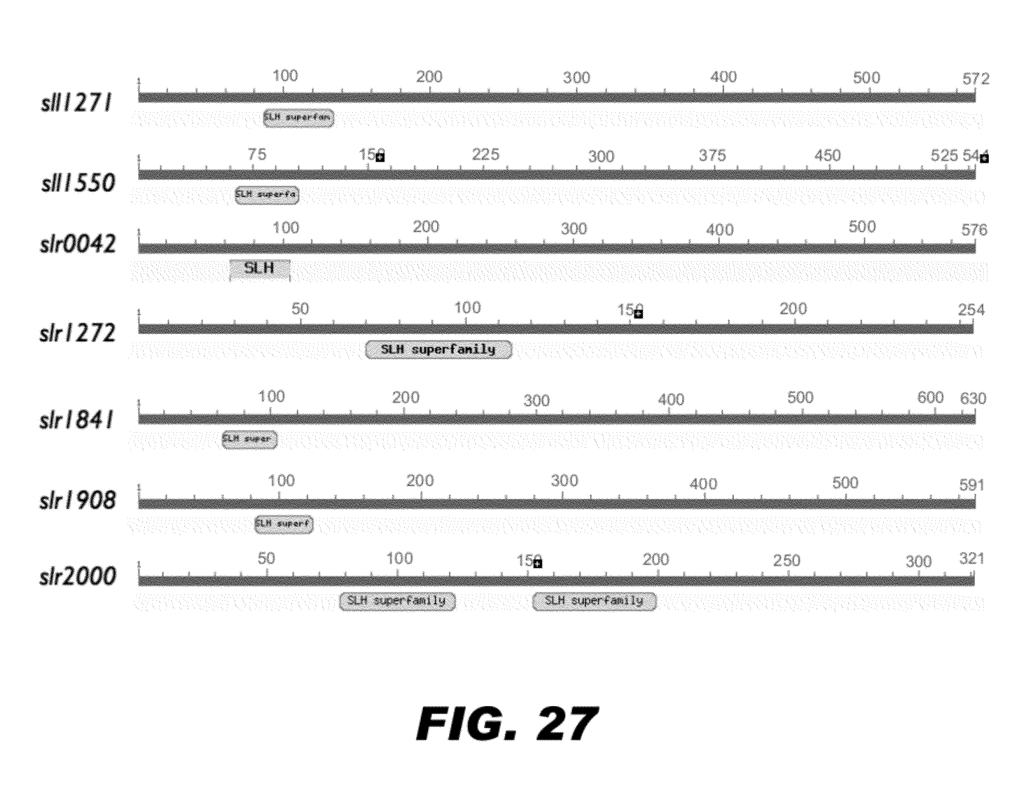

FIG. 27 depicts the surface layer protein candidates in 6803 that are carrying SLH domains.

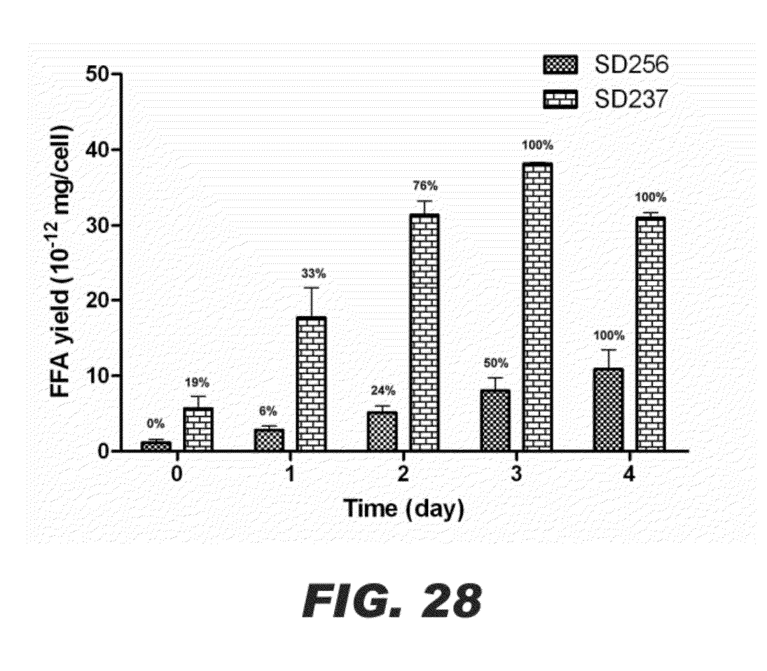

FIG. 28 depicts FFA yields of SD256 and SD237 during Green Recovery. Ten CO₂-limitating flasks with 16 ml cultures were set in the same conditions on day zero for each strain. Everyday, the whole cultures in duplicate flasks were extracted by hexane for FFA yields. The cell membrane damage was observed after Sytox staining, and the permeable cell percentages are indicated above the columns.

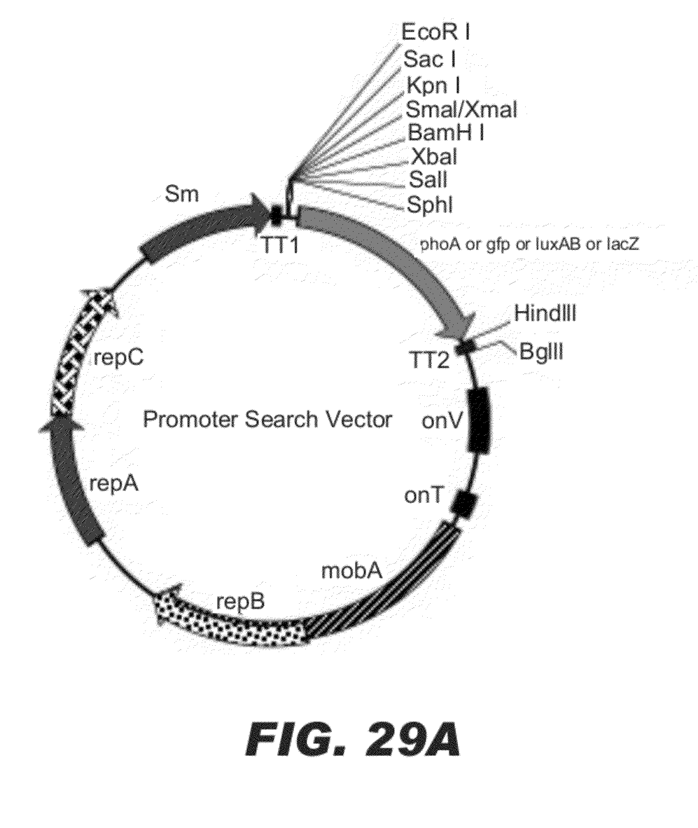

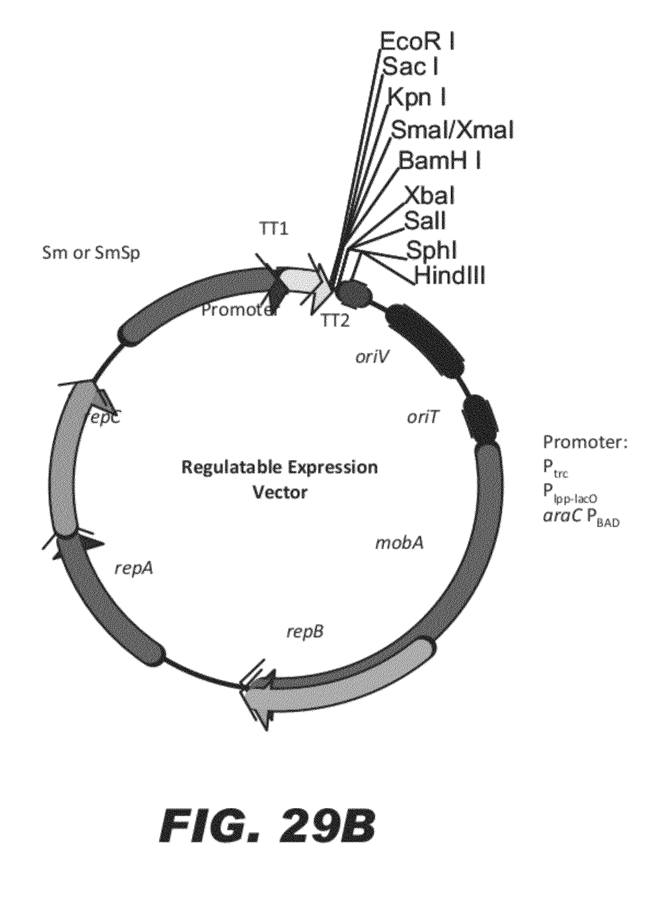

FIG. 29 depicts diagrams of (A) promoter search vectors and (B) regulatable expression vectors. These plasmid systems may increase the rapidity of evaluating optimal genetic modifications to use in strain improvement. Even more importantly, their use may speed the discovery of new features of Synechocystis biology. Strain 6803 possesses seven plasmids with some being dispensable. In addition, shuttle vectors to facilitate analysis of genes in suitable strains of E. coli as well as in 6803 derivative strains with and without modifications may be developed to enhance their performance and activities in 6803 derivative strains engineered for improved FFA production and secretion.

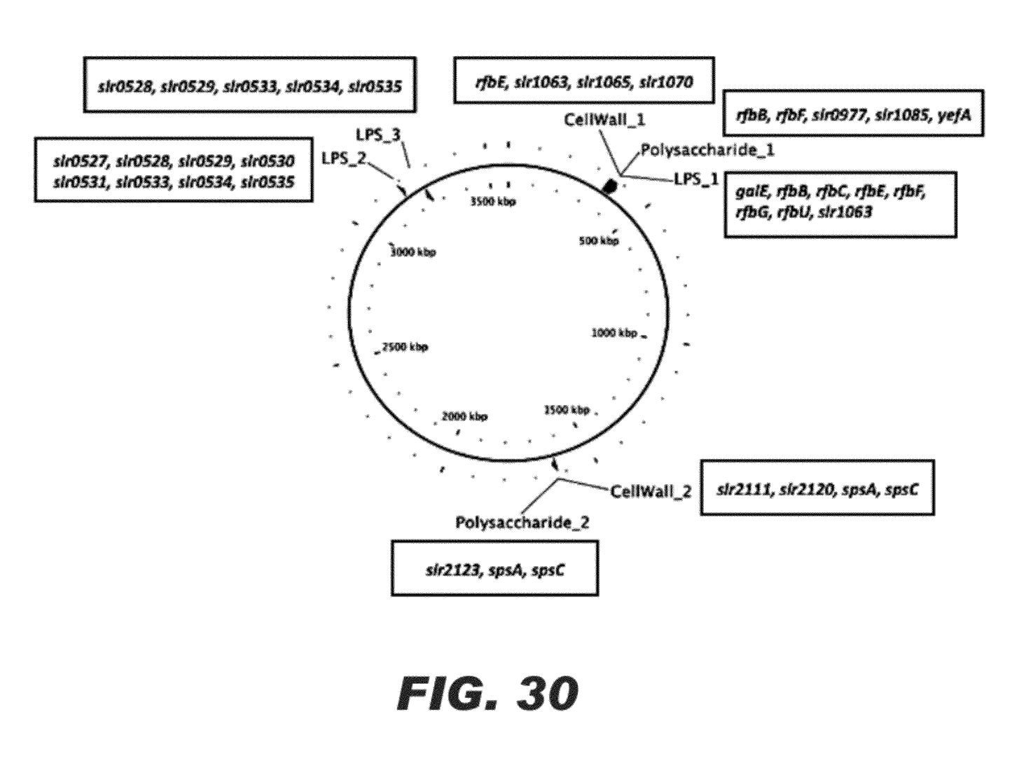

FIG. 30 depicts the putative cell surface related operons on the Synechocystis chromosome identified by bioinformatic searches.

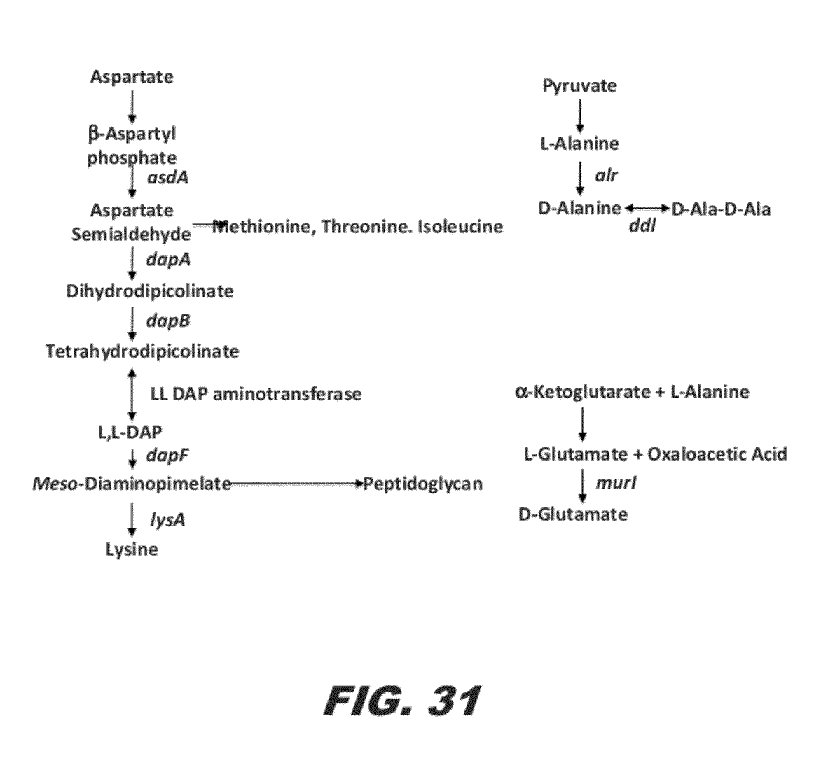

FIG. 31 depicts the biosynthetic pathways required for the synthesis of diaminopimelic acid (DAP) and other peptidoglycan cell wall components.

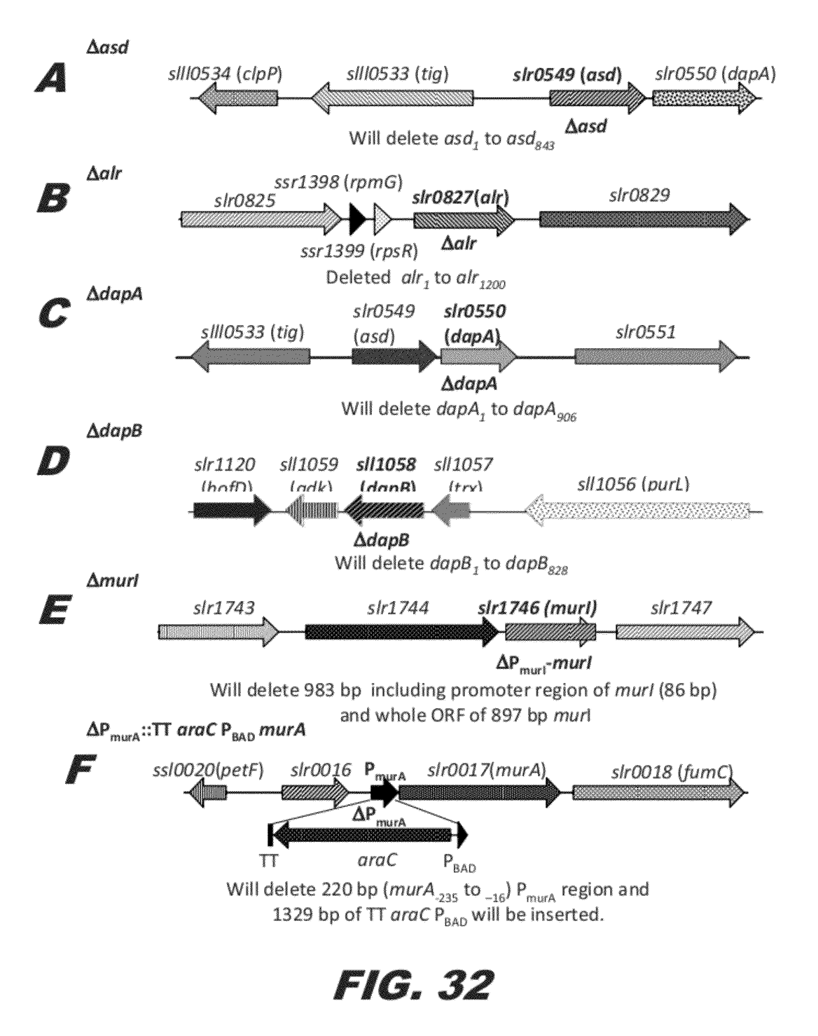

FIG. 32 depicts deletions of the (A) asd, (B) alr, (C) dapA, (D) dapB and (E) murl genes. (A) shows the deletion of asd₁ to asd₈₄₃. (B) shows the deletion of alr₁ to afr₁₂₀₀. (C) shows the deletion of dapA₁ to dapA₉₀₆ (D) shows the deletion of dapB₁ to dapB₈₂₈. (E) shows the deletion of 983 bp including the promoter region of murl (86 bp) and the whole ORF of 897 bp murl. (F) shows the deletion of 220 bp (murA₋₂₃₅ to murA₋₁₆) of P_{murA} promoter region and insertion of 1329 bp of TT araC P_{BAD} sequence from E. coli.

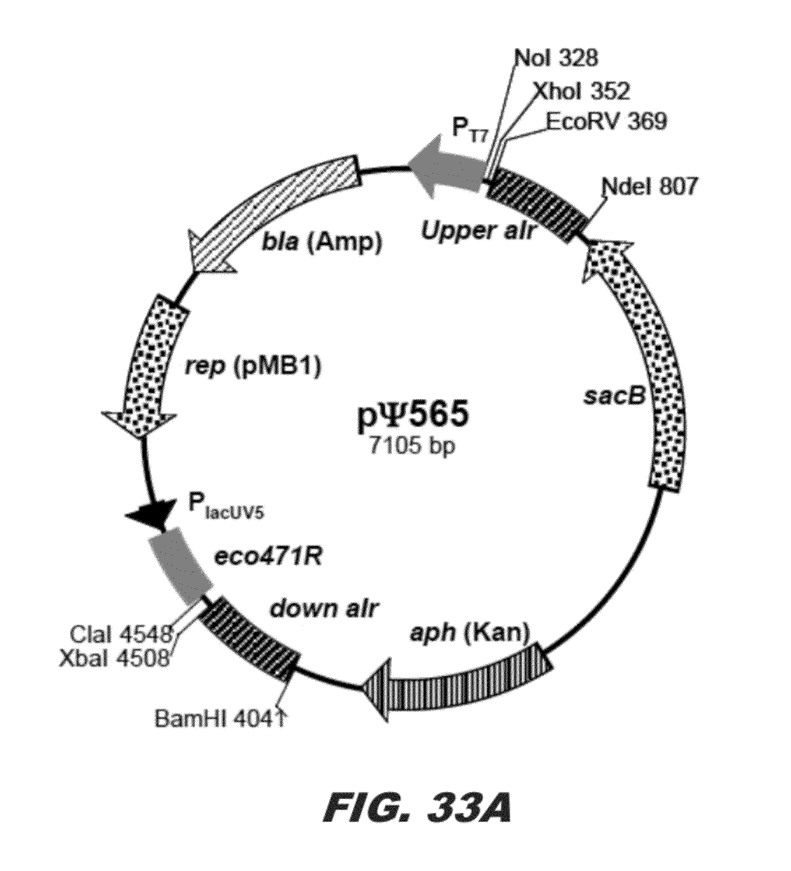

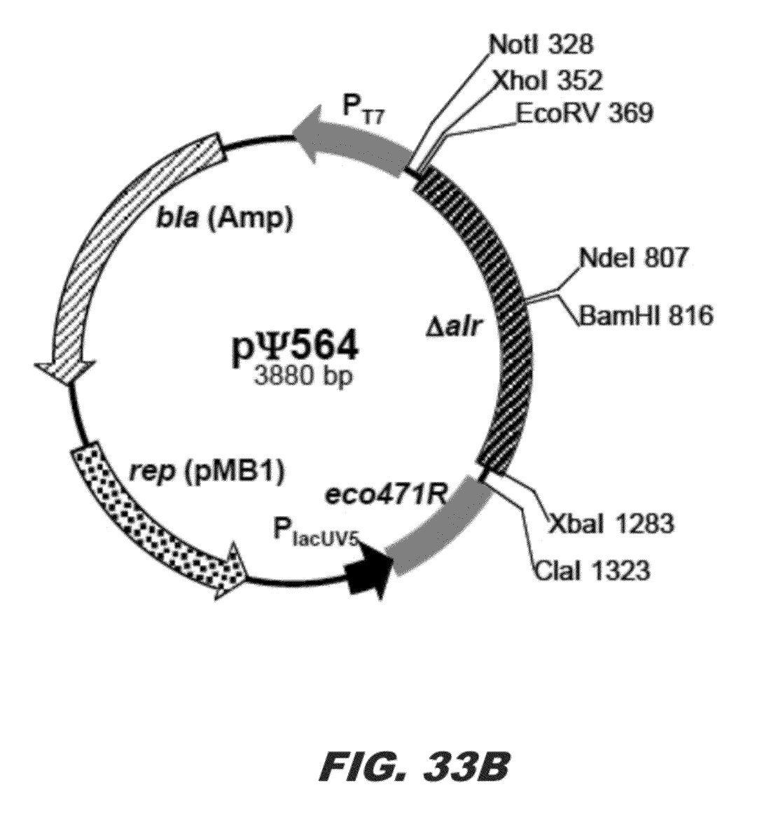

FIG. 33 depicts the suicide vectors (A) pΨ565 and (B) pΨ564 for the construction of Δalr in Synechocystis 6803.

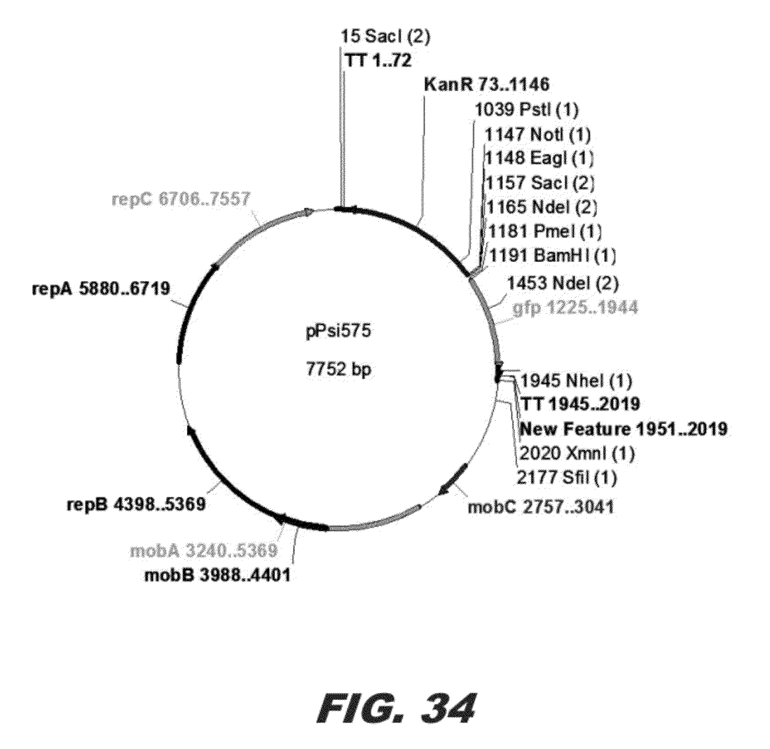

FIG. 34 depicts the promoter search vector pΨ575, a derivative of the broad host-range plasmid RSF1010. pΨ575 uses GFP as a reporter.

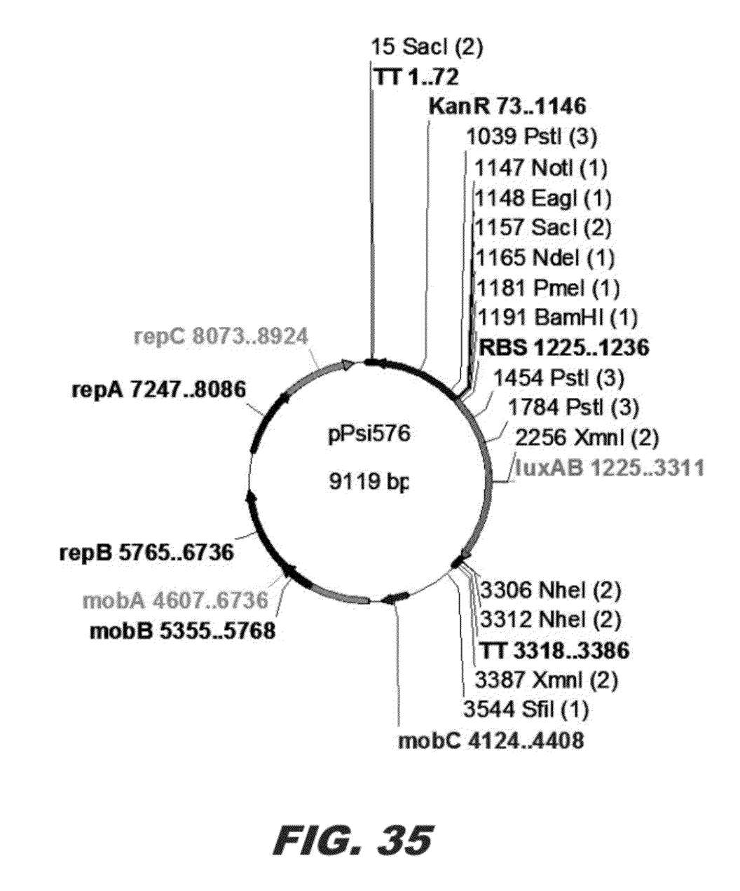

FIG. 35 depicts the promoter search vector pΨ576, a derivative of the broad host-range plasmid RSF1010. pΨ576 uses luxAB as a reporter.

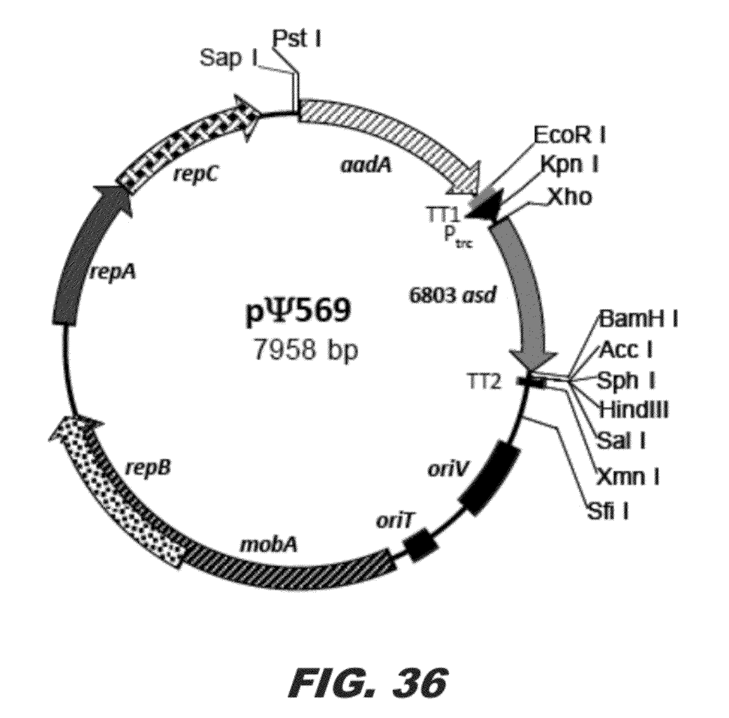

FIG. 36 depicts the 6803 Asd⁺ expression vector pΨ569 for Synechocystis.

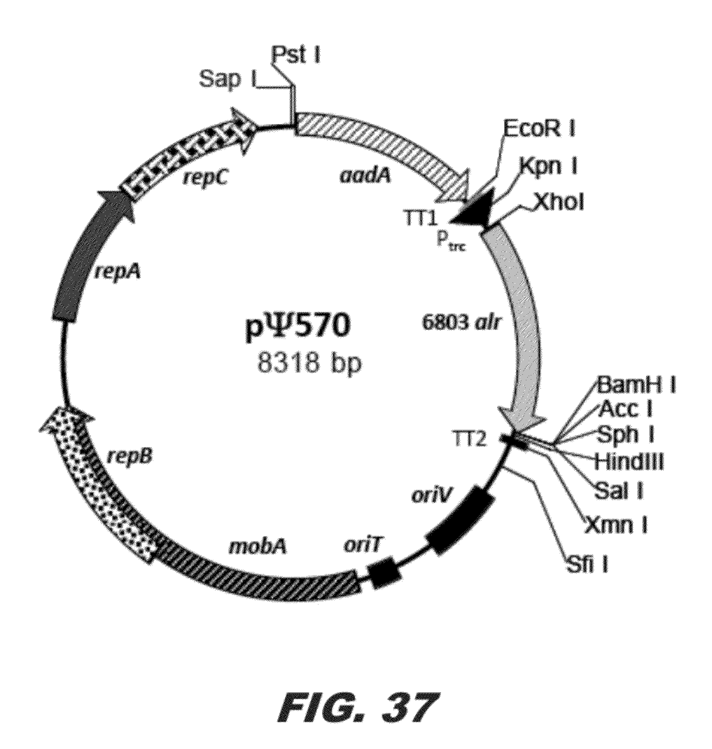

FIG. 37 depicts the 6803 Alr⁺ expression vector pΨ570 for Synechocystis.

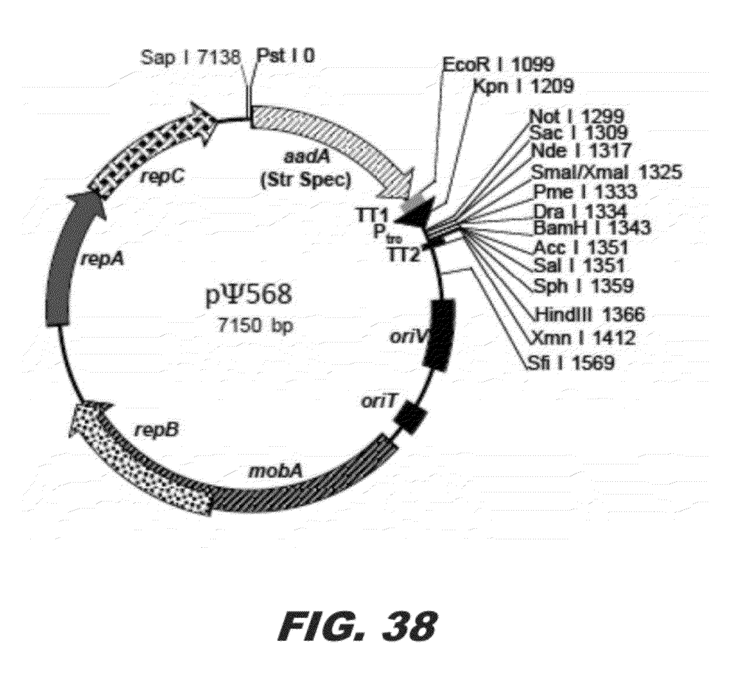

FIG. 38 depicts the shuttle expression vector pΨ568 for Synechocystis and E. coli.

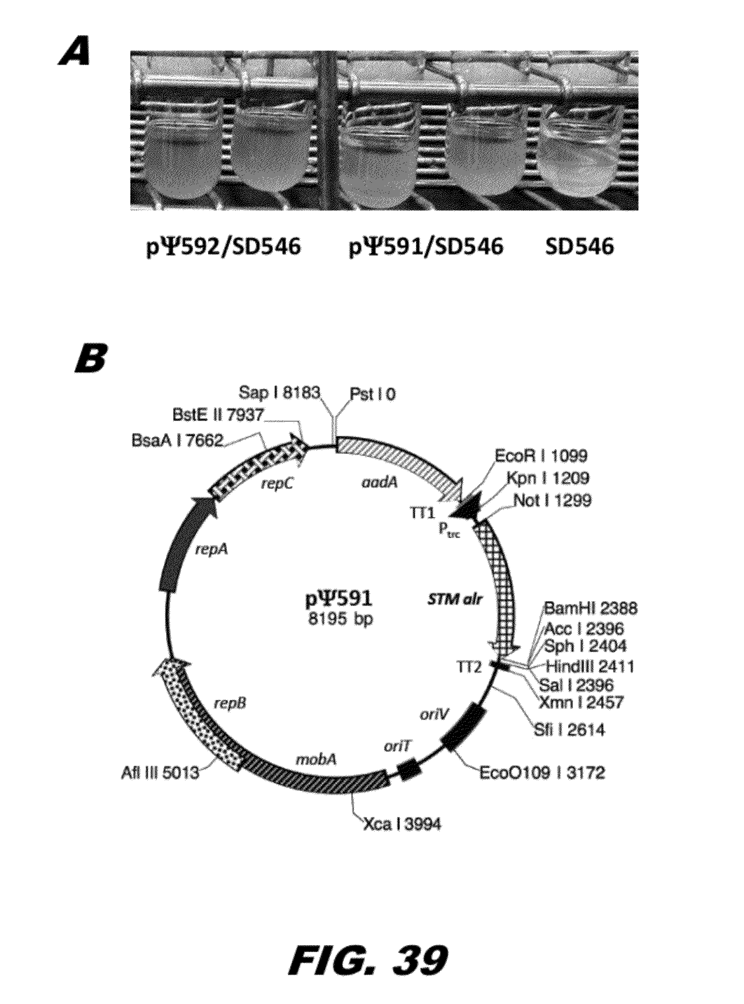

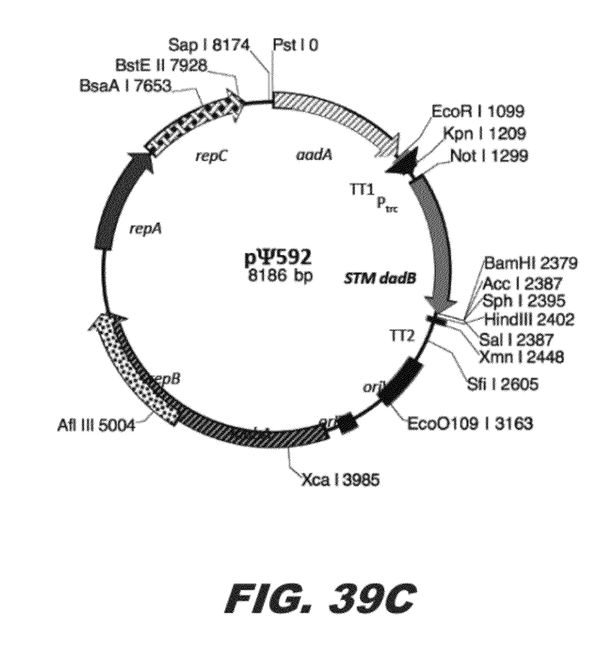

FIG. 39 depicts an experiment showing that Salmonella Alr and DadB complement ΔAlr in a Synechocystis sp. PCC 6803 strain. (A) shows that when pΨ570, pΨ591 and pΨ592 are transformed into the Δalr strain SD546 they grow well in the BG-11 media without the supplement (D-alanine) while SD546 dies by lysis in BG-11 media without D-alanine as a supplement. (B) shows the Salmonella alanine racemases alr gene inserted into pΨ568 to yield pΨ591. (C) the Salmonella alanine racemase dadB gene inserted into pΨ568 to yield pΨ592.

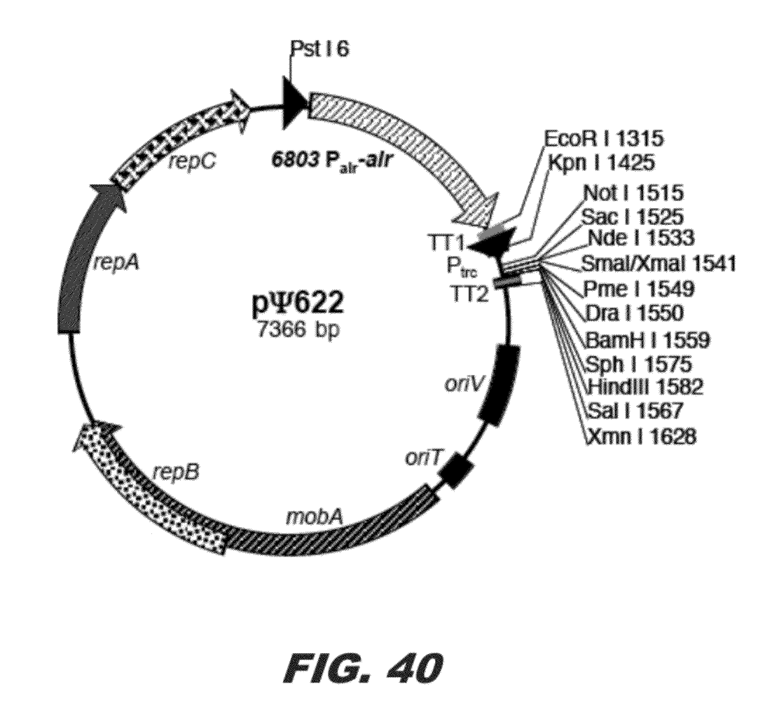

FIG. 40 depicts the balanced-lethal Alr⁺ vector pΨ622 for Synechocystis.

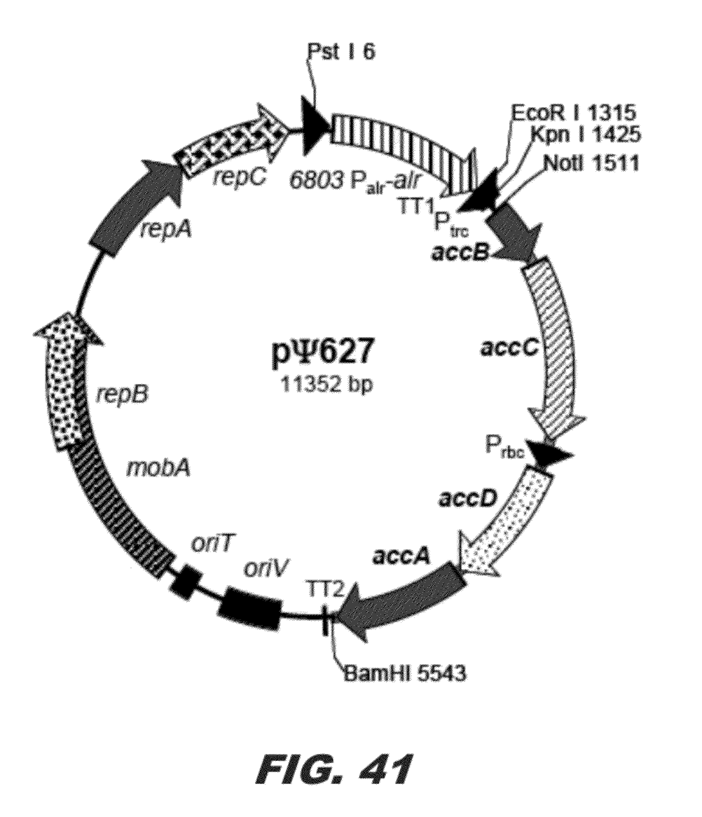

FIG. 41 depicts the plasmid pΨ627 containing the Synechocystis reconstructed operon accBCDA.

### DETAILED DESCRIPTION OF THE INVENTION

The present invention provides a bacterium capable of producing fatty acids. In particular, a bacterium of the invention may be used to generate fatty acids and release the fatty acids into the culture medium. Advantageously, the released fatty acids may be used as a biofuel precursor. Consequently, the invention also provides methods of producing fatty acids, and methods of harvesting the fatty acids.

### I. Recombinant Bacterium

A recombinant bacterium of the invention may comprise one or more alterations to increase fatty acid production, to enable fatty acid secretion, to enable fatty acid release, and to modulate fatty acid structure. These mutations are discussed in more detail below.

A bacterium of the invention is typically a cyanobacterium. In some embodiments, a bacterium belongs to the order Chroococcales. In a preferred embodiment, the bacterium is derived from the species Synechocystis. For instance, a bacterium of the invention may be derived from Synechocystis PCC sp. 6803.

### (a) Alterations to Increase Fatty Acid Generation

A recombinant bacterium of the invention may comprise one or more alterations to increase fatty acid generation. For instance, a bacterium may comprise an alteration that enables the synthesis of an acyl-ACP thioesterase, that inhibits fatty acid degradation, that channels resources into fatty acid synthesis, that down-regulates or eliminates competing pathways, that decreases repression or feedback inhibition, and that maintains stationary phase fatty acid production. Each of these alterations are described in more detail below.

i. Acyl-ACP Thioesterase

A bacterium of the invention may comprise an alteration that enables the synthesis of at least one acyl-ACP thioesterase (hereinafter TE). Methods of altering a bacterium to synthesize a TE are known in the art. For instance, a bacterium may be altered to express a nucleic acid encoding a TE. Such a nucleic acid may be operably linked to a regulated promoter or a constitutive promoter. In certain embodiments, a bacterium may synthesize one, two, three, four or five TEs. A nucleic acid encoding a TE may be chromosomally integrated, or may be expressed on an extrachromosomal vector. Suitable vectors are known in the art. Similarly, methods of chromosomally inserting a nucleic acid are known in the art. For additional details, see the Examples.

In some embodiments, a bacterium may synthesize a TE that is restricted to the cytosol of the bacterium. For instance, in one embodiment, a bacterium of the invention may synthesize a variant of TesA that is restricted to the cytosol of the bacterium. By way of non-limiting example, a bacterium may synthesize *TesA. The expression of a nucleic acid encoding TesA may be regulated or constitutive. For instance, the nucleic acid may be operably linked to an inducible promoter. Non-limiting examples of a suitable inducible promoters may include P_{nrsB}, P_{cmpA}, P_{isiA}, P_{sigE}, P_{lrtA}, or P_{sbD2}. P_{nrsB} is nickel inducible, P_{cmpA} is inducible by CO₂ limitation, P_{isiA} is includucible under low Fe conditions, P_{sigE} is inducible during the stationary phase, P_{lrtA} P is dark inducible, and P_{sbD2} may be induced by strong light.

Alternatively, the nucleic acid encoding a TE may be operably linked to a constitutive promoter, such as P_{psbA2}, P_{cpc}, P_{rbc}, P_{petB}, P_{psaAB}, P_{hspA}, or P_{sigA}.

Other TE enzymes are known in the art and may be used in the present invention. For instance, a TE from Cinnamomum camphorum, Umbellularia californica, or Cuphea hookeriana may be used. In another embodiment, a TE outlined in WO 2009/076559 may be used. In still another embodiment, a TE detailed in Table 7 below may be used.

The selection of the TE may be determined by the desired chain length of the resulting free fatty acid. For instance, see Table 7 below. In one embodiment, a TE with a preference for shorter free fatty acids may be used. For instance, a TE with a preference for C16, C14, C12, C10 or C8 fatty acids may be used.

A nucleic acid encoding a TE may be modified for high-level expression in a bacterium of the invention. As used herein, “modified” refers to an alteration of a nucleic acid sequence that results in a change in the level of transcription of a nucleic acid sequence, or that results in a change in the level of synthesis of encoded protein. For instance, in one embodiment, modify may refer to altering the start codon of a nucleic acid sequence. Generally speaking, a GTG or TTG start codon, as opposed to an ATG start codon, may decrease translation efficiency ten-fold. In another embodiment, modify may refer to altering the Shine-Dalgarno (SD) sequence of a nucleic acid sequence. The SD sequence is a ribosomal binding site (RBS) generally located 6-7 nucleotides upstream of the start codon. The SD/RBS consensus sequence is AGGAGG, and variations of the consensus sequence may alter translation efficiency. In yet another embodiment, modify may refer to altering the distance between the SD sequence and the start codon. In still another embodiment, modify may refer to altering the −35 sequence for RNA polymerase recognition. In a similar embodiment, modify may refer to altering the −10 sequence for RNA polymerase binding. In an additional embodiment, modify may refer to altering the number of nucleotides between the −35 and −10 sequences. In an alternative embodiment, modify may refer to optimizing the codons of the nucleic acid sequence to alter the level of translation of the mRNA. For instance, non-A rich codons initially after the start codon of a nucleic acid sequence may not maximize translation of the corresponding mRNA. Similarly, the codons of the nucleic acid sequence may be altered so as to mimic the codons in genes encoding highly synthesized proteins of a particular organism. In a further embodiment, modify may refer to altering the GC content of the nucleic acid sequence to change the level of translation of the corresponding mRNA. Additionally, modify may refer to alterations in the DNA sequence of a gene so that the transcribed mRNA is stabilized with a reduced rate of degradation but still able to specify a protein of the original amino acid sequence. In still another embodiment, a nucleic acid may be optimized by altering the nucleic acid such that the ability of the encoded protein to form efficient enzyme complexes is affected.

Methods of altering a nucleic acid as described above are known in the art. For more details, see the Examples.

ii. Inhibiting Fatty Acid Degradation

A recombinant bacterium of the invention may comprise an alteration that inhibits fatty acid degradation. For instance, the acyl-ACP synthetase (AAS) nucleic acid may be modified to decrease or eliminate expression of the nucleic acid. As described below the aas gene used to be referred to as the fadD gene. In one embodiment, aas is modified by replacing the aas chromosomal sequence with another sequence, such as a nucleic acid encoding a TE.

iii. Alterations that Channel Resources into Fatty Acid Synthesis

In some embodiments, a recombinant bacterium of the invention may comprise one or more alterations that channel resources into fatty acid synthesis. In certain embodiments, this may mean decreasing or eliminating expression of a nucleic acid that is not necessary for fatty acid synthesis. For instance, a bacterium may comprise a mutation that decreases or eliminates expression of a nucleic acid encoding a polyhydroxyalkanoate (PHA) synthesis enzyme. Non-limiting examples may include slr1993 and slr1994. In another embodiment, a bacterium may comprise a mutation that alters synthesis of an S-layer protein. Non-limiting examples may include mutations in sll1951, such as Δsll1951. In each of the above embodiments, the mutations should not alter the fitness of the bacterium in such a way as to reduce fatty acid synthesis.

Another way to channel resources into fatty acid synthesis is to increase the expression level of nucleic acid sequences encoding proteins in the primary free fatty acid production pathway. For instance, the expression of a nucleic acid encoding a protein involved in the generation of pyruvate may be increased. By way of non-limiting example, the expression of sll0587 or sll1275 may be increased. In another embodiment, the expression of a nucleic acid encoding a protein involved in the synthesis of acetyl-CoA from pyruvate, such as pdh or odh may be increased. In yet another embodiment, a nucleic acid sequence encoding a protein involved in the synthesis of malonyl-CoA from acetyl-CoA may be altered, such as accBCDA. Alternatively, a bacterium may be altered such that ACC may be overproduced by introducing a synthetic operon. Typically, the transcripts of the nucleic acids encoding the ACC subunits should be produced in relatively equal molar ratios. Non-limiting examples may include altering the bacterium to include the operon P_{cpc} accB accC P_{rbc} accD accA. In still another embodiment, the expression of a nucleic acid sequence encoding a protein involved in the synthesis of fatty acyl-ACP may be increased. For instance, the expression of a fab nucleic acid sequence may be increased (e.g. fabD, fabF, fabG, fabZ and fabl). In an alternative embodiment, the expression of a nucleic acid encoding an acyl carrier protein (such as ssl2084) may be increased. In a further embodiment, the expression of pyk may be increased.

Yet another way to channel resourses into fatty acid synthesis is to optimize expression of a nucleic acid encoding a protein involved in fatty acid synthesis. For instance, the expression may be optimized as described in section (a) i above. Alternatively, the expression may be optimized by altering the nucleic acid sequence to increase mRNA stability. For instance, the sequence may be altered to remove stem-loop structures.

iv. Alterations to Competing Pathways

A bacterium of the invention may be altered to reduce or eliminate the expression of a nucleic acid sequence encoding a protein that competes with fatty acid synthesis for reactants. By way of non-limiting example, the expression of slr1176 for the glycogen synthesis pathway; slr0301 (pps) for the phosphoenolpyruvate pathway; slr1192 for the predicted alcohol synthesis pathway; slr0089 and slr0090 for the vitamin E pathway; sll0920 for the oxaloacetate pathway; sll0891 for the malate synthesis pathway; sll1564 for the leucine synthesis pathway, sll0401 for the citrate pathways, and slr2132, sll1299, sll0542, slr0091 and sll0090 for the acetate pathways may be reduced or eliminated. Similarly, the expression of a nucleic acid sequence encoding a cyanophycin synthetase (such as slr2002 or slr2001) can be reduced or eliminated.

Expression of the above nucleic acid sequences may be reduced by altering the promoter, SD sequence, and/or start codon, etc. as described in section (a)i. above.

v. Alterations that Decrease Repression or Feedback Inhibition

A bacterium of the invention may be altered to decrease repression of fatty acid synthesis or to decrease feedback inhibition of fatty acid synthesis. For instance, expression of a TE, as described above, may be used to decrease inhibition of ACC, FabH, and Fabl. Similarly, repression may be decreased by altering the promoters of nucleic acids encoding proteins involved in fatty acid syntheis so that they do not include the binding sequences for repressors. Further examples of methods of decreasing feedback inhibition and repression are described in the Examples.

vi. Alterations to Maintain Stationary Phase Fatty Acid Production

A recombinant bacterium of the invention may be altered so as to allow secretion of fatty acids during stationary growth phase. Generally speaking, such alterations may include supplying a bacterium with a nucleic acid sequence encoding a protein involved in fatty acid synthesis, wherein the nucleic acid is operably linked to a promoter with increased activity in the stationary phase. Non-limiting examples of such promoters are detailed in the Examples.

### (b) Fatty Acid Secretion

A bacterium of the invention may comprise alterations to enable and/or increase fatty acid secretion. In one embodiment, a polar cell layer of the bacterium may be altered so as to increase fatty acid secretion. By way of example, the peptidoglycan layer, the outer membrane layer, and/or the S layer of a bacterium may be altered to enable increased fatty acid secretion. For instance, the expression of a nucleic acid encoding an S-layer protein, such as sll1951, may be decreased or eliminated. In one embodiment, a bacterium may comprise the mutation Δsll1951.

In another embodiment, the polypeptidoglycan layer of a bacterium may be weakened to enable increased fatty acid secretion. Methods of weakening the polypeptidoglycan layer may include administering an antibiotic, such as ampicillin, to the bacterium. Care should be taken, however, to balance the ability to secrete fatty acids with the potential for cell lysis. Such a balance may be experimentally determined as detailed in the Examples.

Another method to weaken the peptidoglycan layer may comprise down-regulating the transcription efficiency of nucleic acids encoding protein involved in peptidoglycan synthesis, such as those in the mur (e.g., slr0017, slr1423, slr1656 and sll2010) and ldh (e.g., slr0528 and slr1656) families to weaken the polypeptidoglycan layer structures. In another embodiment, a nucleic acid encoding a penicillin-binding protein such as ftsl (sll1833), mrcB (slr1710) and ponA (sll0002) may be deleted or modified. These proteins are required for the assembly of the peptidoglycan.

Yet another method to interfere with peptidoglycan synthesis is by substituting a nucleic acid for a central step in an essential pathway with one from another bacterial species, such as using an exogenous asd or alr gene. Still yet another way to weaken the polypeptidoglycan layer is to introduce one or more nucleic acid sequence encoding an endolysin from a bacteriophage. Such a nucleic acid sequence may then be expressed at low levels. Endolysins are peptidoglycan-degrading enzymes that attack the covalent linkages of the peptidoglycans that maintain the integrity of the cell wall. For instance, the endolysin gp19 from Salmonella phage P22 is able to degrade the 6803 polypeptidoglycan layers, and the endolysin R from E. coli phage λ is able to compromise the 6803 polypeptidoglycan layers. These sequences may be expressed with different promoters with variant low transcription efficiencies to limit adverse growth effects.

Yet another method to increase fatty acid secretion is to express or overexpress a nucleic acid sequence encoding a transporter or porin to make channels for the lipid. Many transport and efflux proteins serve to excrete a large variety of compounds, and these can possibly be modified to be selective for fatty acids. For example, E. coli outer membrane protein FadL is a membrane-bound fatty acid transporter, which binds long chain fatty acid with a high affinity. Other suitable transport proteins may include efflux proteins and fatty acid transporter proteins (FATP). Suitable non-limiting examples may be found in Table 9.

(c) Fatty Acid Release from Cellular Membranes

Another aspect of the invention encompasses the discovery that the regulated expression of a nucleic acid encoding a protein capable of hydrolyzing the lipid membranes to free fatty acids may be used to disrupt the cells and release intracellular fatty acids. Hence, in one embodiment, the invention encompasses a cyanobacterium comprising an inducible promoter operably-linked to a nucleic acid encoding a first protein capable of hydrolyzing the lipid membranes of the bacterium and at least one endolysin protein. In another embodiment, the invention encompasses a cyanobacterium comprising a first nucleic acid, wherein the first nucleic acid comprises a first inducible promoter operably-linked to a nucleic acid encoding a first protein capable of hydrolyzing the lipid membranes of the bacterium; and a second nucleic acid, wherein the second nucleic acid comprises a second promoter operably-linked to a nucleic acid encoding at least one endolysin protein.

In certain instances, the invention encompasses a cyanobacterium comprising more than one integrated nucleic acid construct of the invention. For instance, the invention may encompass a cyanobacterium comprising a first inducible promoter operably-linked to a nucleic acid encoding a first protein capable of hydrolyzing the lipid membranes of the bacterium, a second inducible promoter operably-linked to a different nucleic acid encoding a first protein capable of hydrolyzing the lipid membranes of the bacterium, and at least two endolysin proteins. In a further embodiment, the nucleic acid sequences encoding the endolysin proteins may be operably linked to a constitutive promoter.

Methods of making a cyanobacterium of the invention are known in the art. Generally speaking, a cyanobacterium is transformed with a nucleic acid contstruct of the invention. Methods of transformation are well known in the art, and may include electroporation, natural transformation, and calcium choloride mediated transformation. Methods of screening for and verifying chromosomal integration are also known in the art.

In one embodiment, a method of making a cyanobacterium of the invention may comprise first transforming the bacterium with a vector comprising, in part, an antibiotic-resistance marker and a negative selection marker. Chromosomal integration may be selected for by selecting for antiobiotic resistance. Next, the antibiotic-resistant strain is transformed with a similar vector comprising the target genes of interest. Chromosomal integration of the target genes may be selected for by selecting for the absence of the negative marker. For instance, if the negative marker is sacB, then one would select for sucrose resistance. For more details, see Kang et al., J Bacteriol. (2002) 184(1):307-12 and Sun et al., Appl. Environ. Microbiol. (2008) 74:4241-45, hereby incorporated by reference in their entirety.

i. Nucleic Acid Constructs

The present invention encompasses a nucleic acid construct that, when introduced into a bacterium, may be used in a method for inducing the degradation of lipid membrane or the peptidoglycan layer of a bacterial cell wall. In one embodiment, the nucleic acid comprises an inducible promoter operably-linked to a nucleic acid sequence encoding a first protein capable of hydrolyzing bacterial lipid membranes into free fatty acids. In another embodiment, the nucleic acid comprises an inducible promoter operably-linked to a nucleic acid sequence encoding a first protein capable of forming a lesion in a bacterial lipid membranes. In yet another embodiment, the nucleic acid comprises a promoter operably-linked to at least one endolysin. In another embodiment, the nucleic acid comprises an inducible promoter operably-linked to both a nucleic acid sequence encoding a first protein and a nucleic acid sequence encoding at least one endolysin. In still another embodiment, the nucleic acid comprises an inducible promoter operably-linked to a nucleic acid sequence encoding a first protein and a second promoter operably-linked to a nucleic acid sequence encoding at least one endolysin. Each component of the above nucleic acid constructs is discussed in more detail below.

Methods of making a nucleic acid construct of the invention are known in the art. Additional information may be found in Sambrook et al., Molecular Cloning: A Laboratory Manual (New York: Cold Spring Harbor Laboratory Press, 1989)

ii. Promoters

A nucleic acid construct of the present invention comprises a promoter. In particular, a nucleic acid construct comprises a first inducible promoter. In some embodiments, a nucleic acid also comprises a second promoter. When a nucleic acid comprises a first and a second promoter, the promoters may read in opposite directions, or may read in the same direction.

A. First Inducible Promoter

In certain embodiments, a nucleic acid of the invention encompasses a first inducible promoter. Non-limiting examples of inducible promoters may include, but are not limited to, those induced by expression of an exogenous protein (e.g., T7 RNA polymerase, SP6 RNA polymerase), by the presence of a small molecule (e.g., IPTG, galactose, tetracycline, steroid hormone, abscisic acid), by absence of small molecules (e.g., CO₂, iron, nitrogen), by metals or metal ions (e.g., copper, zinc, cadmium, nickel), and by environmental factors (e.g., heat, cold, stress, light, darkness), and by growth phase. In each of the above embodiments, the inducible promoter is preferably tightly regulated such that in the absence of induction, substantially no transcription is initiated through the promoter. Additionally, induction of the promoter of interest should not typically alter transcription through other promoters. Also, generally speaking, the compound or condition that induces an inducible promoter should not be naturally present in the organism or environment where expression is sought.

In one embodiment, the inducible promoter is induced by limitation of CO₂ supply to the cyanobacteria culture. By way of non-limiting example, the inducible promoter may be variant sequences of the promoter sequence of Synechocystis PCC 6803 that are up-regulated under the CO₂-limitation conditions, such as the cmp genes, ntp genes, ndh genes, sbt genes, chp genes, and rbc genes.

In one embodiment, the inducible promoter is induced by iron starvation or by entering the stationary growth phase. By way of non-limiting example, the inducible promo'ter may be variant sequences of the promoter sequence of the Synechocystis PCC 6803 isiA gene. In some embodiments, the inducible promoter may be variant sequences of the promoter sequence of cyanobacterial genes that are up-regulated under Fe-starvation conditions such as isiA, or when the culture enters the stationary growth phase, such as isiA, phrA, sigC, sigB, and sigH genes.

In one embodiment, the inducible promoter is induced by a metal or metal ion. By way of non-limiting example, the inducible promoter may be induced by copper, zinc, cadmium, mercury, nickel, gold, silver, cobalt, and bismuth or ions thereof. In one embodiment, the inducible promoter is induced by nickel or a nickel ion. In an exemplary embodiment, the inducible promoter is induced by a nickel ion, such as Ni²⁺. In another exemplary embodiment, the inducible promoter is the nickel inducible promoter from Synechocystis PCC 6803. In another embodiment, the inducible promoter may be induced by copper or a copper ion. In yet another embodiment, the inducible promoter may be induced by zinc or a zinc ion. In still another embodiment, the inducible promoter may be induced by cadmium or a cadmium ion. In yet still another embodiment, the inducible promoter may be induced by mercury or a mercury ion. In an alternative embodiment, the inducible promoter may be induced by gold or a gold ion. In another alternative embodiment, the inducible promoter may be induced by silver or a silver ion. In yet another alternative embodiment, the inducible promoter may be induced by cobalt or a cobalt ion. In still another alternative embodiment, the inducible promoter may be induced by bismuth or a bismuth ion.

In some embodiments, the promoter is induced by exposing a cell comprising the inducible promoter to a metal or metal ion. The cell may be exposed to the metal or metal ion by adding the metal to the bacterial growth media. In certain embodiments, the metal or metal ion added to the bacterial growth media may be efficiently recovered from the media. In other embodiments, the metal or metal ion remaining in the media after recovery does not substantially impede downstream processing of the media or of the bacterial gene products.

In one embodiment, the nucleic acid comprises a metal or metal ion inducible promoter operably-linked to a nucleic acid sequence encoding a first protein capable of hydrolyzing a bacterial lipid membrane. In another embodiment, the nucleic acid comprises a metal or metal ion inducible promoter operably-linked to both a nucleic acid sequence encoding a first protein and a nucleic acid sequence encoding at least one endolysin. In yet another embodiment, the nucleic acid comprises a metal or metal ion inducible promoter operably-linked to at least one endolysin. In still another embodiment, the nucleic acid comprises a metal or metal ion inducible promoter operably-linked to a nucleic acid sequence encoding a first protein and a second promoter operably-linked to a nucleic acid sequence encoding at least one endolysin.

B. Second Promoter

Certain nucleic acid constructs of the invention may comprise a second promoter. The second promoter may be an inducible promoter, or may be a constitutive promoter. If the second promoter is an inducible promoter, it may or may not be induced by the same compound or condition that induces the first inducible promoter. In one embodiment, the same compound or condition induces both the first and the second inducible promoters. In another embodiment, the first inducible promoter is induced by a different compound or condition than the second inducible promoter. Non-limiting examples of inducible promoters that may be used are detailed in section I(a)(i) above.

Constitutive promoters that may comprise the second promoter are known in the art. Non-limiting examples of constitutive promoters may include constitutive promoters from Gram-negative bacteria or a bacteriophage propogating in a Gram-negative bacterium. For instance, promoters for genes encoding highly expressed Gram-negative gene products may be used, such as the promoter for Lpp, OmpA, rRNA, and ribosomal proteins. Alternatively, regulatable promoters may be used in a strain that lacks the regulatory protein for that promoter. For instance P_{lac}, P_{tac}, and P_{trc} may be used as constitutive promoters in strains that lack Lad. Similarly, P22 P_{R} and P_{L} may be used in strains that lack the P22 C2 repressor protein, and λ P_{R} and P_{L} may be used in strains that lack the λ C1 repressor protein. In one embodiment, the constitutive promoter is from a bacteriophage. In another embodiment, the constitutive promoter is from a Salmonella bacteriophage. In yet another embodiment, the constitutive promoter is from a cyanophage. In some embodiments, the constitutive promoter is a Synechocystis promoter. For instance, the constitutive promoter may be the P_{psbAII} promoter or its variant sequences, the P_{rbc} promoter or its variant sequences, the P_{cpc} promoter or its variant sequences, and the P_{mpB} promoter or its variant sequences.

In one embodiment, a nucleic acid of the invention comprises a metal or metal ion inducible promoter operably-linked to a nucleic acid sequence encoding a first protein and a second constitutive promoter operably-linked to a nucleic acid sequence encoding at least one endolysin. In another embodiment, a nucleic acid of the invention comprises a metal or metal ion inducible promoter operably-linked to a nucleic acid sequence encoding a first protein and a second inducible promoter operably-linked to a nucleic acid sequence encoding at least one endolysin.

iii. First Protein

A nucleic acid construct of the invention also comprises a sequence encoding at least one first protein. Generally speaking, a first protein is a protein capable of degrading the lipid membranes into free fatty acid and release intracellular fatty acids. For instance, the first protein may be a lipolytic enzyme that is able to hydrolyze acylglycerols. In one embodiment, the first protein may be a lipolytic enzyme that hydrolyzes diacylglycerols, including MGDG (monogalactosyl diacylglycerol), DGDG (digalactosyl diacylglycerol), PG (phosphatidylglycerol), and SQDG (sulfoquinovosyl diacylglycerol). In another embodiment, the first protein may be a lipase that hydrolyzes triacylglycerols. In yet another embodiment, the first protein may be a lipolytic enzyme that hydrolyzes monoacylglycerols. In still yet another embodiment, the first protein may be a lipolytic enzyme from a bacterium, e.g., Staphylococcus hyicus. In a further embodiment, the first protein may be a lipolytic enzyme from a fungus, e.g., Fusarium oxysporum. In one embodiment, the first protein may be a lipolytic enzyme from an animal, e.g, guinea pig.

In other embodiments, a first protein is a protein capable of hydrolyzing a lipid membrane, such that the endolysin has access to the peptidoglycan layer of the cell wall. In these embodiments, the first protein may be a bacteriophage protein. For instance, the first protein may be a bacteriophage holin protein. In one embodiment, the first protein is a holin from a bacteriophage that infects gram-negative bacteria. In another embodiment, the first protein is a holin from a bacteriophage that infects gram-positive bacteria. In certain embodiments, the first protein is a holin from a cyanophage. In one embodiment, the first protein is a holin from a bacteriophage that infects Synechocystis. In another embodiment, the first protein may be from a bacteriophage that infects Salmonella. In still another embodiment, the first protein may be from a P22 phage. For example, the first protein may be gene 13 of the P22 phage. In yet another embodiment, the first protein may be from a λ phage. For example, the first protein may be encoded by gene S of the λ phage. In still another embodiment, the first protein may be from an E. coli phage. For instance, the first protein may be encoded by gene E of E. coli phage PhiX174. In certain embodiments, a nucleic acid of the invention may comprise at least two holins. In one embodiment, a nucleic acid may comprise a holin from P22 and a holin from λ phage. For instance, the nucleic acid may comprise gene 13 and gene S.

Additionally, a first protein may be a holin described above with at least one, or a combination of one or more, nucleic acid deletions, substitutions, additions, or insertions which result in an alteration in the corresponding amino acid sequence of the encoded holin protein, such as a homolog, ortholog, mimic or degenerative variant. For instance, a first protein may be a holin described above encoded by a nucleic acid with codons optimized for use in a particular bacterial strain, such as Synechocystis. Such a holin may be generated using recombinant techniques such as site-directed mutagenesis (Smith Annu. Rev. Genet. 19. 423 (1985)), e.g., using nucleic acid amplification techniques such as PCR (Zhao et al. Methods Enzymol. 217, 218 (1993)) to introduce deletions, insertions and point mutations. Other methods for deletion mutagenesis involve, for example, the use of either BAL 31 nuclease, which progressively shortens a double-stranded DNA fragment from both the 5′ and 3′ ends, or exonuclease III, which digests the target DNA from the 3′ end (see, e.g., Henikoff Gene 28, 351 (1984)). The extent of digestion in both cases is controlled by incubation time or the temperature of the reaction or both. Point mutations can be introduced by treatment with mutagens, such as sodium bisulfite (Botstein et al. Science 229, 1193 (1985)). Other exemplary methods for introducing point mutations involve enzymatic incorporation of nucleotide analogs or misincorporation of normal nucleotides or alpha-thionucleotide by DNA polymerases (Shortle et al. Proc. Natl. Acad. Sci. USA 79,1588 (1982)). PCR-based mutagenesis methods (or other mutagenesis methods based on nucleic acid amplification techniques), are generally preferred as they are simple and more rapid than classical techniques (Higuchi et al. Nucleic Acids Res. 16, 7351 (1988); Vallette et al. Nucleic Acids Res. 17,723 (1989)).

In addition to having a substantially similar biological function, a homolog, ortholog, mimic or degenerative variant of a holin suitable for use in the invention will also typically share substantial sequence similarity to a holin protein. In addition, suitable homologs, ortholog, mimic or degenerative variants preferably share at least 30% sequence homology with a holin protein, more preferably, 50%, and even more preferably, are greater than about 75% homologous in sequence to a holin protein. Alternatively, peptide mimics of a holin could be used that retain critical molecular recognition elements, although peptide bonds, side chain structures, chiral centers and other features of the parental active protein sequence may be replaced by chemical entities that are not native to the holin protein yet, nevertheless, confer activity.

In determining whether a polypeptide is substantially homologous to a holin polypeptide, sequence similarity may be determined by conventional algorithms, which typically allow introduction of a small number of gaps in order to achieve the best fit. In particular, “percent homology” of two polypeptides or two nucleic acid sequences is determined using the algorithm of Karlin and Altschul. Such an algorithm is incorporated into the NBLAST and XBLAST programs of Altschul, et al. (J. Mol. Biol. 215, 403 (1990)). BLAST nucleotide searches may be performed with the NBLAST program to obtain nucleotide sequences homologous to a nucleic acid molecule of the invention. Equally, BLAST protein searches may be performed with the XBLAST program to obtain amino acid sequences that are homologous to a polypeptide of the invention. To obtain gapped alignments for comparison purposes, Gapped BLAST is utilized as described in Altschul, et al. (Nucleic Acids Res. 25, 3389 (1997)). When utilizing BLAST and Gapped BLAST programs, the default parameters of the respective programs (e.g., XBLAST and NBLAST) are employed. See www.ncbi.nlm.nih.gov for more details.

In one embodiment, a nucleic acid of the invention comprises a metal or metal ion inducible promoter operably-linked to a nucleic acid sequence encoding a P22 phage holin. In another embodiment, the nucleic acid comprises a metal or metal ion inducible promoter operably-linked to both a nucleic acid sequence encoding a P22 phage holin and a nucleic acid sequence encoding at least one endolysin. In yet another embodiment, the nucleic acid comprises a metal or metal ion inducible promoter operably-linked to a nucleic acid sequence encoding a P22 phage holin and a second promoter operably-linked to a nucleic acid sequence encoding at least one endolysin.

iv. Endolysin

In some embodiments, a nucleic acid of the invention comprises at least one endolysin. In other embodiments, a nucleic acid of the invention comprises at least two endolysins. In yet another embodiment, a nucleic acid of the invention comprises at least three endolysins. In still another embodiment, a nucleic acid of the invention may comprise at least four endolysins. As used herein, “endolysin” refers to a protein capable of degrading the peptidoglycan layer of a bacterial cell wall. Generally speaking, the term endolysin encompasses proteins selected from the group consisting of lysozyme or muramidase, glucosaminidase, transglycosylase, amidase, and endopeptidase. Exemplary endolysins do not affect the cell until after the first protein creates lesions in the lipid membranes. Stated another way, the accumulation of endolysins in the cytosol of a bacterium will typically not substantially impair the growth rate of the bacterium. In another exemplary embodiment, the endolysin has a high enzymatic turnover rate. In yet another exemplary embodiment, the endolysin is from a gram positive bacterium. Because the cell walls of gram positive bacteria typically have a thicker peptidoglycan layer, an endolysin from a gram positive bacteria might be expected to have a higher enzymatic turnover rate.

In one embodiment, at least one endolysin is from a bacteriophage. In certain embodiments, suitable endolysins may be from phages detailed in section I(c)(ii) above in reference to the first protein. In another embodiment, at least one endolysin is from a Salmonella bacteriophage. In yet another embodiment, at least one endolysin is from a P22 phage. In still yet another embodiment, at least one endolysin is from a λ phage. In an alternative embodiment, at least one endolysin is gp19 from a P22 phage. In another alternative, a nucleic acid of the invention comprises gp19 and gp15 from a P22 phage. In some embodiments, at least one endolysin is R from a λ phage. In other embodiments, a nucleic acid of the invention comprises R and Rz from a λ phage. In certain embodiments, a nucleic acid of the invention comprises gp19, gp15, R, and Rz.

Additionally, an endolysin may be a protein described above with at least one, or a combination of one or more, nucleic acid deletions, substitutions, additions, or insertions which result in an alteration in the corresponding amino acid sequence of the encoded endolysin protein, such as a homolog, ortholog, mimic or degenerative variant. Such an endolysin may be generated using recombinant techniques such as those described in section I(c)(ii) above in reference to a first protein. In addition to having a substantially similar biological function, a homolog, ortholog, mimic or degenerative variant of an endolysin suitable for use in the invention will also typically share substantial sequence similarity to an endolysin protein. In addition, suitable homologs, ortholog, mimic or degenerative variants preferably share at least 30% sequence homology with an endolysin protein, more preferably, 50%, and even more preferably, are greater than about 75% homologous in sequence to an endolysin protein. Alternatively, peptide mimics of an endolysin could be used that retain critical molecular recognition elements, although peptide bonds, side chain structures, chiral centers and other features of the parental active protein sequence may be replaced by chemical entities that are not native to the endolysin protein yet, nevertheless, confer activity. Percent homology may be calculated as described in section I(c) above.

v. Additional Components

In certain embodiments, nucleic acids of the invention may further comprise additional components, such as a marker, a spacer domain, and a flanking sequence.

A. Markers

In one embodiment, a nucleic acid of the invention comprises at least one marker. Generally speaking, a marker encodes a product that the host cell cannot make, such that the cell acquires resistance to a specific compound, is able to survive under specific conditions, or is otherwise differentiable from cells that do not carry the marker. Markers may be positive or negative markers. In some embodiments, a nucleic acid of the invention may comprise both a positive marker and a negative marker. In certain embodiments, the marker may code for an antibiotic resistance factor. Suitable examples of antibiotic resistance markers may include, but are not limited to, those coding for proteins that impart resistance to kanamycin, spectromycin, streptomycin, neomycin, gentamicin (G418), ampicillin, tetracycline, and chloramphenicol. Additionally, the sacB gene may be used as a negative marker. The sacB gene is lethal in many bacteria when they are grown on sucrose media. Additionally, fluorescent proteins may be used as visually identifiable markers. Generally speaking, markers may be present during construction of the strains, but are typically removed from the final constructs. Proteins can also be marked by adding a sequence such as FLAG, HA, His tag, that can be recognized by a monoclonal antibody using immunological methods. In some embodiments, a marker may be a unique identifier of a genetically modified cyanobacterium.

B. Spacer Domain

Additionally, a nucleic acid of the invention may comprise a Shine-Dalgarno sequence, or a ribsome binding site (RBS). Generally speaking, a RBS is the nucleic acid sequence in the mRNA that binds to a 16s rRNA in the ribosome to initiate translation. For Gram-negative bacteria, the RBS is generally AGGA. The RBS may be located about 8 to about 11 bp 3′ of the start codon of the first structural gene. One skilled in the art will realize that the RBS sequence or its distance to the start codon may be altered to increase or decrease translation efficiency.

C. Flanking Sequence

Nucleic acid constructs of the invention may also comprise flanking sequences. The phrase “flanking sequence” as used herein, refers to a nucleic acid sequence homologous to a chromosomal sequence. A construct comprising a flanking sequence on either side of a construct (i.e., a left flanking sequence and a right flanking sequence) may homologously recombine with the homologous chromosome, thereby integrating the construct between the flanking sequences into the chromosome. Generally speaking, flanking sequences may be of variable length. In an exemplary embodiment, the flanking sequences may be between about 300 and about 500 bp. In another exemplary embodiment, the left flanking sequence and the right flanking sequence are substantially the same length. For more details, see the Examples.

vi. Plasmids

A nucleic acid construct of the invention may comprise a plasmid suitable for use in a bacterium. Such a plasmid may contain multiple cloning sites for ease in manipulating nucleic acid sequences. Numerous suitable plasmids are known in the art.

Non-limiting examples of first inducible promoters, first proteins, second promoters, and endolysin combinations are listed in Table A below.

TABLE A

| First promoter | | Second | |
|---|---|---|---|
| induced by | First protein | promoter | Endolysin |
| Metal or metal | Cyanophage holin | — | At least one Cyanophage endolysin |
| ion | | | |
| Metal or metal | Cyanophage holin | — | At least one λ phage endolysin |
| ion | | | |
| Metal or metal | Cyanophage holin | — | P22 gene 19 |
| ion | | | |
| Metal or metal | Cyanophage holin | — | P22 gene 15 |
| ion | | | |
| Metal or metal | Cyanophage holin | — | P22 gene 19 and P22 gene 15 |
| ion | | | |
| Metal or metal | Cyanophage holin | — | At least one λ phage endolysin and at least one |
| ion | | | P22 phage endolysin |
| Metal or metal | P22 gene13 | — | At least one Cyanophage endolysin |
| ion | | | |
| Metal or metal | P22 gene13 | — | At least one λ phage endolysin |
| ion | | | |
| Metal or metal | P22 gene13 | — | P22 gene 19 |
| ion | | | |
| Metal or metal | P22 gene13 | — | P22 gene 15 |
| ion | | | |
| Metal or metal | P22 gene13 | — | P22 gene 19 and P22 gene 15 |
| ion | | | |
| Metal or metal | P22 gene13 | — | At least one λ phage endolysin and at least one |
| ion | | | P22 phage endolysin |
| Metal or metal | λ phage holin | — | At least one Cyanophage endolysin |
| ion | | | |
| Metal or metal | λ phage holin | — | At least one λ phage endolysin |
| ion | | | |
| Metal or metal | λ phage holin | — | P22 gene 19 |
| ion | | | |
| Metal or metal | λ phage holin | — | P22 gene 15 |
| ion | | | |
| Metal or metal | λ phage holin | — | P22 gene 19 and P22 gene 15 |
| ion | | | |
| Metal or metal | λ phage holin | — | At least one λ phage endolysin and at least one |
| ion | | | P22 phage endolysin |
| Metal or metal | A λ phage holin and a | — | At least one Cyanophage endolysin |
| ion | P22 phage holin | | |
| Metal or metal | λ phage holin and a | — | At least one λ phage endolysin |
| ion | P22 phage holin | | |
| Metal or metal | λ phage holin and a | — | P22 gene 19 |
| ion | P22 phage holin | | |
| Metal or metal | A λ phage holin and a | — | P22 gene 15 |
| ion | P22 phage holin | | |
| Metal or metal | A λ phage holin and a | — | P22 gene 19 and P22 gene 15 |
| ion | P22 phage holin | | |
| Metal or metal | A λ phage holin and a | — | At least one λ phage endolysin and at least one |
| ion | P22 phage holin | | P22 phage endolysin |
| Metal or metal | Cyanophage holin | constitutive | At least one Cyanophage endolysin |
| ion | | | |
| Metal or metal | Cyanophage holin | constitutive | At least one λ phage endolysin |
| ion | | | |
| Metal or metal | Cyanophage holin | constitutive | P22 gene 19 |
| ion | | | |
| Metal or metal | Cyanophage holin | constitutive | P22 gene 15 |
| ion | | | |
| Metal or metal | Cyanophage holin | constitutive | P22 gene 19 and P22 gene 15 |
| ion | | | |
| Metal or metal | Cyanophage holin | constitutive | At least one λ phage endolysin and at least one |
| ion | | | P22 phage endolysin |
| Metal or metal | P22 gene13 | constitutive | At least one Cyanophage endolysin |
| ion | | | |
| Metal or metal | P22 gene13 | constitutive | At least one λ phage endolysin |
| ion | | | |
| Metal or metal | P22 gene13 | constitutive | P22 gene 19 |
| ion | | | |
| Metal or metal | P22 gene13 | constitutive | P22 gene 15 |
| ion | | | |
| Metal or metal | P22 gene13 | constitutive | P22 gene 19 and P22 gene 15 |
| ion | | | |
| Metal or metal | P22 gene13 | constitutive | At least one λ phage endolysin and at least one |
| ion | | | P22 phage endolysin |
| Metal or metal | λ phage holin | constitutive | At least one Cyanophage endolysin |
| ion | | | |
| Metal or metal | λ phage holin | constitutive | At least one λ phage endolysin |
| ion | | | |
| Metal or metal | λ phage holin | constitutive | P22 gene 19 |
| ion | | | |
| Metal or metal | λ phage holin | constitutive | P22 gene 15 |
| ion | | | |
| Metal or metal | λ phage holin | constitutive | P22 gene 19 and P22 gene 15 |
| ion | | | |
| Metal or metal | λ phage holin | constitutive | At least one λ phage endolysin and at least one |
| ion | | | P22 phage endolysin |
| Metal or metal | A λ phage holin and a | constitutive | At least one Cyanophage endolysin |
| ion | P22 phage holin | | |
| Metal or metal | λ phage holin and a | constitutive | At least one λ phage endolysin |
| ion | P22 phage holin | | |
| Metal or metal | λ phage holin and a | constitutive | P22 gene 19 |
| ion | P22 phage holin | | |
| Metal or metal | A λ phage holin and a | constitutive | P22 gene 15 |
| ion | P22 phage holin | | |
| Metal or metal | A λ phage holin and a | constitutive | P22 gene 19 and P22 gene 15 |
| ion | P22 phage holin | | |
| Metal or metal | A λ phage holin and a | constitutive | At least one λ phage endolysin and at least one |
| ion | P22 phage holin | | P22 phage endolysin |
| Metal or metal | Cyanophage holin | inducible | At least one Cyanophage endolysin |
| ion | | | |
| Metal or metal | Cyanophage holin | inducible | At least one λ phage endolysin |
| ion | | | |
| Metal or metal | Cyanophage holin | inducible | P22 gene 19 |
| ion | | | |
| Metal or metal | Cyanophage holin | inducible | P22 gene 15 |
| ion | | | |
| Metal or metal | Cyanophage holin | inducible | P22 gene 19 and P22 gene 15 |
| ion | | | |
| Metal or metal | Cyanophage holin | inducible | At least one λ phage endolysin and at least one |
| ion | | | P22 phage endolysin |
| Metal or metal | P22 gene13 | inducible | At least one Cyanophage endolysin |
| ion | | | |
| Metal or metal | P22 gene13 | inducible | At least one λ phage endolysin |
| ion | | | |
| Metal or metal | P22 gene13 | inducible | P22 gene 19 |
| ion | | | |
| Metal or metal | P22 gene13 | inducible | P22 gene 15 |
| ion | | | |
| Metal or metal | P22 gene13 | inducible | P22 gene 19 and P22 gene 15 |
| ion | | | |
| Metal or metal | P22 gene13 | inducible | At least one λ phage endolysin and at least one |
| ion | | | P22 phage endolysin |
| Metal or metal | λ phage holin | inducible | At least one Cyanophage endolysin |
| ion | | | |
| Metal or metal | λ phage holin | inducible | At least one λ phage endolysin |
| ion | | | |
| Metal or metal | λ phage holin | inducible | P22 gene 19 |
| ion | | | |
| Metal or metal | λ phage holin | inducible | P22 gene 15 |
| ion | | | |
| Metal or metal | λ phage holin | inducible | P22 gene 19 and P22 gene 15 |
| ion | | | |
| Metal or metal | λ phage holin | inducible | At least one λ phage endolysin and at least one |
| ion | | | P22 phage endolysin |
| Metal or metal | A λ phage holin and a | inducible | At least one Cyanophage endolysin |
| ion | P22 phage holin | | |
| Metal or metal | λ phage holin and a | inducible | At least one λ phage endolysin |
| ion | P22 phage holin | | |
| Metal or metal | λ phage holin and a | inducible | P22 gene 19 |
| ion | P22 phage holin | | |
| Metal or metal | A λ phage holin and a | inducible | P22 gene 15 |
| ion | P22 phage holin | | |
| Metal or metal | A λ phage holin and a | inducible | P22 gene 19 and P22 gene 15 |
| ion | P22 phage holin | | |
| Metal or metal | A λ phage holin and a | inducible | At least one λ phage endolysin and at least one |
| ion | P22 phage holin | | P22 phage endolysin |
| CO₂ | Cyanophage holin | — | At least one Cyanophage endolysin |
| CO₂ | Cyanophage holin | — | At least one λ phage endolysin |
| CO₂ | Cyanophage holin | — | P22 gene 19 |
| CO₂ | Cyanophage holin | — | P22 gene 15 |
| CO₂ | Cyanophage holin | — | P22 gene 19 and P22 gene 15 |
| CO₂ | Cyanophage holin | — | At least one λ phage endolysin and at least one |
| | | | P22 phage endolysin |
| CO₂ | P22 gene13 | — | At least one Cyanophage endolysin |
| CO₂ | P22 gene13 | — | At least one λ phage endolysin |
| CO₂ | P22 gene13 | — | P22 gene 19 |
| CO₂ | P22 gene13 | — | P22 gene 15 |
| CO₂ | P22 gene13 | — | P22 gene 19 and P22 gene 15 |
| CO₂ | P22 gene13 | — | At least one λ phage endolysin and at least one |
| | | | P22 phage endolysin |
| CO₂ | λ phage holin | — | At least one Cyanophage endolysin |
| CO₂ | λ phage holin | — | At least one λ phage endolysin |
| CO₂ | λ phage holin | — | P22 gene 19 |
| CO₂ | λ phage holin | — | P22 gene 15 |
| CO₂ | λ phage holin | — | P22 gene 19 and P22 gene 15 |
| CO₂ | λ phage holin | — | At least one λ phage endolysin and at least one |
| | | | P22 phage endolysin |
| CO₂ | A λ phage holin and a | — | At least one Cyanophage endolysin |
| | P22 phage holin | | |
| CO₂ | λ phage holin and a | — | At least one λ phage endolysin |
| | P22 phage holin | | |
| CO₂ | λ phage holin and a | — | P22 gene 19 |
| | P22 phage holin | | |
| CO₂ | A λ phage holin and a | — | P22 gene 15 |
| | P22 phage holin | | |
| CO₂ | A λ phage holin and a | — | P22 gene 19 and P22 gene 15 |
| | P22 phage holin | | |
| CO₂ | A λ phage holin and a | — | At least one λ phage endolysin and at least one |
| | P22 phage holin | | P22 phage endolysin |
| CO₂ | Cyanophage holin | constitutive | At least one Cyanophage endolysin |
| CO₂ | Cyanophage holin | constitutive | At least one λ phage endolysin |
| CO₂ | Cyanophage holin | constitutive | P22 gene 19 |
| CO₂ | Cyanophage holin | constitutive | P22 gene 15 |
| CO₂ | Cyanophage holin | constitutive | P22 gene 19 and P22 gene 15 |
| CO₂ | Cyanophage holin | constitutive | At least one λ phage endolysin and at least one |
| | | | P22 phage endolysin |
| CO₂ | P22 gene13 | constitutive | At least one Cyanophage endolysin |
| CO₂ | P22 gene13 | constitutive | At least one λ phage endolysin |
| CO₂ | P22 gene13 | constitutive | P22 gene 19 |
| CO₂ | P22 gene13 | constitutive | P22 gene 15 |
| CO₂ | P22 gene13 | constitutive | P22 gene 19 and P22 gene 15 |
| CO₂ | P22 gene13 | constitutive | At least one λ phage endolysin and at least one |
| | | | P22 phage endolysin |
| CO₂ | λ phage holin | constitutive | At least one Cyanophage endolysin |
| CO₂ | λ phage holin | constitutive | At least one λ phage endolysin |
| CO₂ | λ phage holin | constitutive | P22 gene 19 |
| CO₂ | λ phage holin | constitutive | P22 gene 15 |
| CO₂ | λ phage holin | constitutive | P22 gene 19 and P22 gene 15 |
| CO₂ | λ phage holin | constitutive | At least one λ phage endolysin and at least one |
| | | | P22 phage endolysin |
| CO₂ | A λ phage holin and a | constitutive | At least one Cyanophage endolysin |
| | P22 phage holin | | |
| CO₂ | λ phage holin and a | constitutive | At least one λ phage endolysin |
| | P22 phage holin | | |
| CO₂ | λ phage holin and a | constitutive | P22 gene 19 |
| | P22 phage holin | | |
| CO₂ | A λ phage holin and a | constitutive | P22 gene 15 |
| | P22 phage holin | | |
| CO₂ | A λ phage holin and a | constitutive | P22 gene 19 and P22 gene 15 |
| | P22 phage holin | | |
| CO₂ | A λ phage holin and a | constitutive | At least one λ phage endolysin and at least one |
| | P22 phage holin | | P22 phage endolysin |
| CO₂ | Cyanophage holin | inducible | At least one Cyanophage endolysin |
| CO₂ | Cyanophage holin | inducible | At least one λ phage endolysin |
| CO₂ | Cyanophage holin | inducible | P22 gene 19 |
| CO₂ | Cyanophage holin | inducible | P22 gene 15 |
| CO₂ | Cyanophage holin | inducible | P22 gene 19 and P22 gene 15 |
| CO₂ | Cyanophage holin | inducible | At least one λ phage endolysin and at least one |
| | | | P22 phage endolysin |
| CO₂ | P22 gene13 | inducible | At least one Cyanophage endolysin |
| CO₂ | P22 gene13 | inducible | At least one λ phage endolysin |
| CO₂ | P22 gene13 | inducible | P22 gene 19 |
| CO₂ | P22 gene13 | inducible | P22 gene 15 |
| CO₂ | P22 gene13 | inducible | P22 gene 19 and P22 gene 15 |
| CO₂ | P22 gene13 | inducible | At least one λ phage endolysin and at least one |
| | | | P22 phage endolysin |
| CO₂ | λ phage holin | inducible | At least one Cyanophage endolysin |
| CO₂ | λ phage holin | inducible | At least one λ phage endolysin |
| CO₂ | λ phage holin | inducible | P22 gene 19 |
| CO₂ | λ phage holin | inducible | P22 gene 15 |
| CO₂ | λ phage holin | inducible | P22 gene 19 and P22 gene 15 |
| CO₂ | λ phage holin | inducible | At least one λ phage endolysin and at least one |
| | | | P22 phage endolysin |
| CO₂ | A λ phage holin and a | inducible | At least one Cyanophage endolysin |
| | P22 phage holin | | |
| CO₂ | λ phage holin and a | inducible | At least one λ phage endolysin |
| | P22 phage holin | | |
| CO₂ | λ phage holin and a | inducible | P22 gene 19 |
| | P22 phage holin | | |
| CO₂ | A λ phage holin and a | inducible | P22 gene 15 |
| | P22 phage holin | | |
| CO₂ | A λ phage holin and a | inducible | P22 gene 19 and P22 gene 15 |
| | P22 phage holin | | |
| CO₂ | A λ phage holin and a | inducible | At least one λ phage endolysin and at least one |
| | P22 phage holin | | P22 phage endolysin |

### (d) Fatty Acid Acyl Chain Structure Modification

A recombinant bacterium of the invention may be altered so as to modify the structure of the fatty acids produced. For instance, the chain length, the chain saturation, and the branching of the fatty acid may be modified. In certain embodiments, chain length may be altered by the choice of TE, as detailed above and in the examples. Furthermore, the expression of a TE may alter chain saturation.

A bacterium of the invention may be altered to produce branch chain fatty acids. Typically, such a bacterium will express one or more nucleic acid sequences encoding a protein involved in the production of branch chain fatty acids, such as a branched-chain amino acid aminotransferase, a branched-chain α-keto acid dehydrogenase complex, β-ketoacyl-ACP synthase III, acyl carrier protein, and β-ketoacyl-ACP synthase II. Suitable, non-limiting examples are detailed in Table 9 below.

### (e) Inducible Lipolytic Enzymes

A bacterium of the invention may also be altered so as to express a nucleic acid encoding a lipolytic enzyme. Such an enzyme may degrade membrane lipids into free fatty acids. This increases the amount of free fatty acids harvestable from a bacterium, and makes the harvest less labor intensive. Suitable lipolytic enzymes may include a galactolipase and/or a phospholipase. Examples of galactolipases and phospholipases are known in the art. In one embodiment, a lipolytic enzyme from Staphylococcus hyicus may be used. In another embodiment, a lipolytic enzyme from Fusarium oxysporum may be used. In yet another embodiment, an enzyme derived from guinea pigs (GPLRP2) may be used. In addition, a lipase encoded by the Synechocystis gene lipA (sll1969) can also be used to degrade membrane lipids.

To ensure that the synthesis of a lipolytic enzyme does not result in the premature lysis of a bacterium, the enzyme may be placed under the control of an inducible promoter. Suitable promoters may include a nickel inducible promoter and a CO₂ inducible promoter. For more details, see the Examples.

### II. Balanced Lethal System

The present invention also provides a recombinant bacterium comprising at least one chromosomally encoded essential nucleic acid sequence, wherein the essential nucleic acid sequence is altered so that it is not expressed, and at least one extrachromosomal vector. An “essential nucleic acid” is a native nucleic acid whose expression is necessary for cell viability. Consequently, a bacterium of the invention is non-viable if an essential nucleic acid sequence is not expressed. Therefore, the bacterium of the invention further comprises at least one extrachromosomal vector. The vector comprises a nucleic acid sequence, that when expressed, substantially functions as the essential nucleic acid. Hence, the bacterium is viable when the vector is expressed. This promotes stable maintenance of the vector in the absence of any exogenously supplied selective condition.

(a) Chromosomally Encoded Essential Nucleic Acid that is Altered so that it is not Expressed

A recombinant bacterium of the invention comprises at least one chromosomally encoded essential nucleic acid sequence, wherein the essential nucleic acid sequence is altered so that it is not expressed. As described above, an essential nucleic acid is a native nucleic acid whose expression is necessary for cell viability. In some embodiments, an individual nucleic acid sequence is not essential, but the combination of one or more sequences, together, is essential. Stated another way, if the nucleic acid sequences in an essential combination are altered, so that they are not expressed, the cell is non-viable.

A nucleic acid sequence that encodes a protein necessary for the formation of the peptidoglycan layer of the cell wall may be an essential nucleic acid. In one embodiment, an essential nucleic acid encodes a protein involved in D-alanine synthesis. For example, an essential nucleic acid may encode one or more alanine racemase proteins. In another embodiment, an essential nucleic acid may encode a protein involved in D-glutamate synthesis. In yet another embodiment, an essential nucleic acid may encode a protein involved in muramic acid synthesis. Such nucleic acid sequences are known in the art, and non-limiting examples may include asd, murA, murl, dapA, dapB, and alr. In an alternative embodiment, a nucleic acid sequence that encodes a protein whose metabolic activity is essential for growth or cell division may be an essential nucleic acid. Such nucleic acid sequences are also known in the art, and non-limiting examples may include pol nucleic acid sequences encoding DNA polymerases, rpo nucleic acid sequences encoding RNA polymerases, and rps and rpl nucleic acid sequences encoding ribosomal protein subunits. Since Synechocystis is a photoautotroph normally living in fresh water and marine environments it is capable of synthesizing all amino acids necessary for protein synthesis, all purines and pyrimidines needed for nucleic acid syntheses and all vitamins needed as co-factors for various enzymes essential for metabolic activities and survival. As such, nucleic acid sequences encoding functions for amino acid synmthesis, purine synthesis, pyrimidine synthesis and vitamin synthesis are essential nucleic acids.

A recombinant bacterium of the invention may comprise more than one chromosomally encoded essential nucleic acid sequence that is altered so that it is not expressed. For instance, a recombinant bacterium may comprise two, three, four, five, or more than five different chromosomally encoded altered essential nucleic acid sequences.

Methods of making a recombinant bacterium comprising a chromosomally encoded essential nucleic acid sequence that is altered so that it is not expressed are known in the art and detailed in the examples. Non-limiting examples of suitable alterations are detailed below.

i. Essential Nucleic Acid Encoding a Protein Involved in D-Alanine Synthesis

In one embodiment, an essential nucleic acid may encode a protein involved in D-alanine synthesis, since D-alanine is a required constituent of the peptidoglycan layer of a bacterial cell wall. Gram-positive bacteria comprise only one alanine racemase, an enzyme necessary for D-alanine synthesis. Consequently, if the essential nucleic acid sequence encoding the Gram-positive alanine racemase is altered so that it is not expressed, the bacterium is non-viable. Gram-negative bacteria, however, may comprise one more than one alanine racemases. Consequently, when the Gram-negative bacteria comprises two alanine racemases, it is the combination of both sequences that is essential, and the nucleic acid sequences encoding both alanine racemases need to be altered so that both sequences are not expressed. Suitable alterations may include deletion of two nucleic acid sequences encoding an alanine racemase. For instance, the combination of the deletions Δalr and ΔdadB will alter the essential combination such that neither racemase is expressed. Advantageously, an extrachromosomal vector need only encode one racemase to restore to the Gram-negative bacterium. In preferred embodiments, the Gram-negative bacterium is Synechocystis, and the essential nucleic acid encoding a protein involved in D-alanine synthesis is alr encoding alanine racemase.

ii. Essential Nucleic Acid Encoding a Protein Involved in Muramic Acid Synthesis

In another embodiment, an essential nucleic acid may encode a protein involved in muramic acid synthesis, as muramic acid is another required constituent of the peptidoglycan layer of the bacterial cell wall. For example, an essential nucleic acid may be murA. It is not possible to alter murA by deletion, however, because a ΔmurA mutation is lethal and can not be isolated. This is because the missing nutrient required for viability is a phosphorylated muramic acid that cannot be exogenously supplied because most, if not all, bacteria cannot internalize it. Consequently, the murA nucleic acid sequence may be altered to make expression of murA dependent on a nutrient that can be supplied during the growth of the bacterium. For instance, cyanobacteria may be altered to make expression of murA dependent on arabinose. For example, the alteration may comprise a ΔP_{murA}.:TT araC P_{BAD} murA deletion-insertion mutation. During in vitro growth of the bacterium, this type of mutation makes synthesis of muramic acid dependent on the presence of arabinose in the growth medium. During growth of the bacterium in culture, arabinose may be supplied to allow for growth of the bacterium. However, the bacterium is non-viable in a natural environment where arabinose is not present. Since cyanobacteria are unable to internalize arabinose, the bacterium may be altered to enable uptake of arabinose. For instance, cyanobacteria may be altered by introducing an araE gene from E. coli that encodes an arabinose-uptake protein.

iii. Essential Protein Involved in D-Glutamate Synthesis

In yet another embodiment, an essential nucleic acid may encode a glutamate racemase, an enzyme essential for the synthesis of D-glutamic acid, which is another required constituent of the peptidoglycan layer of the bacterial cell wall. An essential nucleic acid encoding a glutamate racemase may be altered by deletion. For instance, the mutation Δmurl alters the nucleic acid sequence encoding glutamate racemase so that it is not expressed. In some instances, isolation of such mutations might require introduction of a nucleic acid sequence encoding a glutamate transporter, such as gltS, to facilitate uptake of glutamate. Such modifications are likely to facilitate introducing mutl mutations into cyanobacteria since cyanobacteria are seldom in an environment with exogenous amino acids and therefore often lack enzymes to facilitate amino acid uptake.

iv. Essential Protein Involved in DAP Synthesis

In still another embodiment, an essential nucleic acid may encode a protein involved in the synthesis of diaminopimelic acid (DAP). Various nucleic acid sequences are involved in the eventual synthesis of DAP (FIG. 31), including asd, dapA, dapB, the Synechocystis sll0480 nucleic acid sequence encoding LL-DAP aminotransferase involved in the pathway from the DAP precursor L-tetrahydrodipicolonic acid, and ddI (slr1874 in Synechocystis 6803) encoding D-alanyl-D-alanine synthase. Methods of altering an essential nucleic acid encoding a protein involved in the synthesis of DAP are known in the art. For instance, one of skill in the art may use the teachings of U.S. Pat. No. 6,872,547, hereby incorporated by reference in its entirety, for alterations that abolish DAP synthesis. In one example, the essential nucleic acid asdA may be altered by a ΔasdA mutation, so that asdA is not expressed. This eliminates the bacterium's ability to produce β-aspartate semialdehyde dehydrogenase, an enzyme essential for the synthesis of methionine, threonine, DAP and lysine in cyanobacteria. Thus the asdA deletion mutation in Synechocystis can be used as an essential gene since free amino acids are essentially absent in aqueous environments inhabited by cyanobacteria. As another examples, the essential nucleic acids dapA, dapB and sll0480 encoding LL-DAP aminotransferase may be deleted to impose a requirement for DAP.

v. More than One Chromosomally Encoded Essential Nucleic Acid that is Altered

In exemplary embodiments of the invention, a recombinant bacterium may comprise more than one chromosomally encoded essential nucleic acid sequence that is altered so that it is not expressed and at least one extrachromosomal vector.

For instance, in one embodiment, a recombinant bacterium may comprise a first chromosomally encoded essential nucleic acid that is altered so that the first essential nucleic acid is not expressed, a second chromosomally encoded essential nucleic acid that is altered so that the second essential nucleic acid is not expressed, a first extrachromosomal vector, the vector comprising a nucleic acid comprising a nucleic acid sequence, that when expressed, substantially functions as the first essential nucleic acid sequence, and a second extrachromosomal vector, the vector comprising a nucleic acid sequence, that when expressed, substantially functions as the second essential nucleic acid sequence.

In another embodiment, a recombinant bacterium may comprise a first chromosomally encoded essential nucleic acid that is altered so that the first essential nucleic acid is not expressed, a second chromosomally encoded essential nucleic acid that is altered so that the second essential nucleic acid is not expressed, a third chromosomally encoded essential nucleic acid that is altered so that the third essential nucleic acid is not expressed, a first extrachromosomal vector, the vector comprising a nucleic acid comprising a nucleic acid sequence, that when expressed, substantially functions as the first essential nucleic acid sequence, a second extrachromosomal vector, the vector comprising a nucleic acid sequence, that when expressed, substantially functions as the second essential nucleic acid sequence, and a third extrachromosomal vector, the vector comprising a nucleic acid sequence, that when expressed, substantially functions as the third essential nucleic acid sequence.

In yet another embodiment, a recombinant bacterium may comprise a first chromosomally encoded essential nucleic acid that is altered so that the first essential nucleic acid is not expressed, a second chromosomally encoded essential nucleic acid that is altered so that the second essential nucleic acid is not expressed, a third chromosomally encoded essential nucleic acid that is altered so that the third essential nucleic acid is not expressed, a fourth chromosomally encoded essential nucleic acid that is altered so that the fourth essential nucleic acid is not expressed, a first extrachromosomal vector, the vector comprising a nucleic acid comprising a nucleic acid sequence, that when expressed, substantially functions as the first essential nucleic acid sequence, a second extrachromosomal vector, the vector comprising a nucleic acid sequence, that when expressed, substantially functions as the second essential nucleic acid sequence, a third extrachromosomal vector, the vector comprising a nucleic acid sequence, that when expressed, substantially functions as the third essential nucleic acid sequence, and a fourth extrachromosomal vector, the vector comprising a nucleic acid sequence, that when expressed, substantially functions as the fourth essential nucleic acid sequence.

In other embodiments, a recombinant bacterium may comprise more than four chromosomally encoded essential nucleic acid sequences that are each altered so that they are not expressed, and more than four corresponding extrachromosomal vectors.

By way of non-limiting example, suitable alterations in essential nucleic acid sequences may include an alteration selected from the group consisting of ΔasdA, a ΔdapA mutation, a ΔdapB mutation, a ΔdapA mutation with a ΔdapB mutation, a Δalr mutation, a ΔP_{murA}::TT araC P_{BAD} murA deletion-insertion mutation, a Δmurl mutation, a ΔaroA mutation, a ΔaroC mutation, a ΔaroD mutation, a ΔilvC mutation, and a ΔilvE mutation. For instance, a bacterium may comprise two, three, four, five, or more than five alterations in an essential nucleic acid sequence selected from the group consisting of ΔasdA, any Δdap mutation, a Δalr mutation, a ΔP_{murA}::TT araC P_{BAD} murA deletion-insertion mutation, a Δmurl mutation, a ΔaroA mutation, a ΔaroC mutation, a ΔaroD mutation, a ΔilvC mutation, and a ΔilvE mutation.

### (b) Extrachromosomal Vector

A recombinant bacterium of the invention also comprises an extrachromosomal vector. The vector comprises a nucleic acid sequence that when expressed, substantially functions as the chromosomally encoded essential nucleic acid that is not expressed. Furthermore, the vector typically also comprises one or more nucleic acid sequences that encode metabolic activity capable of enhancing the productivity of the cyanobacterial strain. As used herein, “vector” refers to an autonomously replicating nucleic acid unit. The present invention may be practiced with any known type of vector, including viral, cosmid, phasmid, and plasmid vectors. The most preferred type of vector is a plasmid vector. An exemplary type of plasmid may use one or more of the small plasmid replicons normally present in a Synechocystis species as described in Section III below.

The term “extrachromosomal,” as used herein, refers to the fact that the vector is not contained within the bacterium's chromosomal DNA. The vector may comprise some sequences that are identical or similar to chromosomal sequences of the bacterium, however, the vectors used herein do not integrate with chromosomal sequences of the bacterium.

As is well known in the art, plasmids and other vectors may possess a wide array of promoters, multiple cloning sequences, transcription terminators, etc., and vectors may vary in copy number per bacterium. Selection of a vector may depend, in part, on the desired level of expression of the nucleic acid sequence substantially functioning as the essential nucleic acid or functioning to specify the metabolic activity of importance to enhance the productivity of the cyanobacterial strain.

Additionally, vector copy number may be increased by selecting for mutations that increase plasmid copy number. These mutations may occur in the bacterial chromosome but are more likely to occur in the vector.

### (c) Toxin-Antitoxin Stability System

In some embodiments, a toxin-antitoxin stability system may be used to generate the balanced-lethal system. The antitoxins may be proteins or antisense RNAs that counteract the toxins. Non-limiting examples of toxin-antitoxin gene families from cyanobacteria may be the antisense RNA-regulated toxin-antitoxin gene families hok/sok and ldr, or SOS-induced toxins such as SymE.

### (d) Inhibiting Recombination

Although extrachromosomal vectors, such as plasmids, may be designed with unique nucleotide sequences, there is some potential for vector-vector recombination to occur that might lead to deletion of and/or alterations in one or more nucleic acid sequences encoding metabolic activity capable of enhancing the productivity of the cyanobacterial strain. Accordingly, in some embodiments, a recombinant bacterium of the invention may be deficient in one or more of the enzymes that catalyzes recombination between extrachromosomal vectors. If a bacterium comprises only a single extrachromosomal vector, then such mutations are not necessary. If two or more extrachromosomal vectors are used, however, then the recombinant bacterium may be modified so that one or more recombination enzymes known to catalyze vector-vector recombination are rendered non-functional.

In certain embodiments, the recombination enzymes do not participate in recombinations involving chromosomal nucleic acid sequences. For instance, the recombinant bacterium may comprise a ΔrecF and a ΔrecJ mutation. These mutations do not alter the attributes of the recombinant bacterium. One of skill in the art will appreciate that other recombination enzymes known to catalyze vector-vector recombination but not to participate in recombinations involving chromosomal nucleic acid sequences may be targeted for deletion or mutation in addition to RecF and RecJ.

Alternatively, the recombinant bacterium may be modified by introducing a ΔrecA mutation that prevents all recombination, whether between vectors or chromosomal nucleic acid sequences. Introducing a ΔrecA mutation may enhance sensitivity of the bacteirum to ultraviolet radiation in sunlight and render the cyanobacterial strain unsuitable for producing fatty acids or other biofuels or biofuel precursors. Alternatively, the recombinant bacterium may be modified by introducing mutations into recF and or recJ since they do not confer sensitivity to ultraviolet light.

### III. Cyanobacteria-Based Plasmids

The present invention also provides plasmids derived from one or more of the small plasmid replicons normally present in a Synechocystis species. For instance, gene functions responsible for stable maintenance of the small plasmid replicon in Synechocystis may be identified using methods known in the art. In addition, it may be determined if loss of the native Synechocystis plasmid is lethal or the plasmid is dispensable.

In some embodiments, a shuttle vector may be developed that will replicate in both E. coli and Synechocystis.

Vectors of the invention generally possess a multiple cloning site for insertion of a nucleic acid sequence that may be operably-linked to the promoter sequence and generally posses a transcription terminator (TT) sequence after a coding region. Preferably, vectors used herein do not comprise antibiotic resistance markers to select for maintenance of the vector.

### IV. Method of Producing Fatty Acids

Another aspect of the present invention is a method for the production of fatty acids. Generally speaking, a method of the invention comprises culturing a bacterium of the invention as detailed in section I above. Methods of culturing a cyanobacterium are known in the art and detailed in the examples. Fatty acids produced by the bacterium may be extracted from the culture media or culture biomass.

Methods of extracting the secreted fatty acids from the extracellular milieu are know in the art and detailed in the Examples. For instance, the fatty acids may be pipetted, filtered, and/or skimmed from the culture media. In addition, the culture media may be treated to extract any remaining fatty acids dissolved in the media. Such treatment is described in the Examples. Briefly, the media may be acidified and extracted with an organic solvent, such as hexane. The organic phase may then be separated and dried to give the fatty acids. In some embodiments, the media is extracted more than once with the organic solvent. For instance, the media may be extracted two, three, four or five times.

Unsecreted intracellular FFAs and lipids may also be extracted using means known in the art and detailed in the Examples.

The yield of fatty acids from a bacterium of the invention will generally be about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400 or more than 400 times the yield of fatty acids from a wild-type bacterium. In one embodiment, the yield is greater than 100, 200, 300, 400, 500, 600, 700, 800, 900, or 1000 times the yield of a wild-type bacterium. In another embodiment, the yield is greater than about 1000, 1500, 2000, 2500, 3000, or 3500 times the yield of a wild-type bacterium.

### DEFINITIONS

The term “cell wall”, as used herein, refers to the peptidoglycan layer of the cell wall. Stated another way, “cell wall” as used herein refers to the rigid layer of the cell wall.

The term “operably-linked”, as used herein, means that expression of a gene is under the control of a promoter with which it is spatially connected. A promoter may be positioned 5′ (upstream) of a gene under its control. The distance between the promoter and a gene may be approximately the same as the distance between that promoter and the gene it controls in the gene from which the promoter is derived. As is known in the art, variation in this distance may be accommodated without loss of promoter function.

The term “promoter”, as used herein, may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. In some embodiments, activators may bind to promoters 5′ of the −35 RNA polymerase recognition sequence, and repressors may bind 3′ to the −10 ribosome binding sequence.

The following examples are included to demonstrate preferred embodiments of the invention. It should be appreciated by those of skill in the art that the techniques disclosed in the examples that follow represent techniques discovered by the inventors to function well in the practice of the invention. Those of skill in the art should, however, in light of the present disclosure, appreciate that many changes can be made in the specific embodiments that are disclosed and still obtain a like or similar result without departing from the spirit and scope of the invention, therefore all matter set forth or shown in the accompanying drawings is to be interpreted as illustrative and not in a limiting sense.

### EXAMPLES

The following examples illustrate various iterations of the invention.

### Example 1

### Development of Means for Genetic Manipulation and Analysis

6803 is an ideal organism to genetically manipulate due to its high natural transformation efficiency and high double crossover homologous recombination efficiency (Kufryk, Sachet et al. 2002). Cai and Wolk have introduced a method to counter-select cells that retain the drug markers, thus enabling construction of the desired multiple recombinants (Cai and Wolk 1990). Suicide vectors harboring a positive selection marker (e.g., Km^{r}) and a counter selection marker (e.g., sacB) are widely applied in a two-step gene deletion and insertion for 6803 without leaving any drug marker residuals (FIG. 1). However, since rapidly growing cyanobacteria contain multiple chromosomes and only one chromosome is involved in the initial recombination event, segregation is necessary for obtaining a genetically homogeneous strain, where all the chromosomes contain the same sequence. The phenomena of segregation and phenotypic lags are well known in bacterial genetics (Hayes, 1968).

Conditions for transformation and isolation of a recombinant by selection for a dominant phenotype have been well documented (Zang, Liu et al. 2007), and include DNA concentrations, cell competence, DNA uptake timing, and light dependence. However, few papers discuss the optimal conditions and efficiency of the counter selecting transformation and selection by a recessive phenotype (e.g., sucrose resistance by removal of sacB). By testing different plasmid concentrations, transformation and segregation times, and selection pressure levels, the inventors significantly improved the overall transformation efficiency for generating recombinants. A highly optimized experimental protocol for 10²-10⁵ transformants/μg DNA, which is sufficient for genetic engineering purposes, was developed. For example, after transformation, segregation without applying selective pressure is necessary to permit formation of ‘homozygous’ mutants that will phenotypically display the selected marker. The phenotypic and segregation lags for sucrose survival depend on the toxic or adverse effects of the insertion genes on the growth rate of the resulting mutant. Toxic genes or those causing slow growth rate usually require a longer segregation period. Additionally, the colonies grown from fully segregated single cells were isolated and tested to ensure that all the resulting strains are genetically homogenous. The details of optimized transformation are described further below. By using the optimized genetic manipulation methods, a series of strains with FFA secreting ability were constructed (Table 1).

TABLE 1
SD strains constructed

| SD No. | Genotype | Construction | Remarks |
|---|---|---|---|
| SD100 | Synechocystis sp. PCC | | From Dr. Wim Vermaas' lab, |
| | 6803 wild-type | | School of Life Science, Arizona |
| | | | State University |
| SD102 | ΔnrsBAC::13 sacB Km^{r} | Transform SD100 with | This is an intermediate strain with |
| | | pψ102 (ΔnrsBAC::13 | a Km^{r}-sacB cassette insertion |
| | | sacB Km^{r}) and select for | replacing nsrBAC, which can be |
| | | kanamycin resistance | replaced by a further insertion. |
| SD121 | ΔnrsBAC::13 15 19 | Transform SD102 with | Inducible lysis strain. P22 lysis |
| | | pψ121 (ΔnrsBAC::13 15 | cassette (13 15 19) is controlled |
| | | 19) and select for sucrose | by the nickel inducible promoter |
| | | survival | P_{nrsB} |
| SD122 | ΔnsrBAC::S R Rz | Transform SD102 with | Inducible lysis-releasing strain. λ |
| | | pψ122 (ΔnrsBAC::S R | lysis cassette (S R Rz) is |
| | | Rz) and select for | controlled by the nickel inducible |
| | | sucrose survival | promoter P_{nrsB} |
| SD123 | ΔnsrBAC::13 TT P_{psbAII} 15 | Transform SD102 with | Optimized P22 inducible lysis |
| | 19 | pψ123 (ΔnsrBAC::13 TT | strain. The holin gene 13 is |
| | | P_{psbAII} 15 19) and select | controlled by Ni²⁺, endolysin |
| | | for sucrose survival | genes 19 and 15 were |
| | | | transcribed by a constitutive |
| | | | promoter (P_{psbAII}). A |
| | | | transcriptional terminator (TT) |
| | | | was inserted to eliminate |
| | | | interference in gene expression. |
| SD200 | ΔlipA::sacB Km^{r} | Transform SD100 with | Intermediate strain for neutral lipid |
| | | pψ200 (ΔnrsBAC::13 | production. The foreign genes to |
| | | Km^{r}-sacB) and select for | be introduced at the lipA locus |
| | | kanamycin resistance | |
| SD201 | ΔlipA::P_{psbAII} atfA phaP1 | Transform SD200 with | Neutral lipid producing strain, the |
| | | pψ201 (ΔlipA::P_{psbAII} atfA | triglyceride synthesis gene atfA |
| | | phaP1) and select for | and lipid body gene phaP1 are |
| | | sucrose survival | inserted at the lipA locus and |
| | | | driven by a strong 6803 promoter |
| SD207 | Δ(slr1993 slr1994)::sacB | Transform SD100 with | Intermediate strain for deleting |
| | Km^{r} | pψ207 (Δ(slr1993 | PHA synthesis pathway, the PHA |
| | | slr1994)::sacB Km^{r}) and | synthesis genes in 6803 were |
| | | select for kanamycin | deleted by Km^{r}-sacB insertion |
| | | resistance | |
| SD210 | Δ(slr1993 slr1994)::P_{rbcLXS}- | Transform SD207 with | Optimized strain for neutral lipid |
| | aftA1-phaP1 | pψ210 (Δ(slr1993 | production. The sequences of |
| | | slr1994)::P_{rbcLXS} (aftA1- | atfA and phaP1 are modified by |
| | | phaP1) and select for | codon optimization and inserted |
| | | sucrose survival | in Δ(slr1993 slr1994) mutation. |
| SD214 | ΔfadD::sacB Km^{r} (ΔfadD | Transform SD100 with | Intermediate strain for deleting |
| | re-named Δaas) | pψ214 (Δaas::sacB Km^{r}) | fatty acid degrading pathway. The |
| | | and select for kanamycin | Acyl-ACP synthase gene (aas) in |
| | | resistance | 6803 was deleted by Km^{r}-sacB |
| | | | insertion |
| SD215 | ΔnsrBAC::P_{nrsR} ′tesA-HA | Transform SD102 with | Inducible FFA-secreting strain. E. coli |
| | | pψ215 (ΔnsrBAC::P_{nrsR} | *tesA gene fused with an HA |
| | | ′tesA-HA) and select for | tag is controlled by the Ni |
| | | sucrose survival | inducible promoter. |
| SD216 | ΔfadD::P_{psbA2} ′tesA-HA | Transform SD214 with | Constitutive FFA-secreting strain. |
| | (ΔfadD re-named Δaas) | pψ216 (Δaas::P_{psbA2} | E. coli *tesA gene fused with an |
| | | ′tesA-HA) and select for | HA tag is driven by P_{psbA2}, with |
| | | sucrose survival | the same orientation of Pₐₐₛ, and |
| | | | the aas gene is deleted. |
| SD219 | ΔnsrBAC::P_{nrsR} ′tesA-HA | Transform SD215 with | Intermediate strain for adding |
| | Δ(slr1993 slr1994)::sacB | pψ207 (Δ(slr1993 | multiple genes onto SD215. The |
| | Km^{r} | slr1994)::sacB Km^{r}) and | PHA synthesis genes in SD215 |
| | | select for kanamycin | were deleted by Km^{r}-sacB |
| | | resistance | insertion |
| SD220 | ΔfadD::P_{psbA2} ′tesA-HA | Transform SD216 with | Intermediate strain for adding |
| | Δ(slr1993 slr1994)::sacB | pψ207 (Δ(slr1993 | multiple genes onto SD216. The |
| | Km^{r} (ΔfadD re-named | slr1994)::sacB Km^{r}) and | PHA synthesis genes in SD216 |
| | Δaas) | select for kanamycin | were deleted by Km^{r}-sacB |
| | | resistance | insertion. |
| SD223 | ΔnsrBAC::P_{nrsR} ′tesA-HA | Transform SD215 with | 2^{nd} generation inducible FFA- |
| | Δ(slr1993 | pψ223 (Δ(slr1993 | secreting strain. ACC |
| | slr1994)::P_{cpc}accBC | slr1994)::P_{cpc}accBC | overproduction and PHA deletion |
| | P_{rbc}accDA | P_{rbc}accDA) and select for | were incorporated into SD215 |
| | | sucrose survival | |
| SD225 | ΔfadD::P_{psbA2} ′tesA-HA | Transform SD216 with | 2^{nd} generation constitutive FFA- |
| | Δ(slr1993 slr1994)::P_{cpc} | pψ223 (Δ(slr1993 | secreting strain. ACC |
| | accBC P_{rbc} accDA (ΔfadD | slr1994)::P_{cpc} accBC P_{rbc} | overproduction and PHA deletion |
| | re-named Δaas) | accDA) and select for | were incorporated into SD216 |
| | | sucrose survival | |
| SD228 | ΔnsrBAC::P_{nrsR} ′tesA-HA | Transform SD223 with | Intermediate strain for adding |
| | Δ(slr1993 slr1994)::P_{cpc} | pψ228 (Δsll1951::sacB | multiple genes into SD223. The |
| | accBC P_{rbc} accDA | Km^{r}) and select for | S-layer gene (sll1951) in SD223 |
| | Δsll1951::sacB Km^{r} | kanamycin resistance | was deleted by Km^{r}-sacB |
| | | | insertion |
| SD229 | ΔfadD::P_{psbA2} ′tesA-HA | Transform SD225 with | Intermediate strain for adding |
| | Δ(slr1993 slr1994)::P_{cpc} | pψ228 (Δsll1951::sacB | multiple genes onto SD225. The |
| | accBC P_{rbc} accDA | Km^{r}) and select for | S-layer gene (sll1951) in SD225 |
| | Δsll1951::sacB Km^{r} (ΔfadD | kanamycin resistance | was deleted by Km^{r}-sacB |
| | re-named Δaas) | | insertion |
| SD232 | ΔfadD::P_{psbA2} ′tesA-HA | Transform SD229 with | 3^{rd} generation constitutive FFA- |
| | Δ(slr1993 slr1994)::P_{cpc} | pψ231 (Δsll1951::*P_{psbA2} | secreting strain. Plant middle |
| | accBC P_{rbc} accDA | Uc fatB1 *P_{rbc} Ch fatB2) | chain thioesterase overproduction |
| | Δsll1951::*P_{psbA2} Uc fatB1 | and select for sucrose | and S-layer deletion were |
| | *P_{rbc} Ch fatB2 (ΔfadD re- | survival | incorporated into SD225 |
| | named Δaas) | | |
| SD240 | Δaas:: P_{psbA2} ′tesA | Transform SD232 with | Intermediate strain for adding |
| | Δ(slr1993-slr1994)::P_{cpc} | pψ240 (Δ(slr2001-slr2002) | genes into SD232. The |
| | accBC P_{rbc} accDA | ::sacB Km^{R}) and | cyanophycin genes (slr2001 |
| | Δsll1951::*P_{psbA2} Uc fatB1 | select for kanamycin | slr2002) in SD232 were deleted |
| | P_{rbc} Ch fatB2 Δ(slr2001-slr2002) | resistance | by sacB Km^{R} insertion |
| | ::sacB Km^{R} | | |
| SD243 | Δaas::P_{psbA2} ′tesA | Transform SD240 with | 4^{th} generation constitutive FFA- |
| | Δ(slr1993-slr1994)::P_{cpc} | pψ243 (Δ(slr2001-slr2002) | secreting strain. Plant C8:0, |
| | accBC P_{rbc} accDA | :: *P_{psbA2} Ch | C10:0 thioesterase |
| | Δsll1951::*P_{psbA2} Uc fatB1 | fatB2) and select for | overproduction and cyanophycin |
| | P_{rbc} Ch fatB2 Δ(slr2001-slr2002) | sucrose survival | deletion were incorporated into |
| | ::*P_{psbA2} Ch fatB2 | | SD232 |
| SD248 | Δaas::P_{psbA2} ′tesA | Transform SD243 with | Intermediate strain for adding |
| | Δ(slr1993-slr1994)::P_{cpc} | pψ248 (Δslr1710::sacB | genes into SD249. The penicillin |
| | accBC P_{rbc} accDA | Km^{R}) and select for | binding protein 2 gene (slr1710) |
| | Δsll1951::*P_{psbA2} Uc fatB1 | kanamycin resistance | in SD243 were deleted by sacB |
| | P_{rbc} Ch fatB2 Δ(slr2001-slr2002) | | Km^{R} insertion |
| | ::*P_{psbA2} Ch fatB2 | | |
| | Δslr1710:: sacB Km^{R} | | |
| SD249 | Δaas::P_{psbA2} ′tesA | Transform SD248 with | 5^{th} generation constitutive FFA- |
| | Δ(slr1993-slr1994):: | pψ249 (Δslr1710::P_{psbA2*} | secreting strain. Plant C14:0 |
| | P_{cpc}accBC P_{rbc} accDA | Cc fatB1) and select for | thioesterase overproduction and |
| | Δsll1951::*P_{psbA2} Uc fatB1 | sucrose survival | penicillin binding protein 2 |
| | P_{rbc} Ch fatB2 Δ(slr2001-slr2002) | | deletion were incorporated into |
| | ::*P_{psbA2} Ch fatB2 | | SD243 |
| | Δslr1710::P_{psbA2*} Cc fatB1 | | |
| SD265 | Δaas::P_{psbA2} ′tesA | Transform SD249 with | Intermediate strain for adding |
| | Δ(slr1993-slr1994)::P_{cpc} | pψ265 (Δslr2132::sacB | genes into SD249. The |
| | accBC P_{rbc} accDA | Km^{R}) and select for | phosphotransacetylase gene |
| | Δsll1951::*P_{psbA2} Uc fatB1 | kanamycin resistance | (slr2132) in SD249 were deleted |
| | P_{rbc} Ch fatB2 Δ(slr2001-slr2002) | | by sacB Km^{R} insertion |
| | ::*P_{psbA2} Ch fatB2 | | |
| | Δslr1710::P_{psbA2*} Cc fatB1 | | |
| | Δslr2132::Km^{R} sacB | | |
| SD277 | Δaas::P_{psbA2} ′tesA | Transform SD248 with | 6^{th} generation constitutive FFA- |
| | Δ(slr1993-slr1994)::P_{cpc} | pψ274 (Δslr2132::P_{trc} | secreting strain. Codon-optimized |
| | accBC P_{rbc} accDA | tesA137) and select for | tesA137 gene driven by artificial |
| | Δsll1951::*P_{psbA2} Uc fatB1 | sucrose survival | promoter P_{trc} and |
| | P_{rbc} Ch fatB2 Δ(slr2001-slr2002) | | phosphotransacetylase deletion |
| | ::*P_{psbA2} Ch fatB2 | | were incorporated into SD249. |
| | Δslr1710::P_{psbA2*} Cc fatB1 | | |
| | Δslr2132::P_{trc} tesA137 | | |

### Example 2

### Construction of slr1609-Deficiency and PHA-Deficiency Strains

In 6803, the slr1609 gene was previously annoted as encoding a long-chain acyl-CoA ligase and designated as fadD. Acyl-CoA ligase is the first key enzyme in the beta-oxidation pathway, which is the key enzyme for FFA consumption. Based on this information, we decided to delete this gene to save FFA product from being degraded. We therefore replaced slr1609 with P_{psbA2} 'tesA by SacB/KmR intermediate deletion/insertion in the strain SD216 described in Table 1. We developed five more generations of modifications based on strain SD216. Based on studies in yeast, deletion of genes for acyl-CoA synthetase isozymes led to efficient fatty acid secretion (Scharnewski, Pongdontri et al. 2008), and introduction of acyl-CoA synthetase mutation ameliorated a fatty acid secretion phenotype (Michinaka, Shimauchi et al. 2003). Indeed, impairment of long-chain acyl-CoA synthetase activity is expected to increase the fatty acid concentration in the cell and to enhance fatty acid secretion. In early 2010, it was reported that slr1609 is not an acyl-CoA (synthetase) ligase gene as previously thought and actually encodes an acyl-ACP synthetase, which ligates FFA and ACP together as acyl-ACP (Kaczmarzyk and Fulda 2010). The gene name is therefore aas instead of fadD and the strain genotypes in Table 1 have been corrected based on this new information. This aas catalysed reaction goes exactly in the opposite direction of FFA production catalyzed by thioesterases. The paper also showed that just by deleting slr1609, 6803 will lose the ability to recycle FFA and leaks FFA into the medium. The new published data proves that deletion of slr1906 is very important for FFA accumulation and secretion and substantiates the importance of our discovery of the benefits of deleting this gene as a starting point for constructing 6803 stains for improved fatty acid production and secretion. Barring the existence of an as yet unknown fatty acid degradation pathway, accumulation/secretion or conversion to acyl-CoA are the only two main destinations for fatty acids in 6803. Using the Km^{r}-sacB insertion, the full function of slr1609 in SD214 and SD216 and their derived strains (i.e., SD225, SD232, SD243, SD249 and SD277) was interrupted (FIG. 2 and Table 1). Thus only accumulation/secretion of medium- and long-chain fatty acids can occur in these slr1609 (aas)-deficient strains, and indeed increased fatty acid biosynthesis and secretion has been observed.

A polyhydroxyalkanoate (PHA) synthesis deletion strain SD207 was constructed by interrupting two PHA synthesis genes slr1993/1994 in 6803, which encode PHA-specific beta-ketothiolase and PHA-specific acetoacetyl-CoA reductase, respectively. PHA synthesis consumes the carbon resources from the Acetyl-CoA pool, thus inactivation of the PHA synthesis pathways will shut off the carbon flux towards the unnecessary byproducts. PHA inclusions in 6803 can be stained by Nile Red, a neutral lipid specific fluorescence dye. Flow cytometry analysis of the Nile Red stained Δslr1993/1994 strain SD207 showed disappearance of the PHA emission compared to the wild-type 6803 (FIG. 3). The PHA strategy in the FFA-secreting strains was thus applied, resulting in SD220 and its derivative strains (SD225 and SD232) (FIG. 2 and Table 1).

### Example 3

### Construction of FFA-Overproducing Strains

We constructed six generations of 6803 SD strains for FFA secretion (FIG. 2, Table 1). In all cases, insertions were introduced in place of genes with either negative or competing consequences for FFA production. All the strains are fully segregated and genotypically pure, as shown in FIG. 4 for the 5^{th} generation strain SD249 as an example. In the 1^{st} generation SD strains, the E. coli TE gene 'tesA (Cho and Cronan 1995) was expressed in two different strategies (one in SD215 and one in SD216) (FIG. 2). In strain SD215, 'tesA was controlled by a Ni²⁺ inducible regulator, which was turned on by the addition of 7 μM Ni²⁺ in the medium (Liu and Curtiss 2009). In strain SD216, 'tesA was constitutively expressed at high level by the promoter P_{psbA2} (Agrawal, Kato et al. 2001). Also in SD216, the fatty acid activation gene aas (slr1609), encoding an acyl-ACP synthetase (Kaczmarzyk and Fulda 2010), was knocked out by inserting the P_{psbA2} 'tesA cassette into the coding region of slr1609.

Acetyl-CoA carboxylase (ACC) has been postulated as the rate-controlling enzyme in fatty acid biosynthesis (Davis, Solbiati et al. 2000). For the 2^{nd} modification (SD223 and SD225), an artificial operon P_{cpc} accB accC P_{rbc} accD accA was introduced into SD215 and SD216 to overproduce ACC (FIG. 2). P_{cpc} is the promoter of the cpc operon, which encodes the photosynthesis antenna protein phycocyanin (Imashimizu, Fujiwara et al. 2003); P_{rbc} is the promoter of the rbc operon, which encodes ribulose 1,5-bisphosphate carboxylase (Onizuka, Akiyama et al. 2003). In SD223 and SD225, two poly-3-hydroxybutyrate (PHB) synthesis genes (slr1993 and slr1994) were knocked out.

In the 3^{rd} generation strain SD232, Uc fatB1, a 12:0 acyl-ACP TE encoding gene from Umbellularia californica (Pollard, Anderson et al. 1991) was synthesized in an artificial operon *P_{psbA2} Uc fatB1 and inserted to knock out sll1951 (FIG. 2), which encodes the monomer protein of the 6803 surface-layer (Sakiyama, Ueno et al. 2006) and exhibits RTX motifs that are characteristic of many S-layer proteins in various bacterial species including cyanobacteria (Linhartova et al. 2010). In the 4^{th} generation strain SD243, Ch fatB2, an 8:0 and 10:0 acyl-ACP TE encoding gene from Cuphea hookeriana (Dehesh, Jones et al. 1996) was synthesized in an artificial operon *P_{psbA2} Ch fatB2 was inserted to knock out slr2001 and slr2002 (FIG. 2), which encode cyanophycin synthetases (Ziegler, Diener et al. 1998). In the 5^{th} generation strain SD249, Cc fatB1 from Cinnamomum camphorum (Voelker and Davies 1994) was synthesized in an artificial operon *P_{psbA2} Cc fatB1 and inserted to knock out slr1710, a 6803 penicillin-binding protein (PBP2) gene (Voelker and Davies 1994). The plant TE genes were synthesized after sequence optimization. *P_{psbA2} is a modified promoter sequence of psbA2, in which the AT-box (9-18 bp upstream from the ATG start codon) was removed from P_{psbA2} to enhance mRNA stability under dark conditions (Agrawal, Kato et al. 2001). In the sixth-generation strain SD277, an artificial operon P_{trc} tesA137 was inserted to knock out slr2132, a pta gene coding for phosphotransacetylase (Morrison, Mullineaux et al. 2005). The plant TE genes and E. coli tesA137 were synthesized after sequence optimization to enhance expression and mRNA stability.

### Example 4

### Constitutive Versus Inducible FFA Production and Secretion

We initially conducted studies using the nickel-inducible FFA production and secretion strains SD215, SD219, SD223 and SD228 to compare with their constitutive counterpart stains SD216, SD220, SD225 and SD229 (Table 2). Strains with inducible synthesis had negligible FFA either intracellularly or extracellularly until after induction by addition of 7 μM Ni²⁺ to the culture (OD_{730 nm}˜1.0). However, by the time cultures reached 2×10⁹ cells/ml the amounts of intracellular and extracellular FFAs were very similar for each pair with the amount of intracellular FFAs decreasing by 35% for the constitutive strains SD216 versus SD229 and 28% for the inducible strains SD215 versus SD228. In regard to secreted FFAs, there was a 7.7-fold increase for the constitutive strains SD229 compared to SD216 and a 11.4-fold increase for the inducible strains SD228 compared to SD215.

TABLE 2
The secreted FFA and intracellular FFA of SD strains

| | | FFA secreting | | |
|---|---|---|---|---|
| | | efficiency^{b} | Intracellular | |
| FFA | | (mg/day/liter of | FFA^{c} | Genetic modifications |
| Production | Strains^{a} | culture) | (mg/cell) | from parents^{d} |
| Natural | SD100 | 0.3 ± 0.02 | 4.4 × 10⁻¹¹ | Wild type |
| Constitutive | SD216 | 6.2 ± 0.4 | 6.3 × 10⁻¹¹ | ′tesA overexpression and aas |
| | | | | deletion (Δslr1609::P_{psbA2} ′tesA) |
| | SD220 | 8.7 ± 0.5 | 7.5 × 10⁻¹¹ | PHB synthesis deletion |
| | | | | (Δ(slr1993-slr1994)::sacB Km^{R}) |
| | SD225 | 17.0 ± 2.5 | 12.5 × 10⁻¹¹ | ACC overproduction (Δ(slr1993-slr1994) |
| | | | | :: P_{cpc} accBC P_{rbc} accDA) |
| | SD220 | 47.6 ± 3.8 | 4.1 × 10⁻¹¹ | S-layer deletion |
| | | | | (Δsll1951:: sacB Km^{R}) |
| | SD232 | 63.8 ± 5.2 | 4.5 × 10⁻¹¹ | C12:0 thioesterases |
| | | | | (Δsll1951:: *P_{psbA2} Uc fatB1) |
| | SD243 | 85.5 ± 6.4 | 6.8 × 10⁻¹¹ | C8:0 C10:0 thioesterase and |
| | | | | cyanophycin synthesis deletion |
| | | | | (Δ(slr2001-slr2002):: *P_{psbA2} Ch |
| | | | | fatB2) |
| | SD249 | 133 ± 12 | 5.6 × 10⁻¹¹ | C14:0 thioesterase and PBP2 |
| | | | | deletion |
| | | | | (Δslr1710:: P_{psbA2*} Cc fatB1) |
| Inducible | SD215 | 4.5 ± 0.4 | 6.1 × 10⁻¹¹ | Controllable ‘tesA expression |
| | | | | (ΔnsrBAC::P_{nrsB} ‘tesA) |
| | SD219 | 6.5 ± 0.3 | 7.9 × 10⁻¹¹ | PHB synthesis deletion |
| | | | | (Δ(slr1993-slr1994)::sacB Km^{R}) |
| | SD223 | 23.3 ± 4.7 | 13.2 × 10⁻¹¹ | ACC overproduction (Δ(slr1993-slr1994) |
| | | | | ::P_{cpc} accBC P_{rbc} accDA) |
| | SD228 | 51.5 ± 6.3 | 4.4 × 10⁻¹¹ | S-layer deletion |
| | | | | (Δsll1951::sacB Km^{R}) |

^{a}The genotypes and constructions of the strains were confirmed by PCR analysis.
^{b}For the constitutive strains, the late-log phase FFA secreting efficiency was measured for a 24 h interval with the culture density starting at about 1.5 × 10⁸ cells/mL and reaching about 2 × 10⁸ cells/mL. For the inducible strains, the induced FFA secreting efficiency was measured after addition of 7 μM Ni²⁺ to the culture (OD_{730 nm} ~1.0).
^{c}2 × 10⁹ cells in 10 mL hexane treated culture were extracted by Folch method.
^{d}The parent for SD216 and SD215 is wild-type 6803, the parents for the other strains are the strains on the row above the new strain.

### Example 5

### Growth of the SD Strains

During the growth of the FFA-secreting strains (SD232 for example) from a single cell to a 200 mL cell culture (8×10⁸ cells/mL, 1.2 g of dry weight/liter), cell permeability was revealed microscopically after staining cells with the vital dye SYTOX Green (Roth, Poot et al. 1997). Our experiment showed that only 1% of the permeable cells (sorted by flow cytometry for green fluorescence) were able to form colonies on BG-11 agar plates, suggesting that they were damaged.

SD232 colonies (containing 8×10⁵ cells) descended from a single cell and grown on BG-11 agar plates for 10 days contained 0.5% cells permeable to the dye (FIG. 5B). When the entire colonies were inoculated into 1 mL BG-11 medium in a glass tube and grown for 3 days, 0.4% cells were permeable (FIG. 5C). However when 4×10⁸ SD232 cells were inoculated into 200 mL BG-11 medium with 100 mL/min aeration of 1% CO₂-enriched air, the culture showed 14.7% permeable cells on the first day and 33.7% permeable cells on the second day, suggesting that CO₂ bubbling created significant cell damages at these low cell densities. A long lag phase was observed for the SD232 cultures when the cell density was below 10⁷ cells/mL and the permeable percentage was above 22.7% (FIG. 6). In the lag phase, SD232 cells aggregated as clusters of cells, which harbored a large number of viable growing cells (FIG. 7C). After the culture density achieved 10⁷ cells/mL, the cultures started exponential growth. The doubling times of wild-type (7.4±0.8 h), SD125 (8.9±1.0 h), SD126 (11.9±1.1 h) and SD232 (12.4±1.3 h) strains were calculated based on the exponential growth period (FIG. 6). The damaged cell percentage for wild type was below 2% before attainment of 6×10⁸ cells/mL (FIG. 6). However, after completion of the exponential growth phase, the maintenance of cell viability for SD232 (0.39%, 428 h) was much better than for the wild-type 6803 strain SD100 (35.7%, 382 h, FIGS. 7, G and I).

### Example 6

### FFA Overproduction and Secretion

FFA secretion was observed for the constitutively 'tesA expressing strains, including SD216, SD220, SD225, SD229, SD232, SD243, SD249, and SD277 (FIG. 8). Except for SD225 (ACC overproduction addition), most genetic modifications resulted in increased FFA secretion compared to the parent strains, but the intracellular FFA amount did not increase (Table 3). We noticed that deleting the surface-layer protein from the cell envelopes as done in SD229 caused a 3-fold increase in FFA secretion over the parent SD225 and also observed that weakening the peptidoglycan cell-wall layer by deleting the gene encoding PBP2 in SD249 caused a further significant increase in FFA secretion. FIG. 9 shows these cell surface layers and means by which they can be eliminated, damaged or digested to facilitate FFA secretion. The intracellular FFA amount decreased with the surface-layer protein deletion (SD229) and peptidoglycan weakening (SD249, Table 3). These results indicate that removal of or damage to hydrophilic cell wall barriers did facilitate FFA secretion and by decreasing the intracellular pool of fatty acids decreased feedback inhibition of enzymes involved in synthesis of fatty acid precursors, further increasing FFA production. In SD277, insertion of the codon-optimized tesA137 gene driven by the artificial promoter Pₜ, not only increased FFA secretion from 14.3×10⁻¹¹ to 20.0×10⁻¹¹ mg/cell, but also increased the growth rate. The secreted FFA accounts for about 13% of the biomass (the dry weight of an SD100 cell is about 150×10⁻¹¹ mg).

TABLE 3
The secreted FFA and intracellular FFA of SD strains

| | | Final cell | | | | |
|---|---|---|---|---|---|---|
| | Doubling | density^{c} | FFA secretion | Secreted FFA^{e} | Intracellular | |
| Strains^{a} | Time^{b} (Hour) | (cells/ml) | Yields^{d} (mg/L) | (mg/cell) | lipids/FFA^{f} (mg/cell) | Genetic modifications |
| SD100 | 7.4 | 3.2 × 10⁹ | 1.8 ± 0.06 | 0.05 × 10⁻¹¹ | 0.16 × 10⁻¹¹ | Wild type |
| SD216 | 11.9 | 8.3 × 10⁸ | 83.6 ± 8.3 | 8.0 × 10⁻¹¹ | 3.01 × 10⁻¹¹ | ′tesA overexpression and |
| | | | | | | aas deletion |
| | | | | | | (Δslr1609::P_{psbA2} ′tesA) |
| SD225 | 12.1 | 8.3 × 10⁸ | 83.6 ± 11.4 | 8.0 × 10⁻¹¹ | 2.38 × 10⁻¹¹ | PHB synthesis deletion |
| | | | | | | ACC overproduction |
| | | | | | | Δ(slr1993-slr1994):: P_{cpc} |
| | | | | | | accBC P_{rbc} accDA) |
| SD232 | 12.4 | 7.5 × 10⁸ | 90.5 ± 6.4 | 9.4 × 10⁻¹¹ | 1.89 × 10⁻¹¹ | Sll1951 deletion C10:0 |
| | | | | | | C12:0 thioesterases |
| | | | | | | (Δsll1951:: *P_{psbA2} Uc fatB1 |
| | | | | | | P_{rbc} Ch fatB2) |
| SD243 | 14.3 | 1.0 × 10⁹ | 92.9 ± 3.0 | 10.2 × 10⁻¹¹ | 1.24 × 10⁻¹¹ | cyanophycin synthesis |
| | | | | | | deletion C8:0 C10:0 |
| | | | | | | thioesterase (Δ(slr2001-slr2002) |
| | | | | | | :: *P_{psbA2} Ch fatB2) |
| SD249 | 16.7 | 1.3 × 10⁹ | 146 ± 21 | 14.3 × 10⁻¹¹ | 1.05 × 10⁻¹¹ | PBP2 deletion C14:0 |
| | | | | | | thioesterase (Δslr1710:: P_{psbA2*} |
| | | | | | | Cc fatB1) |
| SD277 | 14.1 | 1.0 × 10⁹ | 197 ± 14 | 20.0 × 10⁻¹¹ | 1.12 × 10⁻¹¹ | Phosphotransacetylase |
| | | | | | | deletion codon optimized |
| | | | | | | tesA137 gene ((Δslr2132:: |
| | | | | | | P_{trc} tesA137) |

^{a}The genotypes and constructions of the strains were described in Table 1.
^{b}The doubling times were determined in separate experiments. Briefly, daily colony forming unit (CFU) or OD_{730 nm} measurements for exponential growing cultures (density below 10⁸ cells/ml) were used for the calculation for the doubling times of different strains.
^{c}The final cell density was determined by (CFU).
^{d}20 mL culture was extracted by hexane and analyzed by GC for FFA amount.
^{e}The secreted FFA per cell was calculated from the final cell density and the FFA secretion yield.
^{f}The intracellular unsecreted FFA were extracted Folch method, and calculated with the final cell density.

### Example 7

### Chemical Composition of the FFAs

The chemical composition of the intracellular and secreted FFAs was analyzed by GC (Table 4). First, the percentage of C10:0-C14:0 FFAs increased with successive generations of strain constructions. For example, C14:0 and C12:0 increased from 16.5% (SD216 secretion) to 47.3% (SD249 secretion). Second, it was observed that the percentage of unsaturated fatty acids decreased in the FFA-overproducing strains. Third, we observed that the percentage of middle chain FFAs was higher in the secreted extracts than the intracellular unsecreted extracts (for example, the C12:0 and C14:0 FFA weight % for SD232, SD243 and SD249 in Table 4), suggesting that the middle chain FFAs are easier to secrete and especially when fully saturated.

TABLE 4
GC analysis of the FFA profile of the SD strains

| | Fatty acid Weight Percentage (%)^{b} | | | | | | | | | | | | | |
|---|---|---|---|---|---|---|---|---|---|---|---|---|---|---|
| FFA | SD100 | | | SD216 | | SD225 | | SD232 | | SD243 | | SD249 | | SD277 |
| Type^{a} | Lipids | Secr | Cell | Secr | Cell | Secr | Cell | Secr | Cell | Secr | Cell | Secr | Cell | Secr |
| 10:0 | t | ND^{d} | ND | ND | ND | ND | ND | ND | ND | 2.3 | ND | 1.6 | ND | 0.6 |
| 12:0 | t | 4.7 | 3.3 | 2.5 | 8.2 | 2.3 | 14.3 | 19.5 | 10.6 | 20.5 | 10.2 | 22.3 | 8.3 | 19.9 |
| 14:0 | t | 5.2 | 3.1 | 14.0 | 13.4 | 11.9 | 13.3 | 10.9 | 13.7 | 11.7 | 14.8 | 25.0 | 19.5 | 20.9 |
| 16:1 | 3 | 11.8 | 7.1 | ND | ND | ND | ND | ND | ND | ND | ND | ND | ND | ND |
| 16:0 | 52 | 38.3 | 46.8 | 68.2 | 60.5 | 66.6 | 54.8 | 51.3 | 54.1 | 48.7 | 48.0 | 40.6 | 43.8 | 43.3 |
| 18:3 | 29 | 8.7 | 11.6 | ND | ND | ND | ND | ND | ND | ND | ND | ND | ND | 1.4 |
| 18:2 | 11 | 3.7 | 6.7 | ND | ND | ND | ND | ND | ND | ND | ND | ND | 7.6 | 1.1 |
| 18:1 | 5 | 5.3 | 5.4 | ND | ND | ND | ND | ND | ND | ND | 9.6 | ND | 9.6 | 1.5 |
| 18:0 | t | 22.3 | 16.0 | 15.3 | 17.9 | 19.2 | 17.7 | 18.3 | 21.6 | 16.8 | 17.4 | 10.5 | 11.2 | 11.3 |
| Total | 100 | 100 | 100 | 100 | 100 | 100 | 100 | 100 | 100 | 100 | 100 | 100 | 100 | 100 |

^{a}The number before the colon in the fatty acid name refers to the number of carbons, and the number after the colon refers to the number of double bonds.
^{b}The fatty acid percentages for SD100 membrane lipids were obtained from Wada's report (Wada and Murata 1990) as a baseline for the SD100 fatty acid profile. The fatty acid percentages for the other samples were based on the free fatty acids. Secreted samples (Secr) mean secreted FFAs in the culture medium extracted by hexane without disrupting the cells. Cell samples represent the unsecreted FFAs remaining inside the cells extracted from sedimented cells by the Folch method.
^{c}t, trace amount (less than 4%) (Wada and Murata 1990).
^{d}ND, not detected.

### Example 8

### Weakening Cell Walls to Facilitate FFA Secretion

Cyanobacterial cells often have multiple surface layers including extracellular polysaccaride capsules (Panoff, Priem et al. 1988), surface layers composed of regularly arrayed proteins (Karlsson, Vaara et al. 1983), outer membranes containing lipopolysaccharide (LPS) and numerous outer membrane proteins with diverse functions, rigid cell wall peptidoglycan layers (Hoiczyk and Hansel 2000) and a cytoplasmic membrane (see FIGS. 9 and 13). S-layers are regularly arrayed surface layers composed of a single protein species that provide a protective barrier for cyanobacterial cells (Karlsson, Vaara et al. 1983) and such surface layer proteins have been identified in 6803 (T J Beverage et al. 1997). By using bioinformatic searches, we identified some seven 6803 genes encoding proteins with properties characteristic of S-layer proteins (see Example 13) and because slr1651 encoded a protein with sequence homologies to other well-characterized S-layer proteins and had been demonstrated to encode a prevalent protein coating the entire surface of 6803 cells (Sakiyama, Ueno et al. 2006), we selected it for deletion to eliminate it as a likely barrier to the efficient secretion of FFAs. The FFA secretion efficiency of the surface-layer protein deficient strain SD229 was much higher than that of its parent strain SD225 (Table 3).

Since peptidoglycan layers (Hoiczyk and Hansel 2000) are also polar, imparing their structural integrity should also facilitate the secretion of FFAs. We used the controllable 'tesA strains SD125 and SD228 (S-layer deficient) to evaluate the FFA secretion efficiency after partial inhibition of peptidoglycan layer synthesis by addition of ampicillin (Amp). After induction of 'tesA, the FFA secretion efficiency increased with Amp doses lower than needed to cause cell lysis (Table 5). However, too much damage to the cell walls also killed the cells. Based on these observations, we further genetically weakened peptidoglycan layers by deleting a gene encoding a peptidoglycan assembly protein (PBP2) in SD249, and this also increased FFA secretion (Table 3).

TABLE 5
The FFA secretion of SD215 and SD228 after cell wall treatments

| | | | | Secretion | |
|---|---|---|---|---|---|
| | NiSO₄ | Ampicillin | Secreted FFAs | efficiency^{a} | |
| SD strain | (μM) | (mg/L) | (mg/50 mL) | (mg/l/day) | Observation Remarks |
| SD215 | 0 | 0 | 0.118 | — | No lysis on the third day |
| | 7.0 | 0 | 0.292 | 3.48 | No lysis on the third day |
| | | 1 | 0.376 | 5.16 | No lysis on the third day |
| | | 3 | 0.459 | 6.82 | No lysis on the third day |
| | | 9 | 1.109 | 19.82 | Lysis on the second day |
| | | 25 | — | — | Lysis on the first day |
| SD228 | 0 | 0 | 0.245 | — | No lysis on the third day |
| | 7.0 | 0 | 2.708 | 49.3 | No lysis on the third day |
| | | 1 | 3.572 | 66.5 | No lysis on the third day |
| | | 3 | 3.886 | 72.8 | Lysis on the second day |
| | | 9 | — | — | Lysis on the first day |
| | | 25 | — | — | Lysis on the first day |

^{a}200 mL cultures (about 1.5 × 10⁸ cells/mL) were induced by adding 7.0 μM Ni²⁺ to the medium and treated with ampicillin of various concentrations.

### Discussion for Examples 1-8

We found both disadvantages and advantages of FFA-overproduction to the growth of SD strains. The disadvantage was the fragility of SD cells with CO₂ aeration at low cell density, which caused a long lag phase for FFA secreting SD cultures (e.g., SD232). The permeability to the vital dye SYTOX Green indicated that the cytoplasmic membranes of some cells were damaged when CO₂ aeration started. In addition, elimination of the surface-layer protein and accumulation of intracellular FFAs contributed to cell fragility. However, the high percentage of damaged cells (FIGS. 5, E and F) of SD232 cultures was not observed for exponential or late-lag phase cultures (FIGS. 7, E and G). Proper cell density is therefore important for SD cultures with multiple gene alterations to grow in a healthy manner with added CO₂ aeration. Therefore, we now always maintain cell densities above 10⁷ CFU/mL by stepwise scaling up the culture.

FFA-overproduction strains exhibited less cell damage than wild-type cells at stationary phase (FIG. 6). This damage at stationary phase may be caused by excess electrons from photosynthesis when no significant NADPH consumption is required (Hu, Sommerfeld et al. 2008). The accumulated electrons may induce overproduction of reactive oxygen species, which damage the membranes. We observed much lower cell damage percentage (0.39%) in SD232 culture compared to the wild-type 6803 in the stationary phase of growth (FIGS. 7, G and I). This suggested that FFA-secretion might be able to relax the over-reduced photosynthetic electron transport chain and make the cells healthier in stationary growth phase. This advantage is beneficial for the continuous FFA production using stationary-phase cyanobacterial cultures.

We showed that two factors significantly increased the FFA secretion quality and quantity. One factor is increasing the cytosol TE activities. Our data have shown that introducing extra TEs into 6803 did increase the FFA secretion efficiency (Table 3, from SD216 to SD249), and shorten the FFA chain length (Table 4, from SD216 to SD249). However, the product chain length of plant TEs in 6803 did not totally match their substrate preference in plants or E. coli. For example, Ch FatB2 produced substantive C12:0 in SD243 (Table 4), while it accumulated C10:0 and C8:0 in Cuphea hookeriana plants and in recombinant E. coli (Dehesh, Jones et al. 1996). Extra TEs also increased the fraction of fully saturated FFAs (Table 4). We hypothesize that the 6803 fatty acid dehydrogenases are located in the di-glycerolipid membranes. When the FFAs were released from acyl-ACP, they would not be incorporated into membrane lipids for further desaturation. This phenomenon is beneficial for biofuel production, since unsaturated carbon chains result in a lower octane rating, and they are less stable and could potentially compromise storage.

Another important factor is weakening cell envelopes to facilitate FFA export. As Hamilton and colleagues have shown, long chain fatty acids perform fast free diffusion by ‘flip-flop’ in the phospholipid bilayer (Hamilton 2007). As membranes are not substantial barriers to FFA secretion, we focused on compromising the layers made of polar molecules, i.e., surface-layer proteins and peptidoglycan layers. Quite possibly as detailed in Example 17, alteration or elimination of some LPS components of the outer membrane will also facilitate FFA secretion. Removal of FFAs from the cyanobacterial cell interior will increase intracellular FFA production, as removing the final products from a reaction system into a metabolic sink will push the equilibrium toward products (Fell 1996) as well as relieve the consequences of feedback inhibition.

In SD277, insertion of the codon-optimized tesA137 gene driven by the artificial promoter P_{trc} not only increased FFA secretion from 14.3×10⁻¹¹ to 20.0×10⁻¹¹ mg/cell, but also restored growth rate (Table 3). The secreted FFA accounts for about 13% of the biomass (the dry weigh of an SD100 cell is about 150×10⁻¹¹ mg). We are continuing to investigate a diversity of means for further genetic improvements in our strains to give increased production and secretion of fatty acids. For example, we will enhance the primary FFA pathway genes, and eliminate or down regulate the competing pathway genes (FIG. 10). We will also make further improvements of growth conditions (CO₂ concentration, culture medium, temperature, illumination, pH and cell density) to enhance FFA yields. We are optimistic that additional modifications to SD277 will further increase the total FFA yields to result in economical biofuel production.

### Materials and Methods for Examples 1-8

Bacterial strains, media and growth conditions. All SD strains are derived from Synechocystis sp. PCC 6803. SD strains were grown at 30° C. in BG-11 medium (Rippka et al. 1979) under continuous illumination (140 μmol photons m⁻² s⁻¹) and bubbled with 1% CO₂-enriched air. The details for growing an SD culture from a colony descended from a single cell are described below. For plating and transformant selection, 50 μg/mL kanamycin or 4.5% (wt/vol) sucrose is added to 1.5% agar plates (wt/vol) and plates were grown under continuous illumination (50 μmol photons m⁻² s⁻¹). All of our strains are maintained as concentrated cultures in BG-11 medium with 20% glycerol and stored at −80° C.

Growth and Cell Damage Measurement.

Bacterial growth in liquid culture was monitored spectrophotometrically or by flow cytometry and/or by plating. The relationship between 6803 culture optical density and cell density is used for conversion (FIG. 11). Staining with 5 μM SYTOX Green nucleic acid stain (Invitrogen Molecular Probes, Inc. OR, USA) (Roth et al. 1997) for 5 min was used to detect damaged cells. Cells were observed under an Axioskop40 fluorescence microscope (Zeiss, Germany). Green cells are sorted in a FACSAria flow cytometer (BD Biosciences, CA, USA) and counted as damaged.

Synthetic Molecular Procedures.

Methods for DNA manipulation are standard (Sambrook et al. 1989). The primers for constructions and genotype verifications are listed in Table 14. DNA sequences were analyzed in the DNA Sequence Laboratory at Arizona State University. Gene segments are synthesized at Genscript (Piscataway, N.J., USA). The nucleic acid sequences of heterogenous genes were redesigned by codon optimization based on the codon frequencies of highly expressed 6803 genes (Table 15) Also the stem-loop hairpins in the predicted mRNA secondary structure were removed to smooth the transcription and to stabilize mRNA by prolonging its half-life (Hamilton 2007).

Gene Deletion and Introducing New Genes into 6803.

Multiple gene modifications are applied into SD strains by using a sacB-Km^{R} cassette (Liu and Curtiss, 2009). Detailed methods are described below.

FFA Separation and Measurement.

The FFAs in the medium are quantitatively separated from the culture medium by hexane, which is unable to release FFAs and other lipids from intact SD100 cells. Twenty mL of culture is acidified by 0.4 mL H₃PO₄ (1 M) containing 0.4 g NaCl, and extracted with 10 mL hexane. For the unsecreted intracellular FFAs and lipids, the cells are extracted by the Folch method (Folch et al. 1957) for total lipids. The FFA samples were analyzed by GC (Lalman and Bagley, 2004) (FIG. 12).

To measure the FFA secretion of the constitutively producing strains, the accumulation of FFAs were measured for late-log phase cultures with a density of about 10⁹ cells/mL. Briefly, during the continuous cultivation of a 50 mL culture, aeration was switched from air to 1% CO₂ enriched air when culture density reached about 1.5×10⁸ cells/mL. After the cell density reached about 10⁹ cells/mL with 1% CO₂ aeration (2-3 days later), a 20 ml sample was extracted by hexane. To measure the FFA secretion efficiencies of the nickel inducible strains, the secretion efficiencies in one day were calculated from the difference of the FFA secretion values between the before induction and 24 h after induction. In the experiment to evaluate the effect of weakening cell walls on FFA secretion, five subcultures (about 1.5×10⁸ cells/mL) of 200 mL were induced by adding 7.0 μM Ni²⁺ to the medium and treated with 0, 1, 3, 9, and 25 μg/mL ampicillin.

Statistical Analysis.

Most data were expressed as means±standard deviation. The means were evaluated with one-way Analysis of Variance (ANOVA) for multiple comparisons among groups. Student's T-test was used for pair-wise comparisons. P<0.05 was considered statistically significant.

Growth of an SD Culture Started from a Single Cell Descended Colony.

As described above, cultures of extensively modified SD strains suffered a long lag phase when started at low cell density, probably because a significant percentage of the cells was damaged. A principle mechanism for culturing extensively modified SD strains is therefore not to start cultures below a density of 10⁷ cells/mL, since low cell densities will create a long lag phase prior to exponential growth. We used the following procedure to grow a 6803 culture from a colony descended from a single cell. A single SD colony is picked by a sterilized needle and used to inoculate 1 mL BG-11 medium buffered by 10 mM N-[tris(hydroxymethyl)methyl]-2-aminoethanesulfonic acid (TES) NaOH (pH 8.2) in a glass test tube. The tube is incubated with illumination and intermittent shaking for 2-4 days. These starter cultures can be scaled up by ‘1 into 10’ inoculations after achieving an OD_{730 nm} of 0.6 (10⁸ cells/mL) by adding 10 mL BG-11 medium culture grown in a 50-mL flask with 50 rpm rotation. We added 90 mL BG-11 medium to the 10 mL culture, and grew it in a 250-mL flask with 100 mL/min aeration with air and without shaking. Then we added 900 mL BG-11 medium to the 100 mL culture, and grew it in a 2-L flask with 300 mL/min air sparged with an air stone. Once a 1 L culture achieves OD_{730 nm}˜0.6, aeration is switched from air to 1% CO₂-enriched air. This protocol uses TES buffer and air aeration to keep the pH around 8 at the beginning inoculation stages to minimize the lag phase. However, TES buffer reduces FFA production, so TES buffer is not used after the cell density achieves 10⁷ cells/mL. Free fatty acid (FFA)-producing strains need a sufficient CO₂ supply and a pH above 8 to maximize FFA-secretion yields. When the 6803 cell density achieves 10⁸ cells/mL, the culture is able to maintain its pH above 8 and can be supplied with CO₂-enriched air.

Transformation Procedures for 6803.

We optimized the current genetic modification techniques for 6803 gene deletion, insertion and substitution (Liu and Curtiss, 2009). Suicide vectors harboring a positive selection marker (e.g., Km^{R}) and a counter selection marker (e.g., sacB) are widely applied in a two-step gene deletion and insertion for 6803 without leaving any drug marker residuals (FIG. 1).

i. Transformation of Suicide Vectors Containing sacB-Km^{R} Cassette.

About 10⁶ SD cells in 10 μl BG-11 medium are mixed with 400 ng suicide vector DNA containing the sacB-Km^{R} cassette and incubated for 5 h. Then the mixtures were plated onto a filter membrane (Whatman PC MB 90mM 0.4 μm) layered on a BG-11 agar plate. After segregation on the BG-11 plate for about 24 h, the membrane carrying the cyanobacteria was transferred onto a BG-11 plate containing 50 μg/mL of kanamycin. Generally, the colonies appear 4-5 days later. Individual colonies are restreaked with a sterile loop onto a kanamycin BG-11 agar plate and a 4.5% sucrose BG-11 agar plate. Those patches growing on a kanamycin BG-11 agar plate and not growing on a 4.5% sucrose BG-11 agar plate have the correct insertions with the sacB-Km^{R} cassette.

ii. Transformation with Markerless Constructs.

To replace the sacB-Km^{R} selective marker with target gene segments, about 10⁶ sacB-Km^{R} cells in 10 μl BG-11 medium are mixed with 400 ng suicide vector DNA containing the target genes and incubated for 5 h. The mixtures are inoculated into 2 mL buffered BG-11 medium and grown for 3-4 days. 1 mL inoculation is plated onto a 4.5% sucrose-containing BG-11 agar plate. Generally, the colonies appear 5-8 days later. Individual colonies are restreaked onto a kanamycin BG-11 agar plates and a 4.5% sucrose BG-11 agar plates. The patches growing on sucrose plates and not growing on kanamycin plates are positive candidates for further evaluation by PCR.

iii. Confirmation of Replacement.

Cells from a colony are resuspended in 2 μl water in a 200-μl PCR tube. The cell suspension is frozen at −80° C. for 2 min, and then thawed in a 60° C. water bath. This freeze-thaw cycle needs to be performed three times. 1 μl frozen-thawed cell suspension is used as the PCR template for a 30 μl PCR reaction including the primers specific for the inserted gene segments or the deleted region.

Genetic Stability Tests.

When a foreign gene is introduced into 6803, it may cause an adverse effect on growth of the culture and be subjected to gene loss or modification, since any cell losing the genetic alteration will likely have a higher growth rate to eventually take over the population. The genetic stability of foreign genes in 6803 is therefore tested by growing a culture of the strain with periodic dilution and subculturing for at least two months. After this time, the cells from the culture are plated onto BG-11 agar plates to obtain single isolated colonies. One hundred single colonies are picked and tested for all genetic attributes and confirmed for the presence of the foreign gene by PCR as described above. The percentage of positive colonies in the culture reflects the genetic stability of the foreign gene. Genes found to be unstable can be modified to eliminate synthesis of non-functional hydrophobic domains that often are responsible for poor growth due to association with and impairment of cytoplasmic membrane function.

GC for the FFA Produced by the SD Strains.

GC was performed to determine the FFA amount in the hexane extracts (Lalman and Bagley 2004). After 6000 g×10 min centrifugation, 5 ml hexane was taken out from the upper organic layer, filled in a glass tube (13×100 mm, Fisherbrand), and dried on a nitrogen evaporator (N-EVAP111, Organomation Associates Inc.). The dried samples were then re-dissolved by a known volume of hexane and analyzed by gas chromatography (Shimadzu GC 2010) equipped with a Supelco Nukol capillary column (30 m×0.53 mm×0.50 μm) and flame ionization detector (FID).

For the unsecreted intracellular FFAs, the cells are collected by centrifugation, and extracted by the Folch method (Folch, Lees et al. 1957) for total lipids. The intracellular unsecreted FFA were extracted from the cell pellet after hexane extraction, and calculated based on the final cell density.

GC operating conditions were as follows: split ratio 1:5; inject volume 1 μL; helium carrier gas with constant flow rate 30 ml/min; H₂ 40 ml/min, Air 400 ml/min, make up gas (helium) 5 ml/min; injector and detector temperature 250° C.; and oven temperature started at 150° C. and increased at a rate of 10° C./min to 220° C. and held for 10 min. Each FFA compound was identified by comparing its retention time with that of standard (Sigma, St. Louis, Mo.). Compound concentrations in samples were quantified based on the area under the chromatogram peak in comparison with the standards.

### Example 9

### Optimize Metabolic Pathways to Improve FFA Yields and Quality

Preliminary results have shown that 6803 is able to overproduce and secrete FFA after genetic modification, and the FFA chain length can be adjusted for the production of biodiesel. On the basis of the FFA secreting 6803 strain, the whole fatty acid synthesis pathway may be genetically optimized to increase the production yields of FFA. The optimization for higher yields includes channeling carbon flow to fatty acid synthesis, attenuating or eliminating the competing pathways, and decreasing repression and feedback inhibition. The structure of secreted fatty acids may also be modified to match the requirement for high quality biofuels.

### Channeling Carbon Flow

The 6803 fatty acid synthesis pathways are different from those in heterotrophic bacteria. Starting with photosynthesis, the cyanobacterial carbon source for fatty acid biosynthesis comes from the Calvin-Benson-Bassham cycle rather than from glycolysis cycles (FIG. 10). The expression level of the genes in the primary pathway toward FFA production (e.g., pyk, pdh, odh, acc, and fab) may be increased. By the following modifications, the 6803 carbon flow may be expanded to FFA to increase the FFA production/secretion.

As shown in FIG. 10, the primary pathway genes for FFA overproducing may be overexpressed by the methods described in the Materials and Methods below and by use of the plasmid systems also described in Materials and Methods below. These primary pathway genes include sll0587 and sll1275 for the generation of pyrvate; pdh and odh for the synthesis of acetyl-CoA from pyruvate; accBCDA for the synthesis of malonyl-CoA from acetyl-CoA; fab genes (fabD, fabF, fabG, fabZ and fabl) for the synthesis of fatty acyl-ACP; and various thioesterase genes (Ch fatB2, Uc fatB1, Cc fatB1, and *tesA) uncoupling FFA synthesis from the long chain fatty acyl-ACP pathway. Some other genes, which are not shown in FIG. 10, may also be modified to increase carbon flow into FFA. For example, 6803 acyl carrier protein gene (acp, encoded by ssl2084) may also be overexpressed to determine whether that will increase fatty acid synthesis and secretion.

To overexpress the primary pathway genes, the coding sequences of these genes may be optimized by the synthetic procedures described in the Materials and Methods below. The codon optimization is necessary even for some of the native 6803 genes, because by removing the possible stem-loop mRNA structures, the mRNAs may be stabilized, and the transcription and translation may be enhanced, thus increasing the enzyme synthesis efficiency. Then the optimized enzyme encoding open reading frame may be operably linked to a strong promoter or tandem promoter active in different growth phases or under different environmental conditions or in response to different stresses or responsive to different activators or repressors (see Materials and Methods below) resulting in a constitutive or regulatable overexpression cassette. By the methods described in Materials and Methods below, the primary pathway gene overexpression cassettes may be inserted into the 6803 chromosome or contained on a plasmid vector.

Once an overexpression strain is constructed, the genetic stability of the introduced overexpression cassette(s) may be tested by the methods described Materials and Methods above and below. If the overexpressed cassette is genetically stable in 6803, the FFA production efficiency of this strain may be measured by the methods described in the Materials and Methods below, and compared with its parent strain to evaluate the success of the modification(s).

Overexpression of a gene within a pathway does not necessarily result in maximum protein levels. To optimize the whole pathway, the amount of a pathway enzyme should match the other enzymes in this pathway and the overall material-product flow. For example, the four subunits in the ACC complex should have correct stoichiometry to keep the appropriate relative equal molar ratio for maximum synthesis efficiency (Davis, Solbiati et al. 2000). The amount of the ACC proteins also needs to agree with the substrate acetyl-CoA supply and the product malonyl-CoA demand. As was stated before, straight over production of ACCs without presence of exogenous thioesterases in 6803 will not lead to increase of lipid or fatty acid contents, but cause significant adverse effects on the normal growth of 6803. For this reason, the optimal transcriptional and translational levels of the FFA primary pathway genes will be tested to match the maximum FFA-overproduction carbon flow in 6803.

### Attenuating Competing Nonessential Pathways and Synthetic Activities

As discussed above, in the FFA-secreting 6803 strains, the FFA synthetic pathway starts at the Calvin-Benson-Bassham cycle and ends at TEs (FIG. 10). Besides this primary pathway, many competing pathways are supposed to filter off the carbon flow from FFA synthesis to some unnecessary carbon byproducts, such as glycogen, lactate, malate, leucine, citrate, PHA, and acetate, thus decreasing the overall conversion efficiency from solar energy and CO₂ into FFA. Preliminary results have shown that deleting the PHA synthesis pathway genes in 6803 did improve the FFA overproduction efficiency (Table 2, SD216 and SD225). Therefore, the carbon/energy conversion efficiency for FFA may be further improved by attenuating and/or deleting genes encoding competing pathways.

By using optimized genetic approaches (Materials and Methods below), we will knock out or knock down the competing pathway genes by using the Km^{r}-sacB double-crossover mediated gene interruption. The competing genes that may be attenuated include slr1176 for glycogen synthesis; sll0920 for the oxaloacetate pathway; sll0891 for malate synthesis; sll1564 for leucine synthesis, sll0401 for the citrate pathways, slr2132, sll1299, sll0542, slr0091 and sll0090 for the acetate pathways. Since there is reason to believe that these synthetic functions cannot be totally deleted, constructions to reduce gene expression levels (attenuation) may be made by altering the promoter, SD sequence, and/or start codon, etc. and suicide vector methods may be used to make these alterations. Cyanophycin (a non-ribosomally produced amino acid polymer composed of an aspartic acid backbone and arginine side groups) synthetase genes (encoded by slr2002 and slr2001) may be deleted in 6803 to limit the competing carbon flow.

We identified more than 40 dispensible genes in the chrosome of Synechocystis 6803 (Table 12). These genes are all deletable in 6803 without significant adverse effects on the cell growth. Some deletions increased FFA production or secretion. See example 14 for details. The deficient strain(s) may be tested for growth rate and FFA production. By deleting a competing pathway, the FFA production may not be significantly increased. However, if the deficient mutant grows well and has no decrease in FFA production compared with its parent strain, the mutation causing this loss of function may be kept in the 6803 chromosome and serve as an integration platform to insert gene cassettes specifying a beneficial new activity. In this case, the intermediate Km^{r}-sacB insertion may be replaced by other positive FFA-production gene cassettes.

### Decreasing Repression and Feedback Inhibition

Eliminating repression of expression of genes is important in channeling resources toward maximal synthesis and secretion of FFAs. If it occurs, the repression may likely be eliminated by the adopted experimental design. For instance, substituting promoters with high activities may be used to maximize gene expression. In so doing wild-type promoters of genes may be replaced with improved promoters. Thus potential for binding of repressors may be eliminated as a problem. In this regard, using active constitutive promoters from other gram-negative bacteria such as E. coli and S. enterica will invariably eliminate the potential for repression of transcription. Contending with feedback inhibition is another matter. To accomplish this, the following strategy is proposed based on the predicted ability of a fusion protein subject to feedback inhibition to lead to an inability to synthesize the peptidoglycan layer of the cell wall resulting in cell death, a powerful selection force. Diaminopimelic acid (DAP), is a unique essential constituent of the peptidoglycan layer of the cell wall. Strains with deletion mutations of the asdA gene are unable to synthesize threonine, methionine and DAP (which by decarboxylation is converted to lysine). Such mutants in the absence of DAP undergo DAP-less death and lyse. A plasmid specifying synthesis of the Asd enzyme from Streptococcus mutans could complement and allow the growth of ΔasdA mutants of either E. coli or Salmonella (Nakayama et al. 1988). Interestingly, if a ΔasdA Salmonella culture containing a plasmid with the S. mutans asdA gene was vigorously shaken, cells lysed. This was because the S. mutans enzyme was unable to form a functional enzyme complex with the Salmonella aspartate kinases that produced the unstable β-aspartyl phosphate which is the substrate for the Asd enzyme. This original observation is serving as an additional means to interfer with peptidoglycan synthesis and thus facilitate secretion of FFAs (see Example 10). To develop the selective enrichment for feedback-resistant mutants may require several steps. First the 6803 asdA gene may be deleted as is also described in Example 10. The efficiency of complementation of this defect may then be determined by introducing plasmids (Materials and Methods below) encoding the 6803, Salmonella and S. mutans asdA genes and determining the complementation efficiency dependant on means of culture growth. Assuming that complementation and survival for slow growth and lethality and lysis for vigorous growth may be observed in some of these complementing pairs, the appropriate asdA gene may be chosen, and a fusion with a 6803 gene whose product is subject to feedback inhibition by intracellular FFAs may be made. For example, the accB gene may be fused with the selected asdA gene on a plasmid vector and the plasmid introduced into a ΔasdA 6803 strain maximally producing FFA precursors but lacking *TesA or other FFA export attributes. It is expected that feedback inhibition will decrease the activity of the fused Asd enzyme and lead to some degree of cell lysis. Feedback inhibition resistant mutants should, however, survive. Minor adjustments may be empirically determined to optimize enzymes with varying degrees of feedback inhibition in WT 6803.

### Modify the Chain Structure of the Fatty Acid Products

The 6803 FFA products are useful as biofuel precursors. The chain structure of the FFA products can be modified to contain different branch points, level of saturation, and carbon chain length, thus making these products desirable starting materials for the biofuel application. Preliminary results have shown that the saturation level of FFA has been increased from the native 6803 fatty acid profiles, which was postulated by the fact that the 6803 desaturases are located in the cytoplasmic membranes. The chemical composition of 6803 secreted FFAs also showed that introducing middle chain preferring thioesterases enabled 6803 to produce shorter chain FFAs. The quality of 6803 secreted FFAs for biofuel production may be further improved by increasing the amount of shorter chain FFA production (this is discussed in Materials and Methods below) and by engineering 6803 to produce, or to overproduce branched chain fatty acids (brFA).

As described in Materials and Methods below, exogenous genes for the three steps of brFA synthesis may be introduced into 6803 to engineer 6803 to produce brFAs. The first step in forming brFAs is the production of the corresponding α-keto acids by a branched-chain amino acid aminotransferase (IlvE; EC 2.6.1.42). The ilvE gene (slr0032) may be overexpressed in 6803 by the methods described in Materials and Methods below. The ilvE candidates from other bacteria, which are listed in Table 6 may also be introduced. However, in Salmonella there is a series of balanced-lethal and balanced-attenuation plasmid vector-host systems and one of these is dependent on use of IlvE⁺ plasmid vectors. The aminotransferase reaction encoded by the ilvE gene should be the rate limiting step in brFA biosynthesis in 6803.

The second step, the oxidative decarboxylation of the α-keto acids to the corresponding branched-chain acyl-CoA, is catalyzed by a branched-chain α-keto acid dehydrogenase complex (bkd; EC 1.2.4.4) (Denoya, Fedechko et al. 1995). This complex consists of E1α/β (decarboxylase), E2 (dihydrolipoyltransacylase) and E3 (dihydrolipoyl dehydrogenase) subunits. The bkd genes may also be introduced into 6803. The bkd candidates are listed in Table 6.

Because 6803 does not naturally make brFA, heterogeneous components of fatty acid synthesis machinery with specificity for brFAs need to be introduced into 6803 in the final step. For example, the initiation of brFA biosynthesis utilizes β-ketoacyl-ACP synthase 111 (FabH; EC 2.3.1.41) with specificity for branched chain acyl CoAs (Li et al 2005). Additionally, other components of fatty acid synthesis machineries with specificity for brFAs need to be introduced into 6803, such as acyl carrier protein (ACP) and β-ketoacyl-ACP synthase II (FabF; EC 2.3.1.41). The brFA synthesis machinery gene candidates we will overexpress in 6803 are listed in Table 6.

TABLE 6
brFA genes from selected microorganisms with brFAs

| Organism | Gene | Genbank Accession # |
|---|---|---|
| 6803 | ilvE (slr0032) | NP_442721 |
| E. coli | ilvE | YP_026247 |
| Lactococcus lactis | ilvE | AAF34406 |
| Streptomyces coelicolor | ilvE | NP_629657 |
| | bkdA1 (E1α) | NP_628006 |
| | bkdB1 (E1β) | NP_628005 |
| | bkdC1 (E2) | NP_628004 |
| | bkdA2 (E1α) | NP_733618 |
| | bkdB2 (E1β) | NP_628019 |
| | bkdC2 (E2) | NP_628018 |
| | fabH1 | NP_626634 |
| | ACP | NP_626635 |
| | fabF | NP_626636 |
| Strptomyces avermitilis | bkdA (E1α) | BAC72074 |
| | bkdB (E1β) | BAC72075 |
| | bkdC (E2) | BAC72076 |
| | bkdF (E1α) | BAC72088 |
| | bkdG (E1β) | BAC72089 |
| | bkdH(E2) | BAC72090 |
| | fabH3 | NP_823466 |
| | fabC3 (ACP) | NP_823467 |
| | fabF | NP_823468 |
| Psedomonas putida | ilvE | NP_745648 |
| | bkdA1 (E1α) | AAA65614 |
| | bkdA2 (E1β) | AAA65615 |
| | bkdC (E2) | AAA65617 |
| Bacillus subtilis | bkdAA (E1α) | NP_390288 |
| | bkdAB (E1β) | NP_390289 |
| | bkdB (E2) | NP_390290 |
| | fabH_A | NP_389015 |
| | fabH_B | NP_388898 |
| | ACP | NP_389474 |
| | fabF | NP_389016 |

By the methods described in Materials and Methods below, the brFA synthesis genes from Streptomyces coelicolor may be introduced and overexpressed in the chromosome of one of the FFA-secreting strains (e.g., SD 232). The FFA chain structure of the resulting strain may be analyzed. If brFAs can be produced and secreted by 6803, that will justify the hypothesis of improving fuel quality by branching the FFA chain structure. Depending on the quality and yields of the brFAs produced by the modified 6803, other brFA synthesis genes from other organisms and their optimal expression levels may be tested by a 6803 plasmid expression system which will be discussed in Materials and Methods below.

### Example 10

### Optimize and Duplicate Means to Maximize FFA Secretion

In addition to directly enhancing FAS, multiple means may be used to increase uncoupling FFA from FAS, even when the cells enter the stationary growth phase, and to facilitate the secretion of FFAs. The optimization means include the following three points.

Duplication of Enzyme Activities from Multiple Organisms Using Chromosomal and Plasmid-Specified Gene Activities

As proven by preliminary results, introducing and overproducing exogenous Acyl-ACP TE in 6803 will change the metabolic destination of a portion of fatty acids. Some fatty acids may be decoupled from the FAS and membrane lipid synthesis resulting in FFAs. TEs are the essential enzymes in our FFA overproducing 6803 strains. Therefore, multiple TEs may be overproduced to improve the FFA overproduction and determine their optimal expression levels. Our most recent construction of SD277 completely validates this approach.

In bacteria and plants, each FAS reaction is catalyzed by a discrete, monofunctional enzyme and the growing acyl chain is bound to ACP. This FAS version has been termed type II (FASII). Thus plant and bacterial FAS systems resemble each other in machinery and the plant fatty acid synthase resides in the chloroplast (Ohlrogge, Kuhn et al. 1979), which is considered to be of prokaryotic (cyanobacterial) origin. Therefore, the common ancestry of plant and bacterial FAS results in their structural and functional similarities (Ohlrogge 1982). Thus the plant FAS enzymes are usually functional in cyanobacteria, which also has been demonstrated by our results. For this reason, a variety of additional plant TEs may be tested in 6803 to achieve maximum FFA production yields with optimal chain lengths.

The known plant TEs can be divided into two main classes, based on their specificity for acyl-ACPs of different chain lengths and degrees of unsaturation. The “FatA” type of plant TE has preferential activities on oleoyl-ACP (C18:1). The “FatB” type of plant TE has preferential activity on saturated acyl-ACPs with different chain length preferences. The TE candidates are listed in Table 7. Besides the plant TEs, various TEs from other bacteria such as S. enterica may also be used.

TABLE 7
Acyl-ACP Thioesterases

| | | Preferential | Accession |
|---|---|---|---|
| Source organism | Gene | Product | Number |
| E. coli | tesA | C18:1 | ACC73596 |
| | without leader | | |
| | sequence | | |
| Desulfovibrio vulgaris | acyl-ACP | unknown | YP_002435844 |
| | thioesterase | | |
| Alkaliphilus | acyl-ACP | unknown | YP_001512437 |
| oremlandii OhlLAs | thioesterase | | |
| Anaeromyxobacter | acyl-ACP | unknown | YP_002494610 |
| dehalogenans 2CP-1 | thioesterase | | |
| Streptococcus | acyl-ACP | unknown | NP_721775 |
| mutans UA159 | thioesterase | | |
| Cinnamonum | fatB | C14:0 | Q39473 |
| camphorum | | | |
| Arabidopsis thaliana | fatA | C18:1 | NP_189147; |
| | | | NP_193041 |
| Arabidopsis thaliana | fatB | C16:1 | CAA85388 |
| Bradyrhiizobium | fatA | C18:1 | CAC39106 |
| japonicum | | | |
| Cuphea hookeriana | fatA | C18:1 | AAC72883 |

Each of the genes listed in Table 7 may be cloned into pSD504 and into pSD505 (Materials and Methods below) to investigate introduction of one or more combinations of two genes introduced into a suitable strain of 6803 and determine the effect on level and chain length of synthesized FFAs and on the composition of secreted FFAs. pSD504 confers resistance to gentamycin and cloned genes are induced for expression by addition of arabinose (see Table 13). pSD505 confers resistance to streptomycin and spectinomycin and cloned genes are expressed under the control of P_{trc}, which is inducible with IPTG. Thus both plasmids may be selected and maintained independently and their cloned genes regulated for expression independently. The two plasmids share sequence homology and can undergo recombination at a low frequency but this will not alter the structure and regulation of inserted genes. A more likely possibility would be incompatibility in maintenance of the two plasmids. However, since 6803 has some 6 to 8 chromosome copies it is expected that the number of the RSF1010 derived plasmids will be sufficient to force co-maintenance by presence of the selective antibiotics to which the plasmids confer resistance.

The results may enable us to determine which combination of tes and fat genes will generate the most productive strain. Of course, the choice of genes may also be influenced by the specific chain length desired to be secreted by a given 6803 strain. The gene may then be optimized for high-level expression (transcription and translation) and the optimal site within the chromosome or in one of the indispensible large plasmids to maximize stability and level of expression may be investigated. In most cases, the insertion site may be chosen to inactivate a gene or genes encoding a pathway that competes for carbon flow that may be channelled to synthesize and secrete FFAs.

### Genetically Modifying the Cells to Maintain Continuous FFA Production and Secretion During Stationary Phase for Periods of Long Duration

One aim of our research is to enhance secreting FFA from 6803 cells throughout their life cycle, including in stationary growth phase. The objective is that more of the energy derived from photosynthetic CO₂ fixation can be used for fatty acid production and less applied toward growth of non-lipid biomass. For this purpose, most of the FFA-overproducing genes need to be driven by promoters that initiate high-level protein synthesis even in the stationary growth phrase.

Three methods to identify and redesign the 6803 stationary phase promoters are proposed. (1) According to the 6803 mRNA microarray data (Foster, Singh et al. 2007), the fluctuation in expression of many but presumably not all genes during various growth stages is known. Some genes maintain their expression levels and (of interest to the research) some increase transcription levels when cells enter stationary growth phase. Table 8 shows the top 10 stationary phase up-regulated genes, most of which are not up-regulated in the ΔsigB mutant. Bioinformatics tools may be used to analyze the promoters for these genes that are not decreased in the stationary growth phase to identify the consensus sequence for the stationary promoters. (2) The 5′ upstream regions of genes that contain consensus sequences recognized by the group 2 sigma factors (especially SigB and SigC), which might be important in facilitating transcription in stationary phase, may be searched and compared. (3) Four promoter trap vectors (Materials and Methods below) were designed with different reporters to search for promoters with maximum transcription efficiencies in stationary phase. Libraries of short DNA sequences in pSD502 may therefore be made such that the promoters would drive expression of the gfp gene such that we could use flow cytometry and cell sorting to recover cells with plasmid clones giving maximum GFP activity in stationary phase.

Promoters identified in these studies may be used to fuse to genes for any step in the FFA production and secretion pathway that would benefit from improved gene expression in stationary phase. It is also anticipated that hybrid as well as sequential promoters may be designed to maximize expression levels.

TABLE 8
The top 10 stationary up-regulated genes in 6803 and their performances
in a ΔsigB mutant

| | | | ΔsigB | |
|---|---|---|---|---|
| Gene | General Pathway | Specific Pathway | st/ex | WT st/ex |
| sll0856 | Transcription | RNA polymerase | −1.03 | 11.03 |
| | | sigma-E factor (rpoE) | | |
| slr0854 | DNA replication, | deoxyribopyrimidine | 1.06 | 10.79 |
| | restriction | photolyase (phr) | | |
| sll0247 | Photo-synthesis | iron-stress chlorophyll- | −1.14 | 8.66 |
| | and respiration | binding protein (isiA) | | |
| sll0858 | none | none | 1.3 | 8.24 |
| sll0867 | none | none | 1.38 | 7.36 |
| sll1722 | none | none | −1.03 | 6.99 |
| sll0314 | none | none | 1.03 | 5.51 |
| slr0887 | none | none | 1.04 | 5.28 |
| sll0470 | none | none | −1.15 | 5.17 |
| slr1968 | none | none | −1.33 | 5.09 |

### Investigating Means of Membrane and Cell Surface Modifications to Further Enhance Release of Fatty Acids

It is has been proven that increasing synthesis of fatty acids will automatically lead to increased secretion of fatty acids. However, results have shown that the FFA-secreting 6803 strains overproduce FFAs with a portion retained inside the cells to inhibit cell functions (by feedback inhibition) or introduce membrane leakage. Our results also showed that if the polypeptidoglycan layers of fatty acid secreting cyanobacteria are compromised by ampicillin, there was significant leakage/export of FFAs outside the cells. Removal of the S-layer also facilitated FFA secretion with an overall increased FFA yield. This implies that facilitating the removal of intracellular FFA releases the product inhibition on the FFA producing equilibrium. Therefore, it is important to further facilitate fatty acid secretion into the culture medium through the cyanobacterial cell envelope without causing damage that would lead to leakage of diverse intracellular contents.

The cyanobacterial cell envelope is composed of four layers (Lounatmaa, Vaara et al. 1980); the external surface layers, such as S-layers and carbohydrate structures (Karlsson, Vaara et al. 1983), outer membrane, peptidoglycan layer (Hoiczyk and Hansel 2000), and cytoplasmic membrane (FIG. 13). Of the four layers, cyanobacterial outer membrane and cytoplasmic membrane are composed of phospholipids and galactolipids. Long chain fatty acids have permeabilities through such membrane lipids that are many orders of magnitude higher than glucose, amino acids, and ions (FIG. 12). Hamilton and colleagues have shown uncharged fatty acids perform fast free diffusion by ‘flip-flop’ in the phospholipid bilayer thereby obviating the need for a specific protein to promote transmembrane movement (Hamilton 2007). On this premise, the hydrophilic cell envelop polypeptidoglycan and surface-layer protein layers are the main barriers for FFA secretion. However, the LPS component of the outer membrane may also impede FFA secretion/export and thus mutant strains have been constructed to test this hypothesis. Since the surface-protein layer has already been deleted, further research is focusing on facilitating the FFAs crossing the polypeptidoglycan layers. To do this, two strategies are proposed. First, the cell-wall peptidoglycan may be weakened to improve release of FFAs to the external media. Second, membrane transporter proteins, specifically for hydrophobic molecules, may be introduced into 6803 cell envelope layers to facilitate FFA export.

To weaken the peptidoglycan layer, which actually is composed of several layers, three solutions are proposed. The first solution is to reduce peptidoglycan synthesis in 6803 by down-regulating the efficiency of peptidoglycan synthesis genes, such as those in the mur (essential peptidoglycan synthesis and ligation genes, e.g., slr0017, slr1423, slr1656 and sll2010) and ldh (involved in peptidoglycan precursor UDP-N-acetylmuramyl-pentapeptide synthesis, e.g., slr0528 and slr1656) families to weaken the polypeptidoglycan layer structures. In addition, some of the penicillin-binding protein genes, such as ftsl (sll1833), mrcB (slr1710) and ponA (sll0002) which are required in assembly of the peptidoglycan, may be deleted or modified to favor the release of fatty acids. In this regard, we have already deleted the slr1710 gene encoding PBP2 and included that mutation in SD248 and desendants (Table 1). We also deleted PBP3 in SD263, and PBP4 in SD264. Actually, we deleted combinations of all three PBP genes but were unable to incorporate more than one mutation into 6803 strains to retain good growth properties. For example, we deleted PBP2 and PBP3 in SD255, but this strain grew very slowly with a doubling time of 22 h. We also deleted PBP2 and PBP4 in SD259, and this strain also grew very slowly with a doubling time above 20 h.

Another way to interfere with peptidoglycan synthesis is by substituting a gene for a central step in an essential pathway for synthesis of an essential cell wall component with one from another bacterial species, such as using a foreign asd or alr gene. The asdA gene from Streptococcus mutans (Cardineau and Curtiss 1987) may be expressed in E. coli to enable synthesis of DAP (diaminopimelic acid) for incorporation into peptidoglycan. However, due to the fact that a foreign enzyme does not generate a stable poly-enzyme complex to efficiently catalyze the entire biosynthetic pathway to synthesize DAP rapid growth with vigorous aeration destabilizes the peptidoglycan layer in E. coli. Addition of pSD506 plasmid derivatives encoding asdA genes from diverse bacterial species into a FFA producing and secreting ΔasdA 6803 mutant may be compared to evaluate whether FFA secretion is increased. In this regard, we have cloned the 6803 alr (for alanine racemase to synthesize D-alanine required for peptidoglycan synthesis) and asdA genes and expressed them in E. coli and S. Typhimurium strains with deletion alr and dadX (E. coli) or dadB (Salmonella) (Lobockam M. et al 1994, Lilley, P. E. et al 1993, and Wasserman, S. A. et al 1983) and asdA mutations to enable synthesis of peptidoglycan and growth. E. coli and Salmonella have two genes encoding alanine rasemases whereas 6802 only has a single alr gene. We have also generated His-tagged Alr, AsdA and Murl (glutamate racemase) (Doublet et al., 1993) proteins to enable production of rabbit antibodies to enable immunological detection. Lastly, we have made suicide vectors to enable allele replacement mutagenesis to generate 6803 Δalr (Δslr0827), ΔasdA (Δslr0549) and Δmurl (Δslr1746) mutants (FIG. 15) to completely validate this approach for enhancing FFA secretion/export. In addition, these mutations will have other important uses as described in the Examples below.

The third solution to weaken the peptidoglycan layer may be to introduce endolysin genes from bacteriophages into 6803 and to express such genes at a low level. Endolysins are peptidoglycan-degrading enzymes that attack the covalent linkages of the peptidoglycans that maintain the integrity of the cell wall (Loessner 2005). It has been demonstrated that the endolysin gp19 from Salmonella phage P22 is able to degrade the 6803 polypeptidoglycan layers, and the endolysin R from E. coli phage λ is able to compromise the 6803 polypeptidoglycan layers. Thus, the 19 or R endolysin genes in 6803 may be expressed at very low levels to weaken the peptidoglycan to facilitate fatty acid secretion. Different promoters with variant low transcription efficiencies of the endolysin genes may be attempted to limit adverse growth effects. We can also insert infrequently used codons into the genes encoding these endolysins to reduce levels of endolysin synthesis.

Besides weakening the cell envelopes to facilitate lipid secretion, transporter or porin genes may also be expressed or overexpressed to make channels for the lipid. Most bacterial fatty acid translocation studies were focused on fatty acid uptake, that is, the transmembrane movement of fatty acids from the outside to the inside of the cells (Black and DiRusso 2003). However, a few studies showed evidence that, in some bacteria, intracellularly synthesized lipids were transported outside the cell by transporter proteins. For example, the cell envelop of Mycobacterium tuberculosis includes a thick layer of lipids on the outer part of the cell, which protects the tubercle bacillus from the host's immune system. Mycolic acids, the major constituents of this protective layer, are the long chain fatty acids found in the bacteria of the Mycobacterium genus. The precursor of mycolic acids, trehalose monomycolate (TMM), is synthesized inside the cell, and then transported outside the cell (into the outer membrane) by the ATP-Binding Cassette (ABC) transporter (Rv1272c Rv1273c). Use of these genes in 6803 may be examined to determine whether they enhance FFA secretion.

Many transport and efflux proteins serve to excrete a large variety of compounds, and these may possibly be modified to be selective for fatty acids. For example, E. coli outer membrane protein FadL is a membrane-bound fatty acid transporter, which binds long chain fatty acid with a high affinity (Dirusso and Black 2004). Other suitable transport proteins include, efflux proteins (Thompson, Lobo et al. 2009), and fatty acid transporter proteins (FATP) (Hirsch, Stahl et al. 1998). These proteins or their derivatives may be introduced into the fatty acid producing 6803 to determine whether they facilitate lipid secretion. Some transporter proteins are listed in Table 9.

TABLE 9
Potential fatty acid transporter proteins

| Gene | Function | Accession | Organism |
|---|---|---|---|
| msbA | msbA fused lipid | NP_415434 | Escherichia coli K- |
| | transporter of ABC | | 12 |
| | superfamily | | |
| fadL | long-chain fatty acid | NP_416846 | Escherichia coli K- |
| | outer membrane | | 12 |
| | transporter | | |
| acrF | Acriflavine resistance | NP_417732 | Escherichia coli K- |
| | protein F | | 12 |
| msbA | lipid A ABC transporter, | NP_794717 | Pseudomonas |
| | ATP-binding/permease | | syringae |
| | protein | | |
| Rv1272c Rv1273c | Drugs-transport | NP_215788 | Mycobacterium |
| | transmembrane ABC | NP_215789 | tuberculosis |
| | transporter | | |
| ABC transporter | Alkane transporter | AAN73268 | Rhodocuccus |
| | | | erythropolis |
| RHA1_ro05645 | Bifunctional ABC lipid A | YP_705582 | Rhodococcus jostii |
| RHA1_ro06043 | transporter: ATP- | YP_705978 | RHA1 |
| | binding/permease | | |
| Fatty acid | transporter, short-chain | ABP78560 | Pseudomonas |
| transporter | fatty acid transporter | | stutzeri A1501 |
| | family | | |
| Fatty acid | transporter, short-chain | AAN68732 | Pseudomonas |
| transporter | fatty acid transporter | | putida KT2440 |
| | family | | |
| CER5 | Wax transporter | At1g51500, | Arabidopsis |
| | | AY734542, | thaliana |
| | | At3g21090, | |
| | | At1g51460 | |
| fadL | long-chain fatty acid | YP_002347691 | Yersinia pestis |
| | outer membrane | | CO92 |
| | transporter | | |
| Efflux pump | ABC transporter | ZP_04509251 | Yersinia pestis |
| | multidrug efflux pump | EEO90963 | Pestoides |
| acrA | Multidrug-efflux | YP_007549 | Candidatus |
| acrB | transporter | YP_007550 | amoebophila |
| | | | UWE25 |
| acrE | Transmembrane | YP_312213 | Shigella sonnei |
| | protein affects septum | | Ss046 |
| | formation and cell | | |
| | membrane permeability | | |
| tll1618 | Multidrug efflux | NP_682408 | Thermosynechococcus |
| tll1619 | transporter | NP_682409 | elongates BP-1 |
| tll0139 | | NP_680930 | |

### Example 11

### Engineer Strains for “Green Release” of FFA by Inducible Expression of Lipolytic Enzyme Genes

Traditional downstream recovery of microbial lipids, requires physical cell lysis followed by chemical solvent extraction and accounts for 70-80% of the total cost of biofuel production (Molina Grima, Belarbi et al. 2003). We propose a more cost-effective way to harvest lipids from cyanobacterial biomass for biofuel production that precludes the need for mechanically disrupting the cells. To this end, we developed a Green Recovery system in which lipolytic enzymes degrade the membrane lipids into free fatty acids (FFA) with the collapse of cells. The Green Recovery system controls the synthesis of lipolytic enzymes using CO₂-limitation inducible promoters, which induce expression of the lipolytic genes upon cessation of CO₂ aeration. This same system described below could be adapted for Green Recovery of lipids and other biofuel precursors from a diversity of photosynthetic microorganisms not limited to cyanobacteria and including micro algae, unicellular algae, diatoms and purple-sulfur bacteria and even from non-photosynthestic bacteria such as Escherichia coli and members of the Enterobacteriaceae and Pseudomonaceae. In these cases, one could use oxygen or nitrogen regulated expression of genes encoding lipases instead of using CO₂ regulated gene expression.

The lipolytic enzymes (EC 3.1.1) including galactolipase and phospholipase B (Svendsen 2000) hydrolyze the carboxylic ester bonds to release the fatty acids from diacylglycerols. Galactolipase (EC 3.1.1.26) catalyzes the hydrolysis of galactolipids by removing one or two fatty acids (Helmsing 1969). Phospholipase B is an enzyme with a combination of both Phospholipase A1 (EC 3.1.1.32) and Phospholipase A2 (EC 3.1.1.4) activities, which can cleave acyl chains from both the sn-1 and sn-2 positions of a phospholipid (Kohler, Brenot et al. 2006). For the purpose of fatty acid recovery from membrane lipids, we tested the performance of three lipolytic enzymes (from a bacterium, a fungus, and an herbivorous animal, respectively) in cyanobacterium Synechocystis sp. PCC 6803. The lipase from Staphylococcus hyicus (Shl) was selected because it has a very broad substrate specificity ranging from triacylglycerol lipids of various chain lengths to phospholipids and lysophospholipids (Rosenstein and Gotz 2000). The second candidate was a modified fungal phospholipase from Fusarium oxysporum (Fol) that exhibited galactolipase activity as well as increased phospholipase activity (Rapp 1995). Third, the guinea pig lipase (Gpl, also called GPLRP2, guinea-pig pancreatic lipase-related protein 2) from the digestive juice of guinea pig (Andersson, Carriere et al. 1996) was also tested. Gpl shows the highest galactolipase activity known to date, and plays a dual role in the digestion of galactolipids and phospholipids, the most abundant lipids occurring in plant thylakoid membranes (Andersson, Carriere et al. 1996).

We also developed a nickel inducible lysis system for 6803, which is able to induce phage lysis genes to break down cell walls by adding nickel as an inducer (Liu and Curtiss 2009). This system shows the feasibility of controlling lethal genes by inducible promoters. However, nickel is toxic to the environment and also adds cost. Therefore, we developed a 6803 CO₂ limitation response mechanism (McGinn, Price et al. 2003) for an inducible transcription system regulated by CO₂ rather than nickel. It has been reported that aeration of illuminated 6803 cells with CO₂-free air for 30 min depleted the culture CO₂ concentration to near zero levels. Under these conditions, transcripts for the three inducible inorganic carbon uptake systems, ndhF3, sbtA, and cmpA, showed near-maximal abundance after 15 min under CO₂ limitation (McGinn, Price et al. 2003). By utilizing the promoter sequences for these genes to control the lipase genes, Green Recovery of lipids can be initiated by CO₂ limitation resulting from stopping aeration of the 6803 culture. Aeration to the photobioreactor is necessary and easy to regulate, thus limiting the CO₂ supply is an economical and environmentally friendly method to initiate lipid hydrolysis.

We constructed a series of 6803 strains for testing and optimizing Green Recovery (FIG. 16 and Table 10). By using the Km^{R}/sacB intermediate double crossover recombination method (Liu and Curtiss, 2009), we inserted synthesized genes fol, shl, gpl, and an artificial operon fol RBS (ribosome binding site) gpl into 6803 wild-type after P_{cmp} and before the ATG of the cmpA gene, resulting in strains SD256, SD257, SD258, and SD237, respectively. The gpl gene was inserted into wild-type and SD237 after P_{sbt} and before the ATG of the sbtA gene to result in SD252 and SD253, respectively. To incorporate the Green Recovery system into the FFA-secretion strains (Examples 1 to 8) we inserted the artificial operon fol RBS shl into an FFA-secretion strain SD232 (Table 1) after P_{cmp} to result in SD239. To achieve rapid membrane damage we inserted another artificial operon gpl RBS 13 19 15, in which 13 19 15 are lysis genes from Salmonella phage P22 (Liu and Curtiss 2009), into SD239 after P_{sbt} to result in SD254 and SD262, respectively. The growth of wild-type based strains (e.g., SD256, SD257, and SD237) was similar to the wild-type, with doubling times of about 9 hours. The growth of the SD232-based strains (e.g., SD239, SD254, and SD262) was also similar to SD232, with doubling times of about 12 hours.

TABLE 10
SD strains constructed for Green Recovery studies

| SD No. | Genotype^{a} | Construction | Remarks |
|---|---|---|---|
| SD100 | Synechocystis sp. PCC | | See Table 1 |
| | 6803 wild-type | | |
| SD200 | ΔlipA::Km^{R} sacB | Transform SD100 with | Deletion of a putative 6803 |
| | | pψ200 (Δsll1969:: Km^{R} | lipase gene sll1969. From |
| | | sacB) and select for | 908392 to 908941 in the |
| | | kanamycin resistance | chromosome (Cyanobase). |
| SD232 | Δslr1609::P_{psbA2} ′tesA | Constructed in previous | 3^{rd} generation constitutive |
| | Δ(slr1993-slr1994)::P_{cpc} | research. | FFA-secreting strain. Plant |
| | accBC P_{rbc} accDA | | (Umbellularia californic and |
| | Δsll1951::*P_{psbA2} Uc fatB1 | | Cuphea hookeriana) and E. coli |
| | P_{rbc} Ch fatB2 | | thioesterases and ACC |
| | | | were overproduced, and an |
| | | | acyl-ACP synthetase gene, |
| | | | PHB synthesis genes, and a |
| | | | surface layer gene were |
| | | | deleted in SD100. |
| SD234 | P_{cmp}::Km^{R} sacB | Transform SD100 with | Intermediate strain for |
| | | pψ234 (P_{cmp}::Km^{R} sacB) | inserting genes under the |
| | | and select for kanamycin | control of P_{cmp} on the basis of |
| | | resistance. | SD100. |
| SD235 | Δslr1609::P_{psbA2} ′tesA | Transform SD232 with | Intermediate strain for |
| | Δ(slr1993-slr1994)::P_{cpc} | pψ234 (P_{cmp}::Km^{R} sacB) | inserting genes under the |
| | accBC P_{rbc} accDA | and select for kanamycin | control of P_{cmp} on the basis of |
| | Δsll1951::*P_{psbA2} Uc fatB1 | resistance. | SD232. |
| | P_{rbc} Ch fatB2 | | |
| | P_{cmp}::Km^{R} sacB | | |
| SD237 | P_{cmp}::fol RBS shl | Transform SD234 with | Green Recovery strain with |
| | | pψ237 (P_{cmp}::fol RBS shl) | fol and shl controlled by P_{cmp}. |
| | | and select for sucrose | |
| | | survival. | |
| SD239 | Δslr1609::P_{psbA2} ′tesA | Transform SD235 with | Combination of SD232 and |
| | Δ(slr1993-slr1994)::P_{cpc} | pψ237 (P_{cmp}::fol RBS shl) | SD237, a FFA secretion plus |
| | accBC P_{rbc} accDA | and select for sucrose | Green Recovery strain |
| | Δsll1951::*P_{psbA2} Uc fatB1 | survival. | |
| | P_{rbc} Ch fatB2 | | |
| | P_{cmp}::fol RBS shl | | |
| SD244 | P_{sbt}::Km^{R} sacB | Transform SD100 with | Intermediate strain for |
| | | pψ244 (P_{sbt}::Km^{R} sacB) | inserting genes under the |
| | | and select for kanamycin | control of P_{sbt} on the basis of |
| | | resistance. | SD100. |
| SD245 | P_{cmp}::fol RBS shl | Transform SD237 with | Intermediate strain for |
| | P_{sbt}::Km^{R} sacB | pψ244 (P_{sbt}::Km^{R} sacB) | inserting genes under the |
| | | and select for kanamycin | control of P_{sbt} on the basis of |
| | | resistance. | SD237. |
| SD246 | Δslr1609::P_{psbA2} ′tesA | Transform SD239 with | Intermediate strain for |
| | Δ(slr1993-slr1994)::P_{cpc} | pψ244 (P_{sbt}::Km^{R} sacB) | inserting genes under the |
| | accBC P_{rbc} accDA | and select for kanamycin | control of P_{sbt} on the basis of |
| | Δsll1951::*P_{psbA2} Uc fatB1 | resistance. | SD239. |
| | P_{rbc} Ch fatB2 | | |
| | P_{cmp}::fol RBS shl | | |
| | P_{sbt}::Km^{R} sacB | | |
| SD252 | P_{sbt}::gpl | Transform SD244 with | Green Recovery strain with |
| | | pψ252 (P_{sbt}::gpl) and | gpl controlled by P_{sbt}. |
| | | select for sucrose | |
| | | survival. | |
| SD253 | P_{cmp}::fol RBS shl | Transform SD245 with | Combination of SD237 and |
| | P_{sbt}::gpl | pψ252 (P_{sbt}::gpl) and | SD252, with 3 lipolytic genes. |
| | | select for sucrose | |
| | | survival. | |
| SD254 | Δslr1609::P_{psbA2} ′tesA | Transform SD246 with | Combination of SD232, |
| | Δ(slr1993-slr1994)::P_{cpc} | pψ252 (P_{sbt}::gpl) and | SD237 and SD252, with 3 |
| | accBC P_{rbc} accDA | select for sucrose | lipolytic genes and FFA |
| | Δsll1951::*P_{psbA2} Uc fatB1 | survival. | secretion. |
| | P_{rbc} Ch fatB2 | | |
| | P_{cmp}::fol RBS shl | | |
| | P_{sbt}::gpl | | |
| SD256 | P_{cmp}::fol | Transform SD234 with | Green Recovery strain with |
| | | pψ256 (P_{cmp}::fol) and | individual lipolytic enzyme |
| | | select for sucrose | Fol. |
| | | survival. | |
| SD257 | P_{cmp}::shl | Transform SD234 with | Green Recovery strain with |
| | | pψ257 (P_{cmp}::shl) and | individual lipolytic enzyme |
| | | select for sucrose | Shl. |
| | | survival. | |
| SD258 | P_{cmp}::gpl | Transform SD234 with | Green Recovery strain with |
| | | pψ258 (P_{cmp}::gpl) and | individual lipolytic enzyme |
| | | select for sucrose | Gpl. |
| | | survival. | |
| SD262 | Δslr1609::P_{psbA2} ′tesA | Transform SD246 with | On the basis of SD254, the |
| | Δ(slr1993-slr1994)::P_{cpc} | pψ262 (P_{sbt}::gpl RBS 13 | Salmonella phage P22 lysis |
| | accBC P_{rbc} accDA | 19 15) and select for | cassette 13 19 15 was added |
| | Δsll1951::*P_{psbA2} Uc fatB1 | sucrose survival. | under the control of P_{sbt}. |
| | P_{rbc} Ch fatB2 | | |
| | P_{cmp}::fol RBS shl | | |
| | P_{sbt}::gpl RBS 13 19 15 | | |

^{a}Genetic information: lipA (sll1969), a putative lipase gene in 6803 chromosome; sacB, sacB gene, which is lethal for cyanobacteria in the presence of sucrose; Km^{R}, kanamycin resistance gene; slr1609, the 6803 fatty acid activation gene, encoding an acyl-ACP synthetase (Kaczmarzyk and Fulda 2010)); P_{psbA2}, the promoter of 6803 psbA2 gene, a 6803 strong promoter (Agrawal, Kato et al. 2001); ′tesA, an E. coli thioesterase gene without the export signal sequence (Cho and Cronan 1995); slr1993 and slr1994, two poly-3-hydroxybutyrate (PHB) synthesis genes; P_{cpc}, the promoter of 6803 cpc operon (Imashimizu, Fujiwara et al. 2003); P_{rbc}, the promoter of 6803 rbc operon (Onizuka, Akiyama et al. 2003); accB, accC, cD, and accA, four genes coding for 6803 acetyl-CoA carboxylase (ACC) subunits (Davis, Solbiati et al. 2000); sll1951, encoding a hemolysin-like protein, which is a 6803 cell surface layer protein (Sakiyama, Ueno et al. 2006); *P_{psbA2}, an improved promoter from P_{psbA2}; Uc fatB1, a TE gene from Umbellularia californica (Pollard, Anderson et al. 1991); Ch fatB2, a TE gene from Cuphea hookeriana (Dehesh, Jones et al. 1996); P_{cmp}, the promoter of 6803 cmp operon; fol, a synthesized gene based on the amino acid sequence of the fungal phospholipase from Fusarium oxysporum (Fol); shl, a synthesized gene based on he amino acid sequence of the lipase from Staphylococcus hyicus (Shl); gpl, a synthesized gene based on the amino acid sequence of guinea pig pancreatic lipase-related protein 2 (GPLRP2); P_{sbt}, the promoter of 6803 sbtA gene; 13 19 15, Salmonella phage P22 lysis cassette.

Limiting the CO₂ supply to the Green Recovery strain cultures (density >10⁸ cells/ml) resulted in culture discoloration (FIG. 17) and membrane permeability as revealed by Sytox Green staining (Liu and Curtiss 2009) (FIGS. 18 and 19). A comparison between the Sytox Green analysis for membrane permeability and the colony forming unit (CFU) analysis for cell viability showed that cell death happened before the membrane became noticeably permeable (FIG. 20). When the membrane permeable cell percentage increased to about 50%, the CFU dropped to below 0.1% of the starting CFU. Strains synthesizing the fungal lipase Fol (in SD256) or the bacterial lipase Shl (in SD267) had high levels of permeability when incubated under CO₂-limiting conditions compared to wild-type (Table 11). The membrane damage rate in SD237 with both fol and shl (45.2%/day) was faster than those of the strains synthesizing a single lipase (35.1%/day for SD256 and 33.8%/day for SD257). The guinea pig lipase gene gpl resulted in a damage rate of 9.4%/day when controlled by P_{cmp} in SD258 and a faster damage rate of 13.5%/day when controlled by P_{sbt} in SD252 (Table 11). Together these results strongly suggest that the condition of CO₂ limitation causes the expression of genes encoding lipases that result in increased membrane permeability.

TABLE 11
Membrane damage and FFA yields of SD strains for Green Recovery

| | | Starting cell | Membrane | | FFA yield |
|---|---|---|---|---|---|
| | Genetic | density | damage^{b} | Recovered | (10⁻¹² mg/ |
| Strain | Description^{a} | (CFU/ml) | (%/day) | FFA^{c} (mg/L) | cell) |
| SD100 | Wild Type | 3.2 × 10⁹ | 8.9 | 16.5 ± 2.5 | 5.1 ± 0.8 |
| SD200 | ΔlipA::sacB Km^{R} | 3.4 × 10⁹ | 2.5 | 2.7 ± 1.1 | 0.8 ± 0.3 |
| SD256 | P_{cmp}::fol | 1.6 × 10⁹ | 35.1 | 19.3 ± 1.0 | 12.4 ± 0.6 |
| | | 3.9 × 10⁸ (¼)^{d} | 94.7 | 5.5 ± 0.7 | 14.0 ± 1.8 |
| SD257 | P_{cmp}::shl | 2.9 × 10⁹ | 33.8 | 22.7 ± 1.0 | 7.9 ± 0.4 |
| | | 7.2 × 10⁸ (¼)^{d} | 83.4 | 5.7 ± 1.2 | 8.0 ± 1.7 |
| SD237 | P_{cmp}::fol RBS shl | 1.5 × 10⁹ | 45.2 | 23.6 ± 1.1 | 15.7 ± 0.7 |
| | | 3.8 × 10⁸ (¼)^{d} | 91.3 | 10.6 ± 0.1 | 28.0 ± 0.4 |
| SD258 | P_{cmp}::gpl | 3.0 × 10⁸ | 9.4 | 5.2 ± 0.6 | 17.4 ± 2.0 |
| SD252 | P_{sbt}::gpl | 3.5 × 10⁸ | 13.5 | 3.1 ± 1.4 | 8.8 ± 4.1 |
| SD239 | Δslr1609::P_{psbA2 ′tesA} | 5.8 × 10⁸ | 56.7 | 23.6 ± 0.02^{e} | 40.7 ± 0.03^{e} |
| | Δ(slr1993-slr1994):: | | | 44.6 ± 2.8^{f} | 76.8 ± 4.7^{f} |
| | P_{cpc} accBC P_{rbc} | | | 21.0 ± 2.8^{g} | 36.1 ± 4.7^{g} |
| | accDA | | | | |
| | Δsll1951::*P_{psbA2} Uc | | | | |
| | fatB1 P_{rbc} Ch fatB2 | | | | |
| | P_{cmp}::fol RBS shl | | | | |
| SD254 | Δslr1609::P_{psbA2} ′tesA | 7.3 × 10⁸ | 34.5 | 45.1 ± 0.4^{e} | 61.7 ± 0.5^{e} |
| | Δ(slr1993-slr1994):: | | | 63.6 ± 0.4^{f} | 87.2 ± 0.5^{f} |
| | P_{cpc} accBC P_{rbc} | | | 18.6 ± 0.4^{g} | 25.4 ± 0.5^{g} |
| | accDA | | | | |
| | Δsll1951::*P_{psbA2} Uc | | | | |
| | fatB1 P_{rbc} Ch fatB2 | | | | |
| | P_{cmp}::fol RBS shl | | | | |
| | P_{sbt}::gpl | | | | |
| SD262 | Δslr1609::P_{psbA2} ′tesA | 8.9 × 10⁸ | 67.3 | 47.0 ± 0.7^{e} | 52.8 ± 0.8^{e} |
| | Δ(slr1993-slr1994):: | | | 73.4 ± 1.4^{f} | 82.5 ± 1.5^{f} |
| | P_{cpc} accBC P_{rbc} | | | 26.5 ± 1.4^{g} | 29.7 ± 1.5^{g} |
| | accDA | | | | |
| | Δsll1951::*P_{psbA2} Uc | | | | |
| | fatB1 P_{rbc} Ch fatB2 | | | | |
| | P_{cmp}::fol RBS shl | | | | |
| | P_{sbt}::gpl RBS 13 19 | | | | |
| | 15 | | | | |

^{a}Detailed genetic information is described in Table 10.
^{b}Membrane damage was detected by Sytox staining. The damage rates (%/day) were estimated from starting time point to the 50% permeable cell time point, when over 99.9% cells were dead at last. If the permeable cell percentage did not drop to 50%, the damage rates were estimated over the entire experimental period.
^{c}Recovered FFAs were extracted by 10 ml hexane from 16 ml cultures after four days of CO₂ limitation.
^{d}The data for 1/16 dilutions were similar to the data for ¼ dilutions.
^{e}The secreted FFAs before CO₂ limitation.
^{f}Total FFAs after CO₂ limitation, including secreted FFAs and released FFAs.
^{g}Calculated released FFAs for the combination strains by subtracting the total amount from the secreted amount.

Wild-type 6803 also showed membrane damage at high cell density under CO₂ limitation conditions (FIG. 19). This suggests that the wild-type 6803 cells have a background autolysis at high cell density because of the native lipase gene(s), but in the Green Recovery strains where exogenous lipolytic genes are controlled by CO₂ limitation inducible promoters, the inducible membrane destruction is much stronger than the background autolysis. We therefore constructed a lipA deficient strain SD200 by interrupting the putative lipase gene (lipA, sll1969) with the Km^{R}/sacB cassette. Compared with wild-type, SD200 showed much less background autolysis under these conditions (FIG. 19 and Table 11). This supports the conclusion that the lipA gene is responsible for background autolysis, and that the presence of the lipA gene contributes to the efficiency of Green Recovery. We assume that the Green Recovery system can be more tightly controlled by exchanging the native promoter of lipA with a CO₂ limitation inducible promoter. Importantly, we will use the CO₂ limitation inducible promoters to control the native lipase lipA gene (slr1969), and we believe that will result in tighter control of the Green Recovery and a higher lysing rate for the process.

We tested several optimal conditions for Green Recovery, such as cell density of the cultures (FIG. 21), intensity of illumination (FIG. 22), and agitation of the cultures (FIG. 22). It was observed that the membrane damage was faster and FFA release was higher after a ¼ dilution (about 10⁸ cells/ml) than at the original cell density (about 10⁹ cells/ml) (Table 11 and FIG. 21). Light is essential for Green Recovery, which is negligible in the dark (less than 2 μmol photons m⁻² s⁻¹), and much slower in low light (20 μmol photons m⁻² s⁻¹) than in higher light levels (140 μmol photons m⁻² s⁻¹) (FIG. 22). A probable explanation for these observations is that energy from photosynthesis is required for gene expression, synthesis and function of lipase enzymes. Because of self-shading, photosynthetic cells have a higher metabolic activity at low cell density than at high cell density. This is consistent with the higher FFA yields that we observed at low cell concentrations (Table 11). The intensity of agitation did not affect the rate of lysis, for example, continuous rotation at 60 rpm/min and intermittent shaking (once per day) gave the similar membrane damage curves (FIG. 22).

Gas Chromatography (GC) (Lalman and Bagley 2004) showed that a significant amount of FFAs were released by lipolytic degradation of the membrane lipids (FIG. 23 and Table 11). The FFA yields of Green Recovery were closely related to the membrane damage even under different conditions (Table 11). GC also showed that the profile of the released FFA was close to the fatty acid profile of the 6803 membrane lipids with abundant unsaturated fatty acids (FIG. 24), suggesting that the released FFAs were degradation products of membrane lipids. FFA release occurred concomitantly with membrane damage during Green Recovery, and the released FFA amount reached the maximum when most cells became permeable to the Sytox stain (FIG. 28).

As detailed in Examples 1 through 9, we constructed 6803 strains that secrete FFAs into the culture medium by introducing the acyl-acyl carrier protein (acyl-ACP) thioesterase genes into 6803 along with other genetic modifications. The FFA-secretion strains that harbor the Green Recovery system (SD239, SD254 and SD262 in Table 10) are still able to release membrane lipids as FFAs at rates faster than non-FFA-secretion strains following CO₂ limitation. GC analysis showed that the profile of the FFAs overproduced by acyl-ACP thioesterases by FFA-secretion strains was different from the profile of the membrane-released lipids. The FFAs recovered from overproducing strains are highly saturated and rich in C12:0 and C14:0, whereas FFAs obtained via the Green Recovery system contained substantial amounts of unsaturated fatty acids and only a small portion of C12:0 and C14:0, which is the same composition observed in membrane lipids (FIG. 24). The released FFA amount from membrane lipids is similar to the amount of secreted FFA from thioesterases (Table 11, SD239). As anticipated, the FFAs recovered from the combination strains (e.g., SD239 and SD262) after CO₂ limitation were a mixture of the overproduced FFAs and the released membrane FFAs.

The Green Recovery system was designed for production of scalable and cost-effective renewable biofuels in photobioreators. Productive photobioreators require aeration systems to supply the photosynthetic microorganisms with CO₂. Lipid recovery from biomass by limiting CO₂ supply is clearly an efficient and effective method. The system we describe here does not require traditional biomass processes (Molina Grima, Belarbi et al. 2003), such as cell harvesting, dewatering, cell disruption, solvent extraction or inducer molecules (Liu and Curtiss 2009), thus considerably reducing the cost of lipid recovery. Since continuous agitation is not required for Green Recovery (FIG. 22), this system only needs sunlight and possibly intermittent agitation to convert biomass into FFAs. Another advantage of Green Recovery is that lipolytic enzymes convert diacylglycerols in the membranes into FFAs, which due to their low density and low solubility in water are easier to harvest and refine than the diacylglycerol lipids.

Green Recovery exhibits other advantages when combined with the previously described cyanobacterial FFA secretion system (Examples 1 to 9). The FFA secretion system avoids the energy intensive biomass processes such as concentration and extraction by directly recovering the secreted FFA from the culture medium. However, the FFA secretion system still requires substantial biomass to achieve cost-effective FFA production, which means a significant amount of fixed carbon has to be converted and stored as lipid membranes. It is expected that the Green Recovery system will recover the membrane lipids in the potential spent cyanobacterial biomass generated by the FFA secretion system, and also will cause cell lysis and release of the unsecreted intracellular FFAs. To our surprise, incorporation of the Green Recovery system into the FFA secretion strains resulted in an increased damage rate upon CO₂ limitation (Table 11, SD239 and SD262). We postulate that secretion of FFAs through the cytoplamstic membranes creates some lesions in the membranes, which facilitate the contact of lipolytic enzymes to the acyl glycerol ester bonds. These findings demonstrate the practical combination of the FFA-secretion system and Green Recovery in photobioreactors, where the old cultures or the spent biomass can be utilized for extra FFA yield. We believe that cyanobacterial biofuel will be instrumental in developing a carbon neutral source of sustainable fuels.

### Example 12

### Develop Means to Ensure that Strains Constructed Exhibit Biological Containment Properties to Ensure their Inability to Persist if Inadvertently Released into the Environment

Developing an inexpensive means to achieve effective biological containment for a system designed for very large culture volumes with very high populations is a daunting task. The reason is that even an improbable occurrence like multiple mutations occurring in the same cell starts to increase in probability as the number of cells becomes large. The features of such a system may also depend on whether the eventual photobioreactor system is open or closed. Conceptually, initial focus may be limited on obtaining a better understanding of the enzymology for synthesis of precursors and assembly of the peptidoglycan layer. Some of this will derive from efforts described above in Example 10 to slightly impair synthesis of DAP and thus weaken the peptidoglycan to facilitate FFA secretion. Success in these endeavors may contribute some biological containment attributes. It is also likely that the extensive genetic engineering of 6803 may preclude its ability to survive in most environments encountered outside the photobioreactor. Because of this possibility, such studies may be conducted during the course of constructions. Listed below, are the types of efforts that may be used to achieve biological containment.

### Evaluation on Engineered FFA Producing and Secreting Strains to Survive in Diverse Environments

Strains as constructed may be evaluated for growth attributes in diverse environments as a function of temperature, light intensity, inoculation density and in the presence of competing bacteria of diverse genera. For example, we have found that FFA secreting strains such as SD243 can be grown in media with 0.8 M NaCl with no reduction in production and secretion levels of FFAs and have furthermore found that these salt concentrations very much inhibit growth of many heterotrophs that can contaminate cultures of FFA-producing cyanobacteria. Thus growth of strains in 0.8 M NaCl at pH 10 in the precense of sunlight that delivers UV that kills heterotrophs more than photosynthetic cyanobacteria should very much reduce presence of contaminating heterotrophs that might consume some of the secreted FFA produced. Nevertheless, we are examining other means to out-compete contaminants as well as to engineer resistance to predation by bacteriophages and protozoans such as amoebas.

Development of a Two Strain System with Interdependence on Synthesis and Excretion of One Cell Wall Constituent and the Inability to Synthesize Another

Two 6803 strains with identical genotypes relative to all aspects of FFA biosynthesis and secretion may be obtained. One strain may be designed so that it is unable to synthesize D-alanine (due to a Δalr mutation to eliminate alanine racemase) but be able to secrete either DAP or D-glutamate. The other strain may be unable to synthesize either DAP (due to a ΔasdA mutation) or D-glutamate (due to a Δmurl mutation) but be able to secrete D-alanine. Using this design neither strain could live alone nor even survive together at low cell densities. A potential concern for the success of this system is the possible presence of low levels of D-alanine, DAP or D-glutamate in lakes, streams, soil, etc. due to the death of other bacteria. We will thus measure the concentrations of these amino acids in environmental samples to assess this potential problem. However, as described in Example 10 we have cloned the 6803 alr, asdA and murl genes and expressed them in suitable E. coli mutants, have generated His-tagged Alr, AsdA and Murl proteins to generate antibodies, and have constructed suicide vectors to enable generation of 6803 derivatives with delation mutations in these genes. Other means to generate a system of mutual interdependency to confer biological containment are contemplated.

### Develop a Cell Survival System Dependant on Maintenance of High Cell Density

It may be explored whether a quorum sensing system (not so far described in 6803 or other cyanobacteria) can be introduced into 6803 so that a critical cell function (such as DNA synthesis or cell division) is dependent on maintaining a high cell density.

### Example 13

### Bioinformatic Searches for Genes of Interest Encoding Functions to Improve 6803 Strains for FFA Production, Secretion an/or Recovery

We have made extensive use of bioinformatic searches to identify heterologous genes encoding functions or activities that would be beneficial to express in our biofuel production strains. Similarly, we have used bioinformatic searches using amino acid and nucleotide sequence information of genes with known beneficial functions from other organisms to search the cyanobase data set to identify putative 6803 genes that can be evaluated to determine whether they encode a protein of the desired function. For example, we have used this approach to identify potential cell surface proteins that might constitute a surface-layer protein or be necessary for export or anchoring of such surface proteins.

In this regard, we conducted a global search for S-layer homology (SLH) domains by screening 64 known SLH genes (Beverage et al., 1997; Sara and Sleytr, 2000; McCarren et al., 2005; Kawai et al., 1998; Smit et al., 1992) across all genes in PCC 6803. The Venn diagram in FIG. 25 illustrates the outcome of these bioinformatic searches. Of eight SLH candidate genes, the SLH domain in sll0772 was not included in the Conserved Domain Database (CDD) but has been identified in the study (Lupas, Engelhardt et al. 1994). Six genes were significantly homologous to five known RTX-SLH genes but no significant SLH domain was found. Sll1951 was a RTX gene and possessed an obvious SLH domain identified at the first 100 amino acid region. This indicated its likely uniquiness as a potential surface layer protein on 6803, a conclusion in accord with the results reported by Sakiyama et al. (2006). FIGS. 26 and 27 illustrate identification and analyses of additional potential 6803 surface located proteins. FIG. 26 depicts the alignment of the RTX surface layer gene csxA from Campylobacter rectus (Braun et al. 1999) with its homologous genes found in 6803. The matched segments were colored based on the alignment scores that were obtained from BLASTP. The right two columns are showing the significance of similarity between csxA and the homologous genes in 6803. FIG. 27 lists the surface layer candidates in PCC 6803 that are carrying SLH domains. These genes were selected based on screening known SLH domains across all genes in 6803 by the identity >30% and Evalue <1.0e−4. The regions of SLH domain or SLH super family were designated in the Conserved Domain Database (CDD) from the NCBI website.

Another use of bioinformatic searches is to enable searches of the cyanobase data set to identify genes that are likely dispensible and which can therefore be deleted to establish a potential placement for insertion of heterologous genes to encode some desired function. Such knock-outs of dipensible genes/functions are detailed in Example 14.

### Example 14

### Deletion of Dispensible Genes in Synechocystis for Facilitating FFA Production, Secretion or Recovery

In the study of Synechocystis genetic transformation, we identified some deletable genes in the 6803 chromosome. These genes can be deleted by the Km^{r}/sacB intermediated double crossover recombination without significant adverse effects on the growth of 6803 cells. Some of these deletable genes encode enzymes that would divert energy and cellular resourses away from production, for example, the slr1993 and slr1994 genes for PHB synthesis. Some of these deletable genes encode proteins that are components of cell walls, for example, the sll1951 gene for the surface layer protein. Some of these deletable genes encode enzymes that would direct FFA to other metabolic pathway, for example, the slr1609 for acyl-ACP synthetase. Some of these deletable genes encode proteins with unknown functions, and deletion of them can save the carbon source and energy for synthesizing them. These dispensible genes in 6803 and their functions are described in Table 12.

TABLE 12
Dispensible genes in Syenchocystis 6803

| | First | | |
|---|---|---|---|
| Dispensible | deletion | | |
| gene | strain | Genotype | Description |
| nrsBAC | SD102 | ΔnrsBAC11::P_{nrsB21} 13 Km^{R} | nrsBAC encodes three nickel |
| | | sacB | exporters. |
| lipA | SD200 | ΔlipA22::sacB Km^{R} | lipA encodes a lipase that hydrolyse |
| | | | membrane lipids into FFA |
| slr1993 | SD201 | Δ(slr1993-slr1994)-14::sacB | slr1993 slr1994 encode two PHB |
| slr1994 | | Km^{R} | synthetases |
| slr1609 | SD214 | Δaas-23::sacB Km^{R} | slr1609 encodes acyl-ACP |
| | | | synthetase |
| sll1951 | SD229 | Δaas-23::P_{psbA236} tesA136 | sll1951 encodes the surface layer |
| | | Δ(slr1993-slr1994)-14::P_{cpc39} | protein. |
| | | accB RBS accC70 P_{rbc40} | |
| | | accD RBS accA | |
| | | Δsll1951-15::sacB Km^{R} | |
| slr2001 | SD240 | Δaas-23::P_{psbA236} tesA136 | two cyanophycin synthesis genes |
| slr2002 | | Δ(slr1993-slr1994)-14::P_{cpc39} | |
| | | accB RBS accC70 P_{rbc40} | |
| | | accD RBS accA Δsll1951- | |
| | | 15::P_{psbA210} fatB161(Uc) P_{rbc40} | |
| | | fatB262(Ch) Δ(slr2001-slr2002)- | |
| | | 17::Km^{R} sacB | |
| slr1710 | SD248 | Δaas-23::P_{psbA236} tesA136 | slr1710 encodes penicillin binding |
| | | Δ(slr1993-slr1994)-14::P_{cpc39} | protein 2 |
| | | accB RBS accC70 P_{rbc40} | |
| | | accD RBS accA Δsll1951- | |
| | | 15::P_{psbA210} fatB161(Uc) P_{rbc41} | |
| | | fatB262(Ch) Δ(slr2001-slr2002)- | |
| | | 17::P_{psbA211} | |
| | | fatB262(Ch) Δslr1710- | |
| | | 19::Km^{R} sacB | |
| sll1434 | SD263 | Δaas-23::P_{psbA236} tesA136 | sll1434 encodes penicillin binding |
| | | Δ(slr1993-slr1994)-14::P_{cpc39} | protein 3 |
| | | accB RBS accC70 P_{rbc40} | |
| | | accD RBS accA Δsll1951- | |
| | | 15::P_{psbA210} fatB161(Uc) P_{rbc40} | |
| | | fatB262(Ch) Δ(slr2001-slr2002)- | |
| | | 17::P_{psbA213} | |
| | | fatB262(Ch) Δsll1434- | |
| | | 20::Km^{R} sacB | |
| sll1833 | SD264 | Δaas-23::P_{psbA236} tesA136 | sll1833 encodes penicillin binding |
| | | Δ(slr1993-slr1994)-14::P_{cpc39} | protein 4 |
| | | accB RBS accC70 P_{rbc40} | |
| | | accD RBS accA Δsll1951- | |
| | | 15::P_{psbA210} fatB161(Uc) P_{rbc40} | |
| | | fatB262(Ch) Δ(slr2001-slr2002)- | |
| | | 17::P_{psbA213} | |
| | | fatB262(Ch) Δsll1833- | |
| | | 21::Km^{R} sacB | |
| slr2132 | SD265 | Δaas-23::P_{psbA236} tesA136 | slr2132 (pta) encodes a |
| | | Δ(slr1993-slr1994)-14::P_{cpc39} | phosphotransacetylase |
| | | accB RBS accC70 P_{rbc40} | |
| | | accD RBS accA Δsll1951- | |
| | | 15::P_{psbA210} fatB161(Uc) P_{rbc41} | |
| | | fatB262(Ch) Δ(slr2001-slr2002)- | |
| | | 17::P_{psbA211} | |
| | | fatB262(Ch) Δslr1710-19::P_{psbA210} | |
| | | fatB163(Cc) Δslr2132- | |
| | | 22:: Km^{R} sacB | |
| slr0089-0090- | SD266 | Δaas-23::P_{psbA236} tesA136 | slr0089 and slr0090 encode two |
| 0091 | | Δ(slr1993-slr1994)-14::P_{cpc39} | vitamin E synthase, and slr0091 |
| | | accB RBS accC70 P_{rbc40} | encodes an aldehyde |
| | | accD RBS accA Δsll1951- | dehydrogenase |
| | | 15::P_{psbA210} fatB161(Uc) P_{rbc41} | |
| | | fatB262(Ch) Δ(slr2001-slr2002)- | |
| | | 17::P_{psbA211} | |
| | | fatB262(Ch) Δslr1710-19::P_{psbA210} | |
| | | fatB163(Cc) Δslr0089- | |
| | | 0090-0091-23:: Km^{R} sacB | |
| slr0301 | SD267 | Δaas-23::P_{psbA236} tesA136 | slr0301 (pps) encodes a |
| | | Δ(slr1993-slr1994)-14::P_{cpc39} | phosphoenolpyruvate synthase |
| | | accB RBS accC70 P_{rbc40} | |
| | | accD RBS accA Δsll1951- | |
| | | 15::P_{psbA210} fatB161(Uc) P_{rbc41} | |
| | | fatB262(Ch) Δ(slr2001-slr2002)- | |
| | | 17::P_{psbA211} | |
| | | fatB262(Ch) Δslr1710-19::P_{psbA210} | |
| | | fatB163(Cc) Δslr0301- | |
| | | 24:: Km^{R} sacB | |
| slr1192 | SD268 | Δaas-23::P_{psbA236} tesA136 | slr1192 encodes a probable alcohol |
| | | Δ(slr1993-slr1994)-14::P_{cpc39} | dehydrogenase |
| | | accB RBS accC70 P_{rbc40} | |
| | | accD RBS accA Δsll1951- | |
| | | 15::P_{psbA210} fatB161(Uc) P_{rbc41} | |
| | | fatB262(Ch) Δ(slr2001-slr2002)- | |
| | | 17::P_{psbA211} | |
| | | fatB262(Ch) Δslr1710-19::P_{psbA210} | |
| | | fatB163(Cc) Δslr1192- | |
| | | 26:: Km^{R} sacB | |

### Materials and Methods for Examples 9-14.

### Bacterial Strains, Media and Growth Condition

All strains were derived from Synechocystis sp. PCC 6803 and the SD100 strain used as the parent of all other strains was maintained at −80° C. in BG-11 medium supplemented with 20% glycerol. Defined deletion mutations with and without specific insertions are described in the Examples above and below (see Tables 1 and 10). 6803 wild-type and mutant strains were generally grown at 30° C. in modified BG-11 medium (Rippka, Derulles et al. 1979) under continuous illumination (140 mmol photons m⁻² s⁻¹). Bacterial growth in liquid culture was monitored spectrophotometrically or by flow cytometry and/or by plating. For growth on plates, 10 mM TES-NaOH (pH 8.2) and 0.3% (w/v) sodium thiosulfate were added to BG-11 medium, and the medium is also solidified by addition of 1.5% agar for plating and colony isolation. For transformant selection, 50 μg/ml kanamycin or 4.5% (w/v) sucrose are supplemented in the BG-11 agar plates.

Cell density, CO₂ supply and pH are very important to the growth of 6803, and these factors interact with each other. A typical guideline for SD culture is not to start cultures below a cell density of 10⁷ cells/ml, since low cell densities will create a long lag phase prior to exponential growth. We used the following procedure to grow a 6803 culture from a colony descended from a single cell. A single SD colony is picked by a sterilized needle and used to inoculate 1 ml modified BG-11 medium buffered by 10 mM TES-NaOH (pH 8.2) in a glass test tube. The tube is incubated with illumination and intermittent shaking for 2-4 days. These starter cultures can be scaled up after the OD_{730 nm} reaches 0.6 (10⁸ cells/ml) by inoculating the 1 ml culture into 10 ml buffered BG-11 medium. The culture is grown in 50 ml flasks with 50 rpm rotation. 100 ml buffered BG-11 medium cultures are grown in 250 flasks with 100 ml/min aeration with air, and 1 L modified BG-11 medium cultures are grown without TES buffer with 300 ml/min air sparged with an air stone. Once the 1 L culture achieves OD_{730 nm}˜0.6, aeration is switched from air to CO₂-enriched air. This protocol uses TES buffer and air aeration to keep the pH around 8 at the beginning inoculation stage to minimize the lag phase. FFA-producing strains need a sufficient CO₂ supply and a pH above 8 to maximize FFA secretion yields. When the 6803 cell density achieves 10⁸ cells/ml, the culture is able to maintain their pH above 8, and can be supplied with CO₂-enriched air.

### Synthetic Molecular Procedures.

Methods for DNA isolation, restriction enzyme digestion, DNA cloning and use of PCR for construction and verification of vectors are standard (Sambrook, Fritsch et al. 1989). The E. coli K-12 strains χ6097 and χ6212 were used for initial cloning. DNA sequence analysis was performed in the DNA Sequence Laboratory in the School of Life Sciences. All oligonucleotide and/or gene segment syntheses were done commercially (Genscript USA, Piscataway, N.J.). To increase the transcriptional and translational efficiency of heterogenous genes in 6803, as well as their genetic stabilities, the nucleic acid sequences of gene segments are redesigned by codon optimization based on the codon frequencies of highly expressed 6803 genes. Also the stem-loop hairpins in the predicted mRNA secondary structure are removed to smooth the transcription and to stabilize mRNA by prolonging its half-life. This may involve site-directed mutagenesis to “destroy” RNase E cleavage sites (Smolke 2000; Liou 2001). In some genes, the second codon was replaced by AAA to increase protein translation efficiency (Stenstrom, Jin et al. 2001). HA-tagged or FLAG-tagged proteins are used to obtain anti-protein rabbit antisera for western blot analyses and for quantitating protein synthesis levels in 6803 strains. Stabilization of plasmid constructs may be evaluated by DNA sequencing, and by ability to complement various cyanobacterial mutant strains and synthesize specific proteins as determined by western blot analyses. Stability of genetic modifications may be evaluated by growth under non-selective conditions for at least 50 generations of growth.

Gene Deletion and Introducing New Genes into 6803 by Using Km^{r}-sacB Cassette

Multiple gene modifications were included into the cyanobacteria strains. Suicide vectors with a Km^{r}-sacB cassette and sequences flanking the gene targeted for deletion are inserted with high efficiency into the desired chromosome site. The same vector having a second gene of interest may be used to replace the Km^{r}-sacB cassette with a desired sequence, thus substituting an undesired gene with a gene of interest in just two sequential Km^{r}-sacB transformation steps. The same strategies may be used to delete native promoters and replace them with other constitutive or regulatable or improved promoters. The important feature is to be able to select for kanamycin resistance to make the initial Km^{r}-sacB insertion/interruption and selection for sucrose resistance to introduce the final modification to eliminate the drug-resistance marker. One can thus make a succession of sequential changes in a strain genotype, resulting in a markerless strain with as many modifications as desired.

### Transformation of 6803 by Suicide Vectors to Insert and Replace Kmr-SacB Cassette

6803 is transformable at high efficiency and integrates DNA by homologous double crossover recombination for gene deletion, insertion and modification. General conditions for transformation of 6803 were optimized (Kufryk, Sachet et al. 2002) and in previous studies procedures for efficient segregation, clonal selection, and genetic identification were optimized. These procedures continue to be improved.

i. Transformation of Suicide Vectors Containing Km^{r}/sacB Cassette.

About 10⁶ SD cells in 10 μl BG-11 medium are mixed with 400 ng suicide vector DNA containing the Km^{r}/sacB cassette and incubated for 5 h. Then the mixtures were plated onto a filter membrane (Whatman PC MB 90mM 0.4 μm) layered on a BG-11 agar plate. After segregation on the BG-11 plate for about 24 h, the membrane carrying the cyanobacteria was transferred onto a BG-11 plate containing 50 μg/ml of kanamycin. Generally, the colonies appear 4-5 days later. Individual colonies are picked into a small volume of BG-11 medium and restreaked with a sterile loop onto a kanamycin BG-11 agar plate and a 4.5% sucrose BG-11 agar plate. Those patches growing on the kanamycin BG-11 agar plate and not growing on the 4.5% sucrose BG-11 agar plate are the desired correct insertions with the Km^{r}/sacB cassette.

ii. Transformation with Markerless Constructs.

To replace the Km^{r}/sacB selective marker with target gene segments, about 10⁶ Km^{r}/sacB cells in 10 μl BG-11 medium are mixed with 400 ng suicide vector DNA containing the target genes and incubated for 5 h. The mixtures are inoculated into 2 ml buffered BG-11 medium and grown for 3-4 days. 1 ml is plated onto a BG-11 agar plate containing 4.5% sucrose. Generally, the colonies appear 5-8 days later. Individual colonies are picked into a small volume of BG-11 medium and restreaked onto kanamycin BG-11 agar plates and 4.5% sucrose BG-11 agar plates. The patches growing on sucrose plates and not growing on kanamycin plates are positive candidates for further PCR identification.

iii. Confirmation of Replacement.

Cells from a colony (decended from a sigle cell) are resuspended in 2 μl water in a 200-μl PCR tube. The cell suspension is frozen at −80° C. for 2 min, and then thawed in a 60° C. water bath. This freeze-thaw cycle needs to be performed three times. 1 μl frozen-thawed cell suspension is used as the PCR template for a 30 μl PCR system including the primers specific for the inserted gene segments or the deleted region. The products of the various PCR reactions are separated on gels that are stained with ethidium bromide. If the PCR products of the expected sizes are observed with an absence of fragments that are unexpected, the correction of the construction can be inferred. In some cases, enzyme assays can be performed to demonstate the absence or insertion of a gene. In still other cases, other phenotypes associated with the genetic alterations can be observed or tested for.

iv. Stocking of Strains.

The cells with correct genotype are suspended from plates, transferred into glycerol-BG-11 medium (20% glycerol, v/v), distributed into at least four tubes and frozen at −80° C. and stored in two separate freezers on separate power supply with backup generators.

### Genetic Stability Tests

When a foreign gene is introduced into 6803, it may cause adverse effect on the growth and be subjected to gene loss or modification, since any cell losing the genetic alteration will have a higher growth rate to eventually take over the population. The genetic stability of foreign genes in 6803 is therefore tested by growing a culture of the strain with periodic dilution and subculturing for at least two months. After this time, the cells from the culture are plated onto BG-11 agar plates to obtain single isolated colonies. One hundred single colonies are picked and tested for all genetic attributes and confirmed for the presence of the foreign gene by PCR as described above. The percentage of positive colonies in the culture reflects the genetic stability of the foreign gene. For example, the presence ratio of E. coli *tesA gene driven by P_{psbAII} (in SD216) or P_{nrsB} (SD215) is 100% for two months, thus we can say the E. coli *tesA gene is genetically stable in 6803. Genes found to be unstable can be modified to eliminate non-functional hydrophobic domains that often are responsible for poor growth due to association with and impairment of cytoplasmic membrane function.

### FFA Separation and Measurement

As the FFA-secreting strain grows, FFA will be secreted into the culture medium and form insoluble FFA deposits on top of the culture media. The precipitated FFAs can be directly separated out and recovered by pipetting, filtration and/or skimming. However, some FFAs stay in the medium as dissolved acid anions because the pH of the culture is above 8. We thus developed a quantitative method for separation of the secreted FFAs from the culture medium. One hundred ml of culture is acidified by 2 ml H₃PO₄ (1M) containing 1 g NaCl, and extracted with 100 ml hexane. After 30 min shaking, the mixture is centrifuged, and the organic phase is separated by a separation funnel and dried by a vacuum. The chemical composition of FFAs is analyzed by GC-MS. Briefly, 1 ml 3M methanolic HCl (Supelco, St. Louis, Mo.) was added to the sample, which is heated at 85° C. for 2.5 h. After cooling to room temperature, 0.5 ml water and 1 ml of hexane are added and well mixed. The sample hexane layer with transesterification product FAME (fatty acid methyl ester) is collected and the remaining aqueous phase is twice extracted by 1 ml hexane. In total, 3 ml hexane is collected and mixed for GC analysis. Over 99% of FAMEs can be recovered after three hexane extractions.

Determination of FAMEs was carried out using gas chromatography (Shimadzu GC 2010) equipped with a Supelco SP2380 capillary column (30m×0.25 mm×0.20 μm) and a flame ionization detector (FID) was used to quantify FAMEs. Operating conditions were as follows: split ratio 1:10; inject volume 1 μl; helium carrier gas with constant linear velocity 20 cm/s; H₂ 40 ml/min, air 400 ml/min, make up 30 ml/min; injector and detector temperature 240° C.; oven temperature started at 140° C. for 1 min and increased at rate of 4° C./min to 220° C. and held for 5 min. Supelco 37 Component FAME Mix standard (Supelco, St. Louis, Mo.) was used to make a calibration curve for each FAME compound. The peaks from samples were identified by comparing retention times of unknown compounds with those of standard compounds and were also confirmed by GC-MS. Unknown compounds in samples were quantified based on their specific areas.

For the unsecreted intracellular FFAs, the cells are collected by centrifugation, and extracted by the Folch method (Folch, Lees et al. 1957) for total lipids. GC needs to be performed to analyze the FFAs profile from the total cell lipid extraction, which includes significant quantities of phospho-, galacto-, and sulfo-diglycerides as described above.

### Plasmids for Evaluation of Promoter Strengths Under Specific Cultural Conditions and for Use in Evaluation of Cloned Genes for Beneficial Activities in Increasing FFA Production and Secretion

A series of plasmids may be constructed to facilitate research on the genetic manipulation of 6803. As listed in Table 13 and diagrammed in FIG. 29 derivatives of the IncQ conjugative plasmid RSF1010 are being constructed. The 5.7 kb region of RSF1010 containing three rep genes, A, B, and C is necessary for its replication in the 6803 (Scherzinger, Bagdasarian et al. 1984; Marraccini, Bulteau et al. 1993). RSF1010 is being modified to construct a family of promoter fusion vectors with various reporter genes. For example, the vector pSD500 may harbor a selectable streptomycin-resistance gene and the promoterless reporter gene E. coli phoA. Other constructions with different selectable markers and reporter genes are listed in Table 13 (Wolk, Cai et al. 1991; Mermet-Bouvier and Chauvat 1994; Kunert, Hagemann et al. 2000). It has been reported that phoA exists in 6803 but this gene and the gene product has very low homology to the E. coli phoA (Ray, Bhaya et al. 1991; Hirani, Suzuki et al. 2001). Therefore, the phoA gene (sll0654) may be deleted or inactivated in the chromosome of 6803 when the reporter gene phoA is used in the construct. Random cloning of sequences can be used to search for promoters that are expressed at high level under any condition such as stationary phase. Also replicatable expression vectors with three different regulatable promoters (pSD504-pSD506) may be constructed, and these may be used to express specific cloned genes to complement deleted genes or to determine effect of overexpression of genes. Actually these vectors may be used to determine whether addition of a gene or operon would enhance any property of interest such as FFA secretion, etc. Appropriate 6803 strains may be generated depending on the requirement for each vector construct. The wild-type E. coli araE gene may thus be inserted to facilitate/allow arabinose uptake for use of araC P_{BAD} and the E. coli lacI gene for use of either the P_{trc} or P_{lpp-lacO} promoters.

Transposon vectors with Tn5 and Tn10 and derivatives may also be made with capabilities for operon and protein fusions or for inducible expression of genes downstream from the inserted transposons (Wolk, Cai et al. 1991; Milcamps, Ragatz et al. 1998; Bhaya, Takahashi et al. 2001).

TABLE 13
RSF1010-derived vectors for promoter search and regulatable
expression

| | | | |
|---|---|---|---|
| | | Reporter gene/ | Chromosome |
| pSD number | Ab marker^{a} | Regulatable promoter | requirement |

Promoter search vectors

| | | | |
|---|---|---|---|
| pSD500 | Sm | phoA | Δsll0654 |
| pSD501 | Sm | luxAB | — |
| pSD502 | Sm | gfp | — |
| pSD503 | Sm | lacZ | — |

Regulatable expression vectors

| | | | |
|---|---|---|---|
| pSD504 | Sm | araC P_{BAD} | AraE⁺ |
| pSD505 | Sm-Sp | P_{trc} | LacI⁺ |
| pSD506 | Sm-Sp | P_{lpp-lacO} | LacI⁺ |

^{a}Sm, streptomycin; Sp, spectinomycin.

### Example 15

### Establishment of Balanced-Lethal Plasmid-Host Systems to Enable Stable Plasmid Maintenance and Over-Production of FFA Precursors

We can use the ΔasdA 6803 mutant described in Example 10 to establish a balanced-lethal plasmid-host system that ensures maintenance of the plasmid vector by placing the wild-type asdA gene on a plasmid derivative of pSD505 (Table 13). We would thus replace the gene encoding streptomycin and spectinomycin resistance with the wild-type asdA gene. This plasmid has the strong prompter P_{trc} and if we deleted the lacI gene we could engineer this plasmid by having P_{trc} control over the expression of the accABCD genes to lead to an over production of manonyl Co-A. This technology should further increase FFA production and secretion.

### Example 16

### Genetic Manipulation of Synechocystis for Regulated Lysis for Release of Lipids and Modification of Lipid Composition

As part of efforts towards biofuel production and recovery from cyanobacterial biomass, the current genetic modification techniques for gene deletion, insertion and substitution were optimized. Conditions for transformation and isolation of a recombinant by dominant phenotype have been well documented. However, if a recombinant is selected by a revertant phenotype (e.g., sucrose resistance by removal of sacB), the apparent overall transformation efficiency of Synechocystis 6803 is usually low because of low segregation efficiency. Few papers discuss the optimal conditions and efficiency of the counter selecting transformation. By testing different plasmid concentrations, transformation and segregation times, and selection pressure levels, the overall transformation efficiency was thus significantly improved. A highly optimized experimental protocol for 10²-10⁵ transformants/μg DNA has been developed, which is sufficient for genetic engineering purposes. On the basis of the optimized transformation technique, a series of gene expression systems for Synechocystis 6803 were developed, including both constitutive and inducible expression strategies. Various Synechocystis 6803 native promoters with different transcriptional levels were cloned, e.g., P_{cpcBA}, P_{psbAII}, P_{psaAB}, P_{rbc}, and P_{sigA}, so that the target genes can be transcribed with the desired frequency. Genetic modifications of the native promoters such as removing or mutating the negative regulatory elements to stabilize their transcription levels in the dark or under stress conditions were also performed. For the inducible expression strategy, Synechocystis 6803 promoters responsive to different inducing factors were cloned, such as P_{nrsB}, which is activated by addition of 7-50 μM nickel ion (Liu and Curtiss 2009), P_{isiA}, which is activated by iron deficiency or when the cells grow into stationary-growth-phase (Singh and Sherman 2006), and P_{cmpABCD} and P_{sbt}A, which are activated by CO₂ limitation in the culture media (McGinn, Price et al. 2003).

Using the above techniques, a lysis induction system in Synechocystis 6803 was designed and constructed to facilitate extracting lipids for the production of biodiesel. Several bacteriophage-derived lysis genes were integrated into the Synechocystis 6803 genome and placed downstream of a nickel-inducible signal transduction system (nrsRS-P_{nrsB}). Three strategies were applied to utilize the phage lysis genes. Strategy 1 used the lysozymes from P22 and λ, respectively, to test the lysing abilities of lysozymes from different bacteriophages. Significant autolysis has been induced in the Synechocystis cells with this system by addition of NiSO₄. Strategy 2 was designed to over express the endolysin genes (P22 19 15) under a constitutive promoter P_{psbAII}, while restricting the control of the expression of the holin gene (P22 13). As a result, before induced expression of the holin gene, endolysins accumulate in the cytosol. Once the holin gene is expressed, the holins synthesized would produce holes in the cytoplasmic membrane from within and allow the accumulated endolysins to gain access to the cell wall, resulting in rapid destruction of the murein. Strategy 3 incorporated the lysis genes from λ with P22 lysis genes. A faster lysis rate resulted since different lysozymes attacked different bonds in the cell envelope.

### Example 17

### Bioinformatics Searches and Analyses to Identify Putative Genes and Operons Specifying Cell Wall and Cell Surface Macromolecules

The complete genomes for E. coli K-12, S. Typhimurium LT-2, and Synechocystis 6803 were downloaded from NCBI GenBank and gene information was extracted using Perl scripts. The COG database (Tatusov et. Al. 2000) was also downloaded for use in categorizing genes. All of the features were stored in a MySQL database. For gene identification, BLASTP implemented in NCBI blast-2.2.18 was used to identify possible homologous genes of E. coli or Salmonella in the Synechocystis 6803 genome with a threshold e-value less than 1.0⁻⁴ and identity greater than 35%. Three methods were used to define genes of synthesis and assembly of cell wall components: (1) Based on a set of genes in E. coli K-12 and Salmonella LT-2, the homologous genes in Synechocystis 6803 were determined using BLASTP. (2) A set of genes based on the functional descriptions of the COG database were determined, and then the homologous genes in Synechocystis 6803 were found using BLASTP (3) Functional annotation of Synechocystis 6803 was used to search for genes directly. Using these methods, over 300 putative genes potentially involved in the synthesis and assembly of the Synechocystis 6803 cell wall and cell-surface macromolecular components were identified, and 25 of these genes were arranged into 7 putative operons. These are diagramed in FIG. 30 and placed in their approximate location on the Synechocystis 6803 chromosome map along with their predicted functions in synthesis of cell wall components.

### Example 18

### Genetic and Phenotypic Characterization of the Synthesis and Regulation of Cell Wall Components

There is no comprehensive study of LPS or EPS in Synechocystis 6803, in contrast to the depth and detail published about photosynthesis by this organism. Therefore, genes and operons in LPS or EPS synthesis may be systematically identified and characterized, using a combination of bioinformatic analysis and experimental methods. For the previously characterized genes in pili and surface-protein layer synthesis, knock-outs may be made to assess their role in biofilm formation. Recent work has elucidated synthesis and regulation of pili-mediated motility, but this work has not been assessed in the context of biofilms.

### LPS Components and Assembly.

The LPS and specifically the O-antigen component is normally covered by the surface-layer protein, and therefore does not directly mediate attachment and adhesion of WT cells in biofilms. LPS mutants in S-layer minus strains may likely have modified biofilm characteristics due to their different surface biochemistry and therefore different adhesion characteristics. We have found this to be so. These differences may be important if the Synechocystis strain used for industrial-scale production is a S-layer minus variant. Bioinformatic assessment of Synechocystis 6803 (see Examples 17 and 20) indicates that the O-antigen synthesis pathways (rfb operons) of previously characterized gram-negatives such as E. coli (Paton and Paton 1999) are at least partially conserved in Synechocystis 6803. A putative operon slr0982, slr0983, slr0984, slr0985 (rfbB, rfbF, rfbG, rfbC), has been identified and many additional rfb homologs are found individually or in pairs in the genome. In total, rfbABCDEFG, M, P, U and W each occur at least once, for a total of at least twenty rfb homologs. Synechocystis 6803 LPS may be characterized via LPS gels to identify components of the LPS. These components may be used to focus a bioinformatic search for genes related to LPS synthesis in Synechocystis 6803. These genes may be knocked out individually and in combination in both WT and S-layer minus backgrounds, and mutants may again be assessed using LPS gels. LPS mutations that result in an S-layer shedding phenotype may help identify which O-antigen moieties are required to anchor the S-layer.

### Lipid A Biosynthesis and Modification.

Lipid A is the lipid component of LPS and in most bactreria is essential for viability. We postulate that blocking lipid A synthesis in 6803 can enhance fatty acid production. Each 6803 cell has ≈10⁶ lipid A molecules as a structural component of LPS (Raetz et al., 2009), and each lipid A has about 4 FA molecules, so blocking lipid A synthesis should enhance FFA production. Removal of lipid A may also enhance outer membrane permeability, facilitating secretion of FFA. Surprizingly, deletion of the IpxA (sll0379), IpxD (sll0776) and IpxB (sll0015) genes essential for lipid A synthesis is not lethal in 6803. We have thus constructed strains with both individual deletions and combination deletion mutations to examine for increase in FFA production and potential enhanced secretion. We are also contemplating deleting many other genes associated with lipid A synthesis or modification such as IpxC and msbA since they are also likely to be dispensible and their deletion will reduce unnecessary energy use and facilitate FFA production. Other likely genes identified by bioinformatic searches that could possibly be deleted are the sll1276, sll1725, sll1149, sll1180 and sll0615 genes.

### Assesment of Pili and Pili-Mediated Motility in Cell Attachement.

Characterization of operons for synthesis and activation of pili required for motility has been published in detail (Yoshihara, Geng et al. 2001; Bhaya 2004; Bhaya, Nakasugi et al. 2006); however, the role of pili and motility in attachment and adhesion to surfaces is unknown. Synechocystis 6803 is covered with a dense layer of pili, including thick Type IV pili required for motility, and thinner pili with no known function. Mutations in previously characterized genes may allow us to perform experiments and to understand the role of pili in biofilm formation. Specifically, comparison of mutants that are unpiliated (pilC, slr0162), have paralyzed pili (pilH sll0415), have pili lacking tip adhesins (pilO, sll1276), and lacking Type IV pili (pilN, sll1275) may be compared using the crystal violet assay to identify the role of pili in attachment and adhesion of cells, and development of biofilms.

### EPS Synthesis and Export.

Two putative EPS export proteins were identified and may be inactivated to elucidate their role in biofilm formation. BLASTP of E. coli W3110 wza, a gene coding for an OM EPS export protein (Dong, Beis et al. 2006), shows 28% identity with sll1581 (with e-value of 10⁻¹⁶). sll1581 is annotated as gumB (a gene coding for an EPS export protein) in Synechocystis 6803 from Cyanobase. This protein was identified in the OM proteome of Synechocystis 6803, which would be consistent with its predicted role as a porin for EPS export (Huang, Hedman et al. 2004). wzc, a gene coding for the EPS membrane translocator in E. coli W3110, has 21% identity (with e-value of 10⁻¹⁶) with sll0923. Strains of E. coli, C. crescentus, and X. campestris with deletions in wza homologs are deficient in biofilm formation (Smith, Hinz et al. 2003; Dong, Beis et al. 2006).

BLASTP of E. coli EPS synthesis genes such as wzxE (flippase) and wzyE (polymerase) against the Synechocystis 6803 proteome showed no homologs present. A possible operon or operons for polysaccharide synthesis in Synechocystis 6803 were identified bioinformatically by searching for E. coli EPS homologs. slr0527, slr0528, slr0529, slr0530, slr0531, slr0533, slr0534, and slr0535 were found to be a putative operon, since all are in the same orientation in the genome and encode proteins that have homology to proteins for sugar synthesis and transport. However, deletion of polysaccharide synthesis genes frequently have pleiotropic phenotypes because polysaccharides are used for multiple cellular functions, in addition to EPS.

To characterize EPS in Synechocystis 6803, EPS export protein homologs sll1581 and sll0923 may be knocked out, and these mutants screened along with WT using a lectin library. Since individual lectins bind to a specific sugar moiety, each lectin may indicate whether or not its specific sugar ligand is found in Synechocystis 6803 EPS. Cells treated with each of the fluorescein-conjugated lectins may be examined using fluorescence microscopy to identify which lectins bind to WT and mutant strains. Identification of polysaccharides thus found in Synechocystis 6803 EPS may be used to focus our bioinformatic search for genes related to EPS synthesis. Knockouts of putative EPS synthesis genes may again be characterized by fluorescein-conjugated lectins, as well as by LPS gels, to determine whether the deleted gene contributes to either EPS, LPS, or both. If Synechocystis 6803 strains do bind at least one lectin, then EPS gels may also be used to characterize Synechocystis 6803 EPS.

### Example 19

### Identify the Cell Surface Macromolecules that Contribute to Biofilm Formation and their Role in Biofilm Structure and Function Under Various Physiological Conditions

Insertional knockouts and deletions described above, as well as those isolated from screens of phage-resistant or mutagenesis libraries, may be screened using the crystal violet assay in order to identify structural features important for biofilm formation. Those mutants showing atypical biofilms may be further characterized to elucidate the specific function of each feature in biofilm formation, and whether they are required for initial attachment, adhesion, biofilm maturation and structure, and/or biofilm dispersal. In addition, strains may be compared under different physiological conditions, to see if the deleted structure plays a role in biofilms that is modulated by heterotrophic growth, stress response, light response, or different growth stages, for example. These studies may identify genetic modifications resulting in a strain of Synechocystis that cannot form biofilms, which may be assessed for its suitability for large-scale biodiesel production, in collaboration with the rest of the ASU biodiesel research team. Specifically, the biofilm-minus strain of Synechocystis 6803 may be assessed for ease of lysis using our nickel-induced promoter system, for ease of lipid extraction from lysed cells, for susceptibility to phage and other predators identified by the microbial ecology group of the biodiesel team, for growth rate and general robustness of the strain in photobioreactor growth conditions, and for the amount and quality of lipid per gram of biomass.

### Biofilm Formation and Elucidating the Environmental and Physiological Conditions Affecting Biofilm Formation.

In addition to screening mutants for atypical biofilm formation, the crystal violet assay may also be used in preliminary assessment of biofilms. Specifically, the crystal violet assay may be used to visualize where biofilms form relative to the meniscus of media, to determine whether there is differential attachment for glass compared to other surface materials found in our photobioreactors used for biodiesel production, to quantify the amount of biofilm relative to the optical density and growth phase of the culture, and assess the effects of DNase, proteinase, and other additives on biofilm formation. Also, it may be used in competitive binding assays, to identify which specific EPS moieties (as identified by binding to fluorescent lectins in section Materials and Methods below) contribute to surface adhesion. Cells pretreated with lectins may show less binding or no binding in this assay, compared to untreated cells. Mutants lacking EPS may be characterized using the motility assay to determine whether EPS is required for motility, as has been suggested by the appearance of a corona of translucent extracellular material preceding phototactic cells (Burriesci and Bhaya 2008).

Additional characterization of WT and mutant strains of interest may be performed using a biofilm reactor. This reactor uses larger culture volumes than the crystal violet assay (1000 ml vs 3 ml), and also allows better control of growth conditions, including an option for chemostatic growth, and ability to create both illuminated and dark conditions in the same culture. Biofilm samples may be assessed for stages of development and maturation under various conditions. For example, WT strains cultivated in a reactor with illuminated and unilluminated areas may only form biofilms in the illuminated areas. By allowing these biofilms to develop, and then switching the conditions so that the biofilms are in the dark may allow the assessment of how biofilms respond to a change in light conditions. This experiment may also be conducted with WT and phototaxis mutants that are growing heterotrophically (addition of glucose and DCMU, which inhibits photosynthesis).

Reports of biofilm formation by cyanobacteria are limited to mixed-species biofilms related to environmental microbiology, such as epilithic cyanobacteria that degrade stone Mayan artifacts (Scheerer, Ortega-Morales et al. 2009), hot spring biofilms (Boomer, Noll et al. 2009), or symbionts of marine corals or crops important to agriculture (Arboleda and Reichardt 2009; Zheng, Bergman et al. 2009). There are no studies published characterizing single-species biofilm formation by a cyanobacterium. Thus, detailed studies to characterize biofilm formation by axenic cultures of WT Synechocystis 6803 may be undertaken using confocal laser scanning microscopy of biofilm reactor samples. Time-course experiments may be used to describe the stages of Synechocystis 6803 biofilm formation, from initial attachment and adhesion of cells, to formation of microcolonies, monolayers, biofilm maturation and development of biofilm architechture such as mushroom morphology, and biofilm dispersal. Biofilms may be studied using growth curves, and also under chemostatic conditions. Live-dead staining may be used to identify stratification of biofilm architecture by layers of live cells and dead cells. Mutants of interest identified in screens using the crystal violet assay may be used to clarify the role of specific structures in each stage of biofilm formation, by comparing confocal micrographs of biofilms formed by mutant monocultures with those of WT.

Preliminary work by the microbial ecology group of the biodiesel team has already identified non-Synechocystis species within the biodiesel photobioreactors. These non-Synechocystis species include cyanobacterial predators such as phage and protozoa, as well as heterotrophic and autotrophic bacteria whose roles in the health of the Synechocystis 6803 reactor culture have yet to be established. Studies may be undertaken to assess the role of these invaders on biofilm formation by Synechocystis 6803 in photobioreactor cultures.

### Example 20

### Design and Construct Strains with Regulatable Ability to Flocculate, Aggregate and Float

We propose to use biofuel strains that have been genetically engineered for inducible aggregation and/or flocculation of the biofuel strain with the following benefits:
dewater and collect biomass via flocculation instead of energy-intensive processes such as filtration or centrifugation, which add cost and complexity to biofuel production process;
reeduce evaporation in open pond systems with floating microbial mats;
increased yield of biofuels from biofilm-format reactors;
eliminate need to mitigate biofouling in biofilm-format reactors;
reduce susceptibility of biofuel strain to microbial predators in flocs/biofilms/mats.

We propose to use environmental or physiological signals such as CO₂ limitation or entry into stationary phase in order to induce expression of aggregation/flocculation signals. Specific mechanisms for aggregation/flocculation include but are not limited to modulating the expression of surface protein adhesins or exopolysaccharides.

Proteins that have high binding affinity can act as adhesins and cause cell-cell binding and aggregaton (Miller and Falkow 1988, Isberg and Falkow 1985). Inducible synthesis of homo-dimers such as YadA-YadA (Y pseudotuberclosis) or heterodimers such as invasin-β1-integrin (Y pseudotuberculosis) will display these adhesins on the cell surface, causing aggregation. The Km (binding affinity) of invasin with β1 integrin is the one of the strongest protein-protein interactions known (Leong and Isberg 1990).

In addition to transgenic proteins, the expression of native surface structures of the biofuel strain can also be induced using environmental signals to cause auto-flocculaton or attachment to surfaces. For example, preliminary data indicate that the exopolysaccharide of one biofuel strain (Synechocystis PCC 6803) contributes to biofilm formation in WT cells; over-expression of native exopolysaccharide synthesis genes may increase the aggregation/biofilm formation phenotype. Similarly, inducible expression of exopolysaccharide genes from Caulobacter or other organisms in other biofuel strains may be used to increase the degree and rate of flocculation for biomass harvest. The exopolysaccharide synthesized by Caulobacter has been shown to be strongest adhesive known (Tsang, Li et al 2006). Additional modification to the biofuel strain may include inducible down-regulation of O-antigen biosynthesis genes. Furthermore, our preliminary data show that deleting O-antigen genes slr0977 and slr1610 cause a flocculation phenotype in Synechocystis PCC 6803.

Inducible flocculation or aggregation (cell-cell binding) and biofilm formation (attachment of cells to the surface of reactors or other abiological surfaces) can be used to create microbial mats at the surface of open ponds to reduce evaporation when induced in buoyant biofuel strains. In strains that are not naturally buoyant, synthesis of gas-filled vacuoles may be genetically engineered by expressing vacuole synthesis genes, which include but are not limited to gvpA and gvpC (Hayes et. al. 1988).

Biofilm formation by strains secreting fatty acids or other biofuels may be induced. Growing the biofuel strain as a biofilm instead of a suspended culture negates the need to induce aggregation as a separate step in the biofuel production process. Biofilm photobioreactors have been shown to have higher performance and productivity in both bioenergy and bioremediation applications (Tian, Liao et al 2010; Syed and Henshaw 2003). Additionally, biofilm photobioreactors do not have reduced performance due to biofouling, as is the case in traditional suspended-biomass photobioreactors.

### Example 21

### Develop Systems for Conjugational Transfer from E. coli to Synechocystis 6803 and Means for Transductional Transfer of Genetic Information from One Synechocystis 6803 Strain to Another

Although natural transformation for suicide vectors is very efficient in Synechocystis (Zang, Liu et al. 2007), the observation that double crossover recombination with transformed suicide vectors is far more prevalent than single crossover plasmid insertion (Porter 1986) suggests that single crossover insertion might be lethal. This might occur if plasmids need to be linearized for efficient DNA uptake. This seems logical since the presence of Type IV pili on Synechocystis 6803 cells seems to be required for transformation and competence to take up DNA (Yoshihara, Geng et al. 2001). In fact, plasmid transformation into Synechocystis 6803 is at an exceedingly low frequency (˜10⁻⁸) unless some sequence homologous to a Synechocystis sequence exists (Mermet-Bouvier, Cassier-Chauvat et al. 1993). Therefore, transformation efficiency may be compared using plasmid RSF1010 (Bagdasarian, Lurz et al. 1981) monomer versus dimer molecules stabilized in a recA E. coli mutant. Synechocystis likely has restriction enzymes and we will investigate whether administration of a five to ten minute pulse of 50° C. overcomes this barrier (Middleton and Mojica-a 1971; Mojica and Middleton 1971; Robeson, Goldschmidt et al. 1980). If so, this treatment could also be used to enhance conjugational transfer from E. coli to Synechocystis.

Construction of an E. coli-Synechocystis 6803 Shuttle Vector.

One of the small dispensable Synechocystis plasmids (e.g. pCB2.4, pCC5.2 or pCA2.4) may be cloned into enteric plasmids with p15A ori and pSC101 ori (which may be tested for inability to replicate or be maintained in Synechocystis 6803), and identify, using transformation of monomer or dimer plasmids, a shuttle vector with the desired properties. Several different regulatable promoters and multiple cloning sites followed by a transcription terminator sequence may then be introduced. This shuttle vector may be useful in complementation studies and also in gene regulation studies. As more is learned, a set of plasmids that replicate with different copy numbers in Synechocystis 6803 may be generated.

### Developing Systems for Conjugational Transfer to Synechocystis 6803.

Suicide plasmid donor strains originally described (Miller and Mekalanos 1988) were modified to possess ΔasdA, Δalr and ΔdadB mutations that impose requirements for diaminopimelic acid (DAP) and D-alanine, two essential unique ingredients of the rigid peptidoglycan layer of the cell wall. Thus a donor-recipient conjugating mixture may be moved to medium devoid of DAP and D-alanine and the donor population undergoes cell wall-less death. Since E. coli strains grow much more rapidly than Synechocystis strains, inclusion of dnaA (Ts), dnaB (Ts) and dnaE (Ts) alleles into the E. coli donor may be investigated to arrest cell growth but without diminishing conjugational gene transfer. These E. coli donor strains have an integrated broad host range IncP conjugative plasmid that should facilitate gene transfer to Synechocystis 6803 recipients (Marraccini, Bulteau et al. 1993). However, there exists a collection of other Inc group plasmids (with drug-resistance markers) that can be easily evaluated for conjugational transfer to Synechocystis 6803 should the IncP system not be optimal. Although the potential for conjugational gene transfer of a shuttle vector described above may be examined in liquid medium, it is likely that mating and transfer may be at higher frequencies if recipient and donor cells are impinged on a filter that can be aseptically moved from a permissive agar (at various temperatures) to a non-permissive agar (no DAP and D-alanine). Glucose and LB broth may be added to supplement the BG-11 medium during mating since conjugational transfer in both donor and recipient requires active energy metabolism (Curtiss, Charamella et al. 1968). Contingent on success, conditions may be systematically varied to optimize the frequency of plasmid transfer. Ultimately the possibility of a conjugational system between Synechocystis 6803 mutant strains may be investigated.

### Identification and Development of Generalized Transducing Phages for Synechocystis.

The procedure may involve taking a collection of Synechocystis strains, treating cells with mitomicin C or UV and after a suitable period for induction of putative lysogens, add CHCl₃ and continue aeration for another hour. Cells may then be sedimented and the putative lysates stored over CHCl₃ and then tested for presence of phages causing lysis of different Synechocystis strains imbedded in soft agar overlays. Phage may be propagated on sensitive strains either in liquid medium or by confluent plate lysis and the lysates titered. Lysates may be treated with DNase to eliminate contaminating cyanobacterial DNA. The DNase may then be inactivated or removed and DNA harvested from lysates to screen for the presence of rRNA-encoding DNA sequences whose presence would predict the occurrence of generalized transduction. If multiple successes are achieved, the phage system that gives the highest titers of lysates and the highest frequency of transduction of drug-resistance markers may be determined. In addition, host-range mutants that will specifically propagate on and transduce Synechocystis 6803 may also have to be selected.

Screening of Mutant Libraries and Strains with Modified Surface Molecules for Resistance to Lytic Phage.

Bacteriophages often use surface molecules such as pili, outer membrane proteins, LPS or S-layers to attach to and infect cells (Boyd and Brussow 2002). Thus the library of 6803 mutants with altered pili, EPS, LPS and S-layer features may be screened for resistance to phages isolated from the environment or from screens (Materials and Methods below). To facilitate such screening, virulent non-lysogenizing mutants of temperate phages (Materials and Methods below) may be isolated and recovered as described in Materials and Methods below. Phage-resistant mutants of WT Synechocystis 6803 may also be isolated and screened for defects in biofilm formation (as described in Materials and Methods below). These phage may also be used to determine whether sensitivity and infection of Synechocystis 6803 and its mutants varies depending on stresses and/or growth conditions that favor or inhibit biofilm formation.

### Materials and Methods for Examples 16-21

### Bacterial Strains, Media and Bacterial Growth.

All strains may be derived from the Synechocystis 6803. Defined deletion mutations with and without specific insertions are described in the Examples. Synechocystis 6803 wild-type and mutant strains may be grown at 30° C. in modified BG-11 medium buffered with 10 mM TES-NaOH (pH 8.2) with a supplement of 1.5 g/l NaNO₃ (Rippka, Derulles et al. 1979) and bubbled with a continuous stream of filtered air under continuous illumination (140 mmol photons m⁻² s⁻¹). For growth on plates, 1.5% (w/v) agar and 0.3% (w/v) sodium thiosulfate may be added to BG-11. BG-11 medium is also solidified by addition of 1.5% agar for plating and mutant selection. Antibiotics may be used at the following concentrations: chloramphenicol (Cm) (100 μg/ml), kanamycin (Km) (50 μg/ml), streptomycin (Sm) (50 μg/ml) gentamicin (Gen) (50 μg/ml), and Zeocin (Zeo) (100 μg/ml). Bacterial growth will be monitored spectrophotometrically, by flow cytometry and/or by plating.

### Molecular and Genetic Procedures.

Methods for DNA isolation, restriction enzyme digestion, DNA cloning and use of PCR for construction and verification of vectors are standard (Sambrook, Fritsch et al. 1989). The E. coli K-12 strains χ6097 and χ6212 may be used for initial cloning. DNA sequence analysis may be performed by the DNA Laboratory in the School of Life Sciences, ASU. All oligonucleotide and/or gene segment syntheses may be done commercially. Site-directed mutagenesis was used to optimize codons for translational efficiency in Synechocystis 6803. Plasmid constructs may be evaluated by DNA sequencing, ability to complement various cyanobacterial mutant strains and for ability to specify synthesis of proteins using gel electrophoresis and western blot analyses. His-tagged or GST-tagged proteins may be produced and used for antibody production to perform western blot analyses.

### Strain Characterization.

Multiple gene modifications may be included in the strains and strain attributes may be evaluated after every step in strain construction. If needed −35 and −10 RNA polymerase recognition and binding sites, SD sequences, and start codons may be altered to modulate up or down expression of genes for regulatory proteins or those to sustain cell integrity. Presence of fimbrial adhesins may be assayed using agglutination of appropriate cells and in the presence and absence of sugars as a function of growth conditions, and by transmission electron microscopy (TEM) using negative staining with phosphotungstic acid (Qadri, Hossain et al. 1988).

### Means of Mutant Library Generation.

ColE1 replicons cannot be maintained in Synechocystis 6803. Therefore, pUC vectors may be constructed with Tn5, Tn10, Tn5-with LacI synthesis for ability to generate P_{trc} fusions (inducible by IPTG) and Tn5 reporter fusions with LacZ, Lux, mcherry or Green Fluorescent Protein. The use of fusaric acid selection (Bogosian, Bilyeu et al. 1993) for loss of Tn10 insertions may be investigated as a means to inactivate genes by deletion. Other methods are as described above.

### Means to Enrich to Isolate Mutants Defective in Adhesion to Surfaces.

An enrichment assay to find suppressor mutants for strains defective in adhesion is useful to elucidate the signal transduction pathways regulating EPS, which is likely to be coordinated with phototactic, photosynthetic, and other pathways. For example, a knockout of the wzc homolog (sll0923) or other EPS-export response regulators may be repeatedly subcultured in test tubes. Test tubes may be vortexed vigorously to remove unattached cells, and it would be expected that any attached cells would have acquired suppressor mutations that are in the same pathway as the original wzc mutation. These suppressors may be enriched by adding new media and incubating. Mapping of the suppressor mutations may require either plasmids that replicate independently in Synechocystis 6803, or use of a transducing phage, in order to locate the suppressor mutation by screening complementation libraries, or mapping via co-transduction frequency of marker lysate libraries (Pierce, O'Donnol et al. 2006).

### Crystal Violet Assay of Biofilm Formation.

A crystal violet assay may be adapted from the study of biofilm formation in Caulobacter crescentus to use for studying Synechocystis 6803 biofilms. 12-well plates may be inoculated with log-phase liquid cultures. Glass coverslips may be inserted into the wells as a substrate for biofilm attachment and maturation. For each time point, coverslips may be removed and unattached cells rinsed off with a strong jet of water. Coverslips may be stained in 1% crystal violet solution. Excess stain is removed by repeated rinsing until no purple is observed on paper used to blot the coverslips. Biofilms may be assessed qualitatively and quantitatively. Biofilm quantitation may be performed by eluting the stain from the biofilm by immersing the coverslip in 3 ml of DMSO, and measuring the crystal violet absorbance of the eluant at OD_{600 nm}. This assay can be adapted to 96-well plates, by using microplate readers to screen mutant libraries for adhesion defects to identify important genetic pathways for biofilm formation.

### Motility Assay, Phototaxis Assay.

5-10 μl of log-phase culture may be streaked onto BG-11 plates with 0.4% agar, 15 mM glucose. Unidirectional light at 40 mmol/m² sec may be used for phototaxis, spreading of colonies under uniform light shows general motility; colonies that remain the same size are non-motile (Bhaya, Watanabe et al. 1999).

### Lectin-Binding Assay for Identifying Specific Sugars.

Commercially available libraries of lectins may be used to screen Synechocystis 6803 EPS for specific sugars. For example, the lectin wheat-germ agglutinin has been found to bind specifically to polymers of N-acetyl-glucosamine. This lectin can be conjugated to FITC (fluorescein isothiocyanate) for fluorescence microscopy imaging of EPS localization in Synechocystis 6803 cultures, and for imaging gels of EPS samples (see EPS gel assay below).

### EPS Gel Assay.

This assay may be used to detect EPS with HRP (horse radish peroxidase)-conjugated WGA (wheat germ agglutinin) lectin (Hitchcock and Brown 1983). Solubilize cell pellet in lysis buffer with or without proteinase K. Run samples on PAGE gel without SDS, using a 4% acrylamide stacking layer and 10% acrylamide separating layer. Blot onto nitrocellulose (include stacking layer in blot). Block with 3% BSA and rinse with TBST. Incubate blot in 1:150,000 dilution of HRP-conjugated WGA lectin. Incubate with Dura chemiluminescent substrate from Pierce Protein Research Products.

### DNA Fingerprinting of Mixed-Species Biofilms.

Samples from large-volume and benchtop-volume photobioreactors may be used to characterize the microbial ecology of mixed-species biofilms. Cell pellets may be pre-treated with lysozyme and then genomic DNA extracted using the Qiagen DNeasy kit for gram-positives. Universal primers, and also Synechocystis 6803-specific primers, may be used to amplify 16S RNA encoding DNA sequence, and T-RFLP analysis used to determine the relative prevalence of 16S RNA encoding DNA from each bacterial species in these samples.

### LPS Gel Assay.

The LPS (lipopolysaccharide) may be analyzed on polyacrylamide gels and visualized with silver stain solution (Tsai and Frasch 1982). The cell pellet may be resuspended in 100 ml of 2× dissociation buffer with 5% β-mercaptoethanol and boiled for 10 min. The sample is then centrifuged at 13,000 rpm for 15 min. Supernatant is diluted 1:10 (50 ml) in 2×SDS (sodium dodecyl sulfate) loading buffer and 50 mg/ml Proteinase K. Incubate at room temperature for 1 h. Run samples on PAGE gel without SDS, using a 4% acrylamide stacking layer and 12% acrylamide separating layer. Run gel in SDS running buffer at 100 volts for 1 h. Resolve LPS signature on the gel by shaking the gel for 4 h in a fixative solution. Add 1.4 g of periodic acid to 200 ml of fixative and shake for 10 min. Rinse with 15 ohm ddH₂0 3×. Add silver nitrate mixture for 10 min and rinse 3×. Add developer and shake for 5 min or until bands appear. Rinse once with ddH₂O before adding Stop Solution (a 1% acetic acid solution that stops the development).

### Isolation of Synechocystis Phages.

After filtration through 0.22 mm polycarbonate filter (Whatman), the filtrates suspected of containing cyanophages are inoculated with a variety of Synechocystis cultures from our strain collection including Synechocystis 6803. After amplification on the host strain, chloroform is added to the inoculum and the mixture is agitated for 60 min and then centrifuged (7000×g, 4° C., 15 min). The supernatant is mixed again with chloroform (4 ml/ml) and stored at 20° C. Cyanophages are then titered on the propogating host using soft agar overlays on BG-11 plates. Plaque formation is used to enumerate phages (Wilson, Joint et al. 1993).

### Isolation of Virulent Mutants of Temperate Synechocystis Cyanophage.

The collection of Synechocystis strains may be screened for presence of temperate phages that propagate on Synechocystis 6803 (see Examples above). Isolated phages and especially phages isolated from lysogens are likely to be temperate and have turbid plaques. Virulent mutants forming clear plaques can readily be isolated from temperate phages by making almost confluent lysis plates using the soft agar overlay method screening for areas of clear lysis (Levine and Curtiss 1961). Screening of 20 such plates may identify between one and ten clear areas of lysis, which may be picked by using a sterile needle, plaque-purified on the Synechocystis host and then amplified by propagation in Synechocystis liquid cultures to create lysates. We may also determine whether it is possible to select host-range mutants that will infect Synechocystis 6803 or its biofilm-defective mutants.

### Isolation of Phage-Resistant Cyanobacteria.

Synechocystis 6803 subjected to chemical or other mutagens may be screened for mutants resistant to various phages or their host-range mutants. Virulent cyanophage mutants are used to avoid problems with generating phage-resistant lysogens. Resistant mutants can be selected after prolonged cultivation of strains in the presence of phage or my spreading high titers of phage on BG-11 plates followed by plating the Synechocystis 6803 culture. Mutants may be picked by sterile needle and pure cultures obtained by streaking for isolated colonies on new plates. Mutants may be characterized for loss or alteration of surface structures. TEM may also be used to help identify structures to which given phage strains attach.

### REFERENCES

Agrawal, G. K., H. Kato, et al. (2001). “An AU-box motif upstream of the SD sequence of light-dependent psbA transcripts confers mRNA instability in darkness in cyanobacteria.” Nucleic Acids Res 29(9): 1835-1843.
Andersson, L., F. Carriere, et al. (1996). “Pancreatic lipase-related protein 2 but not classical pancreatic lipase hydrolyzes galactolipids.” Biochim Biophys Acta 1302(3): 236-240.
Arboleda, M. and W. Reichardt (2009). “Epizoic communities of prokaryotes on healthy and diseased scleractinian corals in Lingayen Gulf, Philippines.” Microb Ecol 57(1): 117-128.
Bagdasarian, M., R. Lurz, et al. (1981). “Specific-purpose plasmid cloning vectors. II. Broad host range, high copy number, RSF1010-derived vectors, and a host-vector system for gene cloning in Pseudomonas.” Gene 16(1-3): 237-247.
Beveridge, T. J., P. H. Pouwels, et al. (1997). “Functions of S-layers.” FEMS Microbiol Rev 20(1-2): 99-149.
Bhaya, D. (2004). “Light matters: phototaxis and signal transduction in unicellular cyanobacteria.” Mol Microbiol 53(3): 745-754.
Bhaya, D., K. Nakasugi, et al. (2006). “Phototaxis and impaired motility in adenylyl cyclase and cyclase receptor protein mutants of Synechocystis sp. strain PCC 6803.” J Bacteriol 188(20): 7306-7310.
Bhaya, D., A. Takahashi, et al. (2001). “Novel motility mutants of synechocystis strain PCC 6803 generated by in vitro transposon mutagenesis.” J Bacteriol 183(20): 6140-6143.
Bhaya, D., N. Watanabe, et al. (1999). “The role of an alternative sigma factor in motility and pilus formation in the cyanobacterium Synechocystis sp. strain PCC6803.” Proc Natl Acad Sci USA 96(6): 3188-3193.
Black, P. N. and C. C. DiRusso (2003). “Transmembrane movement of exogenous long-chain fatty acids: proteins, enzymes, and vectorial esterification.” Microbiol Mol Biol Rev 67(3): 454-472, table of contents.
Bogosian, G., K. Bilyeu, et al. (1993). “Genome rearrangements by residual IS10 elements in strains of Escherichia coli K-12 which had undergone Tn10 mutagenesis and fusaric acid selection.” Gene 133(1): 17-22.
Boomer, S. M., K. L. Noll, et al. (2009). “Formation of multilayered photosynthetic biofilms in an alkaline thermal spring in Yellowstone National Park, Wyoming.” Appl Environ Microbiol 75(8): 2464-2475.
Boyd, E. F. and H. Brussow (2002). “Common themes among bacteriophage-encoded virulence factors and diversity among the bacteriophages involved.” Trends Microbiol 10(11): 521-529.
Braun M., Kuhnert P., Nicolet J., Burnens A. P. and Frey J., (1999). “Cloning and characterization of two bistructural S-layer-RTX proteins from Campylobacter rectus,” J. Bacteriol. 81, pp. 2501-2506.
Burriesci, M. and D. Bhaya (2008). “Tracking phototactic responses and modeling motility of Synechocystis sp. strain PCC6803.” J Photochem Photobiol B 91(2-3): 77-86.
Cai, Y. P. and C. P. Wolk (1990). “Use of a conditionally lethal gene in Anabaena sp. strain PCC 7120 to select for double recombinants and to entrap insertion sequences.” J Bacteriol 172(6): 3138-3145.
Cardineau, G. A. and R. Curtiss, 3rd (1987). “Nucleotide sequence of the asd gene of Streptococcus mutans. Identification of the promoter region and evidence for attenuator-like sequences preceding the structural gene.” J Biol Chem 262(7): 3344-3353.
Cho, H. and J. E. Cronan, Jr. (1995). “Defective export of a periplasmic enzyme disrupts regulation of fatty acid synthesis.” J Biol Chem 270(9): 4216-4219.
Curtiss, R., 3rd, L. J. Charamella, et al. (1968). “Parental functions during conjugation in Escherichia coli K-12.” Bacteriol Rev 32(4 Pt 1): 320-348.
Curtiss, R., III, S. B. Porter, et al. (1991). Nonrecombinant and recombinant avirulent Salmonella live vaccines for poultry. Colonization Control of Human Bacterial Enteropathogens in Poultry. New York, Academic Press, Inc.: 169-198.
Davis, M. S., J. Solbiati, et al. (2000). “Overproduction of acetyl-CoA carboxylase activity increases the rate of fatty acid biosynthesis in Escherichia coli.” J Biol Chem 275(37): 28593-28598.
Dehesh, K., A. Jones, et al. (1996). “Production of high levels of 8:0 and 10:0 fatty acids in transgenic canola by overexpression of Ch FatB2, a thioesterase cDNA from Cuphea hookeriana.” Plant J 9(2): 167-172.
Denoya, C. D., R. W. Fedechko, et al. (1995). “A second branched-chain alpha-keto acid dehydrogenase gene cluster (bkdFGH) from Streptomyces avermitilis: its relationship to avermectin biosynthesis and the construction of a bkdF mutant suitable for the production of novel antiparasitic avermectins.” J Bacteriol 177(12): 3504-3511.
Dirusso, C. C. and P. N. Black (2004). “Bacterial long chain fatty acid transport: gateway to a fatty acid-responsive signaling system.” J Biol Chem 279(48): 49563-49566.
Dong, C., K. Beis, et al. (2006). “Wza the translocon for E. coli capsular polysaccharides defines a new class of membrane protein.” Nature 444(7116): 226-229.
Doublet, P., J. van Heijenoort, J. P. Bohin, and D. Mengin-Lecreulx. (1993). “The murl gene of Escherichia coli is an essential gene that encodes a glutamate racemase activity.” J. Bacteriol. 175:2970-2979.
Fell, D. A. (1996). “Understanding the Control of Metabolism, Portland Press, London.”
Folch, J., M. Lees, et al. (1957). “A simple method for the isolation and purification of total lipides from animal tissues.” J Biol Chem 226(1): 497-509. Foster J S, Singh A K, Rothschild L J, Sherman L A. (2007). “Growth-phase dependent differential gene expression in Synechocystis sp. strain PCC 6803 and regulation by a group 2 sigma factor.” Arch Microbiol. 187(4):265-79.
Hamilton, J. A. (2007). “New insights into the roles of proteins and lipids in membrane transport of fatty acids.” Prostaglandins Leukot Essent Fatty Acids 77(5-6): 355-361.
Hayes, W. (1968). “The genetics of bacteria and their viruses.” John Wiley, New York.
Hayes P K, Lazarus C M, Bees A, Walker J E, Walsby A E. (1988). “The protein encoded by gvpC is a minor component of gas vesicles isolated from the cyanobacteria Anabaena flos-aquae and Microcystis sp.” Mol. Microbiol. 1988 September; 2(5):545-52.
Helmsing, P. J. (1969). “Purification and properties of galactolipase.” Biochim Biophys Acta 178(3): 519-533.
Hirani, T. A., I. Suzuki, et al. (2001). “Characterization of a two-component signal transduction system involved in the induction of alkaline phosphatase under phosphate-limiting conditions in Synechocystis sp. PCC 6803.” Plant Mol Biol 45(2): 133-144. Hirsch, D., A. Stahl, et al. (1998). “A family of fatty acid transporters conserved from mycobacterium to man.” Proc Natl Acad Sci USA 95(15): 8625-8629.
Hitchcock, P. J. and T. M. Brown (1983). “Morphological heterogeneity among Salmonella lipopolysaccharide chemotypes in silver-stained polyacrylamide gels.” J Bacteriol 154(1): 269-277.
Hoiczyk, E. and A. Hansel (2000). “Cyanobacterial cell walls: news from an unusual prokaryotic envelope.” J Bacteriol 182(5): 1191-1199.
Hu, Q., M. Sommerfeld, et al. (2008). “Microalgal triacylglycerols as feedstocks for biofuel production: perspectives and advances.” Plant J 54(4): 621-639.
Huang, F., E. Hedman, et al. (2004). “Isolation of outer membrane of Synechocystis sp. PCC 6803 and its proteomic characterization.” Mol Cell Proteomics 3(6): 586-595.
Imashimizu, M., S. Fujiwara, et al. (2003). “Thymine at −5 is crucial for cpc promoter activity of Synechocystis sp. strain PCC 6714.” J Bacteriol 185(21): 6477-6480.
Isberg R R, Falkow S. (1985). “A single genetic locus encoded by Yersinia pseudotuberculosis permits invasion of cultured animal cells by Escherichia coli K-12.” Nature. September 19-25;317(6034):262-4.
Kaczmarzyk, D. and M. Fulda (2010). “Fatty acid activation in cyanobacteria mediated by acyl-acyl carrier protein synthetase enables fatty acid recycling.” Plant Physiol dio:10.1104/pp. 109.148007.
Karlsson, B., T. Vaara, et al. (1983). “Three-dimensional structure of the regularly constructed surface layer from Synechocystis sp. strain CLII.” J Bacteriol 156(3): 1338-1343.
Kawai, E., Akatsuka, H., Idei, A., Shibatani, T., and Omori, K.: (1998) “Serrutia marcescens S-layer protein is secreted extracellularly via an ATP-binding cassette exporter, the Lip system.” Mol. Microbial., 27, 941-952.
Kohler, G. A., A. Brenot, et al. (2006). “Phospholipase A2 and phospholipase B activities in fungi.” Biochim Biophys Acta 1761(11): 1391-1399.
Kufryk, G. I., M. Sachet, et al. (2002). “Transformation of the cyanobacterium Synechocystis sp. PCC 6803 as a tool for genetic mapping: optimization of efficiency.” FEMS Microbiol Lett 206(2): 215-219.
Kunert, A., M. Hagemann, et al. (2000). “Construction of promoter probe vectors for Synechocystis sp. PCC 6803 using the light-emitting reporter systems Gfp and LuxAB.” J Microbiol Methods 41(3): 185-194.
Lalman, J. A. and D. M. Bagley (2004). “Extracting long-chain fatty acids from a fermentation medium.” JAOCS 81: 105-110.
Leong J M, Fournier R S, Isberg R R. (1990). “Identification of the integrin binding domain of the Yersinia pseudotuberculosis invasin protein.” EMBO J. June; 9(6):1979-89.
Levine, M. and R. Curtiss (1961). “Genetic fine structure of the C region and the linkage map of phage P22.” Genetics 46: 1573-1580.
Li, Y., G. Florova, et al. (2005). “Alteration of the fatty acid profile of Streptomyces coelicolor by replacement of the initiation enzyme 3-ketoacyl acyl carrier protein synthase III (FabH).” J Bacteriol 187(11): 3795-3799.
Lilley, P. E., N. P. Stamford, S. G. Vasudevan, and N. E. Dixon. (1993). “The 92-min region of the Escherichia coli chromosome: location and cloning of the ubiA and alr genes.” Gene 129:9-16.
Linhartova, I., L. Bumba, et al. (2010). “RTX proteins: a highly diverse family secreted by a common mechanism.” FEMS Microbiol Rev.
Liou, G. G., W. N. Jane, S. N. Cohen, N. S. Lin, and S. Lin-Chao (2001). “RNA degradosomes exist in vivo in Escherichia coli as multicomponent complexes associated with the cytoplasmic membrane via the N-terminal region of ribonuclease E.” Proc. Natl. Acad. Sci. USA 98: 63-68.
Liu, X., D. Brune, et al. (2010). “Production and secretion of fatty acids in genetically engineered cyanobacteria.” Proc Natl Acad Sci USA.
Liu, X. and R. Curtiss, 3rd (2009). “Nickel-inducible lysis system in Synechocystis sp. PCC 6803.” Proc Natl Acad Sci USA 106(51): 21550-21554.
Loessner, M. J. (2005). “Bacteriophage endolysins—current state of research and applications.” Curr Opin Microbiol 8(4): 480-487.
Lobocka, M., J. Hennig, J. Wild, and T. Klopotowski. (1994). “Organization and expression of the Escherichia coli K-12 dad operon encoding the smaller subunit of D-amino acid dehydrogenase and the catabolic alanine racemase.” J. Bacteriol. 176:1500-1510.
Lounatmaa, K., T. Vaara, et al. (1980). “Ultrastructure of the cell wall of a Synechocystis strain.” Can J Microbiol 26(2): 204-208.
Lupas, A., H. Engelhardt, et al. (1994). “Domain structure of the Acetogenium kivui surface layer revealed by electron crystallography and sequence analysis.” J Bacteriol 176(5): 1224-33.
Marbouty, M., K. Mazouni, et al. (2009). “Characterization of the Synechocystis strain PCC 6803 penicillin-binding proteins and cytokinetic proteins FtsQ and FtsW and their network of interactions with ZipN.” J Bacteriol 191(16): 5123-5133.
Marraccini, P., S. Bulteau, et al. (1993). “A conjugative plasmid vector for promoter analysis in several cyanobacteria of the genera Synechococcus and Synechocystis.” Plant Mol Biol 23(4): 905-909.
McCarren, J., Heuser, J., Roth, R., Yamada, N., Martone, M., Brahamsha, B. (2005). “Inactivation of swmA Results in the Loss of an Outer Cell Layer in a Swimming Synechococcus Strain.” J. Bacteriol. 187: 224-230.
McGinn, P. J., G. D. Price, et al. (2003). “Inorganic carbon limitation and light control the expression of transcripts related to the CO2-concentrating mechanism in the cyanobacterium Synechocystis sp. strain PCC6803.” Plant Physiol 132(1): 218-229.
Mermet-Bouvier, P., C. Cassier-Chauvat, et al. (1993). “Transfer and replication of RSF1010-derived plasmids in several cyanobacteria of the genera Synechocystis and Synechococcus” Current Microbiology 27(6): 323-327.
Mermet-Bouvier, P. and F. Chauvat (1994). “A conditional expression vector for the cyanobacteria Synechocystis sp. strains PCC6803 and PCC6714 or Synechococcus sp. strains PCC7942 and PCC6301.” Curr Microbiol 28(3): 145-148.
Michinaka, Y., T. Shimauchi, et al. (2003). “Extracellular secretion of free fatty acids by disruption of a fatty acyl-CoA synthetase gene in Saccharomyces.” J Biosci Bioeng 95(5): 435-440.
Middleton, R. B. and T. Mojica-a (1971). “Genetics of the enterobacteriaceae. B. Homology in the enterobacteriaceae based on intercrosses between spaces.” Adv Genet 16: 53-79.
Milcamps, A., D. M. Ragatz, et al. (1998). “Isolation of carbon- and nitrogen-deprivation-induced loci of Sinorhizobium meliloti 1021 by Tn5-luxAB mutagenesis.” Microbiology 144 (Pt 11): 3205-3218.
Miller, V. L. and J. J. Mekalanos (1988). “A novel suicide vector and its use in construction of insertion mutations: osmoregulation of outer membrane proteins and virulence determinants in Vibrio cholerae requires toxR.” J Bacteriol 170(6): 2575-2583.
Miller V L, Falkow S. 1988. “Evidence for two genetic loci in Yersinia enterocolitica that can promote invasion of epithelial cells.” Infect Immun. May; 56(5):1242-8.

Mojica, A. T. and R. B. Middleton (1971). “Fertility of Salmonella typhimurium crosses with Escherichia coli.” J Bacteriol 108(3): 1161-1167.
Molina Grima, E., E. H. Belarbi, et al. (2003). “Recovery of microalgal biomass and metabolites: process options and economics.” Biotechnol Adv 20(7-8): 491-515.
Morrison, S. S., C. W. Mullineaux, et al. (2005). “The influence of acetyl phosphate on DspA signalling in the Cyanobacterium Synechocystis sp. PCC6803.” BMC Microbiol 5: 47.
Nakayama, K., S. Shiota, et al. (1988). “Thymineless death in Escherichia coli mutants deficient in the RecF recombination pathway.” Can J Microbiol 34(7): 905-907.
Ohlrogge, J. B. (1982). “Fatty acid synthetase: plants and bacteria have similar organization.” Trends Biochem Sci 7: 386-387.
Ohlrogge, J. B., D. N. Kuhn, et al. (1979). “Subcellular localization of acyl carrier protein in leaf protoplasts of Spinacia oleracea.” Proc Natl Acad Sci USA 76(3): 1194-1198.
Onizuka, T., H. Akiyama, et al. (2003). “CO2 response for expression of ribulose-1,5-bisphosphate carboxylase/oxygenase genes is inhibited by AT-rich decoy in the cyanobacterium.” FEBS Lett 542(1-3): 42-46.
Panoff, J. M., B. Priem, et al. (1988). “Sulphated exopolysaccharides produced by two univellular strains of cyanobacteria, Synechocystis PCC 6803 and 6714.” Archives of Microbiology 150: 558-563.
Paton, A. W. and J. C. Paton (1999). “Molecular characterization of the locus encoding biosynthesis of the lipopolysaccharide 0 antigen of Escherichia coli serotype 0113.” Infect Immun 67(11): 5930-5937.
Pierce, D. L., D. S. O'Donnol, et al. (2006). “Mutations in DivL and CckA rescue a divJ null mutant of Caulobacter crescentus by reducing the activity of CtrA.” J Bacteriol 188(7): 2473-2482.
Pollard, M. R., L. Anderson, et al. (1991). “A specific acyl-ACP thioesterase implicated in medium-chain fatty acid production in immature cotyledons of Umbellularia californica.” Arch Biochem Biophys 284(2): 306-312.
Porter, R. D. (1986). “Transformation in cyanobacteria.” Crit Rev Microbiol 13(2): 111-132.
Qadri, F., S. A. Hossain, et al. (1988). “Congo red binding and salt aggregation as indicators of virulence in Shigella species.” J Clin Microbiol 26(7): 1343-1348.
Raetz C R, et al. (2009) “Discovery of new biosynthetic pathways: the lipid A story.” J Lipid Res. 2009 April; 50 Suppl:S103-8.
Rapp, P. (1995). “Production, regulation, and some properties of lipase activity from Fusarium oxysporum f. sp. vasinfectum.” Enzyme Microb. Technol 17: 832-838.
Ray, J. M., D. Bhaya, et al. (1991). “Isolation, transcription, and inactivation of the gene for an atypical alkaline phosphatase of Synechococcus sp. strain PCC 7942.” J Bacteriol 173(14): 4297-4309.
Rippka, R., J. Derulles, et al. (1979). “Generic assignments, strain histories and properties of pure cultures cyanobacteria.” J Gen Microbiol 111: 1-61.
Robeson, J. P., R. M. Goldschmidt, et al. (1980). “Potential of Escherichia coli isolated from nature to propagate cloning vectors.” Nature 283(5742): 104-106.
Rosenstein, R. and F. Gotz (2000). “Staphylococcal lipases: biochemical and molecular characterization.” Biochimie 82(11): 1005-1014.
Roth, B. L., M. Poot, et al. (1997). “Bacterial viability and antibiotic susceptibility testing with SYTOX green nucleic acid stain.” Appl Environ Microbiol 63(6): 2421-2431.
Sakiyama, T., H. Ueno, et al. (2006). “Purification and characterization of a hemolysin-like protein, Sll1951, a nontoxic member of the RTX protein family from the Cyanobacterium Synechocystis sp. strain PCC 6803.” J Bacteriol 188(10): 3535-3542.
Sambrook, J., E. F. Fritsch, et al. (1989). Molecular cloning: a laboratory manual. Plainview, N.Y., Cold Spring Harbor Laboratory Press.
Sara, M. and U. B. Sleytr (2000). “S-Layer proteins.” J Bacteriol 182(4): 859-68. Scharnewski, M., P. Pongdontri, et al. (2008). “Mutants of Saccharomyces cerevisiae deficient in acyl-CoA synthetases secrete fatty acids due to interrupted fatty acid recycling.” FEBS J 275(11): 2765-2778.
Scheerer, S., O. Ortega-Morales, et al. (2009). “Microbial deterioration of stone monuments—an updated overview.” Adv Appl Microbiol 66: 97-139.
Scherzinger, E., M. M. Bagdasarian, et al. (1984). “Replication of the broad host range plasmid RSF1010: requirement for three plasmid-encoded proteins.” Proc Natl Acad Sci USA 81(3): 654-658.
Singh, A. K. and L. A. Sherman (2006). “Iron-independent dynamics of IsiA production during the transition to stationary phase in the cyanobacterium Synechocystis sp. PCC 6803.” FEMS Microbiol Lett 256(1): 159-164.
Smit, J., H. Engelhardt, S. Volker, S. H. Smith, and W. Baumeister. 1992. “The S-layer of Caulobacter crescentus: three-dimensional image reconstruction and structure analysis by electron microscopy.” J. Bacteriol. 174:6527-6538.
Smith, C. S., A. Hinz, et al. (2003). “Identification of genes required for synthesis of the adhesive holdfast in Caulobacter crescentus.” J Bacteriol 185(4): 1432-1442.
Smolke, C. D., T. A. Carrier, and J. D. Keasling (2000). “Coordinated, differential expression of two genes through directed mRNA cleavage and stabilization by secondary structures.” Appl. Environ. Microbiol. 66: 5399-5405.
Stenstrom, C. M., H. Jin, et al. (2001). “Codon bias at the 3′-side of the initiation codon is correlated with translation initiation efficiency in Escherichia coli.” Gene 263(1-2): 273-284.
Svendsen, A. (2000). “Lipase protein engineering.” Biochim Biophys Acta 1543(2): 223-238.
Syed M A, Henshaw P F. 2003. “Effect of tube size on performance of a fixed-film tubular bioreactor for conversion of hydrogen sulfide to elemental sulfur.” Wat Res 37:1932-1938
Tatusov, R. L., M. Y. Galperin, D. A. Natale, and E. V. Koonin. 2000. “The COG database: a tool for genome-scale analysis of protein functions and evolution.” Nucleic Acids Res 28:33-36.
Thompson, B. R., S. Lobo, et al. (2009). “Fatty acid flux in adipocytes: The in's and out's of fat cell lipid trafficking.” Mol Cell Endocrinol.
Tian X, Liao Q, Zhu X, WangY, Zhang P, Li J, Wang H. 2010. “Characteristics of a biofilm photobioreactor as applied to photo-hydrogen production.” Bioresour Technol 101:977-983
Tsai, C. M. and C. E. Frasch (1982). “A sensitive silver stain for detecting lipopolysaccharides in polyacrylamide gels.” Anal Biochem 119(1): 115-119.
Tsang P H, Li G, Brun Y V, Freund L B, Tang J X. 2006. “Adhesion of single bacterial cells in the micronewton range.” Proc Natl Acad Sci USA. April 11;103(15):5764-8.
Voelker, T. A. and H. M. Davies (1994). “Alteration of the specificity and regulation of fatty acid synthesis of Escherichia coli by expression of a plant medium-chain acyl-acyl carrier protein thioesterase.” J Bacteriol 176(23): 7320-7327.
Wada, H. and N. Murata (1990). “Temperature-Induced Changes in the Fatty Acid Composition of the Cyanobacterium, Synechocystis PCC6803.” Plant Physiol 92(4): 1062-1069.
Wasserman, S. A., C. T. Walsh, and D. Botstein. 1983. “Two alanine racemase genes in Salmonella typhimurium that differ in structure and function.” J. Bacteriol. 153:1439-1450.
Wilson, W. H., I. R. Joint, et al. (1993). “Isolation and Molecular Characterization of Five Marine Cyanophages Propagated on Synechococcus sp. Strain WH7803.” Appl Environ Microbiol 59(11): 3736-3743.
Wolk, C. P., Y. Cai, et al. (1991). “Use of a transposon with luciferase as a reporter to identify environmentally responsive genes in a cyanobacterium.” Proc Natl Acad Sci USA 88(12): 5355-5359.
Yoshihara, S., X. Geng, et al. (2001). “Mutational analysis of genes involved in pilus structure, motility and transformation competency in the unicellular motile cyanobacterium Synechocystis sp. PCC 6803.” Plant Cell Physiol 42(1): 63-73.
Zang, X., B. Liu, et al. (2007). “Optimum conditions for transformation of Synechocystis sp. PCC 6803.” J Microbiol 45(3): 241-245.
Zheng, W., B. Bergman, et al. (2009). “Cellular responses in the cyanobacterial symbiont during its vertical transfer between plant generations in the Azolla microphylla symbiosis.” New Phytol 181(1): 53-61.
Ziegler, K., A. Diener, et al. (1998). “Molecular characterization of cyanophycin synthetase, the enzyme catalyzing the biosynthesis of the cyanobacterial reserve material multi-L-arginyl-poly-L-aspartate (cyanophycin).” Eur J Biochem 254(1): 154-159.

### Example 22

### Generation of Deletion Mutations for Genes Encoding Essential Proteins (Enzymes)

Suicide vectors listed in Table 16 were constructed using the techniques previously described under the section “Materials and methods for examples 1-8” (see sections i-iii) to enable deletion of the essential 6803 asd, alr, dapA, dapB, and murl genes. FIG. 32 depicts the extent of these deletions in terms of the specific nucleotide sequences deleted. The deletion mutants generated by use of these suicide vectors will require threonine, methionine and DAP, D-alanine, D-glutamate or DAP alone, respectively, when grown in BG-11 medium.

TABLE 16
Suicide Vectors

| Vector Number | Gene | Marker |
|---|---|---|
| pΨ562 | asd | Amp |
| pΨ563 | asd | Kan sacB |
| pΨ564 | alr | Amp |
| pΨ565 | alr | Kan sacB |
| pΨ623 | dapA | Amp |
| pΨ624 | dapA | Kan sacB |
| pΨ625 | dapB | Amp |
| pΨ626 | dapB | Kan sacB |

The two suicide plasmids needed to delete the 6803 alr gene are shown in FIG. 33. Table 17 lists the Synechocystis strains that have been engineered to possess the Δalr-23 mutation. Some, such as SD284 and SD249, have been modified to over produce and secrete free fatty acids as biofuel precursors.

TABLE 17
Synechocystis 6803 strains with alr deletion

| Strain | Parent | |
|---|---|---|
| Number | strain | Genotype |
| SD546 | SD100 | Δalr-23 |
| SD555 | SD284 | Δaas::P_{trc}tesA137 Δalr-23 |
| SD568 | SD249 | Δaas-23::P_{psbA236}tesA136 |
| | | Δ(slr1993-slr1994)-14::P_{cpc39} accBRBS accC70 P_{rbc40} |
| | | accDRBSaccA |
| | | Δsll1951-15::P_{psbA210}fatB161(Uc) |
| | | P_{rbc41}fatB262(Ch)Δ(slr2001-slr2002)-17::P_{psbA211} |
| | | fatB262(Ch) |
| | | Δslr1710-19::P_{psbA210}fatB163(Cc) Δalr-23 |

### Example 23

### Generation of Shuttle Plasmid Vectors for E. coli and Cyanobacteria

A series of plasmids based on the IncQ conjugative plasmid RSF1010 may be constructed to facilitate research on the genetic manipulation of E. coli and cyanobacteria. The 5.7 kb region of RSF1010 containing three rep genes, A, B, and C, is necessary for its replication in 6803(Scherzinger, Bagdasarian et al. 1984; Marraccini, Bulteau et al. 1993). RSF1010 was modified to construct a family of promoter fusion vectors with various reporter genes. For example, the promoter search vectorp W575 (FIG. 34) has a gfp reporter, and the promoter search vector pΨ576 (FIG. 35) has a luxAB reporter. Random cloning of DNA sequences has been used to search for promoters that result in reporter gene expression at high level under any condition such as stationary phase, high or low light intensity, CO₂ limitation, etc. to identify promoters useful in constructing cyanobacterial strains improved for production of biofuels or biofuel precursors. Also, RSF1010 derived shuttle vectors may be constructed with three different regulatable promoters (P_{trc}, araC P_{BAD}, and P_{lpp-lacO}) that can be used to express specific cloned genes using the multiple cloning site to complement deleted genes in Synechocystis strains or to determine the effect of over expression of genes encoding enzymes important for biofuel production. To use a vector with the araC P_{BAD} promoter the strain will have to posses a chromosomally inserted E. coli araE gene enabling arabinose transport into the cell. This construction is described in Example 25 below. An example of one such shuttle vector pΨ568 with a LacI regulatable P_{trc} promoter is depicted in FIG. 38. The P_{trc} promoter may be replaced with other promoters such as araC P_{BAD} and P_{lpp-lacO}. These vectors may be used to determine whether addition of a gene or operon would enhance any property of interest such as FFA synthesis and secretion. Appropriate 6803 strains may be readily constructed using methods outlined in above teachings and Examples depending on the requirement for each vector construct. Therefore, the wild-type E. coli araE gene may be inserted into the cyanobacterial chromosome to facilitate/allow arabinose uptake to regulate the araC P_{BAD} promoter and the E. coli lacI gene to cause synthesis of LacI as a repressor to regulate expression of either the P_{trc} or P_{lpp-lacO} promoters.

All of the above-described vectors derived from RSF1010 have the original aadA gene encoding resistance to the antibiotics streptomycin and spectinomycin or the substituted aph gene specifying resistance to kanamycin. Since use of antibiotics to maintain plasmid vectors would be expensive on a commercial scale and be environmentally unacceptable, genes encoding an essential gene may be inserted to complement a deletion of an essential gene in cyanobacteria. In the first step toward making such balanced-lethal vectors, the essential gene may be first inserted and the antibiotic resistance gene may be retained, affording two means of selecting for plasmid vector transfer during strain construction and in maintaining the plasmid after construction. These constructions are illustrated in the next Example.

### Example 24

### Establishment of Balanced-Lethal Plasmid-Host Systems to Enable Stable Plasmid Maintenance

The 6803 asd and alr genes were initially cloned in the low copy E. coli plasmid pWSK29 (pSC101 ori) to yield pv566 and pv567 and their inheritance was selected for by their ability to complement asd and alr mutations in E. coli strain χ6097 (E. coli K-12 F⁻, araΔ[pro-lac] rpsLφ80d lacZ ΔM15ΔasdA4 Δ[zhf-2::Tn10]) and S. Typhimurium strain χ8901 (S. Typhimurium UK-1 Δalr-3 ΔdadB4), respectively. Since the strains grow, it is evident that the 6808 asd⁺ and alr⁺ homologs can functionally complement the asd and alr gene deletions in E. coli and/or Salmonella. Plasmids containing other essential genes such as the 6803 murl⁺ gene may also be constructed to evaluate their use as selective markers on shuttle vectors. The 6808 asd⁺ and alr⁺ genes have also been inserted into RSF1010 to yield pΨ569 (FIG. 36) and pΨ570 (FIG. 37) and these plasmids also complement Δasd and ΔalrΔdadB mutants of S. Typhimurium. As stated above, RSF1010 is a broad host range conjugative plasmid capable of replication in both Synechocystis and E. coli.

The means to transfer any RSF1010 derived plasmid (for example pΨ569 and pΨ570) can be done using the triparental mating method (Elhai, J. & Wolk, C. P. Conjugal transfer of DNA to cyanobacteria. Methods Enzymol 1988.167, 747-754). The three parents are (1) E. coli strains HB101 harboring helper plasmid pRL528 (methylases) and the target plasmid (our Asd⁺ and Alr⁺ expression vectors, for example), (2) E. coli bearing the conjugal plasmid pRL443, and the target 6803 recipient cells that lack either the asd or alr gene. The three strains are mixed and allowed to mate on a nitrocellulose membrane on solid BG-11 medium for 12 h under 40 mmol m⁻² g⁻¹ white light at 30° C. Exconjugants are selected by transferring the filter to solid BG-11 medium supplemented with either DAP plus methionine and threonine or D-Alanine, respectively. Then the filter will be transferred to solid BG-11 medium without supplements to select only the complemented exconjugants.

The Δalr 6803 mutant SD546 (Table 17) was used to establish a balanced-lethal plasmid-host system that ensures maintenance of the plasmid vector by placing the wild-type alr gene in a RSF1010 derivative pΨ568 (FIG. 38) to yield pΨ570 (FIG. 37). These vectors contain both the aadA gene for streptomycin and spectinomycin resistance and the alr⁺ gene such that by using the triparental mating method described above transconjugants may be obtained in SD100 by selecting for either spectinomycin or streptomycin resistance or for growth in the absence of D-alanine. Each type of transconjugant may then be screened for the non-selected phenotypes. In such an experiment, selecting for the inheritance of pΨ570 transferred to SD100, it was found that 100% of the spectinomycin-resistant isolates were also streptomycin resistant and also grew in the absence of D-alanine. Similarly, 100% of the streptomycin-resistant isolates were also spectinomycin resistant and also grew in the absence of D-alanine. Lastly 100% of the Alr⁺ transconjugants were resistant to both spectinomycin and streptomycin.

To test for the reciprocal function of E. coli and Salmonella wild-type essential genes to complement deletion of Synechocystis essential genes, the Salmonella wild-type alanine racemasealr and dadB genes have also been inserted into pΨ568 to yield pΨ591 (FIG. 39B) and pΨ592 (FIG. 39C), respectively. When the pΨ570, pΨ591 and pΨ592 plasmids are transformed into the Δalr strain SD546 (Table 17), they grow well in the BG-11 media without the supplement (D-alanine) while SD546 dies by lysis in BG-11 media without D-alanine as a supplement (FIG. 39A). It is therefore proven that wild-type essential genes from other bacteria may be used to complement for loss by deletion of Synechocystis essential genes.

As the last step in generating a balanced-lethal vector without antibiotic-resistance genes, the aadA gene encoding streptomycin and spectinomycin resistance was replaced with the wild-type 6803 alr gene to yield pΨ642 (FIG. 40). This plasmid has the strong promoter Ptrc and enables regulation of any inserted gene when the recombinant shuttle vector is introduced into an E. coli host with over expression of the lacl gene to synthesize LacI to repress transcription from the P_{trc} promoter. Then, the inducer IPTG may then be added to study synthesis of the enzyme(s) encoded by the cloned genes in E. coli. This permits studies on enzyme function and stability. For example, this plasmid may be engineered such that P_{trc} controls the expression of the accABCD genes in pΨ622 to yield pΨ627 (FIG. 40) to cause over production of manonyl Co-A. This technology should further increase FFA production and secretion. Based on these teachings, it should be obvious how this system can also be used with the wild-type asd, dapA, dapB, and murl genes on shuttle plasmid vectors to establish balanced-lethal systems when the biofuel-producing strain(s) has (have) deletion mutations for the same essential gene.

### Example 25

### Regulatable MurA Synthesis

An essential nucleic acid such as murA may encode a protein involved in muramic acid synthesis, as muramic acid is another required constituent of the peptidoglycan layer of the bacterial cell wall. It is not possible to alter murA by deletion, however, because a ΔmurA mutation is lethal and cannot be isolated. This is because the missing nutrient required for viability is a phosphorylated muramic acid that cannot be exogenously supplied because most, if not all, bacteria cannot internalize it. Consequently, the murA nucleic acid sequence may be altered to make expression of murA dependent on a nutrient (e.g., arabinose) that can be supplied during the growth of the bacterium. In this case, since cyanobacteria are often unable to take up arabinose, the strain may first be genetically modified to enable uptake of arabinose by introducing an araE gene from E. coli that encodes an arabinose-uptake protein. This may be accomplished by cloning the E. coli araE gene and inserting it into a shuttle vector to enable insertion in place of a dispensible Synechocystis gene. Such genes have been identified in preceding Examples and generally encode products that would compete for energy or biosynthetic ability that would decrease ability of strains to produce desired biofuels or biofuel precursors. Such a dispensable/competing gene could be the sll1951 gene encoding an S-Layer protein. Using such an araE⁺ strain enables the introduction of a ΔP_{murA}::TT araC P_{BAD}murA deletion-insertion mutation chromosomal mutation (FIG. 32F) that will result in an arabinose-dependent phenotype. In the absence of arabinose in the growth medium the strain will undergo muramic-less death and lyse. The ΔP_{murA}:TT araC P_{BAD}murA deletion-insertion mutation is therefore a conditional lethal mutation.

Cyanobacterial strains with the ΔP_{murA}:TT araC P_{BAD}murA deletion-insertion mutation may be used to develop an additional balanced-lethal vector-host system. This may be accomplished by cloning the wild-type murA gene and inserting it into a shuttle vector plasmid (FIG. 38) as the sole selective marker in the absence of genes for antibiotic resistance. This plasmid may then be used to express genes to augment synthesis of desirable biofuels or biofuel precursors.

### Example 26

### Selection for Elevation in Plasmid Copy Number as a Means to Increase Synthesis of Enzymes Augmenting Production of Biofuels and/or Biofuel Precursors

The RSF1010 plasmid possesses the aadA gene conferring resistance to both streptomycin and spectinomycin. Inactivation of both antibiotics is by adenylylation and both antibiotics block protein synthesis by binding to two different ribosomal proteins encoded by two separate chromosomal genes. It is also likely that both antibiotics enter bacterial cells by different pathways. Many years ago, many investigators investigated whether levels of antibiotic resistance in bacteria could be increased in R plasmid containing strains. Results were generally positive and several mechanisms for achieving this were discovered. One mechanism resulted in increased plasmid copy numbers (Macrina et al. 1974. J. Bacteriol. 120:1387). The aadA gene in RSF1010 confers resistance to about 30 μg/ml of either streptomycin or spectinomycin and to slightly lower concentrations when added simultaneously to the BG-11 growth medium. Selection for increased resistance to both spectinomycin and streptomycin may selectively enrich for strains synthesizing increased amounts of the AadA enzyme and this may be by mutations increasing aadA gene expression by improvements in promoters, ribosome binding sites or by optimization of codons to improve translation or stabilize mRNA also to increase translation efficiency. These changes may likely be small and incremental whereas mutations that increased plasmid copy number may be expected to have a more significant increase in dual antibiotic resistance. By optimizing the concentrations of the two antibiotics, It may be possible to enrich for mutations to increase plasmid copy number. It should be emphasized that mutations in two chromosomal genes to confer increased resistance to both antibiotics would essentially never occur since the mutation rates to each type of resistance are exceedingly rare being 10⁻¹⁰/bacterium/generation and the mutation rate to both antibiotics would be the product of the individual mutation rates to resistance (10⁻²⁰/bacterium/generation). Since the Synechocystis strain in these examples is ampicillin sensitive, the bla gene encoding β-lactamase may also be added on balanced-lethal or shuttle vectors to enable use of ampicillin to select for high-level ampicillin resistance, which would also be expected to result in plasmid mutants with increased copy numbers. The two selection methods may also be combined to provide even greater selective pressure to yield the desired high copy number plasmids. Upon isolating plasmids with increased copy numbers, their use in various applications of the balanced-lethal vector technologies described in these examples may be investigated.

### Example 27

### Insertion of Genes to Augment FFA Synthesis and Secretion

Genes and operons like the accBCDA, fatB1, fatB2, and rbcLXS may be inserted into multi-copy plasmids stabilized by any one of the balanced-lethal systems to amplify the overall productivity of strains to synthesize and secrete free fatty acids or other biofuels or biofuel precursors. The plasmid pΨ627 containing accBCDA along with the balanced-lethal system has been constructed and is shown in FIG. 41.

It should also be evident that one may investigate use of additional plasmids to establish balanced-lethal systems for the various uses described that are either broad host range being able to replicate in either E. coli or Synechocystis or only in Synechocystis in which case a fusion replicon (Hansen et al. 1981. Infect. Immun. 31:1034) able to replicate in both bacterial hosts may be generated. In this regard, Synechocystis 6803 has three small plasmids that may be exploited in these ways to establish additional balanced-lethal or shuttle vector systems.

TABLE 14
Primers used in this study

| | | |
|---|---|---|
| | | SEQ. |
| | | ID |
| Primer Name | Sequence (5′ to 3′) | NO. |

Construction of pΨ214

| | | |
|---|---|---|
| FadD-F1-A | TAA ACT CTG TAG GCC AGC GGC AA | 1 |
| FadD-F1-S | CGT CAA TGC CTA GAC CTA GCA GTA CC | 2 |
| FadD-F2-S | AAG GAT TTC CGT TTT ATC CCA GCA CCA | 3 |
| FadD-F2-A | GTA ATT GCC ACA GAC AAG CGT ATT CGG | 4 |
| KS-NdeI | ACC ATA TGC ATC CTA GGC CTA TTA ATA TTC CGG | 5 |
| KS-BamHI | GAA TTA GGA TCC GTC GAC CTG CAG G | 6 |

Construction of pΨ215

| | | |
|---|---|---|
| NiF1-S-EcoRI | GAgAA TTc CAG ACG ACT ACG GGC AAA G | 7 |
| NiF1-A-toTesA | AAC GTG TCC GCC ATC ACA CCA CCT CAA ATT G | 8 |
| TesA-S-toNiF1 | GAG GTG GTG TGA TGG CGG ACA CGT TAT TGA T | 9 |
| TesA-A-toNiF2 | CAA AAG GAG CAA TGT GTT ATT TGT CAT CAT CGT CTT | 10 |
| NiF2-S-toTesA | GAT GAT GAC AAA TAA CAC ATT GCT CCT TTT GTG CG | 11 |
| NiF2-A-BamHI | ACG GAT CCG CAA GCA GTG AAA GAT AG | 12 |

Construction of pΨ216

| | | |
|---|---|---|
| TesA-S | CAAATGGCGGACACGTTATTGATTCTG | 13 |
| TesA-A | CTT TGT AGT CTG AGT CAT GAT TTA CTA AAG GCT G | 14 |
| Test-S-to-pA2 | ATTATAACCAAATGGCGGACACGTTA | 15 |
| pA2-S | TCCCCATTGCCCCAAAATACATCC | 16 |
| pA2-A-to-TesA | CAA TAA CGT GTC CGC CAT TTG GTT ATA ATT CCT TA | 17 |

Construction of pΨ207

| | | |
|---|---|---|
| S4F1-S-PstI | GActgcAGGTCATTGCCGATAAAGTTG | 18 |
| S4F1-A-XbaI | AGtctagATAATGTACAGGTCAAGCTGGTCT | 19 |
| S4F2-S-SacI | GAgagcTCATTGACACCGAAATGACTTTGG | 20 |
| S4F2-A-EcoRI | GAGAATTCTTTGCATTTCCGAAACCACCC | 21 |
| S4F2-S-XbaI | GATCTAGAATTGACACCGAAATGACTTTGG | 22 |
| S4F2-A-KpnI | GAGGTACCTTTGCATTTCCGAAACCACCC | 23 |

Construction of pΨ223

| | | |
|---|---|---|
| Pcpc-S | TAG GCT GTG GTT CCC TAG GCA ACA GT | 24 |
| Pcpc-A-to-SynB | TCC GTA AAG TTA ATA GCC ATT GAA TTA ATC TCC TAC TTG AC | 25 |
| SynB-S-to-Pcpc | AGG AGA TTA ATT CAA TGG CTA TTA ACT TTA CGG AAC TGC G | 26 |
| SynB-A-to-SynC | CAT TGA ATT AAT CTC CTC TAG GGT TTA ATC CAC ATT AGG GTT | 27 |
| SynC-S | CCT AGA GGA GAT TAA TTC AAT GCA ATT CGC CAA AAT TTT AAT | 28 |
| | TGC | |
| SynC-A | CTC TCC ATT GAC CTA GGG TGT TAA ATG CTC TTC G | 29 |
| SynC-S-to-SynB | GTG GAT TAA ACC CTA GAG GAG ATT AAT TCA ATG CAA TTC GC | 30 |
| SynC-A-to-Prbc | CTT TAC TTA TGG CAA TGC TCT CCA TTG ACC TAG GGT GTT | 31 |
| Prbc-S | AAC ACC CTA GGT CAA TGG AGA GCA TTG CCA T | 32 |
| Prbc-A-to-SynD | CAA TCA AAT AGA GAC ATC TAG GTC AGT CCT CCA TAA AC | 33 |
| SynD-S-to-Prbc | AGG ACT GAC CTA GAT GTC TCT ATT TGA TTG GTT TGC C | 34 |
| SynD-A-to-SynA | AGT CCT CCT TAA CCA TCT TGA TTG ACG GAA AT | 35 |
| SynA-S | GAC CTA GAT GAG TAA AAG TGA GCG TCG TGT TTT TCT | 36 |
| SynA-S-to-SynD | TCA AGA TGG TTA AGG AGG ACT GAC CTA GAT GAG TAA AAG TGA | 37 |
| SynA-A | TCA TTA CAC CGC CGT TTC TAA AAA TTG ACC CAA ATG | 38 |
| SynB-S-Seq | CCT TCG GCC ATC AAG AGA ATG CAG AG | 39 |
| SynA-A-Seq | TGA CGC AAC TGT TCA GCC CGA CT | 40 |

Construction of pΨ228

| | | |
|---|---|---|
| S5F1S | CAC CAC TTT ACC CAT GAC GGA AGG TGG | 41 |
| S5F1A | TGT CTC GGA GTT GCT TAG GGT AAT CAT AGC A | 42 |
| S5F2S | TCG CGA ATT CCT GTT CAT CAA CAA CGG TG | 43 |
| S5F2A | AAA GCT AAA GCG ACT GAG GAA GTG CCA G | 44 |

Construction of pΨ231

| | | |
|---|---|---|
| Fats-S | AGA TAT CGC GTG CAA GGC CCA GTG | 45 |
| Fats-A | TGA TAT CAT TAA GAG ACC GAG TTT CCA TTG G | 46 |

Construction of pΨ240

| | | |
|---|---|---|
| S7F1-S | GAC TTC CAA AAC GGC GAT CAA GCC AAC C | 47 |
| S7F1-A | GTC CAT TAG GGG AGT GTC CGC CAA CA | 48 |
| S7F2-S | GGT ACC ATG CAC TGG TGG ATT ACG CC | 49 |
| S7F2-A | GGG AAA TTG TTC CGT TAA CTG TTG ATA TTC CCG GT | 50 |

Construction of pΨ243

| | | |
|---|---|---|
| ChFatB-s- | CTG AAC GAA GGA ATT ATA ACC AAA TGG TGG CTG CTG CTG CTA | 51 |
| to-Psba | GTT C | |
| PsbA-a- | GAA CTA GCA GCA GCA GCC ACC ATT TGG TTA TAA TTC CTT CGT | 52 |
| to-ChFatB | TCA G | |

Construction of pΨ248

| | | |
|---|---|---|
| S9F1S | CAA TAG GAT TCG TAG AGA TTG AGA TAC TCC ATG GCG T | 53 |
| S9F1A | AGC CTT TTT TGA GGG CTA CCT TTT GGC TGT T | 54 |
| S9F2S | GGC TCC CTA CTT TTA CGG TTA CAT TTT TGG CGA AT | 55 |
| S9F2A | CTA CAA GGA AGC AAT TTG TCG CAT ATA TTG ACC CCA A | 56 |

Segregation Checking/Sequencing

| | | |
|---|---|---|
| FadD-F2-Seq | ATA AGT TTG GGT TAC CAC TGG TCG TTT GAG CTT C | 57 |
| FadD-F1-Sequ | CTTCCCTTCTTCCTTCCATCTGATTATGGT | 58 |
| S4-seg 100-S | TGGCTCCCTGACCAATTTTTCGG | 59 |
| S4-seg 100-A | CCA GGC AAT TTC CTC CGG TTT ACC | 60 |
| S5100S | TCA TCG TGT TAA CAG CGG TAT GCT TCT AGT CT | 61 |
| S5100A | CAA AGG TAC CGC TAA TAC CTG TAA GTT CTA CGA GG | 62 |
| S7 Seg 51S | GGG GAT CAA TTG CGT CTC TGT GGC | 63 |
| S7 Seg 90A | CAA AGC GTT GAC CGT GCC AGT TTT TGA C | 64 |
| S9-S68 | CCC TAA AAA AAG TCA AAC TAA CCT TTC CCA GGG TGG | 65 |
| S9-A71 | CTT CTT TGG CCA CAT CTT CGC CTA GTA AAT GGT T | 66 |

Construction of pΨ200

| | | |
|---|---|---|
| LipA-S | TACCTGGGTTCAGGGTTGTGAT | 67 |
| LipA-A | CAAGCTTGTGCAACAGTCGGAA | 68 |

Construction of pΨ234

| | | |
|---|---|---|
| cmpR-s | TCT GGT TCC AGA GCC TGA TTC CCT CAT AAT CAA G | 69 |
| cmpA-a | ATC CGC TCG AAT GGC GAG GTA CTC TT | 70 |
| cmpR-a | TTC TAT GAA AAA AGG CAG ACA GAA AAA TTA AAT AAG CGC CA | 71 |
| cmpA-S | GAA AAA ATC AAT CAA AGT TAT GGG TTC ATT CAA TCG ACG | 72 |

Construction of pΨ237, pΨ256, and pΨ257

| | | |
|---|---|---|
| Fol-s | TTG AGG AGG TGT GAT GGA AGT ATC CCA AGA TCT GTT TAA T | 73 |
| Fol-a | CTT CAT TGA ATT AAT CTC CTC ATT AAC TGA AAC CGC CGG CGT | 74 |
| | TA | |
| Shl-s | CAG TTA ATG AGG AGA TTA ATT CAA TGA AGC CTA CCG TTA AAG | 75 |
| | CTG | |
| Shl-a | TCG ATA TCA TTA GGC GTT TTT GGT AGA TTC GGC TTT TTC G | 76 |

Construction of pΨ244

| | | |
|---|---|---|
| S8F1-S | GGT TCG ACA AAT TTG AGA TAG TTT TGT GGC AAA G | 77 |
| S8F1-A | T GTA AAT TGT CTC CTT GGT TGA AAT TTA TGG GGA | 78 |
| S8F2-S | AGG AGA ACA TTA TGG ATT TTT TGT CCA ATT TCT TGA CG | 79 |
| S8F2-A | CCA TAA TCA GCA TCA AGA TGG AAA GTA GTC CTC G | 80 |

Construction of pΨ252 and pΨ258

| | | |
|---|---|---|
| GPLRP2-S | TAT GAA ATT GTT TGC CTG GAC TAT TGG TTT ACT GCT G | 81 |
| GPLRP2-A | TTT AGC AGG GGC TCA GAG TCT GCT CGA CGT T | 82 |

Construction of pΨ262

| | | |
|---|---|---|
| P2213-S | AGG AGG ATA TAA TGC CAG AAA AAC ATG ATC TGT TAA CCG | 83 |
| P2215-A | aTTA TTT TAA GCA CTG ACT CCT GAT GTA CTC CTG CAG | 84 |
| GPL-END-FOR | AAG ACG GGA CCA AAT ATA ACT TCT GCT CCA GCG AT | 85 |
| P2213-BACK | ATG CCC TGT TCC TTT GCC GCC ATC ATG | 86 |

Construction of other deletions

| | | |
|---|---|---|
| S22F1S | GAT GAA AGT TTG GTA AGG CGT TTT ATG TCA GTG C | 87 |
| S22F1A | TTT CCA GCA CCA GAA TAA TGT TGT TAT CAT GTT TGC | 88 |
| S22seg87S | AAA CCA CCC GTA TTG CCT ATT TTC GTC CCA TTA TT | 89 |
| S22F2S | ATA CCG GTA ACA ATA CTT ACA AGG CAG TGC AAA | 90 |
| S22seg81A | TAT TTA ATC CTT GTA AAA TGG GGC CAA TGG CGA | 91 |
| S22F2A | CTA GTT TCA GTC CCT GCT ACT ACT ACC ATG GC | 92 |
| S9F1S | CAA TAG GAT TCG TAG AGA TTG AGA TAC TCC ATG GCG T | 93 |
| S9F1A | AGC CTT TTT TGA GGG CTA CCT TTT GGC TGT T | 94 |
| S9F2S | GGC TCC CTA CTT TTA CGG TTA CAT TTT TGG CGA AT | 95 |
| S9F2A | CTA CAA GGA AGC AAT TTG TCG CAT ATA TTG ACC CCA A | 96 |
| S9-S68 | CCC TAA AAA AAG TCA AAC TAA CCT TTC CCA GGG TGG | 97 |
| S9-A71 | CTT CTT TGG CCA CAT CTT CGC CTA GTA AAT GGT T | 98 |
| S23F2S | TGG CAA GGC TAC TTT TGA CAC ATT GAG CA | 99 |
| S23Seg74A | AAT TAG TTT CCC CCC AAA AGG GTC GTC GTA A | 100 |
| S23F2A | CCT TAC CCC TAA CTA ACT TAC TCT AAC AAG AAG TCC C | 101 |
| S23F1A | CGC TGG CCA TGG TAA GCA AAG AGA AAT AAC A | 102 |
| S23Seg84S | GAT AAA TGG TTT ACC ATG TTA GGC CTA AGC ACG C | 103 |
| S23F1S | CTA GCC AAG GTG AAT CAA AAC CTA GGG GAA C | 104 |
| S24F2S | CTG AGT TTG CTG AAT TTT TAG TGG AAT TGG GCA TTG | 105 |
| S24Seg95A | CAC CTC TGC AAT GCG TAG AAC AGT TTT CAG C | 106 |
| S24F2A | ACA TCC CGA CAA TAT TCG ACT CAA ACA AAT TCC T | 107 |
| S24F1A | GGT ACA TTC ACC CCT TTG TTG GTC AAC TG | 108 |
| S24Seg114S | TTT AAT CCT CTG GTT TGA AGA GGT GGG CAC | 109 |
| S24F1S | CGT AGC TCC AAC GGA GAC CTA ATT ATT ATG CC | 110 |
| S25F1A | GCA AAG GGA GGT TGC CAA TCC AAA ACC | 111 |
| S25Seg95S | AGC AAT CCG GAG AAG TTT TGG GGT GAG | 112 |
| S25F1S | CTG TTG ACT TCA TTA AGT TCA GCG GGG ATT TTT | 113 |
| S25F2S | AAT CCG TTT CAC CGA TGT GTT ACC CAA AAC | 114 |
| S25Seg75A | GAG GCT AAA CTC CGC AAC AGA CGA C | 115 |
| S25F2A | TTG GTA GTG AAA TTA TTA TTG GCG ATC GCC AAT CC | 116 |
| S6-66S | TTT ACC TAG CCT AGG AGC CCC AGT G | 117 |
| S6-99A | GCA CAA CCC TTC AGA AAT AAA GCC CCT | 118 |
| S8-90S | CTG GAA AGA TCA AGC AAA CTG CCG AAG ATT CA | 119 |
| S8-60A | ACT GCA ATT GTC CCA CGA AGT CCG TCA A | 120 |

TABLE 15
Synthesized DNA segments

| | SEQ. |
|---|---|
| SEQUENCE | ID NO. |
| *P_{psbA2} UC fatB1 | 121 |
| AGatATcGCGTGCAAGGCCCAGTGATCAATTTCATTATTTTTCATTATTTCATCTCCATTGTCCCTGAAAATCAGTTGT | |
| GTCGCCCCTCTACACAGCCCAGAACTATGGTAAAGGCGCACGAAAAACCGCCAGGTAAACTCTTCTCAACCCCCAAAA | |
| C | |
| GCCCTCTGTTTACCCATGGAAAAAACGACAATTACAAGAAAGTAAAACTTATGTCATCTATAAGCTTCGTGTATATTAA | |
| 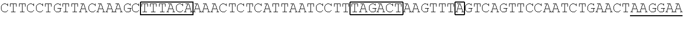 | |
| TTATAACCAA | |
| ATG GCt ACC ACC TCT TTA GCT TCC GCc TTt TGC TCG ATG AAA GCT GTA ATG TTa GCT CGT | |
| GAT GGt CGG GGt ATG AAA CCt CGT AGt AGT GAT TTG CAA CTc CGT GCG GGA AAT GCG CCT | |
| ACC TCT TTG AAA ATG ATC AAT GGG ACC AAA TTC AGT TAT ACG GAG AGC TTG AAA CGG TTG | |
| CCT GAT TGG AGC ATG CTC TTT GCT GTT ATC ACC ACC ATC TTT TCG GCT GCT GAG AAA CAA | |
| TGG ACt AAT CTA GAG TGG AAG CCG AAA CCG AAG CTA CCC CAG TTG CTT GAT GAT CAT TTT | |
| GGA CTG CAT GGG TTA GTT TTC CGG CGC ACC TTT GCC ATC CGG TCT TAT GAa GTT GGA CCT | |
| GAT CGC TCC ACC TCT ATT CTG GCT GTT ATG AAT CAT ATG CAG GAG GCT ACC CTT AAT CAT | |
| GCG AAA AGT GTG GGA ATT CTA GGA GAT GGA TTC GGG ACG ACG CTA GAG ATG AGT AAG CGG | |
| GAT CTG ATG TGG GTT GTT CGG CGC ACG CAT GTT GCT GTT GAA CGG TAC CCT ACT TGG GGT | |
| GAT ACT GTA GAA GTA GAG TGC TGG ATT GGT GCT TCT GGA AAT AAT GGC ATG CGT CGT GAT | |
| TTC CTT GTC CGG GAC TGC AAA ACC GGC GAA ATT CTT ACT CGC TGT ACC AGC CTT TCG GTG | |
| CTG ATG AAT ACT CGC ACt CGt CGT TTG TCC ACC ATt CCT GAT GAA GTT CGT GGt GAa ATA | |
| GGG CCT GCT TTC ATc GAT AAT GTT GCT GTg AAA GAC GAT GAA ATT AAG AAA CTA CAA AAA | |
| CTC AAT GAT AGC ACT GCC GAT TAT ATt CAA GGA GGT TTG ACc CCT CGT TGG AAT GAT TTG | |
| GAT GTC AAT CAA CAT GTT AAC AAC CTC AAA TAC GTT GCC TGG GTT TTT GAG ACC GTC CCc | |
| GAt TCC ATC TTT GAG AGT CAT CAT ATT TCC AGC TTC ACT CTT GAA TAT CGT CGT GAG TGt | |
| ACc CGT GAT AGC GTG CTG CGG TCC CTG ACC ACT GTC TCT GGT GGC TCG TCG GAG GCT GGG | |
| TTA GTT TGC GAT CAT TTG CTC CAA CTT GAA GGT GGG TCT GAG GTA TTG CGT GCC AGA ACT | |
| GAG TGG CGG CCT AAA CTT ACC GAT AGT TTC CGC GGc ATT AGT GTT ATT CCC GCC GAA CCG | |
| CGC GTG TAA tgatatca | |
| *P_{psbA2} Ch fatB2 | 122 |
| AGatATcGCGTGCAAGGCCCAGTGATCAATTTCATTATTTTTCATTATTTCATCTCCATTGTCCCTGAAAATCAGTTGT | |
| GTCGCCCCTCTACACAGCCCAGAACTATGGTAAAGGCGCACGAAAAACCGCCAGGTAAACTCTTCTCAACCCCCAAAA | |
| C | |
| GCCCTCTGTTTACCCATGGAAAAAACGACAATTACAAGAAAGTAAAACTTATGTCATCTATAAGCTTCGTGTATATTAA | |
| 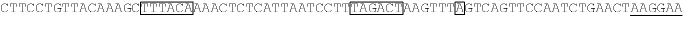 | |
| TTATAACCAA | |
| ATG GTG GCT GCT GCT GCT AGT TCC GCT TTC TTC CCT GTT CCA GCC CCc GGA GCC TCC CCT | |
| AAA CCC GGG AAG TTC GGA AAT TGG CCC AGT AGC TTG AGC CCT TCC TTC AAG CCC AAG TCA | |
| ATC CCC AAT GGC GGA TTT CAG GTT AAG GCT AAT GAC AGC GCC CAT CCA AAa GCc AAt GGT | |
| TCT GCc GTT AGT CTA AAG TCT GGC AGC CTC AAC ACT CAa GAa GAC ACT AGT TCC TCC CCT | |
| CCT CCT CGG ACT TTC CTT CAt CAG TTG CCT GAT TGG AGT CGt CTT CTG ACT GCT ATt ACc | |
| ACC GTG TTC GTG AAA TCT AAG CGT CCT GAC ATG CAT GAT CGG AAA TCC AAG CGT CCT GAC | |
| ATG CTG GTG GAC TCC TTT GGG TTG GAG AGT ACT GTT CAG GAT GGc tTa GTG TTC CGA CAG | |
| AGT TTT TCC ATT CGT TCT TAT GAA ATA GGC ACT GAT CGA ACG GCC TCT ATA GAG ACC CTT | |
| ATG AAC CAC TTG CAG GAA ACC TCT CTC AAT CAT TGT AAG AGT ACC GGT ATT CTC CTT GAC | |
| GGC TTC GGT CGT ACT CTT GAG ATG TGT AAA CGC GAC CTC ATT TGG GTG GTA ATT AAA ATG | |
| CAG ATC AAG GTG AAT CGC TAT CCA GCT TGG GGC GAT ACT GTC GAG ATC AAT ACC CGt TTC | |
| agC CGG TTG GGG AAA ATt GGT ATG GGT CGC GAT TGG CTA ATT AGT GAT TGC AAC ACC GGA | |
| GAA ATT CTT GTA CGG GCT ACG AGC GCG TAT GCC ATG ATG AAT CAA AAG ACG CGG AGA CTC | |
| TCC AAA CTT CCA TAC GAG GTT CAC CAG GAG ATT GTG CCT CTT TTT GTC GAC TCT CCT GTC | |
| ATT GAA GAC AGT GAT CTG AAA GTG CAT AAG TTT AAA GTG AAG ACT GGT GAc agC ATT CAA | |
| AAG GGT CTA ACT CCG GGG TGG AAT GAC TTG GAT GTC AAT CAG CAC GTA AGC AAC GTG AAG | |
| TAC ATT GGG TGG ATT CTC GAG AGT ATG CCA ACA GAA GTT TTG GAG ACC CAG GAG CTA TGC | |
| TCT CTC GCC CTT GAA TAT CGC CGG GAA TGC GGA CGC GAC AGT GTG CTG GAG TCC GTG ACC | |
| GCT ATG GAT CCC TCC AAA GTT GGA GTC CGT TCT CAG TAC CAG CAC CTT CTG CGG CTT GAG | |
| GAT GGG ACT GCT ATC GTG AAC GGT GCT ACT GAG TGG CGG CCG AAG AAT GCA GGA GCT AAC | |
| GGG GCG ATc agC ACG GGA AAG ACT TCC AAT GGA AAC TCG GTC TCT TAA tgatatca | |
| P _{psbA2*} CC fatB1 | 123 |
| AGatATcGCGTGCAAGGCCCAGTGATCAATTTCATTATTTTTCATTATTTCATCTCCATTGTCCCTGAAAATCAGTTGT | |
| GTCGCCCCTCTACACAGCCCAGAACTATGGTAAAGGCGCACGAAAAACCGCCAGGTAAACTCTTCTCAACCCCCAAAA | |
| C | |
| GCCCTCTGTTTACCCATGGAAAAAACGACAATTACAAGAAAGTAAAACTTATGTCATCTATAAGCTTCGTGTATATTAA | |
| 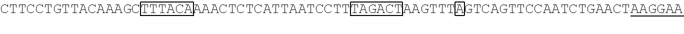 | |
| TTATAACCAA | |
| ATG AAA ACT ACT TCT CTC GCC TCT GCC TTC TGT TCT ATG AAA GCT GTT ATG CTG GCg CGG | |
| GAT GGT CGC GGT ATG AAA CCC CGT TCC AGT GAT CTG CAA TTA CGG GCT GGC AAC GCT CAG | |
| ACC TCC TTG AAG ATG ATT AAC GGC ACT AAA TTC AGT TAT ACC GAA TCT TTG AAG AAA CTC | |
| CCC GAT TGG AGC ATG TTG TTC GCC GTG ATT ACC ACC ATt TTt AGT GCT GCC GAA AAA CAA | |
| TGG ACC AAT CTC GAA TGG AAA CCC AAA CCC AAC CCC CCG CAG CTG CTC GAT GAC CAT TTT | |
| GGc CCC CAC GGC TTG GTG TTT CGG CGT ACC TTC GCT ATC CGG TCT TAT GAA GTC GGT CCC | |
| GAT CGG AGC ACT TCC ATC GTC GCT GTT ATG AAT CAC TTG CAA GAA GCC GCT TTG AAC CAt | |
| GCT AAA tct GTT GGG ATT CTG GGT GAT GGC TTC GGT ACC ACT CTG GAG ATG AGT AAG CGC | |
| GAT CTG ATC TGG GTA GTA AAG CGT ACt CAT GTG GCC GTG GAA CGT TAt CCg GCC TGG GGT | |
| GAT ACC GTA GAA GTG GAG TGT TGG GTA GGC GCC TCC GGT AAC AAC GGT CGG CGT CAC GAC | |
| TTC TTG GTG CGT GAC TGT AAA ACt GGc GAG ATC CTG ACC CGC TGT ACT TCC CTG AGC GTT | |
| ATG ATG AAC ACC CGG ACC CGT CGC TTA TCC AAG ATT CCC GAA GAA GTT CGC GGG GAA ATT | |
| GGt CCt GCT TTC ATT GAT AAC GTT GCT GTT AAG GAT GAG GAG ATT AAA AAG CCG CAA AAG | |
| CTC AAT GAT TCT ACC GCC GAT TAC ATT CAA GGG GGT CTG ACT CCC CGT TGG AAT GAT CTG | |
| GAT ATT AAT CAG CAT GTG AAT AAC ATC AAA TAT GTG GAT TGG ATT CTG GAG ACT GTG CCC | |
| GAC TCT ATT TTC GAG TCC CAC CAC ATT agc AGT TTT ACC ATT GAA TAT CGT CGC GAA TGT | |
| ACT ATG GAC AGT GTT TTG CAA TCC CTG ACC ACC GTC TCC GGC GGT TCC TCT GAA GCT GGC | |
| CTG GTG TGC GAA CAC CTC TTG CAA CTC GAA GGC GGT AGT GAA GTg CTc CGt GCC AAG ACC | |
| GAA TGG CGG CCC AAA TTG ACC GAC TCC TTT CGC GGG ATT TCT GTG ATT CCC GCC GAA TCC | |
| TCC GTC TAA GATATCAT | |
| fol RBS shl | 124 |
| TTCCCAATTTGAGGaGGTGTG | |
| ATG GAA GTa TCC CAa GAT CTG TTT AAT CAA TTC AAT CTG TTC GCT CAA TAC TCC GCg GCT | |
| GCT TAC TGT GGg AAA AAT AAC GAT GCT CCT GCg GGc ACC AAT ATT ACt TGT ACC GGT AAC | |
| GCT TGC CCT GAA GTG GAg AAg GCT GAC GCC ACC TTT TTG TAC AGC TTC GAA GAT AGC GGC | |
| GTA GGC GAT GTG ACT GGt TTC cTc GCC TTG GAT AAT ACC AAT AAA TTG ATT GTT CTC TCT | |
| TTC CGT GGC agt GAc TCC ATT GAG AAT TGG ATT GCC AAC TTG AAc TTt TGG TTG AAG AAA | |
| ATC AAt GAt ATT TGT TCC GGc TGT CGG GGt CAC GAT GGT TTC ACC AGC AGC TGG CGT TCC | |
| GTG GCC GAC ACC CTC CGG CAA AAG GTg GAa GAT GCT GTg CGc GAA CAt CCt GAT TAC CGC | |
| GTT GTT TTT ACC GGG CAC TCC CTG GGC GGG GCC TTG GCC ACC GTA GCC GGT GCT GAC CTG | |
| CGC GGT AAC GGC TAC GAT ATT GAT GTG TTC TCC TAT GGG GCT CCC CGG GTT GGG AAC CGG | |
| GCT TTT GCt GAg TTT TTG ACC GTt CAa ACC GGc GGT ACC TTG TAT CGG ATC ACC CAT ACC | |
| AAT GAT ATT GTT CCC CGC CTG CCC CCT CGT GAA TTC GGT TAT AGC CAC TCC TCC CCC GAA | |
| TAC TGG ATT AAA AGC GGC ACC TTG GTT CCC GTG ACC CGT AAC GAC ATT GTA AAA ATT GAG | |
| GGC ATT GAC GCC ACC GGT GGT AAT AAT CAA CCT AAT ATC CCC GAT ATC TTG GCT CAT CTG | |
| TGG TAT TTT CAA GCC ACC GAC GCT TGT AAC GCC GGC GGT TTC AGT TAA | |
| TGAGGAGATTAATTCA | |
| ATGAAGCCTACCGTTAAAGCTGCTCCCGAGGCTGTTCAGAACCCGGAAAACCCGAAAAACAAGGACCCCTTTGTGTTT | |
| GTGCACGGCTTTACCGGTTTTGTGGGGGAGG | |
| TTGCTGCGAAAGGTGAGAATCACTGGGGCGGCACCAAAGCCAATCTGCGCAACCATTTGCGGAAAGCTGGTTACGAAA | |
| CCTACGAAGCCTCCGTATCCGCCTTGGCCTC | |
| CAATCACGAACGTGCTGTGGAACTGTACTATTATCTGAAAGGTGGTCGGGTAGACTATGGTGCTGCCCATTCCGAAAAA | |
| TATGGCCATGAGCGTTACGGGAAAACTTAT | |
| GAAGGTGTGCTGAAAGATTGGAAACCCGGGCACCCCGTACACTTTATCGGTCATTCCATGGGTGGTCAGACCATTCGG | |
| CTGCTGGAACATTATCTGCGCTTTGGTGATA | |
| AAGCCGAAATTGCCTATCAACAACAGCACGGGGGTATTATTAGCGAATTATTTAAGGGCGGTCAAGACAACATGGTGAC | |
| CTCTATCACTACTATTGCCACCCCTCACAA | |
| TGGTACCCATGCTTCTGACGATATTGGCAATACCCCGACTATCCGGAACATTCTGTATAGCTTCGCCCAAATGTCCAGT | |
| CATCTGGGCACCATCGACTTTGGGATGGAC | |
| CATTGGGGTTTCAAGCGGAAAGATGGCGAGAGTCTGACCGATTATAATAAGCGGATTGCCGAGAGCAAAATCTGGGAT | |
| TCTGAAGATACTGGGCTGTATGACCTGACCC | |
| GTGAAGGCGCCGAGAAAATCAACCAGAAAACCGAATTGAATCCCAATATCTATTACAAAACCTACACTGGGGTGGCTAC | |
| CCATGAAACTCAGTTAGGCAAACACATCGC | |
| GGACCTCGGCATGGAATTTACCAAAATCCTCACCGGCAACTATATCGGGAGCGTAGACGATATTCTGTGGCGGCCCAA | |
| TGATGGTTTGGTGAGCGAAATCTCCAGCCAA | |
| CACCCCAGCGATGAGAAGAACATTTCCGTAGACGAAAACTCCGAACTGCATAAGGGTACCTGGCAGGTCATGCCTACC | |
| ATGAAAGGGTGGGACCACTCCGATTTTATTG | |
| GTAATGACGCCCTGGATACCAAACACTCCGCCATCGAACTCACCAACTTTTATCATAGCATTTCTGACTACTTGATGCG | |
| GATCGAAAAAGCCGAATCTACCAAAAACGC | |
| CTAATGATATCGA | |
| gpl | 125 |
| ATG Aaa TTG TTT GCC TGG ACT ATT GGT TTA CTG CTg ctg GCC ACt GTG CGC GGc GCT GAG | |
| GTc TGT TAT TCT CAt TTG GGC TGc TTT TCC GAC GAG AAA CCG TGG GCg GGT ACC TCT CAA | |
| CGG CCC ATC AAg AGT TTG CCg TCC GAC CCT AAA AAG ATT AAt ACC CGG TTC TTG CTG TAC | |
| ACC AAt GAa AAt CAA AAT TCC TAC CAA CTG ATC ACC GCT ACT GAT ATT GCC ACC ATC AAA | |
| GCC AGT AAC TTC AAT CTc AAC CGc AAA ACC CGC TTC ATT ATT CAC GGT TTC ACC GAC AGC | |
| GGT GAG AAC TCT TGG CTG AGT GAT ATG TGT AAA AAC ATG TTC CAA GTT GAA AAA GTG AAT | |
| TGC ATT TGC GTG GAT TGG AAA GGC GGT TCC AAG GCT CAA TAC AGT CAG GCT TCC CAG AAT | |
| ATT CGG GTG GTC GGT GCC GAA GTT GCC TAT TTA GTG CAA GTA CTG AGC ACC TCC CTG AAC | |
| TAT GCC CCG GAA AAC GTA CAT ATT ATT GGT CAC TCC CTC GGC GCC CAC ACC GCT GGG GAA | |
| GCC GGG AAG CGG CTG AAC GGG CTG GTA GGG CGG ATT ACC GGc cTc GAC CCC GCC GAA CCC | |
| TAC TTT CAA GAT ACC CCC GAA GAA GTC CGG CTG GAT CCC TCT GAC GCT AAA TTT GTG GAC | |
| GTG ATT CAC ACT GAT ATT AGC CCC ATT CTG CCT AGT CTG GGT TTC GGT ATG TCC CAG AAG | |
| GTC GGT CAC ATG GAC TTC TTC CCC AAC GGC GGC AAA GAT ATG CCC GGG TGC AAA ACC GGC | |
| ATC TCC TGC AAC CAC CAC CGG AGT ATT GAA TAT TAT CAC AGC AGT ATT TTG AAC CCC GAA | |
| GGT TTt TTa GGC TAC CCG TGT GCT TCC TAC GAT GAA TTc CAA GAA TCt GGG TGC TTC CCC | |
| TGT CCC GCT AAA GGT TGT CCT AAA ATG GGT CAC TTT GCC GAC CAG TAC CCC GGg AAG ACC | |
| AAT GCT GTC GAA CAA ACC TTc TTC CTC AAC ACC GGg GCg TCC GAT AAT TTC ACC CGG TGG | |
| CGC TAT AAA GTA ACC GTT ACC CTG AGT GGC GAA AAA GAC CCG TCC GGT AAC ATC AAT GTG | |
| GCT TTG TTA GGT AAA AAC GGG AAC TCT GCT CAA TAC CAG GTT TTC AAG GGC ACC CTG AAG | |
| CCC GAC GCC TCC TAT ACT AAT TCC ATT GAT GTC GAA CTC AAC GTT GGG ACC ATT CAA AAA | |
| GTG ACC TTC CTG TGG AAA CGG AGC GGG ATC TCC GTC TCT AAA CCC AAG ATG GGT GCT TCt | |
| CGc ATT ACt GTT CAA AGT GGT AAA GAC GGG ACC AAA TAT AAC TTC TGC TCC AGC GAT ATT | |
| GTG CAA GAA AAC GTC GAG CAG ACT CTG AGC CCC TGC taa | |

What is claimed is:

1. A recombinant bacterium, wherein the bacterium is capable of producing fatty acids and is capable of the inducible release of fatty acids from a cellular membrane.

2. The recombinant bacterium of claim 1, wherein the bacterium further comprises at least one mutation that prevents fatty acid degradation.

3. The recombinant bacterium of claim 1, wherein the inducible release is induced by CO₂ limitation.

4. The recombinant bacterium of claim 1, wherein the inducible release is induced by a metal or metal ion.

5. The recombinant bacterium of claim 1, wherein the bacterium further comprises at least one modified polar cell layer.

6. The recombinant bacterium of claim 5, wherein the at least one polar cell layer is the peptidoglycan layer.

7. The recombinant bacterium of claim 5, wherein the at least one polar cell layer is the outer membrane layer.

8. The recombinant bacterium of claim 5, wherein the modified polar cell layer is eliminated.

9. The recombinant bacterium of claim 1, wherein the bacterium expresses a nucleic acid encoding a thioesterase.

10. A recombinant bacterium, wherein the bacterium is modified to encode one or more thioesterases that specify synthesis and secretion of saturated C10 to C14 chain fatty acids that are secreted more efficiently than saturated and unsaturated C16 and C18 chain fatty acids.

11. The recombinant bacterium of claim 10, wherein the bacterium further comprises at least one mutation that prevents fatty acid degradation.

12. The recombinant bacterium of claim 10, wherein the bacterium further comprises at least one modified polar cell layer.

13. The recombinant bacterium of claim 12, wherein the at least one polar cell layer is the peptidoglycan layer.

14. The recombinant bacterium of claim 12, wherein the at least one polar cell layer is the outer membrane layer.

15. The recombinant bacterium of claim 12, wherein the modified polar cell layer is eliminated.

16. The recombinant bacterium of claim 10, wherein the bacterium is further capable of the inducible release of fatty acids from a cellular membrane.

17. The recombinant bacterium of claim 16, wherein the inducible release is induced by CO₂ limitation.

18. The recombinant bacterium of claim 16, wherein the inducible release is induced by a metal or metal ion.

19. A recombinant bacterium, wherein the bacterium is capable of producing fatty acids and comprises at least one modified polar cell layer.

20. The recombinant bacterium of claim 19, wherein the bacterium further comprises at least one mutation that prevents fatty acid degradation.

21. The recombinant bacterium of claim 19, wherein the at least one polar cell layer is the peptidoglycan layer.

22. The recombinant bacterium of claim 19, wherein the at least one polar cell layer is the outer membrane layer.

23. The recombinant bacterium of claim 19, wherein the modified polar cell layer is eliminated.

24. The recombinant bacterium of claim 19, wherein the bacterium expresses a nucleic acid encoding a thioesterase.

25. The recombinant bacterium of claim 19, wherein the bacterium is further capable of the inducible release of fatty acids from a cellular membrane.

26. The recombinant bacterium of claim 25, wherein the inducible release is induced by CO₂ limitation.

27. The recombinant bacterium of claim 25, wherein the inducible release is induced by a metal or metal ion.

28. A method of producing fatty acids, the method comprising culturing a bacterium of claim 1.

29. A method of producing fatty acids, the method comprising culturing a bacterium of claim 3 under CO₂ limitation.


SEQUENCE LISTING (125 sequences)

SEQ ID NO: 1 - DNA, 23 bp, Artificial Sequence
  SYNTHESIZED
taaactctgtaggccagcggcaa

SEQ ID NO: 2 - DNA, 26 bp, Artificial Sequence
  SYNTHESIZED
cgtcaatgcctagacctagcagtacc

SEQ ID NO: 3 - DNA, 27 bp, Artificial Sequence
  SYNTHESIZED
aaggatttccgttttatcccagcacca

SEQ ID NO: 4 - DNA, 27 bp, Artificial Sequence
  SYNTHESIZED
gtaattgccacagacaagcgtattcgg

SEQ ID NO: 5 - DNA, 33 bp, Artificial Sequence
  SYNTHESIZED
accatatgcatcctaggcctattaatattccgg

SEQ ID NO: 6 - DNA, 25 bp, Artificial Sequence
  SYNTHESIZED
gaattaggatccgtcgacctgcagg

SEQ ID NO: 7 - DNA, 27 bp, Artificial Sequence
  SYNTHESIZED
gagaattccagacgactacgggcaaag

SEQ ID NO: 8 - DNA, 31 bp, Artificial Sequence
  SYNTHESIZED
aacgtgtccgccatcacaccacctcaaattg

SEQ ID NO: 9 - DNA, 31 bp, Artificial Sequence
  SYNTHESIZED
gaggtggtgtgatggcggacacgttattgat

SEQ ID NO: 10 - DNA, 36 bp, Artificial Sequence
  SYNTHESIZED
caaaaggagcaatgtgttatttgtcatcatcgtctt

SEQ ID NO: 11 - DNA, 35 bp, Artificial Sequence
  SYNTHESIZED
gatgatgacaaataacacattgctccttttgtgcg

SEQ ID NO: 12 - DNA, 26 bp, Artificial Sequence
  SYNTHESIZED
acggatccgcaagcagtgaaagatag

SEQ ID NO: 13 - DNA, 27 bp, Artificial Sequence
  SYNTHESIZED
caaatggcggacacgttattgattctg

SEQ ID NO: 14 - DNA, 34 bp, Artificial Sequence
  SYNTHESIZED
ctttgtagtctgagtcatgatttactaaaggctg

SEQ ID NO: 15 - DNA, 26 bp, Artificial Sequence
  SYNTHESIZED
attataaccaaatggcggacacgtta

SEQ ID NO: 16 - DNA, 24 bp, Artificial Sequence
  SYNTHESIZED
tccccattgccccaaaatacatcc

SEQ ID NO: 17 - DNA, 35 bp, Artificial Sequence
  SYNTHESIZED
caataacgtgtccgccatttggttataattcctta

SEQ ID NO: 18 - DNA, 27 bp, Artificial Sequence
  SYNTHESIZED
gactgcaggtcattgccgataaagttg

SEQ ID NO: 19 - DNA, 31 bp, Artificial Sequence
  SYNTHESIZED
agtctagataatgtacaggtcaagctggtct

SEQ ID NO: 20 - DNA, 30 bp, Artificial Sequence
  SYNTHESIZED
gagagctcattgacaccgaaatgactttgg

SEQ ID NO: 21 - DNA, 29 bp, Artificial Sequence
  SYNTHESIZED
gagaattctttgcatttccgaaaccaccc

SEQ ID NO: 22 - DNA, 30 bp, Artificial Sequence
  SYNTHESIZED
gatctagaattgacaccgaaatgactttgg

SEQ ID NO: 23 - DNA, 29 bp, Artificial Sequence
  SYNTHESIZED
gaggtacctttgcatttccgaaaccaccc

SEQ ID NO: 24 - DNA, 26 bp, Artificial Sequence
  SYNTHESIZED
taggctgtggttccctaggcaacagt

SEQ ID NO: 25 - DNA, 41 bp, Artificial Sequence
  SYNTHESIZED
tccgtaaagttaatagccattgaattaatctcctacttgac

SEQ ID NO: 26 - DNA, 40 bp, Artificial Sequence
  SYNTHESIZED
aggagattaattcaatggctattaactttacggaactgcg

SEQ ID NO: 27 - DNA, 42 bp, Artificial Sequence
  SYNTHESIZED
cattgaattaatctcctctagggtttaatccacattagggtt

SEQ ID NO: 28 - DNA, 45 bp, Artificial Sequence
  SYNTHESIZED
cctagaggagattaattcaatgcaattcgccaaaattttaattgc

SEQ ID NO: 29 - DNA, 34 bp, Artificial Sequence
  SYNTHESIZED
ctctccattgacctagggtgttaaatgctcttcg

SEQ ID NO: 30 - DNA, 41 bp, Artificial Sequence
  SYNTHESIZED
gtggattaaaccctagaggagattaattcaatgcaattcgc

SEQ ID NO: 31 - DNA, 39 bp, Artificial Sequence
  SYNTHESIZED
ctttacttatggcaatgctctccattgacctagggtgtt

SEQ ID NO: 32 - DNA, 31 bp, Artificial Sequence
  SYNTHESIZED
aacaccctaggtcaatggagagcattgccat

SEQ ID NO: 33 - DNA, 38 bp, Artificial Sequence
  SYNTHESIZED
caatcaaatagagacatctaggtcagtcctccataaac

SEQ ID NO: 34 - DNA, 37 bp, Artificial Sequence
  SYNTHESIZED
aggactgacctagatgtctctatttgattggtttgcc

SEQ ID NO: 35 - DNA, 32 bp, Artificial Sequence
  SYNTHESIZED
agtcctccttaaccatcttgattgacggaaat

SEQ ID NO: 36 - DNA, 36 bp, Artificial Sequence
  SYNTHESIZED
gacctagatgagtaaaagtgagcgtcgtgtttttct

SEQ ID NO: 37 - DNA, 42 bp, Artificial Sequence
  SYNTHESIZED
tcaagatggttaaggaggactgacctagatgagtaaaagtga

SEQ ID NO: 38 - DNA, 36 bp, Artificial Sequence
  SYNTHESIZED
tcattacaccgccgtttctaaaaattgacccaaatg

SEQ ID NO: 39 - DNA, 26 bp, Artificial Sequence
  SYNTHESIZED
ccttcggccatcaagagaatgcagag

SEQ ID NO: 40 - DNA, 23 bp, Artificial Sequence
  SYNTHESIZED
tgacgcaactgttcagcccgact

SEQ ID NO: 41 - DNA, 27 bp, Artificial Sequence
  SYNTHESIZED
caccactttacccatgacggaaggtgg

SEQ ID NO: 42 - DNA, 31 bp, Artificial Sequence
  SYNTHESIZED
tgtctcggagttgcttagggtaatcatagca

SEQ ID NO: 43 - DNA, 29 bp, Artificial Sequence
  SYNTHESIZED
tcgcgaattcctgttcatcaacaacggtg

SEQ ID NO: 44 - DNA, 28 bp, Artificial Sequence
  SYNTHESIZED
aaagctaaagcgactgaggaagtgccag

SEQ ID NO: 45 - DNA, 24 bp, Artificial Sequence
  SYNTHESIZED
agatatcgcgtgcaaggcccagtg

SEQ ID NO: 46 - DNA, 31 bp, Artificial Sequence
  SYNTHESIZED
tgatatcattaagagaccgagtttccattgg

SEQ ID NO: 47 - DNA, 28 bp, Artificial Sequence
  SYNTHESIZED
gacttccaaaacggcgatcaagccaacc

SEQ ID NO: 48 - DNA, 26 bp, Artificial Sequence
  SYNTHESIZED
gtccattaggggagtgtccgccaaca

SEQ ID NO: 49 - DNA, 26 bp, Artificial Sequence
  SYNTHESIZED
ggtaccatgcactggtggattacgcc

SEQ ID NO: 50 - DNA, 35 bp, Artificial Sequence
  SYNTHESIZED
gggaaattgttccgttaactgttgatattcccggt

SEQ ID NO: 51 - DNA, 46 bp, Artificial Sequence
  SYNTHESIZED
ctgaacgaaggaattataaccaaatggtggctgctgctgctagttc

SEQ ID NO: 52 - DNA, 46 bp, Artificial Sequence
  SYNTHESIZED
gaactagcagcagcagccaccatttggttataattccttcgttcag

SEQ ID NO: 53 - DNA, 37 bp, Artificial Sequence
  SYNTHESIZED
caataggattcgtagagattgagatactccatggcgt

SEQ ID NO: 54 - DNA, 31 bp, Artificial Sequence
  SYNTHESIZED
agccttttttgagggctaccttttggctgtt

SEQ ID NO: 55 - DNA, 35 bp, Artificial Sequence
  SYNTHESIZED
ggctccctacttttacggttacatttttggcgaat

SEQ ID NO: 56 - DNA, 37 bp, Artificial Sequence
  SYNTHESIZED
ctacaaggaagcaatttgtcgcatatattgaccccaa

SEQ ID NO: 57 - DNA, 34 bp, Artificial Sequence
  SYNTHESIZED
ataagtttgggttaccactggtcgtttgagcttc

SEQ ID NO: 58 - DNA, 30 bp, Artificial Sequence
  SYNTHESIZED
cttcccttcttccttccatctgattatggt

SEQ ID NO: 59 - DNA, 23 bp, Artificial Sequence
  SYNTHESIZED
tggctccctgaccaatttttcgg

SEQ ID NO: 60 - DNA, 24 bp, Artificial Sequence
  SYNTHESIZED
ccaggcaatttcctccggtttacc

SEQ ID NO: 61 - DNA, 32 bp, Artificial Sequence
  SYNTHESIZED
tcatcgtgttaacagcggtatgcttctagtct

SEQ ID NO: 62 - DNA, 35 bp, Artificial Sequence
  SYNTHESIZED
caaaggtaccgctaatacctgtaagttctacgagg

SEQ ID NO: 63 - DNA, 24 bp, Artificial Sequence
  SYNTHESIZED
ggggatcaattgcgtctctgtggc

SEQ ID NO: 64 - DNA, 28 bp, Artificial Sequence
  SYNTHESIZED
caaagcgttgaccgtgccagtttttgac

SEQ ID NO: 65 - DNA, 36 bp, Artificial Sequence
  SYNTHESIZED
ccctaaaaaaagtcaaactaacctttcccagggtgg

SEQ ID NO: 66 - DNA, 34 bp, Artificial Sequence
  SYNTHESIZED
cttctttggccacatcttcgcctagtaaatggtt

SEQ ID NO: 67 - DNA, 22 bp, Artificial Sequence
  SYNTHESIZED
tacctgggttcagggttgtgat

SEQ ID NO: 68 - DNA, 22 bp, Artificial Sequence
  SYNTHESIZED
caagcttgtgcaacagtcggaa

SEQ ID NO: 69 - DNA, 34 bp, Artificial Sequence
  SYNTHESIZED
tctggttccagagcctgattccctcataatcaag

SEQ ID NO: 70 - DNA, 26 bp, Artificial Sequence
  SYNTHESIZED
atccgctcgaatggcgaggtactctt

SEQ ID NO: 71 - DNA, 41 bp, Artificial Sequence
  SYNTHESIZED
ttctatgaaaaaaggcagacagaaaaattaaataagcgcca

SEQ ID NO: 72 - DNA, 39 bp, Artificial Sequence
  SYNTHESIZED
gaaaaaatcaatcaaagttatgggttcattcaatcgacg

SEQ ID NO: 73 - DNA, 40 bp, Artificial Sequence
  SYNTHESIZED
ttgaggaggtgtgatggaagtatcccaagatctgtttaat

SEQ ID NO: 74 - DNA, 44 bp, Artificial Sequence
  SYNTHESIZED
cttcattgaattaatctcctcattaactgaaaccgccggcgtta

SEQ ID NO: 75 - DNA, 45 bp, Artificial Sequence
  SYNTHESIZED
cagttaatgaggagattaattcaatgaagcctaccgttaaagctg

SEQ ID NO: 76 - DNA, 40 bp, Artificial Sequence
  SYNTHESIZED
tcgatatcattaggcgtttttggtagattcggctttttcg

SEQ ID NO: 77 - DNA, 34 bp, Artificial Sequence
  SYNTHESIZED
ggttcgacaaatttgagatagttttgtggcaaag

SEQ ID NO: 78 - DNA, 34 bp, Artificial Sequence
  SYNTHESIZED
tgtaaattgtctccttggttgaaatttatgggga

SEQ ID NO: 79 - DNA, 38 bp, Artificial Sequence
  SYNTHESIZED
aggagaacattatggattttttgtccaatttcttgacg

SEQ ID NO: 80 - DNA, 34 bp, Artificial Sequence
  SYNTHESIZED
ccataatcagcatcaagatggaaagtagtcctcg

SEQ ID NO: 81 - DNA, 37 bp, Artificial Sequence
  SYNTHESIZED
tatgaaattgtttgcctggactattggtttactgctg

SEQ ID NO: 82 - DNA, 31 bp, Artificial Sequence
  SYNTHESIZED
tttagcaggggctcagagtctgctcgacgtt

SEQ ID NO: 83 - DNA, 39 bp, Artificial Sequence
  SYNTHESIZED
aggaggatataatgccagaaaaacatgatctgttaaccg

SEQ ID NO: 84 - DNA, 37 bp, Artificial Sequence
  SYNTHESIZED
attattttaagcactgactcctgatgtactcctgcag

SEQ ID NO: 85 - DNA, 35 bp, Artificial Sequence
  SYNTHESIZED
aagacgggaccaaatataacttctgctccagcgat

SEQ ID NO: 86 - DNA, 27 bp, Artificial Sequence
  SYNTHESIZED
atgccctgttcctttgccgccatcatg

SEQ ID NO: 87 - DNA, 34 bp, Artificial Sequence
  SYNTHESIZED
gatgaaagtttggtaaggcgttttatgtcagtgc

SEQ ID NO: 88 - DNA, 36 bp, Artificial Sequence
  SYNTHESIZED
tttccagcaccagaataatgttgttatcatgtttgc

SEQ ID NO: 89 - DNA, 35 bp, Artificial Sequence
  SYNTHESIZED
aaaccacccgtattgcctattttcgtcccattatt

SEQ ID NO: 90 - DNA, 33 bp, Artificial Sequence
  SYNTHESIZED
ataccggtaacaatacttacaaggcagtgcaaa

SEQ ID NO: 91 - DNA, 33 bp, Artificial Sequence
  SYNTHESIZED
tatttaatccttgtaaaatggggccaatggcga

SEQ ID NO: 92 - DNA, 32 bp, Artificial Sequence
  SYNTHESIZED
ctagtttcagtccctgctactactaccatggc

SEQ ID NO: 93 - DNA, 37 bp, Artificial Sequence; identical to SEQ ID NO: 53
  SYNTHESIZED

SEQ ID NO: 94 - DNA, 31 bp, Artificial Sequence; identical to SEQ ID NO: 54
  SYNTHESIZED

SEQ ID NO: 95 - DNA, 35 bp, Artificial Sequence; identical to SEQ ID NO: 55
  SYNTHESIZED

SEQ ID NO: 96 - DNA, 37 bp, Artificial Sequence; identical to SEQ ID NO: 56
  SYNTHESIZED

SEQ ID NO: 97 - DNA, 36 bp, Artificial Sequence; identical to SEQ ID NO: 65
  SYNTHESIZED

SEQ ID NO: 98 - DNA, 34 bp, Artificial Sequence; identical to SEQ ID NO: 66
  SYNTHESIZED

SEQ ID NO: 99 - DNA, 29 bp, Artificial Sequence
  SYNTHESIZED
tggcaaggctacttttgacacattgagca

SEQ ID NO: 100 - DNA, 31 bp, Artificial Sequence
  SYNTHESIZED
aattagtttccccccaaaagggtcgtcgtaa

SEQ ID NO: 101 - DNA, 37 bp, Artificial Sequence
  SYNTHESIZED
ccttacccctaactaacttactctaacaagaagtccc

SEQ ID NO: 102 - DNA, 31 bp, Artificial Sequence
  SYNTHESIZED
cgctggccatggtaagcaaagagaaataaca

SEQ ID NO: 103 - DNA, 34 bp, Artificial Sequence
  SYNTHESIZED
gataaatggtttaccatgttaggcctaagcacgc

SEQ ID NO: 104 - DNA, 31 bp, Artificial Sequence
  SYNTHESIZED
ctagccaaggtgaatcaaaacctaggggaac

SEQ ID NO: 105 - DNA, 36 bp, Artificial Sequence
  SYNTHESIZED
ctgagtttgctgaatttttagtggaattgggcattg

SEQ ID NO: 106 - DNA, 31 bp, Artificial Sequence
  SYNTHESIZED
cacctctgcaatgcgtagaacagttttcagc

SEQ ID NO: 107 - DNA, 34 bp, Artificial Sequence
  SYNTHESIZED
acatcccgacaatattcgactcaaacaaattcct

SEQ ID NO: 108 - DNA, 29 bp, Artificial Sequence
  SYNTHESIZED
ggtacattcacccctttgttggtcaactg

SEQ ID NO: 109 - DNA, 30 bp, Artificial Sequence
  SYNTHESIZED
tttaatcctctggtttgaagaggtgggcac

SEQ ID NO: 110 - DNA, 32 bp, Artificial Sequence
  SYNTHESIZED
cgtagctccaacggagacctaattattatgcc

SEQ ID NO: 111 - DNA, 27 bp, Artificial Sequence
  SYNTHESIZED
gcaaagggaggttgccaatccaaaacc

SEQ ID NO: 112 - DNA, 27 bp, Artificial Sequence
  SYNTHESIZED
agcaatccggagaagttttggggtgag

SEQ ID NO: 113 - DNA, 33 bp, Artificial Sequence
  SYNTHESIZED
ctgttgacttcattaagttcagcggggattttt

SEQ ID NO: 114 - DNA, 30 bp, Artificial Sequence
  SYNTHESIZED
aatccgtttcaccgatgtgttacccaaaac

SEQ ID NO: 115 - DNA, 25 bp, Artificial Sequence
  SYNTHESIZED
gaggctaaactccgcaacagacgac

SEQ ID NO: 116 - DNA, 35 bp, Artificial Sequence
  SYNTHESIZED
ttggtagtgaaattattattggcgatcgccaatcc

SEQ ID NO: 117 - DNA, 25 bp, Artificial Sequence
  SYNTHESIZED
tttacctagcctaggagccccagtg

SEQ ID NO: 118 - DNA, 27 bp, Artificial Sequence
  SYNTHESIZED
gcacaacccttcagaaataaagcccct

SEQ ID NO: 119 - DNA, 32 bp, Artificial Sequence
  SYNTHESIZED
ctggaaagatcaagcaaactgccgaagattca

SEQ ID NO: 120 - DNA, 28 bp, Artificial Sequence
  SYNTHESIZED
actgcaattgtcccacgaagtccgtcaa

SEQ ID NO: 121 - DNA, 1482 bp, Artificial Sequence
  SYNTHESIZED
agatatcgcgtgcaaggcccagtgatcaatttcattatttttcattatttcatctccattgtccctgaaaatcagttgtgtcgcccctctacacagcccagaactatggtaaaggcgcacgaaaaaccgccaggtaaactcttctcaacccccaaaacgccctctgtttacccatggaaaaaacgacaattacaagaaagtaaaacttatgtcatctataagcttcgtgtatattaacttcctgttacaaagctttacaaaactctcattaatcctttagactaagtttagtcagttccaatctgaactaaggaattataaccaaatggctaccacctctttagcttccgccttttgctcgatgaaagctgtaatgttagctcgtgatggtcggggtatgaaacctcgtagtagtgatttgcaactccgtgcgggaaatgcgcctacctctttgaaaatgatcaatgggaccaaattcagttatacggagagcttgaaacggttgcctgattggagcatgctctttgctgttatcaccaccatcttttcggctgctgagaaacaatggactaatctagagtggaagccgaaaccgaagctaccccagttgcttgatgatcattttggactgcatgggttagttttccggcgcacctttgccatccggtcttatgaagttggacctgatcgctccacctctattctggctgttatgaatcatatgcaggaggctacccttaatcatgcgaaaagtgtgggaattctaggagatggattcgggacgacgctagagatgagtaagcgggatctgatgtgggttgttcggcgcacgcatgttgctgttgaacggtaccctacttggggtgatactgtagaagtagagtgctggattggtgcttctggaaataatggcatgcgtcgtgatttccttgtccgggactgcaaaaccggcgaaattcttactcgctgtaccagcctttcggtgctgatgaatactcgcactcgtcgtttgtccaccattcctgatgaagttcgtggtgaaatagggcctgctttcatcgataatgttgctgtgaaagacgatgaaattaagaaactacaaaaactcaatgatagcactgccgattatattcaaggaggtttgacccctcgttggaatgatttggatgtcaatcaacatgttaacaacctcaaatacgttgcctgggtttttgagaccgtccccgattccatctttgagagtcatcatatttccagcttcactcttgaatatcgtcgtgagtgtacccgtgatagcgtgctgcggtccctgaccactgtctctggtggctcgtcggaggctgggttagtttgcgatcatttgctccaacttgaaggtgggtctgaggtattgcgtgccagaactgagtggcggcctaaacttaccgatagtttccgcggcattagtgttattcccgccgaaccgcgcgtgtaatgatatca

SEQ ID NO: 122 - DNA, 1581 bp, Artificial Sequence
  SYNTHESIZED
agatatcgcgtgcaaggcccagtgatcaatttcattatttttcattatttcatctccattgtccctgaaaatcagttgtgtcgcccctctacacagcccagaactatggtaaaggcgcacgaaaaaccgccaggtaaactcttctcaacccccaaaacgccctctgtttacccatggaaaaaacgacaattacaagaaagtaaaacttatgtcatctataagcttcgtgtatattaacttcctgttacaaagctttacaaaactctcattaatcctttagactaagtttagtcagttccaatctgaactaaggaattataaccaaatggtggctgctgctgctagttccgctttcttccctgttccagcccccggagcctcccctaaacccgggaagttcggaaattggcccagtagcttgagcccttccttcaagcccaagtcaatccccaatggcggatttcaggttaaggctaatgacagcgcccatccaaaagccaatggttctgccgttagtctaaagtctggcagcctcaacactcaagaagacactagttcctcccctcctcctcggactttccttcatcagttgcctgattggagtcgtcttctgactgctattaccaccgtgttcgtgaaatctaagcgtcctgacatgcatgatcggaaatccaagcgtcctgacatgctggtggactcctttgggttggagagtactgttcaggatggcttagtgttccgacagagtttttccattcgttcttatgaaataggcactgatcgaacggcctctatagagacccttatgaaccacttgcaggaaacctctctcaatcattgtaagagtaccggtattctccttgacggcttcggtcgtactcttgagatgtgtaaacgcgacctcatttgggtggtaattaaaatgcagatcaaggtgaatcgctatccagcttggggcgatactgtcgagatcaatacccgtttcagccggttggggaaaattggtatgggtcgcgattggctaattagtgattgcaacaccggagaaattcttgtacgggctacgagcgcgtatgccatgatgaatcaaaagacgcggagactctccaaacttccatacgaggttcaccaggagattgtgcctctttttgtcgactctcctgtcattgaagacagtgatctgaaagtgcataagtttaaagtgaagactggtgacagcattcaaaagggtctaactccggggtggaatgacttggatgtcaatcagcacgtaagcaacgtgaagtacattgggtggattctcgagagtatgccaacagaagttttggagacccaggagctatgctctctcgcccttgaatatcgccgggaatgcggacgcgacagtgtgctggagtccgtgaccgctatggatccctccaaagttggagtccgttctcagtaccagcaccttctgcggcttgaggatgggactgctatcgtgaacggtgctactgagtggcggccgaagaatgcaggagctaacggggcgatcagcacgggaaagacttccaatggaaactcggtctcttaatgatatca

SEQ ID NO: 123 - DNA, 1482 bp, Artificial Sequence
  SYNTHESIZED
agatatcgcgtgcaaggcccagtgatcaatttcattatttttcattatttcatctccattgtccctgaaaatcagttgtgtcgcccctctacacagcccagaactatggtaaaggcgcacgaaaaaccgccaggtaaactcttctcaacccccaaaacgccctctgtttacccatggaaaaaacgacaattacaagaaagtaaaacttatgtcatctataagcttcgtgtatattaacttcctgttacaaagctttacaaaactctcattaatcctttagactaagtttagtcagttccaatctgaactaaggaattataaccaaatgaaaactacttctctcgcctctgccttctgttctatgaaagctgttatgctggcgcgggatggtcgcggtatgaaaccccgttccagtgatctgcaattacgggctggcaacgctcagacctccttgaagatgattaacggcactaaattcagttataccgaatctttgaagaaactccccgattggagcatgttgttcgccgtgattaccaccatttttagtgctgccgaaaaacaatggaccaatctcgaatggaaacccaaacccaaccccccgcagctgctcgatgaccattttggcccccacggcttggtgtttcggcgtaccttcgctatccggtcttatgaagtcggtcccgatcggagcacttccatcgtcgctgttatgaatcacttgcaagaagccgctttgaaccatgctaaatctgttgggattctgggtgatggcttcggtaccactctggagatgagtaagcgcgatctgatctgggtagtaaagcgtactcatgtggccgtggaacgttatccggcctggggtgataccgtagaagtggagtgttgggtaggcgcctccggtaacaacggtcggcgtcacgacttcttggtgcgtgactgtaaaactggcgagatcctgacccgctgtacttccctgagcgttatgatgaacacccggacccgtcgcttatccaagattcccgaagaagttcgcggggaaattggtcctgctttcattgataacgttgctgttaaggatgaggagattaaaaagccgcaaaagctcaatgattctaccgccgattacattcaagggggtctgactccccgttggaatgatctggatattaatcagcatgtgaataacatcaaatatgtggattggattctggagactgtgcccgactctattttcgagtcccaccacattagcagttttaccattgaatatcgtcgcgaatgtactatggacagtgttttgcaatccctgaccaccgtctccggcggttcctctgaagctggcctggtgtgcgaacacctcttgcaactcgaaggcggtagtgaagtgctccgtgccaagaccgaatggcggcccaaattgaccgactcctttcgcgggatttctgtgattcccgccgaatcctccgtctaagatatcat

SEQ ID NO: 124 - DNA, 2077 bp, Artificial Sequence
  SYNTHESIZED
ttcccaatttgaggaggtgtgatggaagtatcccaagatctgtttaatcaattcaatctgttcgctcaatactccgcggctgcttactgtgggaaaaataacgatgctcctgcgggcaccaatattacttgtaccggtaacgcttgccctgaagtggagaaggctgacgccacctttttgtacagcttcgaagatagcggcgtaggcgatgtgactggtttcctcgccttggataataccaataaattgattgttctctctttccgtggcagtgactccattgagaattggattgccaacttgaacttttggttgaagaaaatcaatgatatttgttccggctgtcggggtcacgatggtttcaccagcagctggcgttccgtggccgacaccctccggcaaaaggtggaagatgctgtgcgcgaacatcctgattaccgcgttgtttttaccgggcactccctgggcggggccttggccaccgtagccggtgctgacctgcgcggtaacggctacgatattgatgtgttctcctatggggctccccgggttgggaaccgggcttttgctgagtttttgaccgttcaaaccggcggtaccttgtatcggatcacccataccaatgatattgttccccgcctgccccctcgtgaattcggttatagccactcctcccccgaatactggattaaaagcggcaccttggttcccgtgacccgtaacgacattgtaaaaattgagggcattgacgccaccggtggtaataatcaacctaatatccccgatatcttggctcatctgtggtattttcaagccaccgacgcttgtaacgccggcggtttcagttaatgaggagattaattcaatgaagcctaccgttaaagctgctcccgaggctgttcagaacccggaaaacccgaaaaacaaggacccctttgtgtttgtgcacggctttaccggttttgtgggggaggttgctgcgaaaggtgagaatcactggggcggcaccaaagccaatctgcgcaaccatttgcggaaagctggttacgaaacctacgaagcctccgtatccgccttggcctccaatcacgaacgtgctgtggaactgtactattatctgaaaggtggtcgggtagactatggtgctgcccattccgaaaaatatggccatgagcgttacgggaaaacttatgaaggtgtgctgaaagattggaaacccgggcaccccgtacactttatcggtcattccatgggtggtcagaccattcggctgctggaacattatctgcgctttggtgataaagccgaaattgcctatcaacaacagcacgggggtattattagcgaattatttaagggcggtcaagacaacatggtgacctctatcactactattgccacccctcacaatggtacccatgcttctgacgatattggcaataccccgactatccggaacattctgtatagcttcgcccaaatgtccagtcatctgggcaccatcgactttgggatggaccattggggtttcaagcggaaagatggcgagagtctgaccgattataataagcggattgccgagagcaaaatctgggattctgaagatactgggctgtatgacctgacccgtgaaggcgccgagaaaatcaaccagaaaaccgaattgaatcccaatatctattacaaaacctacactggggtggctacccatgaaactcagttaggcaaacacatcgcggacctcggcatggaatttaccaaaatcctcaccggcaactatatcgggagcgtagacgatattctgtggcggcccaatgatggtttggtgagcgaaatctccagccaacaccccagcgatgagaagaacatttccgtagacgaaaactccgaactgcataagggtacctggcaggtcatgcctaccatgaaagggtgggaccactccgattttattggtaatgacgccctggataccaaacactccgccatcgaactcaccaacttttatcatagcatttctgactacttgatgcggatcgaaaaagccgaatctaccaaaaacgcctaatgatatcga

SEQ ID NO: 125 - DNA, 1359 bp, Artificial Sequence
  SYNTHESIZED
atgaaattgtttgcctggactattggtttactgctgctggccactgtgcgcggcgctgaggtctgttattctcatttgggctgcttttccgacgagaaaccgtgggcgggtacctctcaacggcccatcaagagtttgccgtccgaccctaaaaagattaatacccggttcttgctgtacaccaatgaaaatcaaaattcctaccaactgatcaccgctactgatattgccaccatcaaagccagtaacttcaatctcaaccgcaaaacccgcttcattattcacggtttcaccgacagcggtgagaactcttggctgagtgatatgtgtaaaaacatgttccaagttgaaaaagtgaattgcatttgcgtggattggaaaggcggttccaaggctcaatacagtcaggcttcccagaatattcgggtggtcggtgccgaagttgcctatttagtgcaagtactgagcacctccctgaactatgccccggaaaacgtacatattattggtcactccctcggcgcccacaccgctggggaagccgggaagcggctgaacgggctggtagggcggattaccggcctcgaccccgccgaaccctactttcaagatacccccgaagaagtccggctggatccctctgacgctaaatttgtggacgtgattcacactgatattagccccattctgcctagtctgggtttcggtatgtcccagaaggtcggtcacatggacttcttccccaacggcggcaaagatatgcccgggtgcaaaaccggcatctcctgcaaccaccaccggagtattgaatattatcacagcagtattttgaaccccgaaggttttttaggctacccgtgtgcttcctacgatgaattccaagaatctgggtgcttcccctgtcccgctaaaggttgtcctaaaatgggtcactttgccgaccagtaccccgggaagaccaatgctgtcgaacaaaccttcttcctcaacaccggggcgtccgataatttcacccggtggcgctataaagtaaccgttaccctgagtggcgaaaaagacccgtccggtaacatcaatgtggctttgttaggtaaaaacgggaactctgctcaataccaggttttcaagggcaccctgaagcccgacgcctcctatactaattccattgatgtcgaactcaacgttgggaccattcaaaaagtgaccttcctgtggaaacggagcgggatctccgtctctaaacccaagatgggtgcttctcgcattactgttcaaagtggtaaagacgggaccaaatataacttctgctccagcgatattgtgcaagaaaacgtcgagcagactctgagcccctgctaa